(19) United States
(12) Patent Application Publication
(10) Pub. No.: US 2023/0391770 A1
(43) Pub. Date: Dec. 7, 2023

(54) METTL3 INHIBITORY COMPOUNDS

(71) Applicant: Storm Therapeutics Limited, Cambridge (GB)

(72) Inventors: David James Hardick, Cambridge (GB); Wesley Peter Blackaby, Cambridge (GB); Elizabeth Jane Thomas, Cambridge (GB); Frederick Arthur Brookfield, Abingdon (GB); Jon Shepherd, Abingdon (GB); Christian Bubert, Bubert (GB); Mark Peter Ridgill, Abingdon (GB)

(21) Appl. No.: 18/028,286

(22) PCT Filed: Oct. 5, 2021
(86) PCT No.: PCT/GB2021/052573

§ 371 Date: Mar. 24, 2023

(30) Foreign Application Priority Data
Oct. 6, 2020 (GB) 2015832.5
Oct. 8, 2020 (GB) 2015985.1
Jun. 1, 2021 (GB) 2107783.9

(51) Int. Cl. C07D 471/04; C07D 519/00

(52) U.S. Cl. CPC C07D 471/04; C07D 519/00

(57) ABSTRACT

The present invention relates to compounds of formula (I) that function as inhibitors of METTL3 (N6-adenosine-methyltransferase 70 kDa subunit) enzyme activity: X—Y—Z (I) wherein X, Y and Z are each as defined herein. The present invention also relates to processes for the preparation of these compounds, to pharmaceutical compositions comprising them, and to their use in the treatment of proliferative disorders, such as cancer, and autoimmune diseases, as well as other diseases or conditions in which METTL3 activity is implicated.

### FIELD OF THE INVENTION

The present invention relates to certain compounds that function as inhibitors of METTL3 (N6-adenosine-methyltransferase 70 kDa subunit) activity. The present invention also relates to processes for the preparation of these compounds, to pharmaceutical compositions comprising them, and to their use in the treatment of proliferative disorders, such as cancer, autoimmune, neurological, infectious and inflammatory diseases, as well as other diseases or conditions in which METTL3 activity is implicated.

### BACKGROUND OF THE INVENTION

N6-methyladenosine (m6A) is the most common and abundant covalent modification of messenger RNA, modulated by ‘writers’, ‘erasers’ and ‘readers’ of this mark (Meyer & Jaffrey 2014, Niu Y et al, 2013, Yue et al 2015). Approximately 0.1 to 0.5% of all mRNA adenosines are m6A modified (Li Y et al 2015). In vitro data have shown that m6A influences fundamental aspects of mRNA biology, mainly mRNA expression, splicing, stability, localisation and translation (Meyer et al, 2015; Sledz & Jinek 2016). M6A modifications are tissue specific and there is significant variability in their occurrence profiles in non-diseased tissues (eg brain, heart, kidney) and diseased tissues and cells (lung, renal, breast, and leukeamic cancer cells) (Meyer et al 2012).

The m6A modifications and its erasers and writers such as FTO, ALKBH5, methyltransferase like 3 (METTL3) and METTL14 are associated with major diseases such as solid organ cancers, leukaemia, type 2 diabetes, neuropsychiatric behavioural and depressive disorders (Chandola et al 2015; Koranda et al 2018).

The RNA methyltransferase, METTL3, is the major, but not the sole enzyme, that catalyses m6A modification of RNA. It exists as a hetero-trimeric complex with METTL14 (Liu et al 2014, Wang et al 2016) and Wilm's Tumour Associated Protein (WTAP) (Ping et al 2014). Catalytic activity resides in METTL3, which transfers a methyl group from the co-factor S-adenosyl methionine to the substrate RNA and METTL14 facilitates substrate RNA binding. WTAP localises the complex in specific nuclear regions and also localises RNA substrates to the complex (Wang X et al 2016).

METTL3 has been reported to play a role in many aspects of the development of cancer (Fry et al 2018). Genetic knockdown of METTL3 in lung cancer cell lines (A549, H1299 and H1792) and HeLa cells leads to decreased growth, survival and invasion of human lung cancer cells (Lin S et al 2016). METTL3 is significantly up-regulated in human bladder cancer (Cheng et al 2019). Knockdown of METTL3 drastically reduced bladder cancer cell proliferation, invasion, and survival in vitro and tumorigenicity in vivo. AF4/FMR2 family member 4 (AFF4), two key regulators of NF-κB pathway (IKBKB and RELA) and MYC were further identified as direct targets of METTL3-mediated m6A modification. In renal carcinoma cell lines (CAK-1, CAK-2 and ACHN), genetic knockdown reduced cell proliferation via the phosphatidinylinositol 3-kinase (PI3K)/AKT/mammalian target of rapamycin (mTOR) signalling pathway (Li X et al 2017).

Recently Barbieri et al (2017), defined a set of RNA-modifying enzymes that are necessary for AML leukaemia and identified a key leukemic pathway for the METTL3 RNA methyltransferase. In this pathway, METTL3 is stably recruited by the CCAAT-box binding transcription factor CEBPZ to promoters of a specific set of active genes, resulting in m6A methylation of the respective mRNAs and increased translation. One important target is SP1, an oncogene in several cancers, which regulates c-MYC expression. Consistent with these findings, it has been reported that METTL3 can methylate its targets co-transcriptionally.

The pathway described by Barbieri et al., is critical for AML leukaemia, as three of its components are required for AML cell growth: (i) the m6A RNA methyltransferase METTL3; (ii) the transcription factor CEBPZ, which targets this enzyme to promoters; and (iii) SP1, whose translation is dependent upon the m6A modification by METTL3. Together, the observations of Barbieri et al define METTL3 enzymatic activity as a new candidate target for the treatment of AML.

In separate, independent studies it has been reported that METTL3 plays an essential role in controlling myeloid differentiation of mammalian normal hematopoietic and leukemic cells (Vu et al 2017). Forced expression of wild type METTL3, but not a mutant METTL3 (with defect in catalytic activity), significantly promotes cell proliferation and inhibits cell differentiation of human cord blood-derived CD34+ haematopoietic stem/progenitor cells (HSPCs). Genetic knockdown of METTL3 has the opposite effects. METTL3 is highly expressed in AML compared to normal HSPCs or other types of cancers. Knockdown of METTL3 in human AML cell lines significantly induces cell differentiation and apoptosis and inhibits leukemia progression in mice xeno-transplanted with MOLM-13 AML cells. The biological function of METTL3 is likely attributed to the promotion of translation of its mRNA targets such as MYC, BCL-2, and PTEN in an m6A-dependent manner.

Recently, METTL3 mediated m6A modification has been demonstrated to play an important role in T cell homeostasis and signal dependent induction of mRNA degradation in CD4 positive T cell lineages (Li et al 2017). Deletion of METTL3 in mouse T cells disrupts T cell homeostasis and differentiation. In a lymphopenic mouse adoptive transfer model, naive Mett/3-deficient T cells failed to undergo homeostatic expansion and remained in the naive state for up to 12 weeks, thereby preventing colitis. Consistent with these observations, the mRNAs of SOCS family genes encoding the STAT signalling inhibitory proteins SOCS1, SOCS3 and CISH were marked by m6A, exhibited slower mRNA decay and showed increased mRNAs and levels of protein expression in Mett/3-deficient naive T cells. This increased SOCS family activity consequently inhibited IL-7-mediated STAT5 activation and T cell homeostatic proliferation and differentiation. Thus METTL3 mediated m6A methylation has important roles for inducible degradation of Socs mRNAs in response to IL-7 signalling in order to reprogram naive T cells for proliferation and differentiation, pointing to a role in auto-immunity.

Recent studies have revealed that depletion of METTL3 leads to alterations in the propagation of diverse viruses (Winkler et al). Following viral infection or stimulation of cells with an inactivated virus, deletion of the m6A ‘writer’ METTL3 led to an increase in the induction of interferon-stimulated genes. Consequently, propagation of different viruses was suppressed in an interferon-signaling-dependent manner. Significantly, the mRNA of IFNB, was m6A modified and was stabilized following repression of METTL3. m6A serves as a negative regulator of interferon response by dictating the fast turnover of interferon mRNAs and consequently facilitating viral propagation.

METTL3-dependent m6A on HBV and HCV viral genome regulates recognition of the viral genome by RIG-1 RNA sensor. Depletion of METTL3 enhances viral dsRNA recognition and induces an anti-viral immune response (Kim et al.).

Therefore METTL3 inhibitors may provide a novel therapeutic approach to a range of infectious and inflammatory diseases. In particular, they provide a potential treatments for viral diseases (e.g. DNA and RNA viruses).

Furthermore, METTL3-dependent m6A on endogenous mRNAs regulates recognition of by MAVS-dependent RNA sensors. Depletion of METTL3 enhances endogenous dsRNA recognition and induces an auto-immune response (Gao et al.). This implies that an anti-tumour immune response might be enhanced by METTL3 inhibition.

Thus, METTL3 inhibitors may also provide a novel therapeutic approach to enhance an anti-tumour immune response.

### REFERENCES

Barbieri I, Tzelepis K, Pandolfini L, Shi J, Millán-Zambrano G, Robson S C, Aspris D, Migliori V, Bannister A J, Han N, De Braekeleer E, Ponstingl H, Hendrick A, Vakoc C R, Vassiliou G S, Kouzarides T. Nature. 2017 Dec. 7; 552(7683):126-131.
Chandola U, Das R, Panda B. Brief Funct Genomics. 2015 May; 14(3):169-79.
Cheng M, Gao Q, Wu M, Liang Y, Zhu F, Zhang Y, Zhang X, Li Y, Sheng L, Zhang H, Xiong Q, Yuan Q, Oncogene (2019; e-publication ahead of print).
Fry N J, Law B A, Ilkayeva O R, Carraway K R, Holley C L, Mansfield K D. Oncotarget. 2018 Jul. 27; 9(58):31231-31243.
Koranda J L, Dore L, Shi H, Patel M J, Vaasjo L O, Rao M N, Chen K, Lu Z, Yi Y, Chi W, He C, Zhuang X. Neuron. 2018 Jul. 25; 99(2): 283-292.
Li H B, Tong J, Zhu S, Batista P J, Duffy E E, Zhao J, Bailis W, Cao G, Kroehling L, Chen Y, Wang G, Broughton J P, Chen Y G, Kluger Y, Simon M D, Chang H Y, Yin Z, Flavell R A. Nature. 2017 Aug. 17; 548 (7667):338-342
Li X, Tang J, Huang W, Wang F, Li P, Qin C, Qin Z, Zou Q, Wei J, Hua L, Yang H, Wang Z. Oncotarget. 2017 Oct. 10; 8(56):96103-96116.
Li Y, Wang Y, Zhang Z, Zamudio A V, Zhao J C. RNA. 2015 August; 21(8):1511-8.
Lin S, Choe J, Du P, Triboulet R, Gregory R I. Mol Cell. 2016 May 5; 62(3):335-345.
Liu J, Yue Y, Han D, Wang X, Fu Y, Zhang L, Jia G, Yu M, Lu Z, Deng X, Dai Q, Chen W, He C. Nat Chem Biol. 2014 February; 10(2):93-5.
Meyer K D, Patil D P, Zhou J, Zinoviev A, Skabkin M A, Elemento O, Pestova T A, Qian S B, Jaffrey S R. Cell. 2015 Nov. 5; 163(4): 999-1010.
Meyer K D, Jaffrey S R. Nat Rev Mol Cell Biol. 2014 May; 15(5):313-26.
Meyer K D, Saletore Y, Zumbo P, Elemento O, Mason C E, Jaffrey S R. Cell. 2012 Jun. 22; 149(7):1635-46.
Niu Y, Zhao X, Wu Y S, Li M M, Wang X J, Yang Y G. Genomics Proteomics Bioinformatics. 2013 February; 11(1):8-17.
Ping X L, Sun B F, Wang L, Xiao W, Yang X, Wang W J, Adhikari S, Shi Y, Lv Y, Chen Y S, Zhao X, Li A, Yang Y, Dahal U, Lou X M, Liu X, Huang J, Yuan W P, Zhu X F, Cheng T, Zhao Y L, Wang X, Rendtlew Danielsen J M, Liu F, Yang Y G. Cell Res. 2014 February; 24(2):177-89.
Śledź P, Jinek M. Elife. 2016 Sep. 14; 5.
Vu L P, Pickering B F, Cheng Y, Zaccara S, Nguyen D, Minuesa G, Chou T, Chow A, Saletore Y, MacKay M, Schulman J, Famulare C, Patel M, Klimek V M, Garrett-Bakelman F E, Melnick A, Carroll M, Mason C E, Jaffrey S R, Kharas M G. Nat Med. 2017 November; 23(11):1369-1376.
Wang X, Feng J, Xue Y, Guan Z, Zhang D, Liu Z, Gong Z, Wang Q, Huang J, Tang C, Zou T, Yin P. Nature. 2016 Jun. 23; 534(7608):575-8
Wang P, Doxtader K A, Nam Y. Mol Cell. 2016 Jul. 21; 63(2):306-317.
Winkler R, Gillis E, Lasman L, Safra M, Geula S, Soyris C, Nachshon A, Tai-Schmiedel J, Friedman N, Le-Trilling Vu T K, Trilling M, Mandelboim M, Hanna, J H, Schwartz S, Stern-Ginossar N. Nature Immunology (2018, e-publication ahead of print).
Yue Y, Liu J, He C. Genes Dev. 2015 Jul. 1; 29(13):1343-55.
Geon-Woo Kim, Hasan Imam, Mohsin Khan, Aleem Siddiqui, J Biol Chem, (27 Jul. 2020; online publication ahead of print).
Yimeng Gao, Radovan Vasic, Yuanbin Song, Rhea Teng, Chengyang Liu, Rana Gbyli, Giulia Biancon, Raman Nelakanti, Kirsten Lobben, Eriko Kudo, Wei Liu, Anastasia Ardasheva, Xiaoying Fu, Xiaman Wang, Poorval Joshi, Veronica Lee, Burak Dura, Gabriella Viero, Akiko Iwasaki, Rong Fan, Andrew Xiao, Richard A Flavell, Hua-Bing Li, Toma Tebaldi, Stephanie Halene; Immunity (16 Jun. 2020; Volume 52; 6; p 887-1132).
Rosa M Rubio, Daniel P Depledge, Christopher Bianco, Letitia Thompson, Ian Mohr; Genes Dev. 2018 Dec. 1; 32(23-24):1472-1484.

An object of this invention is to provide inhibitors of METTL3 activity.

### SUMMARY OF THE INVENTION

In one aspect, the present invention provides a compound as defined herein, or a pharmaceutically acceptable salt thereof.

In another aspect, the present invention provides a pharmaceutical composition as defined herein which comprises a compound as defined herein, or a pharmaceutically acceptable salt thereof, and one or more pharmaceutically acceptable excipients.

In another aspect, the present invention provides a compound as defined herein, or a pharmaceutically acceptable salt thereof, or a pharmaceutical composition as defined herein, for use in therapy.

In another aspect, the present invention provides a compound as defined herein, or a pharmaceutically acceptable salt thereof, or a pharmaceutical composition as defined herein, for use in the treatment of a proliferative condition.

In another aspect, the present invention provides a compound as defined herein, or a pharmaceutically acceptable salt thereof, or a pharmaceutical composition as defined herein, for use in the treatment of cancer. In a particular embodiment, the cancer is a human cancer.

In another aspect, the present invention provides a compound as defined herein, or a pharmaceutically acceptable salt thereof, or a pharmaceutical composition as defined herein, for use in the inhibition of METTL3 activity.

In another aspect, the present invention provides a compound as defined herein, or a pharmaceutically acceptable salt thereof, or a pharmaceutical composition as defined herein, for use in promoting an immune response (e.g. anti-viral or anti-tumour immune response).

In another aspect, the present invention provides a compound as defined herein, or a pharmaceutically acceptable salt thereof, or a pharmaceutical composition as defined herein, for use in increasing an innate immune response in a subject.

In another aspect, the present invention provides a compound as defined herein, or a pharmaceutically acceptable salt thereof, or a pharmaceutical composition as defined herein, for use in increasing or enhancing an anti-tumour immune response during immune-oncology therapy.

In another aspect, the present invention provides a compound as defined herein, or a pharmaceutically acceptable salt thereof, or a pharmaceutical composition as defined herein, for use in the treatment of an autoimmune disease.

In another aspect, the present invention provides a compound as defined herein, or a pharmaceutically acceptable salt thereof, or a pharmaceutical composition as defined herein, for use in the treatment of a neurological disease.

In another aspect, the present invention provides a compound as defined herein, or a pharmaceutically acceptable salt thereof, or a pharmaceutical composition as defined herein, for use in the treatment of an infectious disease.

In another aspect, the present invention provides a compound as defined herein, or a pharmaceutically acceptable salt thereof, or a pharmaceutical composition as defined herein, for use in the treatment of a viral infection. Suitably, the viral infection is a RNA viral infection. Suitably, the viral infection is human papillomavirus (HPV) or hepatitis.

In another aspect, the present invention provides a compound as defined herein, or a pharmaceutically acceptable salt thereof, or a pharmaceutical composition as defined herein, for use in the treatment of an inflammatory disease.

In another aspect, the present invention provides the use of a compound as defined herein, or a pharmaceutically acceptable salt thereof, in the manufacture of a medicament for use in the treatment of a proliferative condition.

In another aspect, the present invention provides the use of a compound as defined herein, or a pharmaceutically acceptable salt thereof, in the manufacture of a medicament for use in the treatment of cancer. In a particular embodiment, the medicament is for use in the treatment of human cancers.

In another aspect, the present invention provides the use of a compound as defined herein, or a pharmaceutically acceptable salt thereof, in the manufacture of a medicament for the inhibition of METTL3 activity.

In another aspect, the present invention provides the use of a compound as defined herein, or a pharmaceutically acceptable salt thereof, in the manufacture of a medicament for promoting an immune response (e.g. anti-viral or anti-tumour immune response).

In another aspect, the present invention provides the use of a compound as defined herein, or a pharmaceutically acceptable salt thereof, in the manufacture of a medicament for for use in increasing an innate immune response in a subject.

In another aspect, the present invention provides the use of a compound as defined herein, or a pharmaceutically acceptable salt thereof, in the manufacture of a medicament for use in increasing or enhancing an anti-tumour immune response during immune-oncology therapy.

In another aspect, the present invention provides the use of a compound as defined herein, or a pharmaceutically acceptable salt thereof, in the manufacture of a medicament for use in the treatment of an autoimmune disease.

In another aspect, the present invention provides the use of a compound as defined herein, or a pharmaceutically acceptable salt thereof, in the manufacture of a medicament for use in the treatment of a neurological disease.

In another aspect, the present invention provides the use of a compound as defined herein, or a pharmaceutically acceptable salt thereof, in the manufacture of a medicament for use in the treatment of an infectious disease.

In another aspect, the present invention provides the use of a compound as defined herein, or a pharmaceutically acceptable salt thereof, in the manufacture of a medicament for use in the treatment of a viral infection. Suitably, the viral infection is a RNA viral infection.

Suitably, the viral infection is human papillomavirus (HPV) or hepatitis.

In another aspect, the present invention provides the use of a compound as defined herein, or a pharmaceutically acceptable salt thereof, in the manufacture of a medicament for use in the treatment of an inflammatory disease.

In another aspect, the present invention provides a method of inhibiting METTL3 activity in vitro or in vivo, said method comprising contacting a cell with an effective amount of a compound as defined herein, or a pharmaceutically acceptable salt thereof, or a pharmaceutical composition as defined herein.

In another aspect, the present invention provides a method of inhibiting cell proliferation in vitro or in vivo, said method comprising contacting a cell with an effective amount of a compound as defined herein, or a pharmaceutically acceptable salt, or a pharmaceutical composition as defined herein.

In another aspect, the present invention provides a method of inhibiting metastasis in vitro or in vivo, said method comprising contacting a cell with an effective amount of a compound as defined herein, or a pharmaceutically acceptable salt, or a pharmaceutical composition as defined herein.

In another aspect, the present invention provides a method of promoting an immune response (e.g. anti-viral or anti-tumour immune response) in a subject in need thereof, said method comprising administering to the subject a therapeutically effective amount of a compound as defined herein, or a pharmaceutically acceptable salt, or a pharmaceutical composition as defined herein.

In another aspect, the present invention provides a method of increasing an innate immune response in a subject in need thereof, said method comprising administering to the subject a therapeutically effective amount of a compound as defined herein, or a pharmaceutically acceptable salt, or a pharmaceutical composition as defined herein.

In another aspect, the present invention provides a method of increasing or enhancing an anti-tumour immune response during immune-oncology therapy, said method comprising administering to a subject in need thereof a therapeutically effective amount of a compound as defined herein, or a pharmaceutically acceptable salt, or a pharmaceutical composition as defined herein.

In another aspect, the present invention provides a method of treating a proliferative disorder, said method comprising administering to a subject in need thereof a therapeutically effective amount of a compound as defined herein, or a pharmaceutically acceptable salt, or a pharmaceutical composition as defined herein.

In another aspect, the present invention provides a method of treating cancer, said method comprising administering to a subject in need thereof a therapeutically effective amount of a compound as defined herein, or a pharmaceutically acceptable salt, or a pharmaceutical composition as defined herein.

In another aspect, the present invention provides a method of treating an autoimmune disease, said method comprising administering to a subject in need thereof a therapeutically effective amount of a compound as defined herein, or a pharmaceutically acceptable salt, or a pharmaceutical composition as defined herein.

In another aspect, the present invention provides a method of treating a neurological disease, said method comprising administering to a subject in need thereof a therapeutically effective amount of a compound as defined herein, or a pharmaceutically acceptable salt, or a pharmaceutical composition as defined herein.

In another aspect, the present invention provides a method of treating an infectious disease, said method comprising administering to a subject in need thereof a therapeutically effective amount of a compound as defined herein, or a pharmaceutically acceptable salt, or a pharmaceutical composition as defined herein.

In another aspect, the present invention provides a method of treating a viral infection, said method comprising administering to a subject in need thereof a therapeutically effective amount of a compound as defined herein, or a pharmaceutically acceptable salt, or a pharmaceutical composition as defined herein. Suitably, the viral infection is a RNA viral infection. Suitably, the viral infection is human papillomavirus (HPV) or hepatitis.

In another aspect, the present invention provides a method of treating an inflammatory disease, said method comprising administering to a subject in need thereof a therapeutically effective amount of a compound as defined herein, or a pharmaceutically acceptable salt, or a pharmaceutical composition as defined herein.

In one aspect, the present invention provides a combination comprising a compound as defined herein, or a pharmaceutically acceptable salt thereof, with one or more additional therapeutic agents.

The present invention further provides a method of synthesising a compound, or a pharmaceutically acceptable salt, as defined herein.

In another aspect, the present invention provides a compound as defined herein, or a pharmaceutically acceptable salt, obtainable by, or obtained by, or directly obtained by a method of synthesis as defined herein.

In another aspect, the present invention provides novel intermediates as defined herein which are suitable for use in any one of the synthetic methods as set out herein.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “C₁₋₆ alkyl” includes C₁₋₄ alkyl, C₁₋₃ alkyl, propyl, isopropyl and t-butyl. A similar convention applies to other radicals, for example “phenyl(C₁₋₆ alkyl)” includes phenyl(C₁₋₄ alkyl), benzyl, 1-phenylethyl and 2-phenylethyl.

The term “(m-nC)” or “Cm-n”, or “(m-nC) group” or “Cm-n” used alone or as a prefix, refers to any group having m to n carbon atoms.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

An “alkylene” group is an alkyl group that is positioned between and serves to connect two other chemical groups. Thus, “C₁₋₃ alkylene” means a linear saturated divalent hydrocarbon radical of one to three carbon atoms or a branched saturated divalent hydrocarbon radical of three atoms, for example, methylene, ethylene, propylene, and the like.

The term “Cₘ₋ₙ cycloalkyl” means a hydrocarbon ring containing from m to n carbon atoms, for example “C₃₋₆ cycloalkyl” means a hydrocarbon ring containing from 3 to 6 carbon atoms, for example, cyclopropyl, cyclobutyl, cyclopentyl, cyclohexyl. The term “Cₘ₋ₙ cycloalkyl” also encompasses non-aromatic saturated or partially saturated monocyclic, fused, bridged, or spiro bicyclic carbocyclic ring system(s). The term “Cₘ₋ₙ cycloalkyl” includes both monovalent species and divalent species. Monocyclic “Cₘ₋ₙ cycloalkyl” rings contain from about 3 to 12 (suitably from 3 to 8, most suitably from 5 to 6) ring carbon atoms. Bicyclic “Cₘ₋ₙ cycloalkyl” contain from 7 to 17 ring carbon atoms, suitably 7 to 12 ring carbon atoms. Bicyclic “Cₘ₋ₙ cycloalkyl” rings may be fused, spiro (e.g. spiro[3,3]heptane), or bridged ring systems (e.g. bicyclo[2.2.1]hept-2-ene and bicyclo[1.1.1]pentanyl).

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7, most suitably from 5 to 6) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, quinuclidine, 6-azabicyclo[3.1.1]heptane, 8-azabicyclo[3.2.1]octane, bicyclo[3.2.1]octane, 7-oxabicyclo[2.2.1]hept-2-ene and 3-oxa-8-azabicyclo[3.2.1]octane.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzoisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzothienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to compounds of the formula (I), or a pharmaceutically acceptable salt thereof,

X—Y—Z (I)

wherein:
X is selected from:

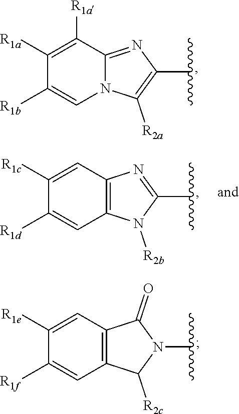

wherein:
R₁ₐ, R_{1b}, R_{1c}, R_{1d}, R₁ₑ and R_{1f} are independently selected from hydrogen, cyano, halo or a group of the formula:

-L₁ₐ-L_{1b}-Q₁

wherein
L₁ₐ is absent or selected from C₁₋₃ alkylene and C₃₋₅ cycloalkylene, wherein C₁₋₃ alkylene and C₃₋₅ cycloalkylene are optionally substituted by one or more substituents selected from aryl, aryl-(1-2C)alkyl, heteroaryl, aryl-(1-2C)alkyl, C₁₋₃ alkyl, cyano, C₁₋₃ alkoxy, halo, hydroxy, C₁₋₃ haloalkoxy, —O—C₃₋₄ cycloalkyl, NH₂ or oxo; wherein any —O—C₃₋₆ cycloalkyl aryl, aryl-(1-2C)alkyl, heteroaryl, aryl-(1-2C)alkyl or C₁₋₃ alkyl is optionally further substituted by one or more substituents selected from cyano, hydroxy, C₁₋₃ alkoxy, halo, C₁₋₃ haloalkoxy, —O—C₃₋₄ cycloalkyl, or NH₂; wherein —O—C₃₋₆ cycloalkyl is optionally further substituted with halo, cyano or hydroxy; or C₁₋₃ alkylene is optionally spiro-fused to a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo or C₁₋₂ haloalkoxy;
L_{1b} is absent or selected from O, S, SO, SO₂, N(Rᵣ), C(O), C(O)O, OC(O), C(O)N(Rᵣ), N(Rᵣ)C(O), N(Rᵣ)C(O)N(Rₛ), S(O)₂N(Rᵣ), or N(Rᵣ)SO₂, wherein Rᵣ and Rₛ are each independently selected from hydrogen or C₁₋₃ alkyl, wherein C₁₋₃ alkyl is optionally further substituted by cyano, hydroxy, C₁₋₂ alkoxy, halo, C₁₋₂ haloalkoxy, NH₂, C₃₋₆-cycloalkyl or a 3 to 6 membered heterocyclyl, wherein the C₃₋₆ cycloalkyl or a 3 to 6 membered heterocyclyl in turn are optionally further substituted by halo, hydroxy, C₁₋₂ alkoxy or C₁₋₂ haloalkoxy; and
Q₁ is hydrogen, cyano, C₁₋₆ alkyl, C₃₋₈ cycloalkyl (including a spirocyclic carbocyclic and a bridged C₃₋₈ cycloalkyl), C₂₋₃ alkenyl, C₂₋₃ alkynyl, aryl, heterocyclyl (including a mono- or bicyclic-heterocyclic ring system, a spirocyclic heterocyclic ring system, or a bridged heterocyclic ring system) or heteroaryl; and wherein Q₁ is optionally substituted by one or more substituents selected from C₁₋₄ alkyl, halo, trifluoromethyl, trifluoromethoxy, amino, oxo, cyano, hydroxy, carboxy, carbamoyl, sulphamoyl, NRₜRᵤ, ORₜ, C(O)Rₜ, C(O)ORₜ, OC(O)Rₜ, C(O)N(Rₜ)Rᵤ, N(Rₜ)C(O)Rᵤ, —S(O)₀₋₂RₜRᵤ, S(O)_{y}Rₜ (where y is 0, 1 or 2), SO₂N(Rₜ)Rᵤ, N(Rₜ)SO₂Rᵤ, or (CH₂)_{z}NRₜRᵤ (where z is 1, 2 or 3), wherein C₁₋₄ alkyl is in turn optionally substituted by one more substituents selected from cyano, hydroxy, C₁₋₂ alkoxy, halo, C₁₋₂ haloalkoxy, —O—C₃ cycloalkyl, wherein —O—C₃ cycloalkyl is optionally substituted with halo, cyano or hydroxy; and wherein Rₜ and Rₐ are each independently selected from hydrogen or C₁₋₄ alkyl; or
Q₁ is optionally substituted by one or more groups of the formula:

-L_{1c}-L_{1d}-Z₁

wherein
L_{1c} is absent or C₁₋₃ alkylene optionally substituted by C₁₋₂ alkyl or oxo;
L_{1d} is absent or selected from C(O), O, C(O)O, OC(O), C(O)N(Rᵥ), N(Rᵥ)C(O), N(Rᵥ)C(O)N(R_{w}), S(O)₂N(Rᵥ), or N(Rᵥ)SO₂, wherein Rᵥ and R_{w} are each independently selected from hydrogen or C₁₋₂ alkyl; and
Z₁ is C₃₋₈ cycloalkyl (including a spirocyclic carbocyclic and a bridged C₃₋₈ cycloalkyl), heterocyclyl (including a mono- or bicyclic-heterocyclic ring system, a spirocyclic heterocyclic ring system, or a bridged heterocyclic ring system), aryl or heteroaryl, wherein Z₁ is optionally substituted by one or more substituents selected from C₁₋₄ alkyl, C₃₋₆ cycloalkyl, heterocyclyl, halo, C₁₋₄ haloalkyl, C₁₋₄ haloalkoxy, C₁₋₄ alkoxy, cyano, hydroxyl, NRₜ₁Rᵤ₁, ORₜ₁, C(O)Rₜ₁, C(O)ORₜ₁, OC(O)Rₜ₁, C(O)N(Rₜ₁)Rᵤ₁, N(Rₜ₁)C(O)Rᵤ₁, —S(O)₀₋₂Rₜ₁Rᵤ₁, S(O)_{y}Rₜ₁ (where y is 0, 1 or 2), SO₂N(Rₜ₁)Rᵤ₁, N(Rₜ₁)SO₂Rᵤ₁ or (CH₂)_{z}NRₜ₁Rᵤ₁ (where z is 1, 2 or 3), wherein Rₜ₁ and Rᵤ₁ are each independently selected from hydrogen or C₁₋₄ alkyl; and when Z₁ is C₃₋₈ cycloalkyl or heterocyclyl, Z₁ is optionally spiro-fused to a C₃₋₆ cycloalkyl or heterocyclyl ring;
R_{1a′} is selected from hydrogen, halo and methyl;
R₂ₐ, R_{2b} and R_{2c} are selected from hydrogen, halo or a group of the formula:

-L₂ₐ-L_{2b}-Q₂

wherein
L₂ₐ is absent or C₁₋₃ alkylene optionally substituted by C₁₋₂ alkyl or oxo;
L_{2b} is absent or selected from O, S, SO, SO₂, N(Rₙ), C(O), C(O)O, OC(O), C(O)N(Rₙ), N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂ alkyl; and
Q₂ is hydrogen, cyano, C₁₋₆ alkyl, C₃₋₆ cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄ alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)_{z}NRₚR_{q} (where z is 1, 2 or 3), wherein Rₚ and R_{q} are each independently selected from hydrogen or C₁₋₄ alkyl;
   Y is selected from:

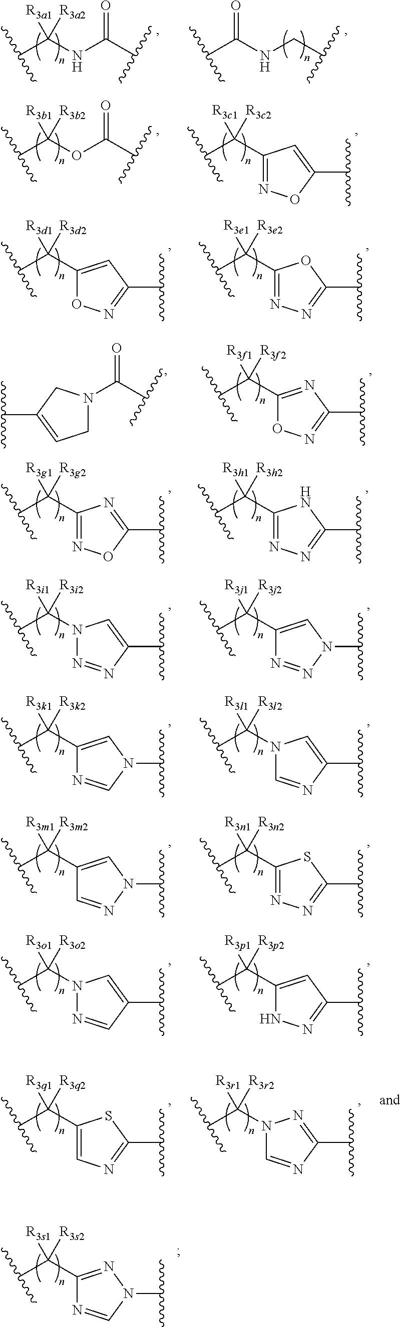

wherein:
R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen (including deuterium), C₁₋₆ alkyl, C₃₋₄ cycloalkyl, hydroxy, and halo; and wherein C₁₋₆ alkyl, or C₃₋₄ cycloalkyl is optionally substituted with one or more substituents selected from halo, amino, cyano, and hydroxy;
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen or halo;
with the proviso that R₃ₐ₁, R_{3b1}, R₃ᵢ₁, R₃ₗ₁, R₃ₒ₁, R₃ᵣ₁, R₃ₐ₂, R_{3b2}, R₃ᵢ₂, R₃ₗ₂, R₃ₒ₂ and R₃ₛ₁ cannot be halo when n=1 or when n=2 and the carbon atom to which they are attached is linked to an oxygen or nitrogen atom;
or R₃ₐ₁ and R₃ₐ₂, R_{3b1} and R_{3b2}, R_{3c1} and R_{3c2}, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, or R₃ᵣ₁ and R₃ᵣ₂ or R₃ₛ₁ and R₃ₛ₂ may be linked such that, together with the carbon atom to which they are attached, they form a spiro-fused C₃₋₄ cycloalkyl which is optionally substituted with one or more substituents selected from halo, methyl, amino, cyano, and hydroxy;
n is 0, 1 or 2; and
   Z is selected from one of the following structures:
i)

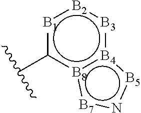

wherein:
B₁ is A₅, wherein A₅ is selected from CR₁₆ and N, wherein R₁₆ is selected from hydrogen, halo, cyano, C₁₋₄ alkyl, C₂₋₄ alkenyl, C₂₋₄ alkynyl, a 5- or 6-membered heteroaryl, C₁₋₄ alkoxy, C₁₋₄ haloalkyl, C₁₋₄ haloalkoxy, C₃₋₄ cycloalkyl, a 3- to 4-membered heterocyclyl and C₃₋₄ cycloalkoxy;
B₂ is A₆, wherein A₆ is selected from N or CR₁₇, wherein R₁₇, R_{H2}, R_{H4} and R_{H5} are selected from hydrogen, hydroxy, halo, cyano, C₁₋₅ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—NR_{q}Rᵣ, —(OCH₂CH₂)ₘ—OCH₃ wherein m is an integer from 1 to 6, NR_{q}Rᵣ, —C(O)—NR_{q}Rᵣ, —C(O)OR_{q},
wherein R_{q} and Rᵣ are each independently hydrogen, C₁₋₅ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring;
wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
B₃ is N or CR_{Z1}, wherein R_{Z1} is selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
B₄ is selected from C or N;
B₅ is selected from CR_{Zi1b} or NR_{B5N}, wherein:
R_{Zi1b} is selected from hydrogen, C₁₋₄ alkyl, cyano, halo, NH₂ and C₁₋₄ alkoxy; and
R_{B5N} is selected from hydrogen or C₁₋₄ alkyl;
B₇ is N, NR_{Z2N} or CR_{Z2}, wherein R_{Z2} is selected from hydrogen, C₁₋₄ alkyl, cyano, halo, NH₂ and C₁₋₄ alkoxy; and R_{Z2N} is selected from hydrogen or C₁₋₄ alkyl;
B₈ is selected from C or N;
with the proviso that no more than four of B₁ to B₈ are N.
   ii)

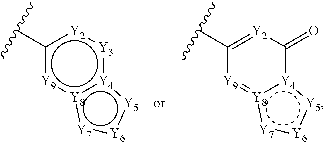

Y₂ is A₇, wherein A₇ is selected from CR₁₈ and N; wherein R₁₈ is selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄ cycloalkoxy;
Y₃ is N or CR_{z1a} wherein R_{Z1a}, is selected from hydrogen, hydroxy, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
Y₄ is C or N
Y₅ is C—R_{Y5} or NR_{Y5N}, wherein:
R_{Y5} is selected from hydrogen, C₁₋₄ alkyl, cyano, halo, NH₂ and C₁₋₄ alkoxy;
R_{Y5N} is selected from hydrogen or C₁₋₄ alkyl;
Y₆ is C—R_{Zi2e} or N, wherein R_{Zi2e} is selected from hydrogen, C₁₋₄ alkyl, cyano, halo, NH₂ and C₁₋₄ alkoxy
Y₇ is O, S, CR_{Z2a} or N, wherein R_{Z2a} is selected from hydrogen, C₁₋₄ alkyl, cyano, halo, NH₂ and C₁₋₄ alkoxy;
Y₈ Is C or N;
Y₉ is CR_{Z3a} or N; wherein
R_{Z3a} is selected from hydrogen, C₁₋₄ alkyl, cyano, halo, NH₂ and C₁₋₄ alkoxy;
with the proviso that no more than four of Y₁ to Y₈ are N.
   (iii)

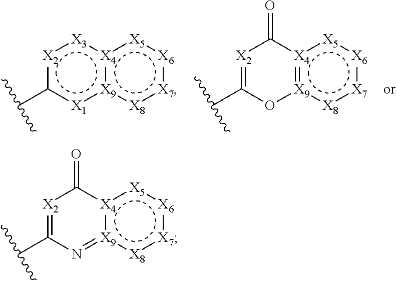

X₁ is N or C—R_{Z9}, wherein R_{Z9} is selected from hydrogen, halo, cyano, C₁₋₄ alkyl, C₁₋₄ haloalkyl, C₁₋₄ alkoxy, C₁₋₄ haloalkoxy;
X₂ is selected from N or CR₄ wherein:
   R₄ is selected from hydrogen, halo, cyano, C1-4 alkyl, C1-4 haloalkyl, C1-4 alkoxy, C1-4 haloalkoxy (e.g. hydrogen, halo, cyano and methyl);
X₃ is N;
X₄ is N or C;
X₅ is selected from N, CR₅ and CRx₅ₐR_{X5b} wherein:
R₅ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
Rx₅ₐ and R_{X5b} are independently selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
either:
X₆ is A₁ and X₇ is A₂; or
X₆ is A₈ and X₇ is A₉ or A₁₁, wherein:
   A₁ is selected from CR₁₂ and N; wherein R₁₂ is selected from selected from hydrogen, halo, cyano, C₁₋₄ alkyl, C₁₋₄ haloalkyl, C₁₋₄ alkoxy and C₁₋₄ haloalkoxy (e.g. hydrogen, halo, cyano and C₁₋₄ alkyl);
   A₂ is selected from CR₁₃ and N; wherein R₁₃ selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano, methoxy and methyl);
   A₈ is selected from CR₁₉R₂₀ and NR₂₁; wherein:
      R₁₉ and R₂₀ are independently selected from hydrogen, halo, cyano, C₁₋₄ alkyl, C₁₋₄ haloalkyl, C₁₋₄ alkoxy, C₁₋₄ haloalkoxy (e.g hydrogen, halo, cyano and C₁₋₄ alkyl);
      R₂₁ is hydrogen or C₁₋₄ alkyl;
   A₉ is selected from CR₂₂R₂₃ and NR₂₄; wherein:
      R₂₂ and R₂₃ are independently selected from selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
      R₂₄ is selected from hydrogen or C₁₋₄ alkyl
   A₁₁ is selected from CR₂₈R₂₉ and NR₃₀; wherein:
      R₂₈ and R₂₉ are selected from hydrogen, halo, methoxy and methyl;
      R₃₀ is selected from hydrogen or C₁₋₄ alkyl.
X₈ is selected from CR₆, N or CR_{X6a}R_{X6b}; wherein:
   R₆ is selected from hydrogen, halo, cyano, C₁₋₄ alkyl, C₁₋₄ haloalkyl, C₁₋₄ alkoxy and C₁₋₄ haloalkoxy;
   Rₓ₆ₐ and R_{x6b} are each independently selected from hydrogen, halo, cyano, C₁₋₄ alkyl, C₁₋₄ haloalkyl, C₁₋₄ alkoxy and C₁₋₄ haloalkoxy;
X₉ is N or C;
with the proviso that no more than four of X₂ to X₉ are N.
   (iv)

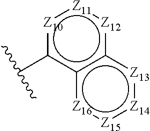

Z₁₀ is N or C—R_{Z10}, wherein R_{Z10} is selected from hydrogen, halo, cyano, C₁₋₄ alkyl, C₁₋₄ haloalkyl, C₁₋₄ alkoxy, C₁₋₄ haloalkoxy;
Z₁₁ is N or C—R_{Z11}, wherein R_{Z11} is selected from hydrogen, halo, cyano, C₁₋₄ alkyl, C₁₋₄ haloalkyl, C₁₋₄ alkoxy, C₁₋₄ haloalkoxy;
Z₁₂ is N or C—R_{Z12}, wherein R_{Z12} is selected from hydrogen, halo, cyano, C₁₋₄ alkyl, C₁₋₄ haloalkyl, C₁₋₄ alkoxy, C₁₋₄ haloalkoxy;
Z₁₃ is N or C—R_{Z13}, wherein R_{Z13} is selected from hydrogen, halo, cyano, C₁₋₄ alkyl, C₁₋₄ haloalkyl, C₁₋₄ alkoxy, C₁₋₄ haloalkoxy;
Z₁₄ is N or C—R_{Z14}, wherein R_{Z14} is selected from hydrogen, halo, cyano, C₁₋₄ alkyl, C₁₋₄ haloalkyl, C₁₋₄ alkoxy, C₁₋₄ haloalkoxy;
Z₁₅ is N or C—R_{Z15}, wherein R_{Z15} is selected from hydrogen, halo, cyano, C₁₋₄ alkyl, C₁₋₄ haloalkyl, C₁₋₄ alkoxy, C₁₋₄ haloalkoxy;
Z₁₆ is N or C—R_{Z16}, wherein R_{Z16} is selected from hydrogen, halo, cyano, C₁₋₄ alkyl, C₁₋₄ haloalkyl, C₁₋₄ alkoxy, C₁₋₄ haloalkoxy;
with the proviso that no more than three of Z₁₀ to Z₁₆ are N;
   (v)

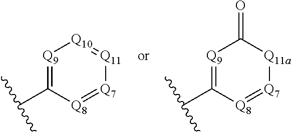

Q₇ is CR₇ or N;
Q₈ is CR₈ or N;
Q₉ is CR₉ or N;
Q₁₀ is CR₁₀ or N;
Q₁₁ is CR₁₁ or N;
Qua is NR_{11N} or CR₁₁ₐR_{11b};
wherein:
   R₇, R₈, R₉, R₁₀, R₁₁, R₁₁ₐ and R_{11b} are each independently selected from hydrogen, NH₂, heterocyclyl, halo, cyano, C₁₋₄ alkoxy, C₁₋₄ haloalkoxy, C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; and R_{11N} is selected from hydrogen, NH₂, halo, cyano, and C₁₋₆ alkyl; or
   R₉ and R₁₀ may be linked together such that, together to the atoms to which they are attached, they form a fused 5- or 6-membered saturated or unsaturated ring system, or R₁₀ and R₁₁ may be linked together such that, together to the atoms to which they are attached, they form a fused 5- or 6-membered saturated or unsaturated ring system,
   wherein either of the fused 5- or 6-membered saturated or unsaturated ring systems, or the heterocyclyl, may be optionally substituted by one or more substituents selected from C₁₋₂ alkyl, cyano, C₁₋₂ haloalkyl, hydroxy, C₁₋₂ alkoxy, halo, C₁₋₂-haloalkoxy, NR₁ᵢₐR₁ⱼₐ or —S(O)₀₋₂R₁ᵢₐR₁ⱼₐ, wherein R₁ᵢₐ and R₁ⱼₐ are H or C₁₋₂ alkyl; with the proviso that no more than three of Q₇ to Q₁₁ are N;
wherein the compound is subject to the following provisos:
   (i) Z is not one of the following structures:

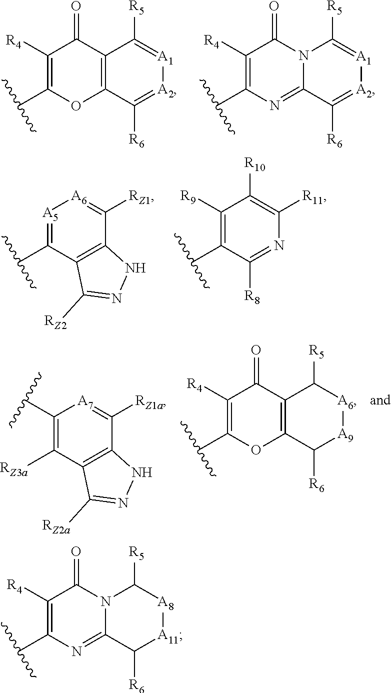

wherein:
R₄, is selected from hydrogen, halo, cyano and methyl;
R₅ is selected from hydrogen, halo, cyano and methyl;
R₆ is selected from hydrogen, halo, cyano and methyl;
R₈, R₉, R₁₀ and R₁₁ are independently selected from hydrogen, NH₂, halo, cyano, and C₁₋₆ alkyl; or
R₉ and R₁₀ may be linked together to form a fused 5- or 6-membered saturated or unsaturated ring system or R₁₀ and R₁₁ may be linked together to form a fused 5- or 6-membered saturated or unsaturated ring system; wherein either of the fused 5- or 6-membered saturated or unsaturated ring system may be optionally substituted by one or more substituents selected from C₁₋₂ alkyl, cyano, C₁₋₂ haloalkyl, hydroxy, C₁₋₂ alkoxy, halo, C₁₋₂ haloalkoxy, NR₁ᵢₐR₁ⱼₐ or —S(O)₀₋₂R₁ᵢₐR₁ⱼₐ, wherein R₁ᵢₐ and R₁ⱼₐ are H or C₁₋₂ alky;
R_{Z1} and R_{Z1a} are selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
R_{Z2} and R_{Z2a} are selected from hydrogen, C₁₋₄ alkyl, cyano, halo, NH₂ and C₁₋₄ alkoxy;
R_{Z3a} is selected from hydrogen, C₁₋₄ alkyl, cyano, halo, NH₂ and C₁₋₄ alkoxy
A₁ is selected from CR₁₂ and N;
A₂ is selected from CR₁₃ and N;
A₅ is selected from CR₁₆ and N;
A₆ is selected from CR₁₇ and N;
A₇ is selected from CR₁₈ and N;
A₈ is selected from CR₁₉R₂₀ and NR₂₁;
A₉ is selected from CR₂₂R₂₃ and NR₂₄;
A₁₁ is selected from CR₂₈R₂₉ and NR₃₀;
R₁₂ is selected from hydrogen, halo, cyano and C₁₋₄ alkyl;
R₁₃ is selected from hydrogen, halo, cyano, methoxy and methyl;
R₁₆ is selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄ cycloalkoxy;
R₁₇ is selected from hydrogen, hydroxy, halo, cyano, C₁₋₅ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—NR_{q}Rᵣ, —(OCH₂CH₂)ₘ—OCH₃ wherein m is an integer from 1 to 6, NR_{q}Rᵣ, —C(O)—NR_{q}Rᵣ, —C(O)OR_{q};
wherein R_{q} and Rᵣ are each independently hydrogen, C₁₋₅ alkyl, C₃₋₆ cycloalkyl, a 3- to 6-membered carbon-linked heterocyclyl, wherein C₁₋₅ alkyl, C₃₋₆ cycloalkyl, a 3- to 6-membered carbon-linked heterocyclyl may be optionally substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
or R_{q} and Rᵣ are linked together such that, together with the nitrogen atom to which they are attached, they form a 3- to 6-membered heterocyclic ring, which may be optionally substituted by one or more substituents selected from C₁₋₂ alkyl, cyano, C₁₋₂ haloalkyl, hydroxy, C₁₋₂ alkoxy, halo, C₁₋₂ haloalkoxy;
wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
R₁₈ is selected from hydrogen, halo, cyano, C₁₋₄ alkyl, C₂₋₄ alkenyl, C₂₋₄ alkynyl, a 5- or 6-membered heteroaryl, C₁₋₄ alkoxy, C₁₋₄ haloalkyl, C₁₋₄ haloalkoxy, C₃₋₄ cycloalkyl, a 3- to 4-membered heterocyclyl and C₃₋₄ cycloalkoxy;
R₁₉ and R₂₀ are selected from hydrogen, halo, cyano and C₁₋₄ alkyl;
R₂₂ and R₂₃ are selected from hydrogen, halo, cyano and methyl;
R₂₈ and R₂₉ are selected from hydrogen, methoxy and methyl;
R₂₁, R₂₄ and R₃₀ are hydrogen;
and
(ii) the compound is not:
4-[1-[(6-methylimidazo[1,2-a]pyridin-2-yl)methyl]triazol-4-yl]isoquinoline; tert-butyl N-[[2-[[4-(4-isoquinolyl)triazol-1-yl]methyl]imidazo[1,2-a]pyridin-6-yl]methyl]carbamate;
N-(cyclobutylmethyl)-1-[2-[[4-(4-isoquinolyl)triazol-1-yl]methyl]imidazo[1,2-a]pyridin-6-yl]methanamine.

In another aspect, the present invention relates to compounds of the formula (I), or a pharmaceutically acceptable salt thereof,

X—Y—Z (I)
wherein:
X is selected from:

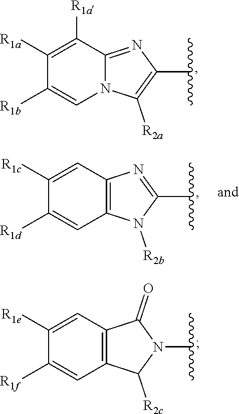

wherein:
   R₁ₐ, R_{1c} and R₁ₑ are selected from hydrogen, halo, C₁₋₄ alkyl, C₂₋₃ alkenyl and —O—C₁₋₄ alkyl R_{1b}, R_{1d} and R_{1f} are selected from:
   (i) C₁₋₄ alkyl or C₁₋₄ alkoxy, each of which being optionally substituted by halo, cyano, hydroxy, C₃₋₆ cycloalkyl, C₁₋₄ alkoxy, C₁₋₄ haloalkoxy, aryl or heteroaryl; or
   (ii) a group of the formula:

—(CR_{1c}R_{1d})ₚ—NR₁ₑR_{1f};
wherein
p is an integer selected from 0, 1, 2 or 3
R₁ₑ and R_{1d} are independently selected from:
   (i) hydrogen (including deuterium),
   (ii) C₁₋₆ alkyl which is optionally substituted by one more substituents selected from cyano, oxo, hydroxy, C₁₋₄ alkoxy, halo, C₁₋₄ haloalkoxy, C₃₋₆ cycloalkyl, —O—C₃₋₆ cycloalkyl, NR_{1ca}R_{1da} or —S(O)₀₋₂R_{1ac}R_{1da}, wherein R_{1ca} and R_{1da} are H or C₁₋₂ alkyl; and wherein C₃₋₆ cycloalkyl and —O—C₃₋₆ cycloalkyl are optionally further substituted with halo, cyano or hydroxy;
   (iii) C₃₋₄ cycloalkyl or 3 to 5 membered heterocyclyl, each of which is optionally substituted by C₁₋₄ alkyl, C₁₋₄ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl; and;
   (iv) or R_{1c} and R_{1d} are linked together such that, together with the carbon atom to which they are attached, they form a 3- to 6-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ca}R_{1da} or —S(O)₀₋₂R_{1ac}R_{1da}, wherein R_{1ca} and R_{1da} are H or C₁₋₂ alkyl;
R₁ₑ and R_{1f} are each independently selected from:
   (i) hydrogen (including deuterium);
   (ii) C₁₋₆ alkyl which is optionally substituted by one more substituents selected from cyano, oxo, hydroxy, C₁₋₂ alkoxy, halo, C₁₋₂-haloalkoxy, NR₁ₑₐR_{1fa} or —S(O)₀₋₂R₁ₑₐR_{1fa}, wherein R₁ₑₐ and R_{1fa} are H or C₁₋₂ alkyl;
   (iii) a group with the formula:

—(CR_{1g}R₁ₕ)_{q}-T₁
wherein:
q is 0, 1, 2, 3, 4, 5 or 6;
R_{1g} and R₁ₕ are independently selected from:
a) hydrogen (including deuterium);
b) C₁₋₆ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
c) an aryl-C₁₋₆ alkyl, heteroarylC₁₋₆ alkyl, C₃₋₆ cycloalkyl or C₃₋₆ cycloalkylC₁₋₆ alkyl group, each of which is optionally substituted by one or more substituents selected from C₁₋₂-alkyl, cyano, C₁₋₂ haloalkyl, hydroxy, C₁₋₂ alkoxy, halo, C₁₋₂-haloalkoxy, NR_{1ga}R₁ₕₐ or —S(O)₀₋₂R_{1ga}R₁ₕₐ, wherein R_{1ga} and R₁ₕₐ are H or C₁₋₂ alkyl; or
d) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
and T₁ is selected from hydrogen, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
R_{1a′} is selected from hydrogen, halo and methyl;
R₂ₐ, R_{2b} and R_{2c} are selected from hydrogen, halo or a group of the formula:

-L₂ₐ-L_{2b}-Q₂
wherein:
L₂ₐ is absent or C₁₋₃ alkylene optionally substituted by C₁₋₂ alkyl or oxo;
L_{2b} is absent or selected from O, S, SO, SO₂, N(Rₙ), C(O), C(O)O, OC(O), C(O)N(Rₙ), N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂ alkyl; and
Q₂ is hydrogen, cyano, C₁₋₆ alkyl, C₃₋₆ cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄ alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)_{z}NRₚR_{q} (where z is 1, 2 or 3), wherein Rₚ and R_{q} are each independently selected from hydrogen or C₁₋₄ alkyl;
Y is selected from:

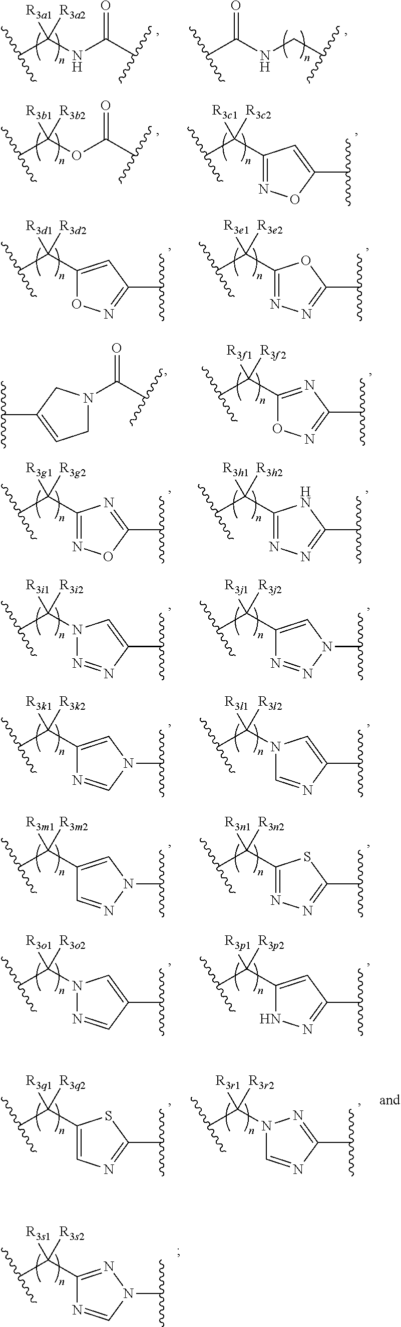

wherein:
R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen (including deuterium), C₁₋₆ alkyl, C₃₋₄ cycloalkyl, hydroxy, and halo; and wherein C₁₋₆ alkyl, or C₃₋₄ cycloalkyl is optionally substituted with one or more substituents selected from halo, amino, cyano, and hydroxy;
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen or halo;
with the proviso that R₃ₐ₁, R_{3b1}, R₃ᵢ₁, R₃ₗ₁, R₃ₒ₁, R₃ᵣ₁, R₃ₐ₂, R_{3b2}, R₃ᵢ₂, R₃ₗ₂, R₃ₒ₂ and R₃ₛ₁ cannot be halo when n=1 or when n=2 and the carbon atom to which they are attached is linked to an oxygen or nitrogen atom;
or R₃ₐ₁ and R₃ₐ₂, R_{3b1} and R_{3b2}, R_{3c1} and R_{3c2}, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, or R₃ᵣ₁ and R₃ᵣ₂ or R₃ₛ₁ and R₃ₛ₂ may be linked such that, together with the carbon atom to which they are attached, they form a spiro-fused C₃₋₄ cycloalkyl which is optionally substituted with one or more substituents selected from halo, methyl, amino, cyano, and hydroxy;
n is 0, 1 or 2; and
Z is selected from:

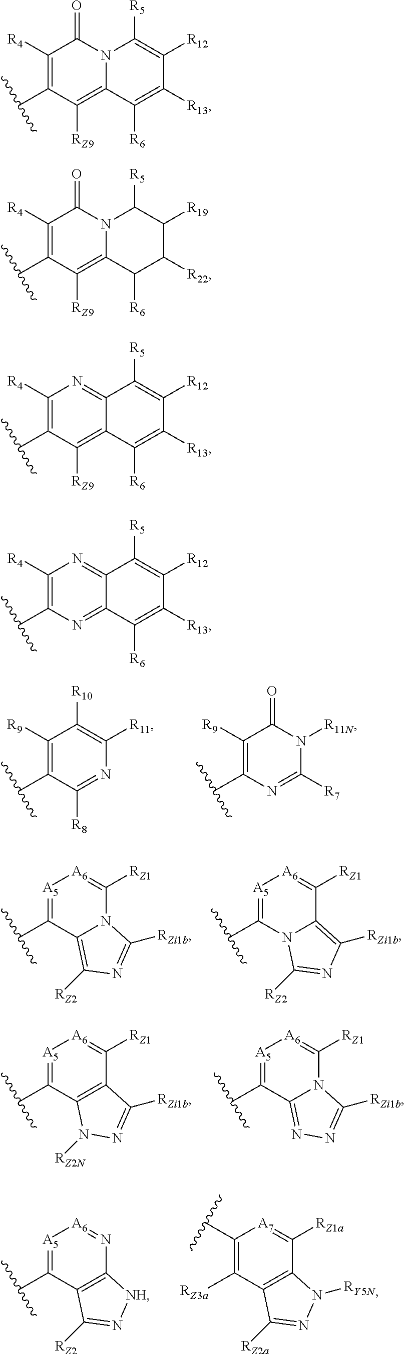

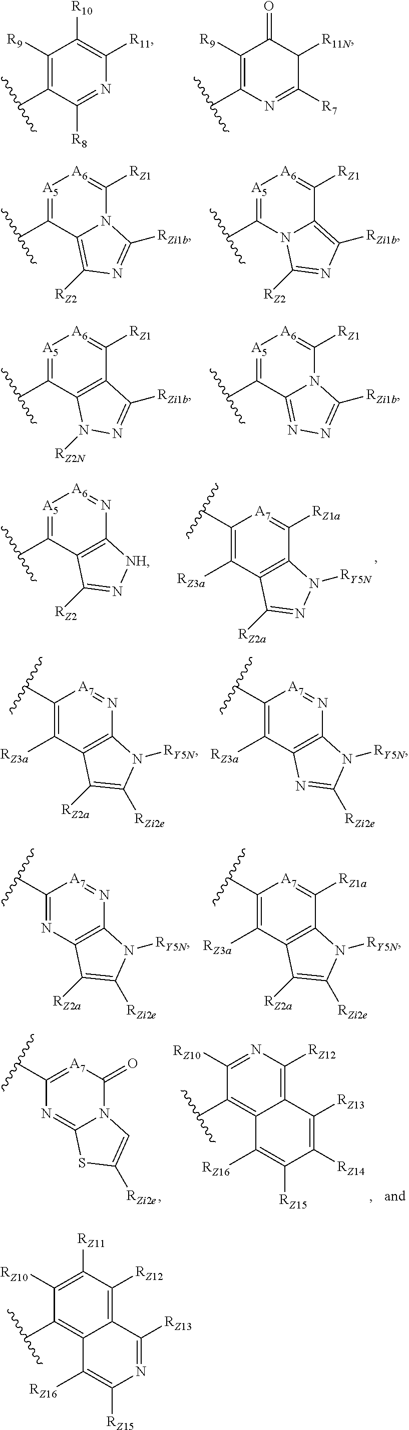

wherein:
R₄ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₅ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₆ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₁₂ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and C₁₋₄ alkyl);
R₁₃ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano, methoxy and methyl);
R₇, R₉ and R₁₁ are independently selected from hydrogen, NH₂, halo, cyano, C₁₋₄ alkoxy, C₁₋₄ haloalkoxy and C₁₋₆ alkyl;
R₈ and R₁₀ are independently selected from halo, cyano, C₁₋₄ alkoxy, C₁₋₄ haloalkoxy, C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl;
R_{11N} is selected from hydrogen and C₁₋₆ alkyl;
R_{Z1} and R_{Z1a} selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
R_{Z2} and R_{Z2a} are selected from hydrogen, C₁₋₄ alkyl, cyano, halo, NH₂ and C₁₋₄ alkoxy;
R_{Z3a} is selected from hydrogen, C₁₋₄ alkyl, cyano, halo, NH₂ and C₁₋₄ alkoxy;
R_{Zi1b} is selected from hydrogen, C₁₋₄ alkyl, cyano, halo, NH₂ and C₁₋₄ alkoxy;
R_{Zi2e} is selected from hydrogen, C₁₋₄ alkyl, cyano, halo, NH₂ and C₁₋₄ alkoxy;
R_{Y5N} is C₁₋₄ alkyl
R_{Z2N} is selected from hydrogen or C₁₋₄ alkyl
R_{Z9} is selected from hydrogen, halo, cyano, C₁₋₄ alkyl, C₁₋₄ haloalkyl, C₁₋₄ alkoxy, C₁₋₄ haloalkoxy;
R_{Z10} is selected from hydrogen, halo, cyano, C₁₋₄ alkyl, C₁₋₄ haloalkyl, C₁₋₄ alkoxy, C₁₋₄ haloalkoxy;
R_{Z11} is selected from hydrogen, halo, cyano, C₁₋₄ alkyl, C₁₋₄ haloalkyl, C₁₋₄ alkoxy, C₁₋₄ haloalkoxy;
R_{Z12} is selected from hydrogen, halo, cyano, C₁₋₄ alkyl, C₁₋₄ haloalkyl, C₁₋₄ alkoxy, C₁₋₄ haloalkoxy;
R_{Z13} is selected from hydrogen, halo, cyano, C₁₋₄ alkyl, C₁₋₄ haloalkyl, C₁₋₄ alkoxy, C₁₋₄ haloalkoxy;
R_{Z14} is selected from hydrogen, halo, cyano, C₁₋₄ alkyl, C₁₋₄ haloalkyl, C₁₋₄ alkoxy, C₁₋₄ haloalkoxy;
R_{Z15} is selected from hydrogen, halo, cyano, C₁₋₄ alkyl, C₁₋₄ haloalkyl, C₁₋₄ alkoxy, C₁₋₄ haloalkoxy;
R_{Z16} is selected from hydrogen, halo, cyano, C₁₋₄ alkyl, C₁₋₄ haloalkyl, C₁₋₄ alkoxy, C₁₋₄ haloalkoxy;
A₅ is selected from CR₁₆ and N;
A₆ is selected from CR₁₇ and N;
A₇ is selected from CR₁₈ and N;
R₁₅ is selected from hydrogen, halo, cyano, methoxy and methyl;
R₁₆ and R₁₈, are selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄-cycloalkoxy;
R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₅ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring; wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O— heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl; R₁₉ is selected from hydrogen, halo, cyano and C₁₋₄ alkyl; and R₂₂ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl).

In another aspect, the present invention relates to compounds of the formula (I), or a pharmaceutically acceptable salt thereof,

X—Y—Z (I)
wherein:
X is selected from:

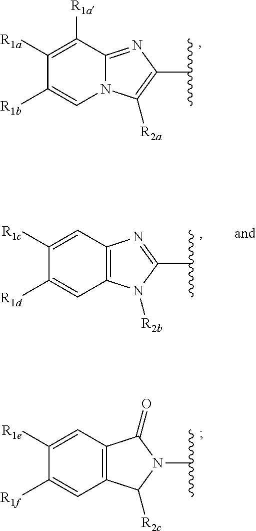

wherein:
R₁ₐ, R_{1c} and R₁ₑ are selected from hydrogen, halo, C₁₋₄ alkyl, C₂₋₃ alkenyl and —O—C₁₋₄ alkyl
R_{1b}, R_{1d} and R_{1f} are selected from:
   (i) C₁₋₄ alkyl or C₁₋₄ alkoxy, each of which being optionally substituted by halo, cyano, hydroxy, C₃₋₆ cycloalkyl, C₁₋₄ alkoxy, C₁₋₄ haloalkoxy, aryl or heteroaryl; or
   (ii) a group of the formula:

—(CR_{1c}R_{1d})ₚ—NR₁ₑR_{1f};
wherein
   p is an integer selected from 0, 1, 2 or 3;
   R₁ₑ and R_{1d} are independently selected from:
      (i) hydrogen (including deuterium),
      (ii) C₁₋₆ alkyl which is optionally substituted by one more substituents selected from cyano, oxo, hydroxy, C₁₋₄ alkoxy, halo, C₁₋₄ haloalkoxy, C₃₋₆ cycloalkyl, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkyl; and wherein C₃₋₆ cycloalkyl and —O—C₃₋₆ cycloalkyl are optionally further substituted with halo, cyano or hydroxy;
      (iii) C₃₋₄ cycloalkyl or 3 to 5 membered heterocyclyl, each of which is optionally substituted by C₁₋₄ alkyl, C₁₋₄ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl; and;
      (iv) or R_{1c} and R_{1d} are linked together such that, together with the carbon atom to which they are attached, they form a 3- to 6-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ca}R_{1d}a or —S(O)₀₋₂R_{1ca}R_{1da}, wherein R_{1ca} and R_{1da} are H or C₁₋₂ alkyl;
      R₁ₑ and R_{1f} are each independently selected from:
         (i) hydrogen (including deuterium);
         (ii) C₁₋₆ alkyl which is optionally substituted by one more substituents selected from cyano, oxo, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
         (iii) a group with the formula:

—(CR_{1g}R₁ₕ)_{q}-T₁
wherein:
q is 0, 1, 2, 3, 4, 5 or 6;
R_{1g} and R₁ₕ are independently selected from:
   a) hydrogen (including deuterium);
   b) C₁₋₆ alkyl which is optionally substituted by one more substituents selected from cyano, oxo, hydroxy, C₁₋₄ alkoxy, halo, C₁₋₄-haloalkoxy, —O—C₃₋₆ cycloalkyl, NR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
   c) an aryl-C₁₋₆ alkyl, heteroarylC₁₋₆ alkyl, C₃₋₆ cycloalkyl or C₃₋₆ cycloalkylC₁₋₆ alkyl group, each of which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl; or
   d) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
and T₁ is selected from hydrogen, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
R_{1a′} is selected from hydrogen, halo and methyl;
R₂ₐ, R_{2b} and R_{2c} are selected from hydrogen, halo or a group of the formula:

-L₂ₐ-L_{2b}-Q₂
wherein
L₂ₐ is absent or C₁₋₃ alkylene optionally substituted by C₁₋₂ alkyl or oxo;
L_{2b} is absent or selected from O, S, SO, SO₂, N(Rₙ), C(O), C(O)O, OC(O), C(O)N(Rₙ), N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂ alkyl; and
Q₂ is hydrogen, cyano, C₁₋₆ alkyl, C₃₋₆ cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄ alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)_{z}NRₚR_{q} (where z is 1, 2 or 3), wherein Rₚ and R_{q} are each independently selected from hydrogen or C₁₋₄ alkyl;
Y is selected from:

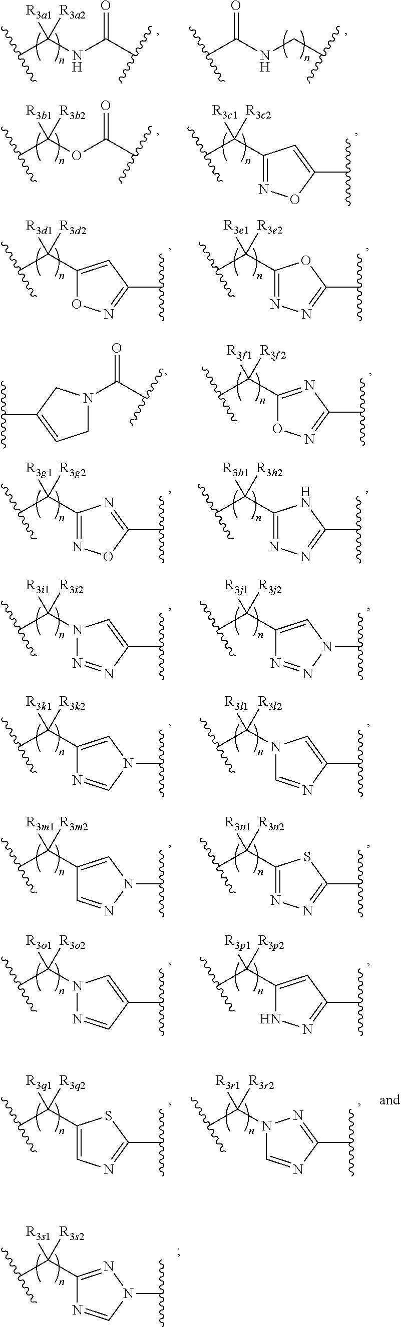

wherein:
R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen (including deuterium), C₁₋₆ alkyl, C₃₋₄ cycloalkyl, hydroxy, and halo; and wherein C₁₋₆ alkyl, or C₃₋₄ cycloalkyl is optionally substituted with one or more substituents selected from halo, amino, cyano, and hydroxy;
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen or halo;
with the proviso that R₃ₐ₁, R_{3b1}, R₃ᵢ₁, R₃ₗ₁, R₃ₒ₁, R₃ᵣ₁, R₃ₐ₂, R_{3b2}, R₃ᵢ₂, R₃ₗ₂, R₃ₒ₂ and R₃ₛ₁ cannot be halo when n=1 or when n=2 and the carbon atom to which they are attached is linked to an oxygen or nitrogen atom;
or R₃ₐ₁ and R₃ₐ₂, R_{3b1} and R_{3b2}, R₃ₐ₁ and R₃ₒ₂, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, or R₃ᵣ₁ and R₃ᵣ₂ or R₃ₛ₁ and R₃ₛ₂ may be linked such that, together with the carbon atom to which they are attached, they form a spiro-fused C₃₋₄ cycloalkyl which is optionally substituted with one or more substituents selected from halo, methyl, amino, cyano, and hydroxy;
n is 0, 1 or 2; and
Z is selected from:

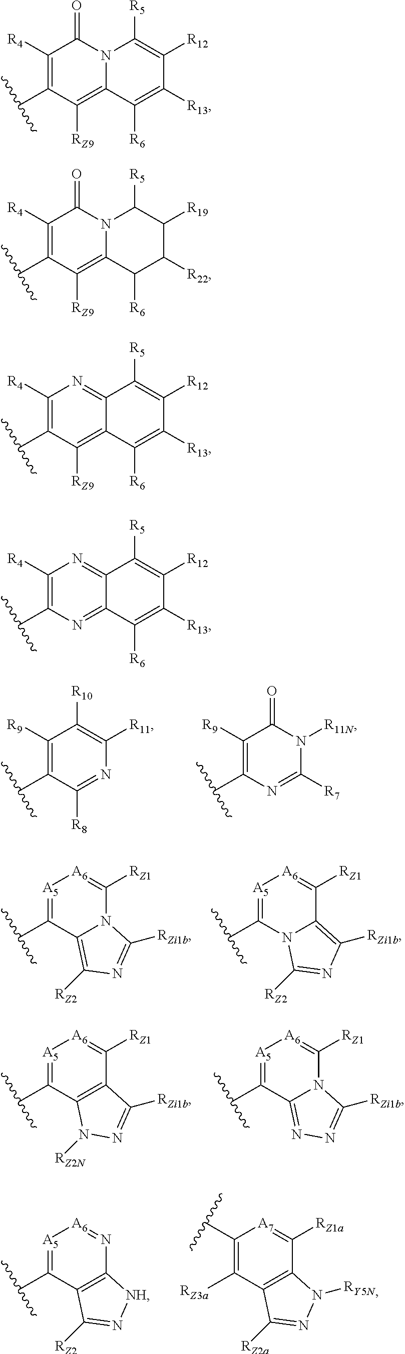

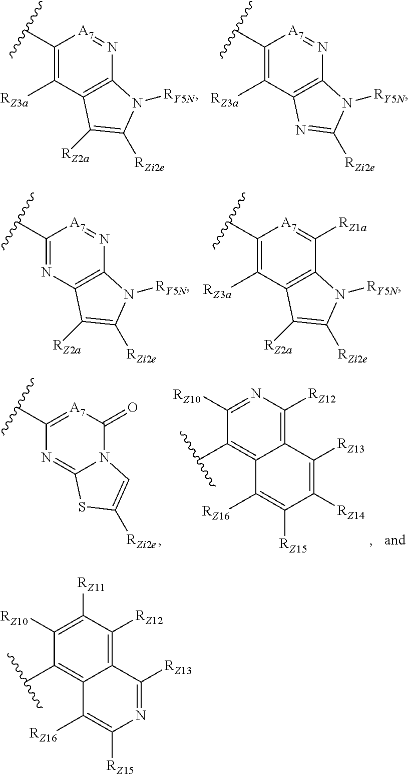

wherein:
R₄ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₅ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₆ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₁₂ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and C₁₋₄ alkyl);
R₁₃ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano, methoxy and methyl);
either:
   a) R₇, R₉ and R₁₁ are independently selected from hydrogen, NH₂, halo, cyano, C₁₋₄ alkoxy, C₁₋₄ haloalkoxy and C₁₋₆ alkyl; R₈ and R₁₀ are independently selected from halo, cyano, C₁₋₄ alkoxy, C₁₋₄ haloalkoxy, C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; or
   b) R₈, R₉, R₁₀ and R₁₁ are independently selected from hydrogen, NH₂, halo, cyano, C₁₋₄ alkoxy, C₁₋₄ haloalkoxy and C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl with the proviso that at least one of R₈, R₉, R₁₀ and R₁₁ is C₁₋₄ alkoxy or C₁₋₄ haloalkoxy;
R_{11N} is selected from hydrogen and C₁₋₆ alkyl;
R_{Z1} and R_{Z1a} selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
R_{Z2} and R_{Z2a} are selected from hydrogen, C₁₋₄ alkyl, cyano, halo, NH₂ and C₁₋₄ alkoxy;
R_{Z3a} is selected from hydrogen, C₁₋₄ alkyl, cyano, halo, NH₂ and C₁₋₄ alkoxy;
R_{Zi1b} is selected from hydrogen, C₁₋₄ alkyl, cyano, halo, NH₂ and C₁₋₄ alkoxy;
R_{Zi2e} is selected from hydrogen, C₁₋₄ alkyl, cyano, halo, NH₂ and C₁₋₄ alkoxy;
R_{Y5N} is C₁₋₄ alkyl
R_{Z2N} is selected from hydrogen or C₁₋₄ alkyl
R_{Z9} is selected from hydrogen, halo, cyano, C₁₋₄ alkyl, C₁₋₄ haloalkyl, C₁₋₄ alkoxy, C₁₋₄ haloalkoxy;
R_{Z10} is selected from hydrogen, halo, cyano, C₁₋₄ alkyl, C₁₋₄ haloalkyl, C₁₋₄ alkoxy, C₁₋₄ haloalkoxy;
R_{Z11} is selected from hydrogen, halo, cyano, C₁₋₄ alkyl, C₁₋₄ haloalkyl, C₁₋₄ alkoxy, C₁₋₄ haloalkoxy;
R_{Z12} is selected from hydrogen, halo, cyano, C₁₋₄ alkyl, C₁₋₄ haloalkyl, C₁₋₄ alkoxy, C₁₋₄ haloalkoxy;
R_{Z13} is selected from hydrogen, halo, cyano, C₁₋₄ alkyl, C₁₋₄ haloalkyl, C₁₋₄ alkoxy, C₁₋₄ haloalkoxy;
R_{Z14} is selected from hydrogen, halo, cyano, C₁₋₄ alkyl, C₁₋₄ haloalkyl, C₁₋₄ alkoxy, C₁₋₄ haloalkoxy;
R_{Z15} is selected from hydrogen, halo, cyano, C₁₋₄ alkyl, C₁₋₄ haloalkyl, C₁₋₄ alkoxy, C₁₋₄ haloalkoxy;
R_{Z16} is selected from hydrogen, halo, cyano, C₁₋₄ alkyl, C₁₋₄ haloalkyl, C₁₋₄ alkoxy, C₁₋₄ haloalkoxy;
A₅ is selected from CR₁₆ and N;
A₆ is selected from CR₁₇ and N;
A₇ is selected from CR₁₈ and N;
R₁₅ is selected from hydrogen, halo, cyano, methoxy and methyl;
R₁₆ and R₁₈ are selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄-cycloalkoxy;
R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₅ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring; wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O— heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
R₁₉ is selected from hydrogen, halo, cyano and C₁₋₄ alkyl; and
R₂₂ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl).

Particular compounds of the invention include, for example, compounds of formula (I), or pharmaceutically acceptable salts thereof, wherein, unless otherwise stated, each of X, Y, Z, R₁ₐ, R_{1b}, R_{1c}, R_{1d}, R₁ₑ, R_{1f}, R_{1a′}, R₂ₐ, R_{2b}, R_{2c}, R₃ₐ₁, R₃ₐ₂, R_{3b1}, R_{3b2}, R_{3c1}, R_{3c2}, R_{3d1}, R_{3d2}, R₃ₑ₁, R₃ₑ₂, R_{3f1}, R_{3f2}, R_{3g1}, R_{3g2}, R₃ₕ₁, R₃ₕ₂, R₃ᵢ₁, R₃ᵢ₂, R₃ⱼ₁, R₃ⱼ₂, R₃ₖ₁, R₃ₖ₂, R₃ₗ₁, R₃ₗ₂, R₃ₘ₁, R₃ₘ₂, R₃ₙ₁, R₃ₙ₂, R₃ₒ₁, R₃ₒ₂, R₃ₚ₁, R₃ₚ₂, R_{3q1}, R_{3q2}, R₃ᵣ₁, R₃ᵣ₂, R₃ₛ₁, R₃ₛ₂, n, R₄, R₅, R₆, R₇, R₈, R₉, R₁₀, R₁₁, R_{11N}, R₁₂, R₁₃, R₁₆, R₁₇, R₁₈, R₁₉, R₂₀, R₂₁, R₂₂, R₂₃, R₂₄, R₂₅, R₂₆, R₂₈, R₂₉, R₃₀, R_{Z1}, R_{Z1a}, R_{Z2}, R_{Z2a}, R_{Z3a}, R_{Zi1b}, R_{Zi2e}, A₅, A₆, A₇, R_{Z2N}, R_{Y5N}, R_{11N} and any associated substituent groups has any of the meanings defined hereinbefore or in any one of paragraphs (1) to (176) hereinafter:—
(1) R₁ₐ, R_{1c} and R₁ₑ are selected from hydrogen, halo, C₁₋₄ alkyl, C₂₋₃ alkenyl and —O—C₁₋₄ alkyl;
(2) R₁ₐ, R_{1c} and R₁ₑ are selected from hydrogen, fluoro, bromo, chloro, methyl, ethyl, propyl, butyl, ethenyl, —O-methyl, —O-ethyl, —O-propyl and —O-butyl;
(3) R₁ₐ, R_{1c} and R₁ₑ are selected from hydrogen, fluoro, bromo, chloro, methyl, ethyl, —O— methyl, ethenyl and —O-ethyl;
(4) R₁ₐ, R_{1c} and R₁ₑ are selected from hydrogen, fluoro, bromo, methyl, ethenyl and —O— methyl;
(5) R₁ₐ, R_{1c} and R₁ₑ are hydrogen;
(6) R_{1b}, R_{1d} and R_{1f} are selected from:
   (i) C₁₋₄ alkyl or C₁₋₄ alkoxy, each of which being optionally substituted by halo, cyano, hydroxy, C₃₋₆ cycloalkyl, C₁₋₄ alkoxy, C₁₋₄ haloalkoxy, aryl or heteroaryl; or
   (ii) a group of the formula:

—(CR_{1c}R_{1d})ₚ—NR₁ₑR_{1f};
wherein
   p is an integer selected from 0, 1, 2 or 3
   R_{1c} and R_{1d} are independently selected from:
      (i) hydrogen (including deuterium),
      (ii) C₁₋₆ alkyl which is optionally substituted by one more substituents selected from cyano, oxo, hydroxy, C₁₋₄ alkoxy, halo, C₁₋₄ haloalkoxy, C₃₋₆ cycloalkyl, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkyl; and wherein C₃₋₆ cycloalkyl and —O—C₃₋₆ cycloalkyl are optionally further substituted with halo, cyano or hydroxy;
      (iii) C₃₋₄ cycloalkyl or 3 to 5 membered heterocyclyl, each of which is optionally substituted by C₁₋₄ alkyl, C₁₋₄ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl; and;
      (iv) or R_{1c} and R_{1d} are linked together such that, together with the carbon atom to which they are attached, they form a 3- to 6-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂-haloalkoxy, NR_{1ca}R_{1da} or —S(O)₀₋₂R_{1ca}R_{1da}, wherein R_{1ca} and R_{1da} are H or C₁₋₂ alkyl;
R₁ₑ and R_{1f} are each independently selected from:
   (i) hydrogen (including deuterium);
   (ii) C₁₋₆ alkyl which is optionally substituted by one more substituents selected from cyano, oxo, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
   (iii) a group with the formula:

—(CR_{1g}R₁ₕ)_{q}-T₁
wherein:
q is 0, 1, 2, 3, 4, 5 or 6;
R_{1g} and R₁ₕ are independently selected from:
   a) hydrogen (including deuterium);
   b) C₁₋₆ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
   c) an aryl-C₁₋₆ alkyl, heteroarylC₁₋₆ alkyl, C₃₋₆ cycloalkyl or C₃₋₆ cycloalkylC₁₋₆ alkyl group, each of which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl; or
   d) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
and T₁ is selected from hydrogen, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
(7) R_{1b}, R_{1d} and R_{1f} are selected from:
   (i) C₁₋₄ alkyl optionally substituted by halo, cyano, hydroxy, C₃₋₆ cycloalkyl, a 3 to 6 membered heterocyclyl, C₁₋₄ alkoxy, C₁₋₄ haloalkoxy, aryl or heteroaryl; or
   (ii) a group of the formula:

—(CR_{1c}R_{1d})ₚ—NR₁ₑR_{1f};
wherein
p is an integer selected from 1 or 2;
R_{1ca}nd R_{1d} are independently selected from:
   (i) hydrogen (including deuterium),
   (ii) C₁₋₃ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkyl; and wherein C₃₋₆ cycloalkyl and —O—C₃₋₆ cycloalkyl are optionally substituted with halo, cyano or hydroxy;
   (iii) C₃₋₄ cycloalkyl or 3 to 5 membered heterocyclyl, each of which is optionally substituted by C₁₋₄ alkyl, C₁₋₄ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl; and;
   (iv) or R_{1c} and R_{1d} are linked together such that, together with the carbon atom to which they are attached, they form a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂-haloalkoxy, NR_{1ca}R_{1da} or —S(O)₀₋₂R_{1ca}R_{1da}, wherein R_{1ca} and R_{1da} are H or C₁₋₂ alkyl;
R₁ₑ and R_{1f} are each independently selected from:
   (i) hydrogen (including deuterium);
   (ii) C₁₋₆ alkyl which is optionally substituted by one more substituents selected from cyano, oxo, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
   (iii) a group with the formula:

—(CR_{1g}R₁ₕ)_{q}-T₁
wherein:
   q is 0, 1, 2 or 3;
   R_{1g} and R₁ₕ are independently selected from:
      a) hydrogen (including deuterium); or
      b) C₁₋₃ alkyl which is optionally substituted by one more substituents selected from cyano, oxo, hydroxy, C₁₋₃ alkoxy, halo, C₁₋₄ haloalkoxy, —O—C₃₋₄ cycloalkyl, wherein —O—C₃₋₄ cycloalkyl is optionally substituted with halo, cyano or hydroxy, NR_{1ca}R_{1da} or —S(O)₀₋₂R_{1ca}R_{1da}, wherein R_{1ca} and R_{1da} are H or C₁₋₂ alkylNR_{1ga}R₁ₕₐ or —S(O)₀₋₂R_{1ga}R₁ₕₐ, wherein R_{1ga} and R₁ₕₐ are H or C₁₋₂ alkyl;
      c) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
   and T₁ is selected from hydrogen, halo, C₁₋₄ alkyl, C₁₋₄ haloalkyl, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, a mono- or bicyclic heteroaryl, a spirocyclic carbocyclic or heterocyclic ring system, a bridged C₃₋₈ cycloalkyl, a bridged bicyclic C₅₋₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂-haloalkoxy, C₃₋₆ cycloalkyl, NR₃ₜR₄ₜ or —S(O)₀₋₂R₃ₜR₄ₜ, wherein R₃ₜ and R₄ₜ are H or C₁₋₂ alkyl;
(iv) or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl;
(8) R_{1b}, R_{1d} and R_{1f} are a group of the formula:

—(CR_{1c}R_{1d})ₚ—NR₁ₑR_{1f};
wherein
p is an integer selected from 1 or 2;
R_{1c} and R_{1d} are independently selected from:
   (i) hydrogen (including deuterium); or
   (ii) C₁₋₃ alkyl which is optionally substituted by one more substituents selected from cyano, oxo, hydroxy, C₁₋₃ alkoxy, halo, C₁₋₃ haloalkoxy, —O—C₃₋₄ cycloalkyl, or NH₂; wherein —O—C₃₋₆ cycloalkyl is optionally substituted with halo, cyano or hydroxy,
   (iii) C₃₋₄ cycloalkyl which is optionally substituted by C₁₋₄ alkyl, C₁₋₄ haloalkyl, cyano, hydroxy, C₁₋₂ alkoxy, halo, C₁₋₂ haloalkoxy or NR_{1ca}R_{1da} and;
   (iv) or R_{1ca}nd R_{1d} are linked together such that, together with the carbon atom to which they are attached, they form a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂-haloalkoxy, NR_{1ca}R_{1da} or —S(O)₀₋₂R_{1ca}R_{1da}, wherein R_{1ca} and R_{1da} are H or C₁₋₂ alkyl;
R₁ₑ and R_{1f} are each independently selected from:
   (i) hydrogen (including deuterium);
   (ii) C₁₋₆ alkyl which is optionally substituted by one more substituents selected from cyano, oxo, hydroxy, C₁₋₂ alkoxy, halo, C₁₋₂ haloalkoxy, NH₂;
   (iii) a group with the formula:

—(CR_{1g}R₁ₕ)_{q}-T₁
wherein:
   q is 0, 1, 2 or 3;
   R_{1g} and R₁ₕ are independently selected from:
      a) hydrogen (including deuterium); or
      b) C₁₋₆ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkylNR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy,
      c) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂ alkyl, C₁₋₂-haloalkyl, cyano, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
   and T₁ is selected from hydrogen, halo, C₁₋₄ alkyl, C₁₋₄ haloalkyl, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, C₃₋₆ cycloalkyl, NR₃ₜR₄ₜ or —S(O)₀₋₂R₃ₜR₄ₜ, wherein R₃ₜ and R₄ₜ are H or C₁₋₂ alkyl;
(iv) or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ wherein R₁ᵢ and R₁ⱼ are H or C₁₋₄ alkyl;
wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.
(9) R_{1b}, R_{1d} and R_{1f} are a group of the formula:

—(CR_{1c}R_{1d})ₚ—NR₁ₑR_{1f};
wherein
p is an integer selected from 1 or 2;
R_{1ca}nd R_{1d} are independently selected from:
   (i) hydrogen (including deuterium),
   (ii) C₁₋₃ alkyl which is optionally substituted by one more substituents selected from cyano, oxo, hydroxy, C₁₋₃ alkoxy, halo, C₁₋₄₃ haloalkoxy, —O—C₃₋₄ cycloalkyl, or NH₂; wherein —O—C₃₋₆ cycloalkyl is optionally substituted with halo, cyano or hydroxy,
   (iii) or R_{1c} and R_{1d} are linked together such that, together with the carbon atom to which they are attached, they form a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl;
R₁ₑ is selected from:
   (i) hydrogen (including deuterium);
   (ii) C₁₋₃ alkyl which is optionally substituted by one more substituents selected from cyano, oxo, hydroxy, C₁₋₂ alkoxy, halo, C₁₋₂ haloalkoxy and NH₂;
and R_{1f} is selected from:
   (i) C₁₋₆ alkyl which is optionally substituted by one more substituents selected from cyano, oxo, hydroxy, C₁₋₂ alkoxy, halo, C₁₋₂ haloalkoxy and NH₂;
   (ii) a group with the formula:

—(CR_{1g}R₁ₕ)_{q}-T₁
wherein:
q is 1, 2 or 3;
R_{1g} and R₁ₕ are independently selected from:
   a) hydrogen (including deuterium); or
   b) C₁₋₆ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkylNR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
   c) or R_{1g} and R₁ₕ are optionally linked together such that, together with the carbon atom to which they are attached, they form a 3- to 4-membered cycloalkyl or heterocyclic ring which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
and T₁ is selected from hydrogen, halo, C₁₋₄ alkyl, C₁₋₄ haloalkyl, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂-haloalkoxy, C₃₋₆ cycloalkyl, NR₃ₜR₄ₜ or —S(O)₀₋₂R₃ₜR₄ₜ, wherein R₃ and R_{4f} are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring; which in turn is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.
(10) R_{1b}, R_{1d} and R_{1f} are a group of the formula:

—(CR_{1c}R_{1d})ₚ—NR₁ₑR_{1f}
wherein
p is an integer selected from 1 or 2;
R_{1ca}nd R_{1d} are independently selected from:
   (i) hydrogen (including deuterium);
   (ii) C₁₋₃ alkyl which is optionally substituted by one more substituents selected from cyano, oxo, hydroxy, C₁₋₃ alkoxy, halo, C₁₋₃ haloalkoxy, —O—C₃₋₄ cycloalkyl, or NH₂; wherein —O—C₃₋₆ cycloalkyl is optionally substituted with halo, cyano or hydroxy,
   (iii) or R_{1c} and R_{1d} are linked together such that, together with the carbon atom to which they are attached, they form a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo or C₁₋₂ haloalkoxy;
R₁ₑ is selected from:
   (i) hydrogen (including deuterium);
   (ii) C₁₋₃ alkyl which is optionally substituted by one more substituents selected from cyano, oxo, hydroxy, C₁₋₂ alkoxy, halo, C₁₋₂ haloalkoxy and NH₂; R_{1f} is a group with the formula:

—(CR_{1g}R₁ₕ)_{q}-T₁
wherein:
q is 1, 2 or 3;
R_{1g} and R₁ₕ are independently selected from:
   a) hydrogen (including deuterium); or
   b) C₁₋₃ alkyl which is optionally substituted by one more substituents selected from cyano, oxo, hydroxy, C₁₋₄ alkoxy, halo, C₁₋₄ haloalkoxy, —O—C₃₋₆ cycloalkyl, wherein —O—C₃₋₆ cycloalkyl is optionally substituted with halo, cyano or hydroxy;
   c) or R_{1g} and R₁ₕ are optionally linked together such that, together with the carbon atom to which they are attached, they form a 3- to 4-membered cycloalkyl or heterocyclic ring which is optionally substituted by one or more substituents selected from C₁₋₂ alkyl, C₁₋₂-haloalkyl, cyano, hydroxy, C₁₋₂ alkoxy, halo or C₁₋₂-haloalkoxy;
and T₁ is selected from halo, C₁₋₄ alkyl, C₁₋₄ haloalkyl, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, C₃₋₆ cycloalkyl, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.
(11) R_{1b}, R_{1d} and R_{1f} are a group of the formula:

—(CR_{1c}R_{1d})ₚ—NR₁ₑR_{1f};
wherein
p is an integer selected from 1 or 2;
R_{1ca}nd R_{1d} are independently selected from:
   (i) hydrogen (including deuterium),
   (ii) C₁₋₃ alkyl which is optionally substituted by one more substituents selected from cyano, oxo, hydroxy, C₁₋₃ alkoxy, halo, C₁₋₃ haloalkoxy, —O—C₃₋₄ cycloalkyl, or NH₂; wherein —O—C₃₋₆ cycloalkyl is optionally substituted with halo, cyano or hydroxy,
   (iii) or R_{1c} and R_{1d} are linked together such that, together with the carbon atom to which they are attached, they form a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo or C₁₋₂ haloalkoxy;
R₁ₑ is selected from:
   (i) hydrogen (including deuterium);
   (ii) C₁₋₃ alkyl which is optionally substituted by one more substituents selected from cyano, oxo, hydroxy, C₁₋₂ alkoxy, halo, C₁₋₂ haloalkoxy and NH₂;
R_{1f} is a group with the formula:

—(CR_{1g}R₁ₕ)_{q}-T₁
wherein:
q is 1 or 2;
R_{1g} and R₁ₕ are independently selected from:
   a) hydrogen (including deuterium); or
   b) C₁₋₃ alkyl, which is optionally substituted by one more substituents selected from cyano, oxo, hydroxy, C₁₋₂ alkoxy, halo, C₁₋₂ haloalkoxy, —O—C₃ cycloalkyl, wherein —O—C₃ cycloalkyl is optionally substituted with halo, cyano or hydroxy;
and T₁ is selected from C₁₋₄ alkyl, C₃₋₈ cycloalkyl, aryl, heterocyclyl, heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy or C₃₋₆ cycloalkyl;
or R₁ₑ and R_{1f} are linked such that, together with the nitrogen atom to which they are attached, they form a mono- or bicyclic-heterocyclic ring, which is optionally substituted by one or more substituents selected from C₁₋₂ alkyl, C₁₋₂ haloalkyl, C₃₋₆ cycloalkyl, cyano, hydroxy, C₁₋₂ alkoxy, halo, C₁₋₂ haloalkoxy, NR₁ᵢR₁ⱼ or —S(O)₀₋₂R₁ᵢR₁ⱼ, wherein R₁ᵢ and R₁ⱼ are H or C₁₋₂ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₂ alkyl, C₃₋₆ cycloalkyl, cyano, hydroxy, C₁₋₂ alkoxy, halo, C₁₋₂ haloalkoxy, NR₁ᵢR₁ⱼ or —S(O)₀₋₂R₁ᵢR₁ⱼ, wherein R₁ᵢ and R₁ⱼ are H or C₁₋₂ alkyl;
wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.
(12) R_{1b}, R_{1d} and R_{1f} are a group of the formula:

—(CR_{1c}R_{1d})ₚ—NR₁ₑR_{1f};
wherein
p is an integer selected from 1 or 2;
R_{1ca}nd R_{1d} are independently selected from:
   (i) hydrogen (including deuterium) or
   (ii) C₁₋₃ alkyl which is optionally substituted by one more substituents selected from cyano, oxo, hydroxy, C₁₋₂ alkoxy, halo, C₁₋₂ haloalkoxy, —O—C₃ cycloalkyl;
R₁ₑ is selected from hydrogen (including deuterium) or C₁₋₂ alkyl; and
R_{1f} is a group with the formula:

—(CR_{1g}R₁ₕ)_{q}-T₁
wherein:
q is 1 or 2;
R_{1g} and R₁ₕ are independently selected from:
   a) hydrogen (including deuterium); or
   b) C₁₋₂ alkyl, which is optionally substituted by one more substituents selected from cyano, oxo, hydroxy, C₁₋₂ alkoxy, halo or C₁₋₂ haloalkoxy;
and T₁ is selected from C₁₋₄ alkyl, C₃₋₈ cycloalkyl, aryl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy or C₃₋₆ cycloalkyl;
or R₁ₑ and R_{1f} are linked such that, together with the nitrogen atom to which they are attached, they form a mono- or bicyclic-heterocyclic ring, which is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy; wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.
(13) R_{1b}, R_{1d} and R_{1f} are a group of the formula:

—(CR_{1c}R_{1d})ₚ—NR₁ₑR_{1f};
wherein:
p is an integer selected from 1 or 2;
R_{1c} and R_{1d} are independently selected from hydrogen (including deuterium) or C₁₋₂ alkyl;
R₁ₑ is selected from hydrogen (including deuterium) or C₁₋₂ alkyl; and
R_{1f} is a group with the formula:

—(CR_{1g}R₁ₕ)_{q}-T₁
wherein:
q is 1 or 2;
R_{1g} and R₁ₕ are independently selected from hydrogen (including deuterium) or C₁₋₂ alkyl;
and T₁ is selected from C₁₋₄ alkyl, C₃₋₄ cycloalkyl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy or C₃₋₆ cycloalkyl;
or R₁ₑ and R_{1f} are linked such that, together with the nitrogen atom to which they are attached, they form a mono- or bicyclic-heterocyclic ring, which is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy; wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.
(14) R_{1b}, R_{1d} and R_{1f} are a group of the formula:

—(CR_{1c}R_{1d})ₚ—NR₁ₑR_{1f};
wherein
p is 1;
R_{1c} and R_{1d} are independently selected from hydrogen (including deuterium) or C₁₋₂ alkyl;
R₁ₑ is selected from hydrogen (including deuterium) or C₁₋₂ alkyl; and
R_{1f} is a group with the formula:

—(CR_{1g}R₁ₕ)_{q}-T₁
wherein:
q is 1 or 2;
R_{1g} and R₁ₕ are independently selected from hydrogen (including deuterium) or C₁₋₂ alkyl;
and T₁ is selected from C₃₋₄ cycloalkyl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂-haloalkoxy or C₃₋₆ cycloalkyl;
or R₁ₑ and R_{1f} are linked such that, together with the nitrogen atom to which they are attached, they form a mono- or bicyclic-heterocyclic ring, which is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, and/or the mono- or bicyclic heterocyclic ring formed by R₁ₑ and R_{1f} is optionally spiro-fused to a C₃₋₆ cycloalkyl or a heterocyclic ring; which in turn is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.
(15) R_{1b}, R_{1d} and R_{1f} are a group of the formula:

—(CR_{1c}R_{1d})ₚ—NR₁ₑR_{1f};
wherein
p is 1;
R_{1ca}nd R_{1d} are independently selected from hydrogen (including deuterium) or C₁₋₂ alkyl;
R₁ₑ is selected from hydrogen (including deuterium) or C₁₋₂ alkyl; and
R_{1f} is a group with the formula:

—(CR_{1g}R₁ₕ)_{q}-T₁
wherein:
q is 1;
R_{1g} and R₁ₕ are independently selected from hydrogen (including deuterium) or C₁₋₂ alkyl;
and T₁ is selected from C₃₋₄ cycloalkyl, heterocyclyl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy or C₃₋₆ cycloalkyl, wherein any alkyl or alkoxy is optionally further substituted by one or more substituents selected from cyano, hydroxy or halo.
(16) R_{1b}, R_{1d} and R_{1f} are a group of the formula:

—(CR_{1c}R_{1d})ₚ—NR₁ₑR_{1f};
wherein
p is 1;
R_{1ca}nd R_{1d} are independently selected from hydrogen (including deuterium) or C₁₋₂ alkyl; and
R₁ₑ and R_{1f} are linked such that, together with the nitrogen atom to which they are attached, they form a mono- or bicyclic-heterocyclic ring, which is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₂ alkyl, C₁₋₂ haloalkyl, cyano, hydroxy, C₁₋₂ alkoxy, halo or C₁₋₂ haloalkoxy.
(17) R_{1b}, R_{1d} and R_{1f} are a group of the formula:

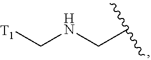

wherein T₁ is as defined herein.
(18) R_{1b}, R_{1d} and R_{1f} are a group of the formula:

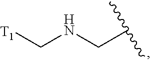

wherein T₁ is selected from C₃₋₄ cycloalkyl, heterocyclyl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo or C₁₋₂ haloalkoxy.
(19) R_{1b}, R_{1d} and R_{1f} are selected from: methoxy;

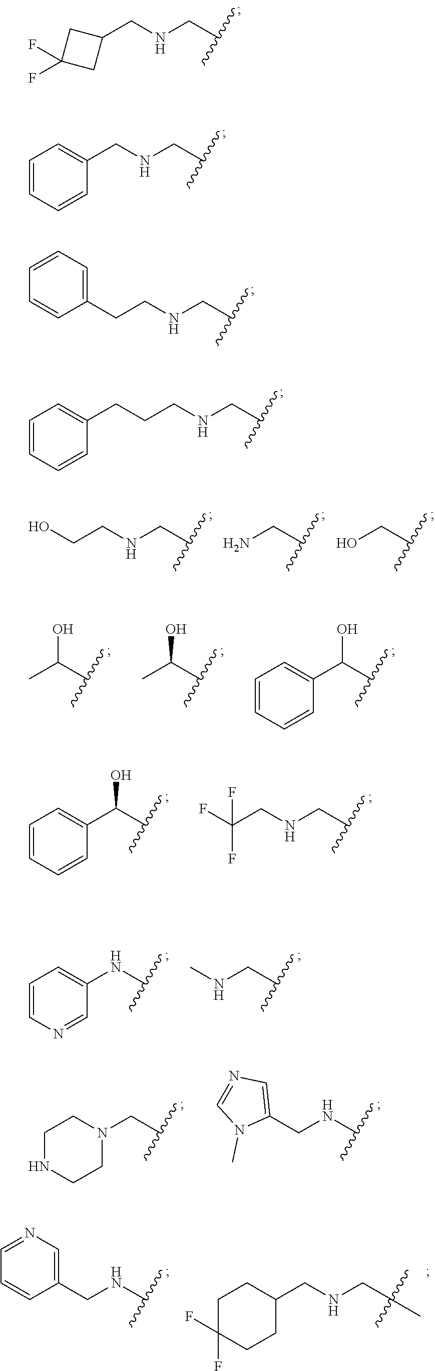

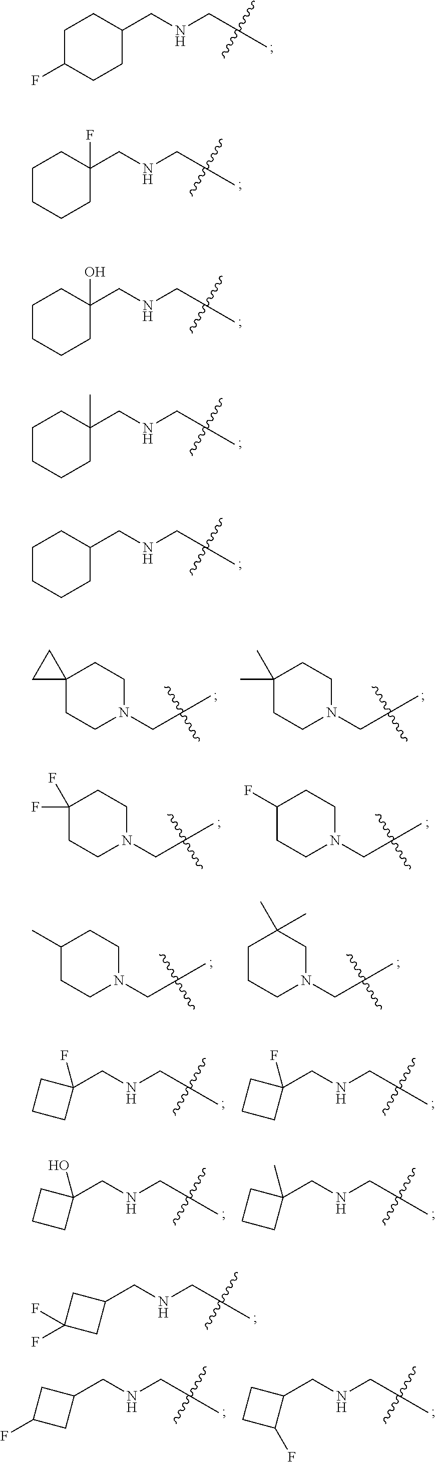

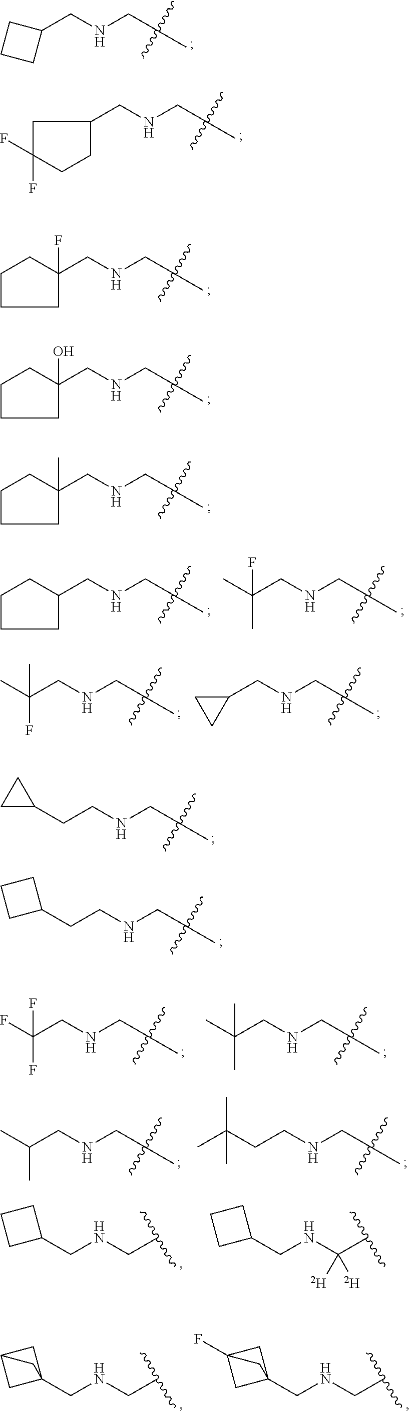

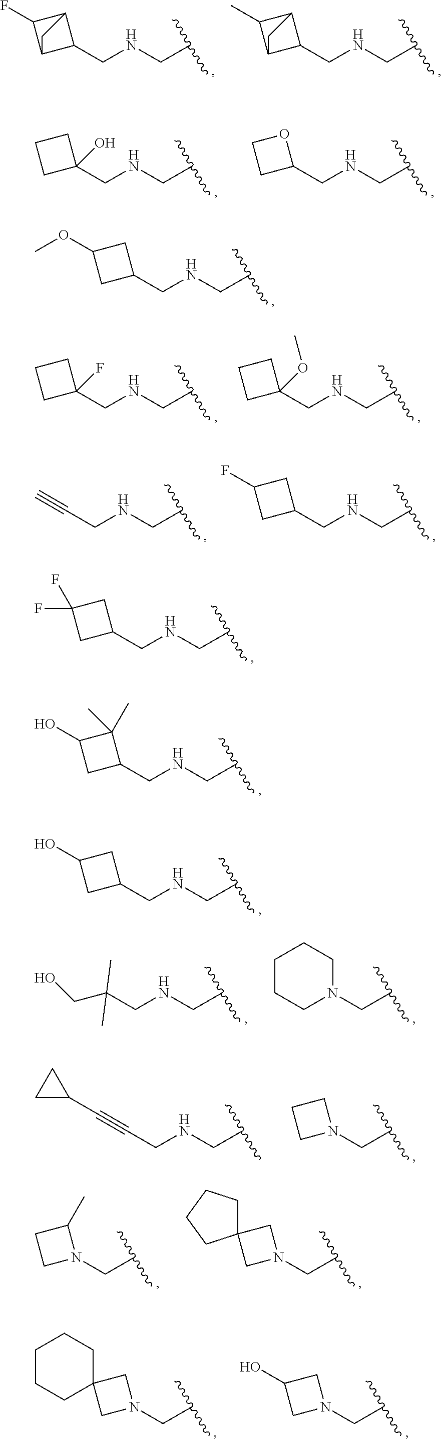

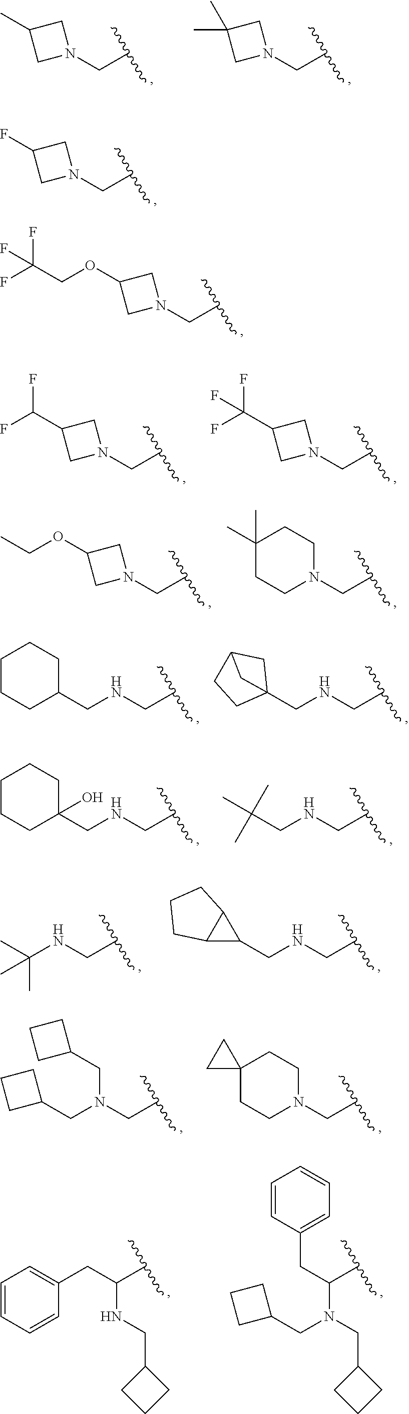

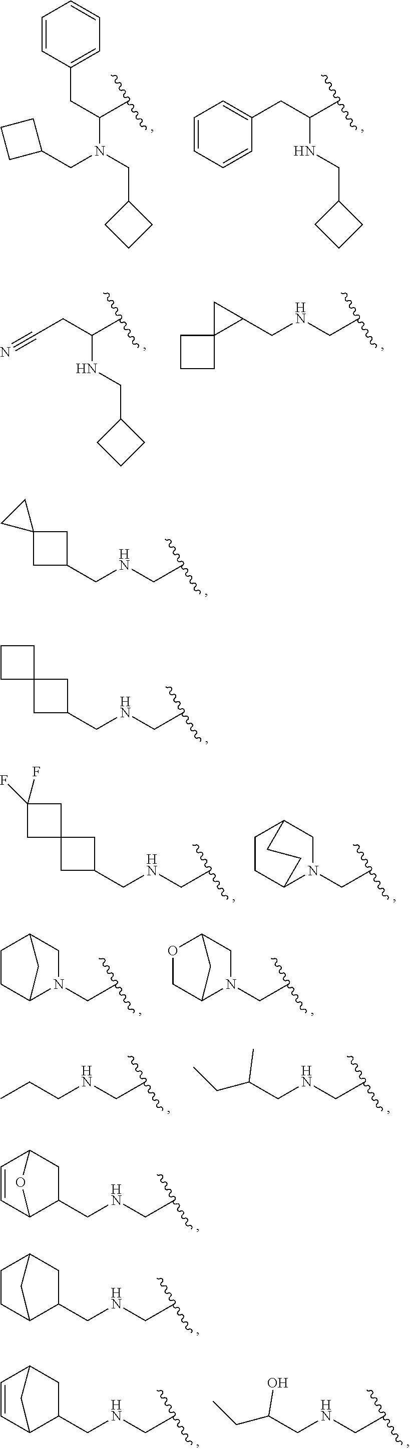

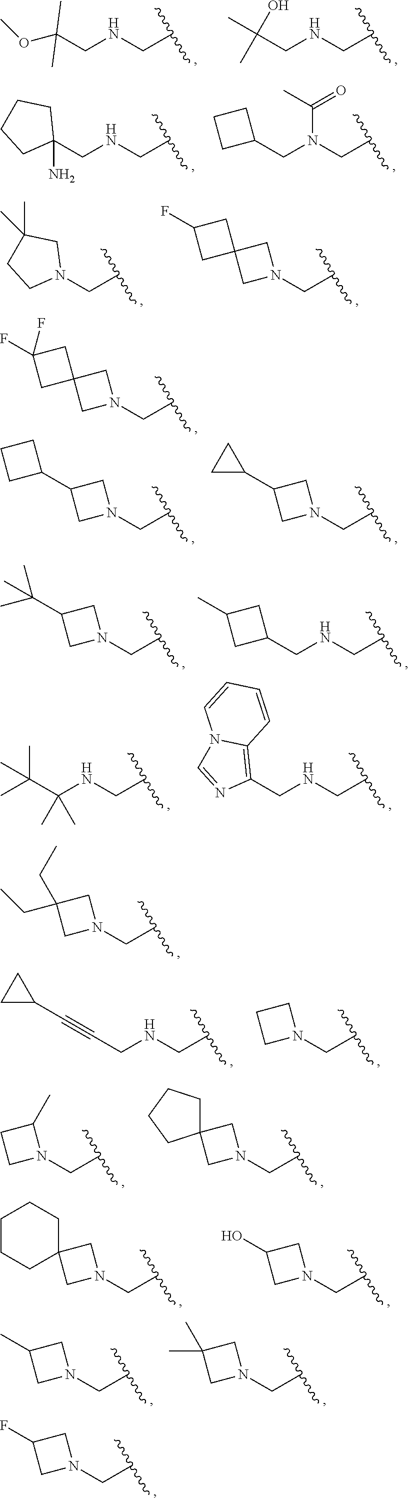

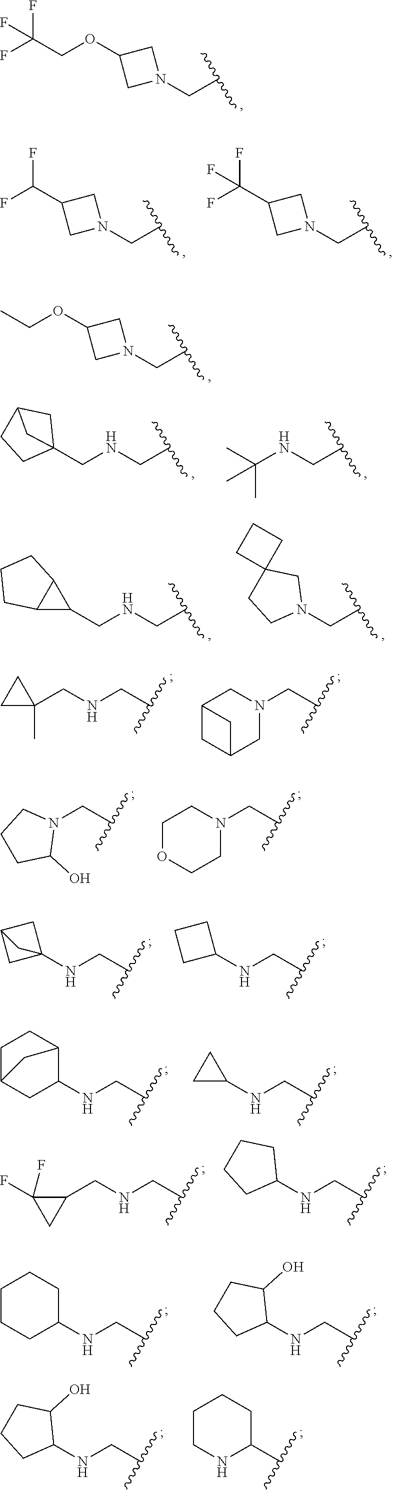

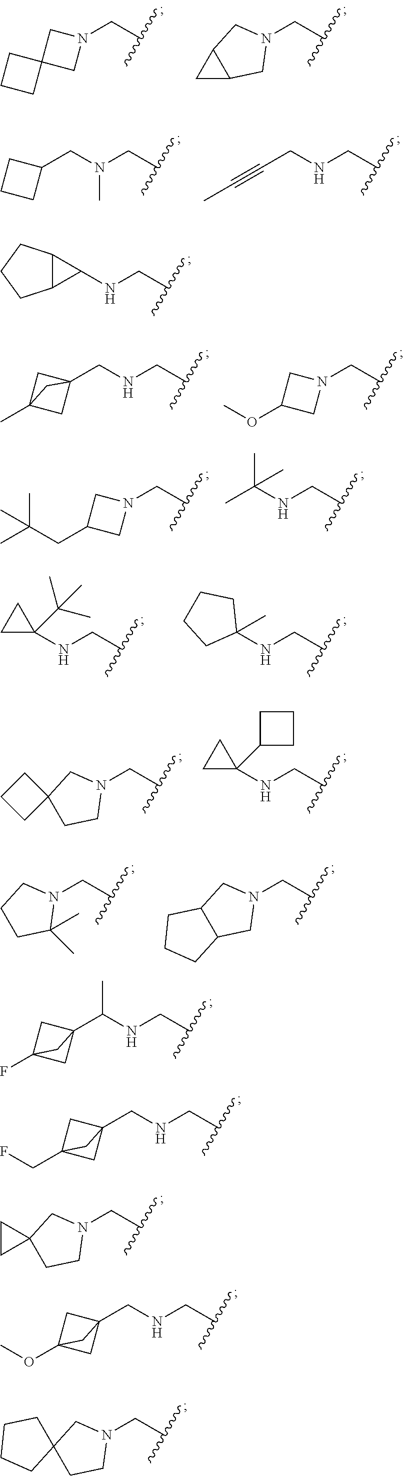

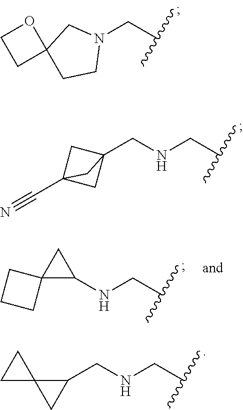

(20) R_{1b}, R_{1d} and R_{1f} are selected from:

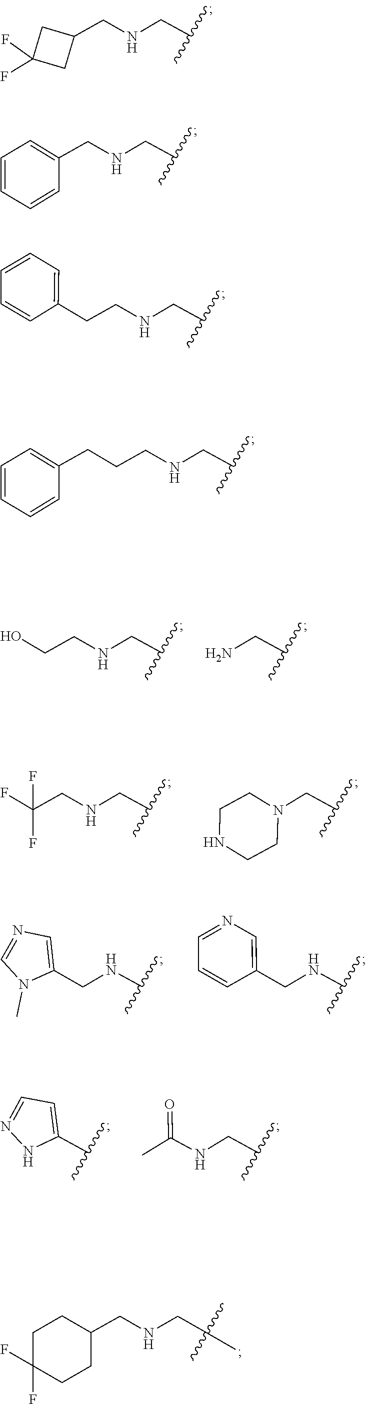

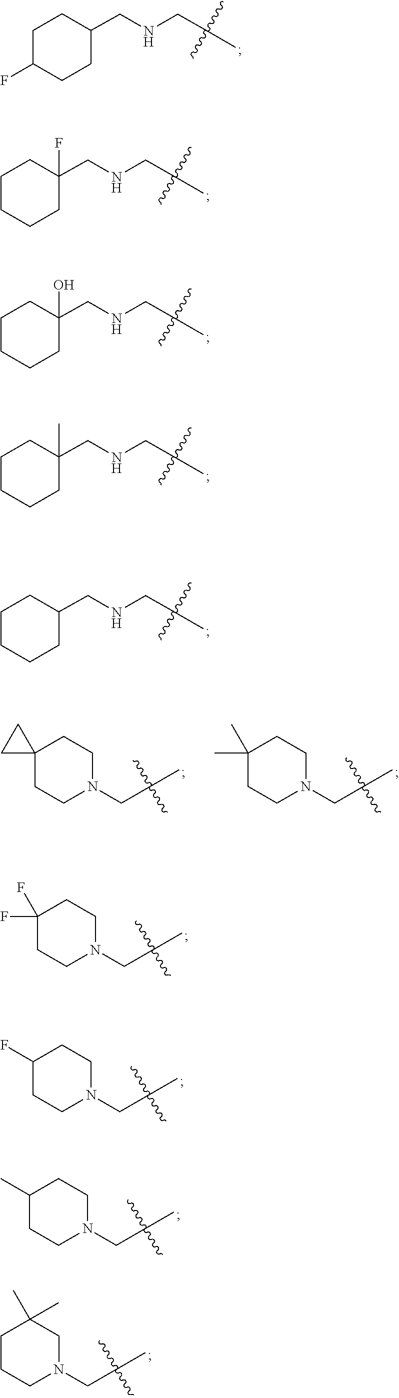

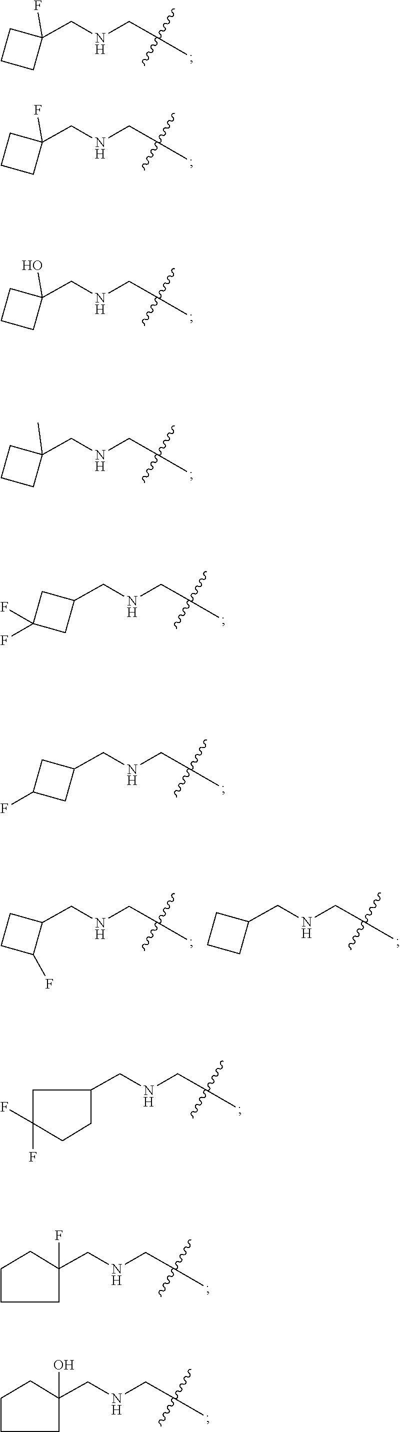

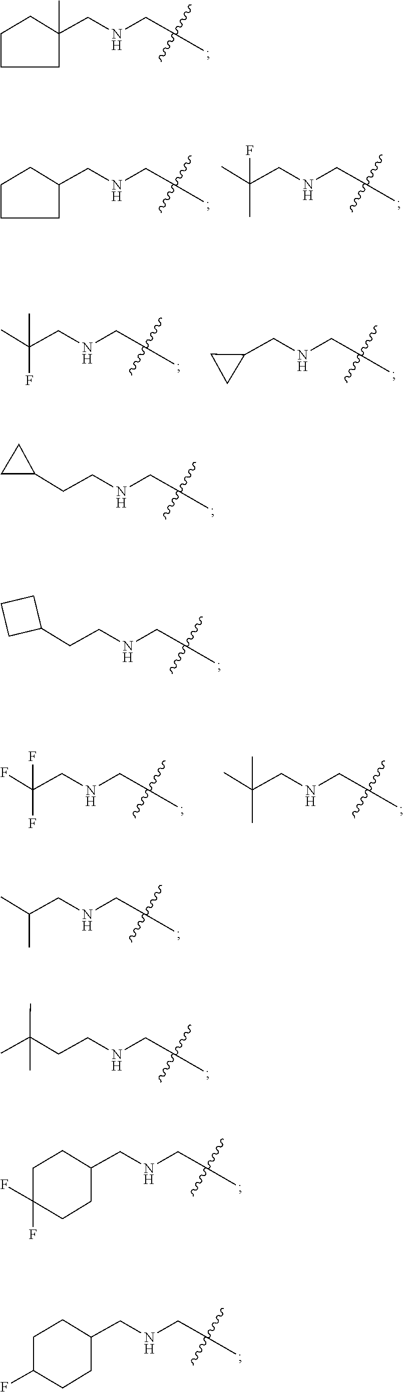

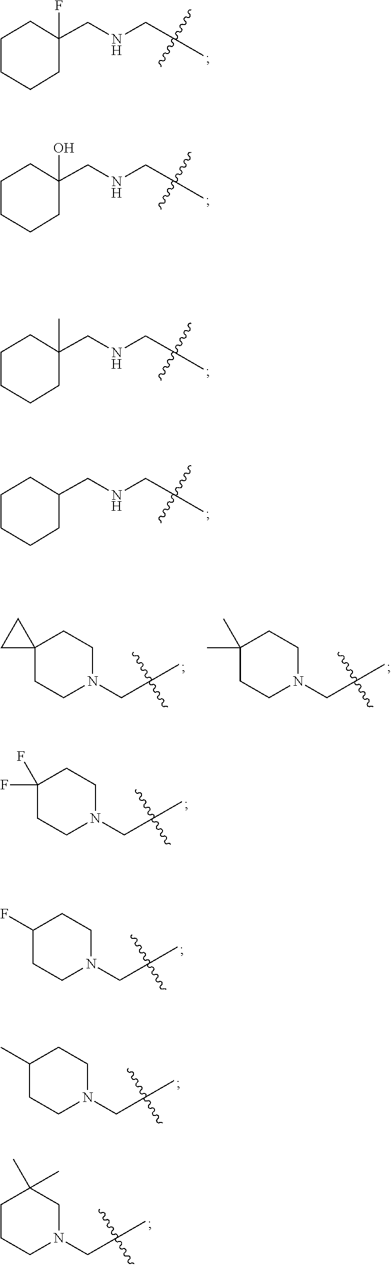

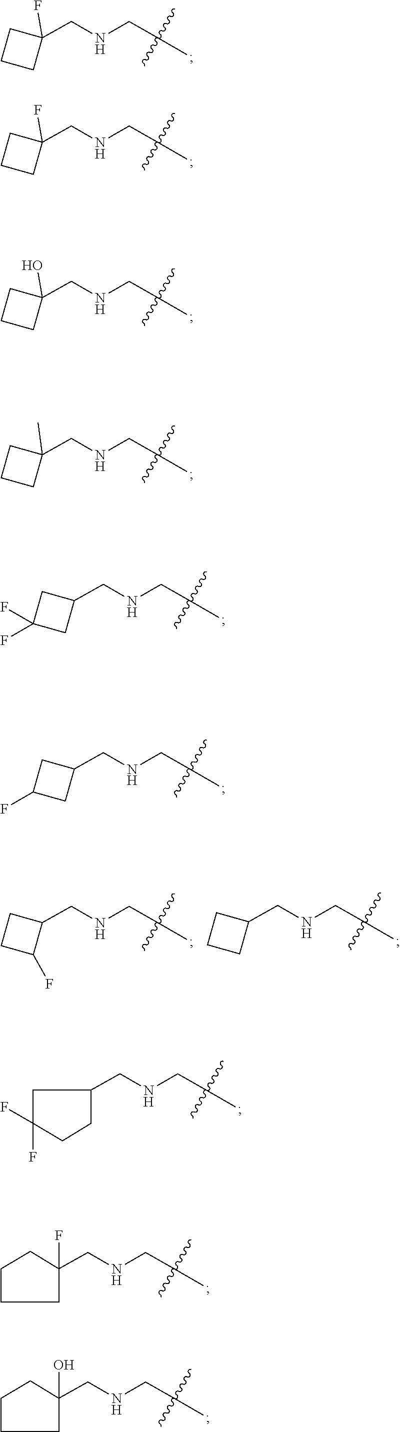

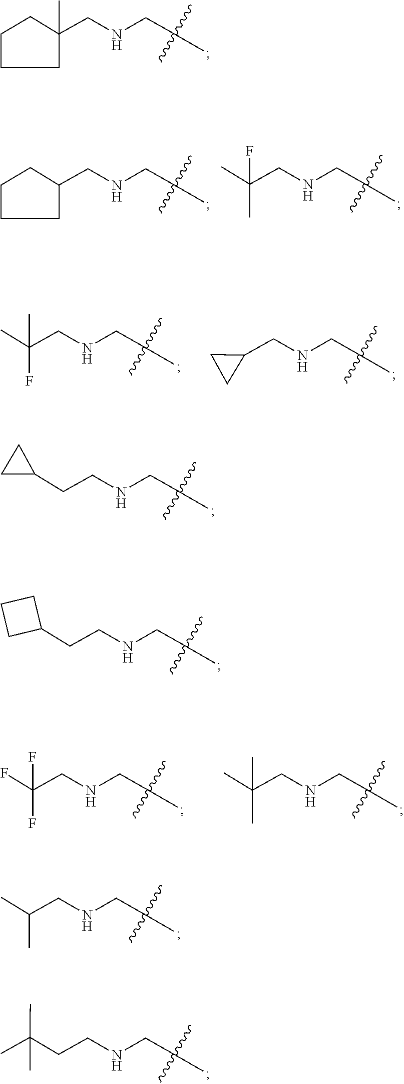

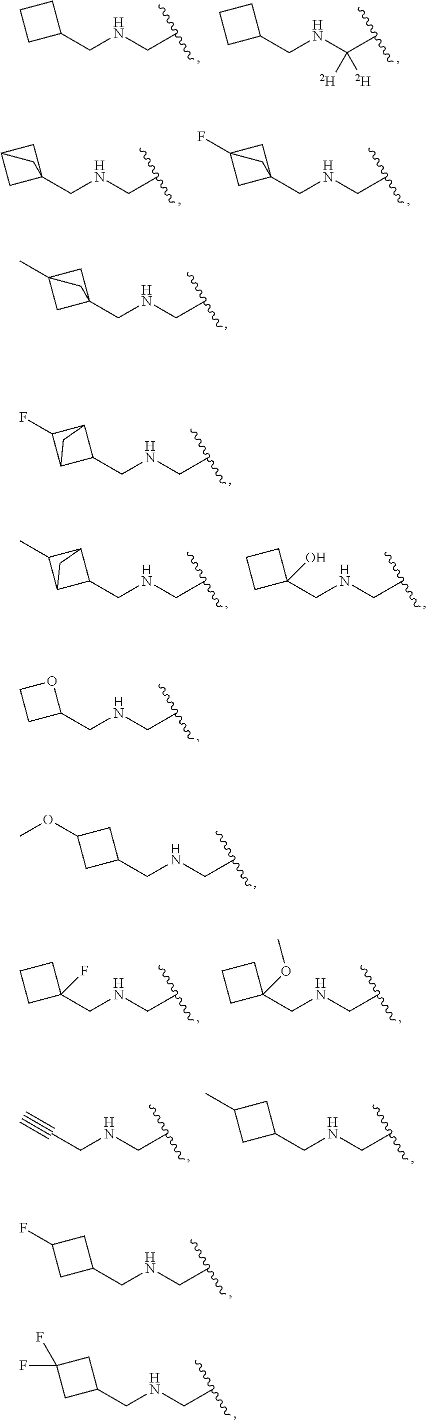

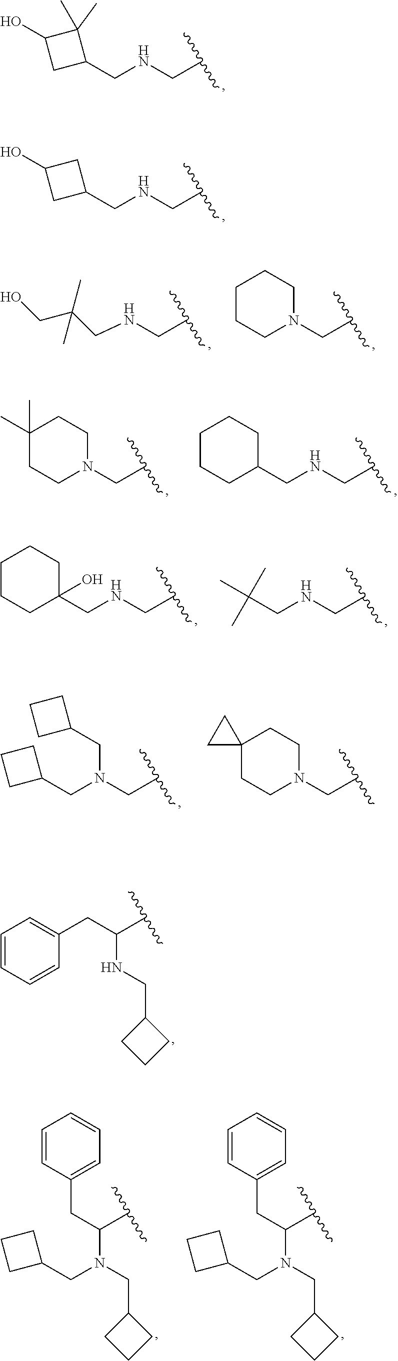

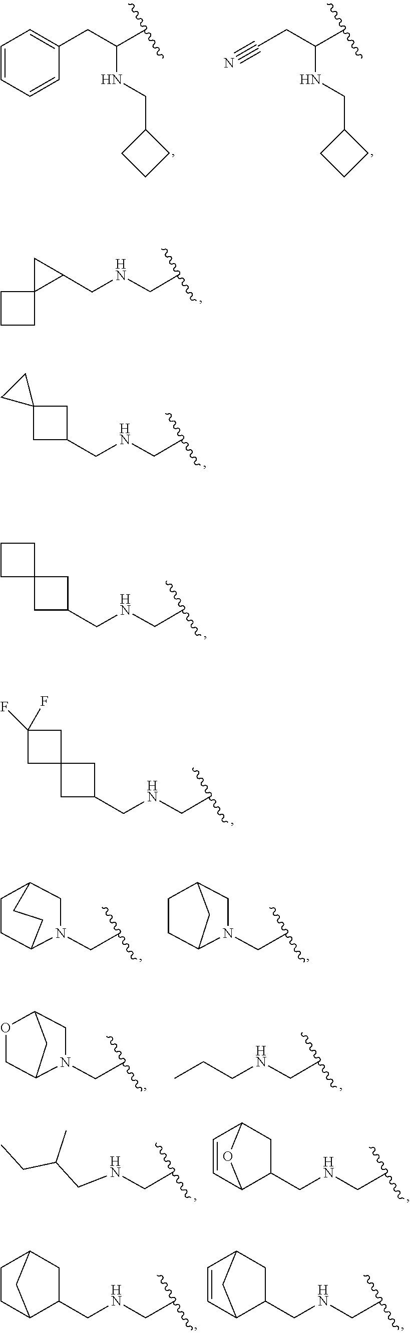

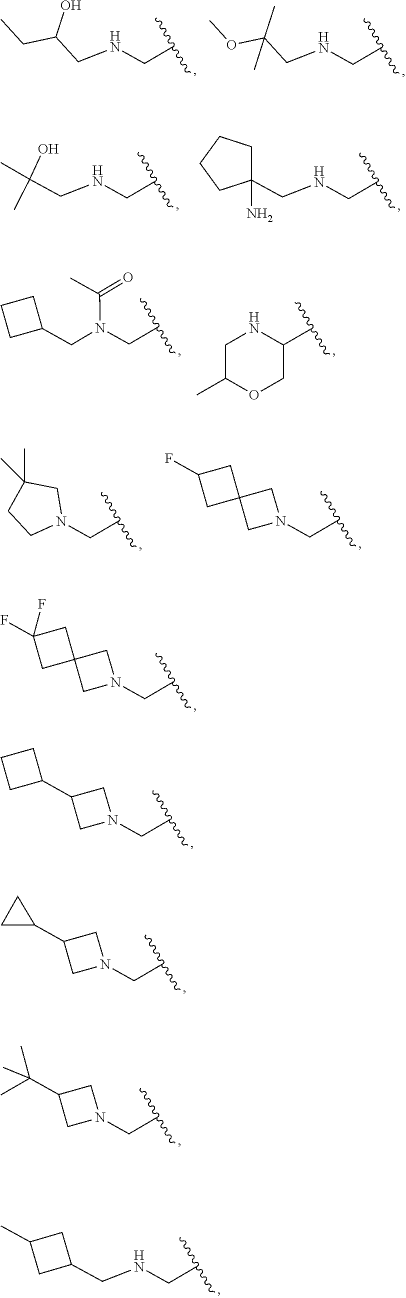

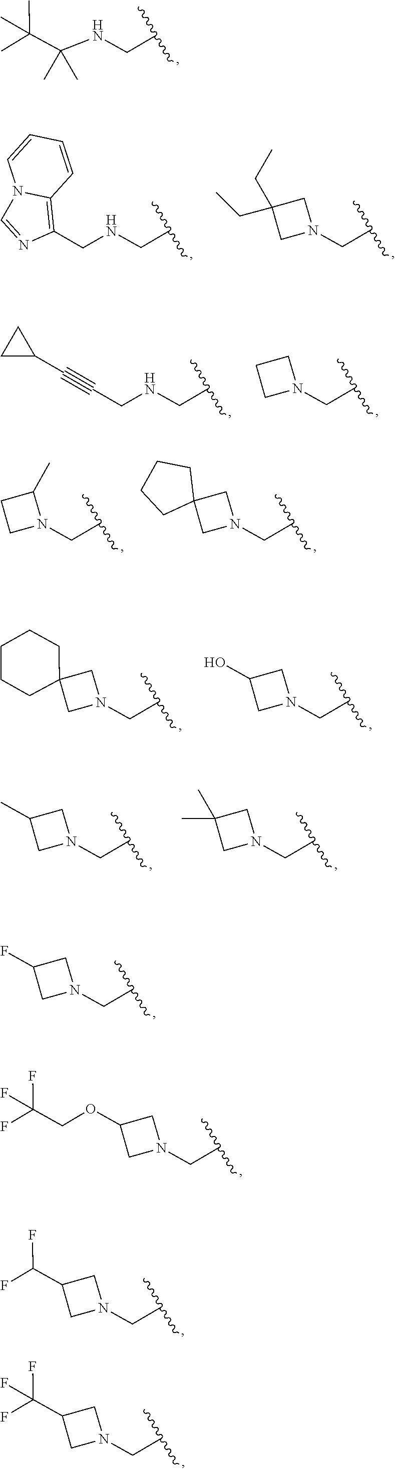

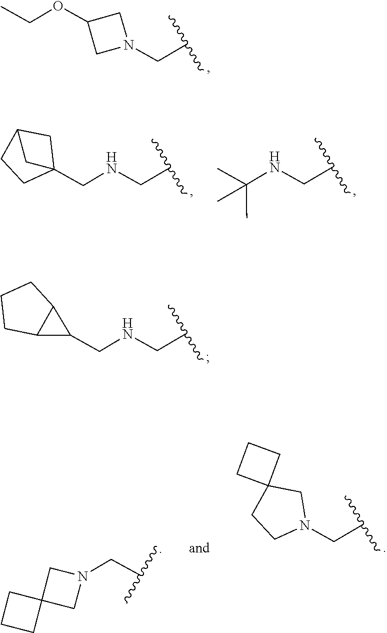

(21) R_{1b}, R_{1d} and R_{1f} are selected from:

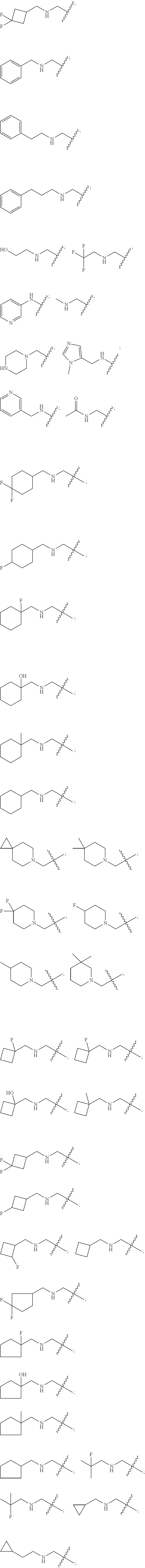

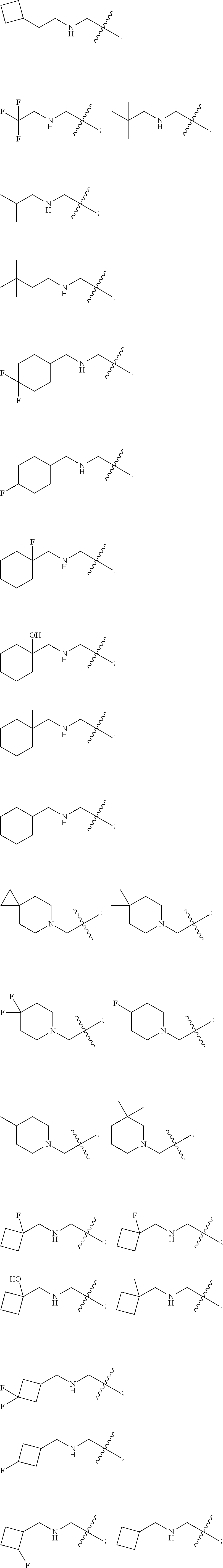

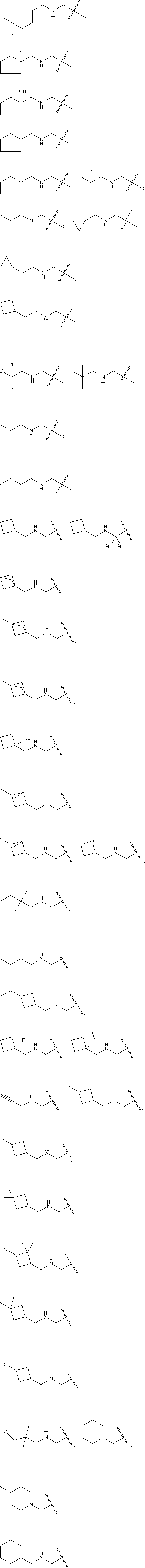

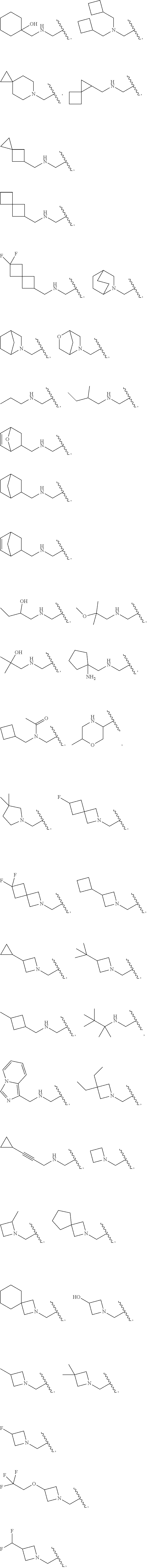

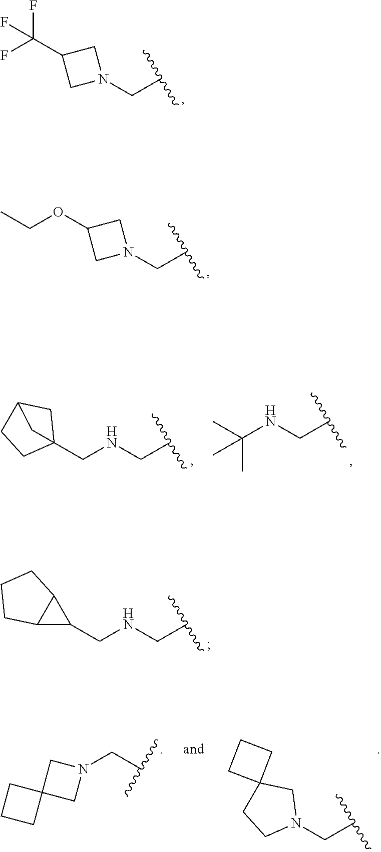

(22) R_{1b}, R_{1d} and R_{1f} are selected from:

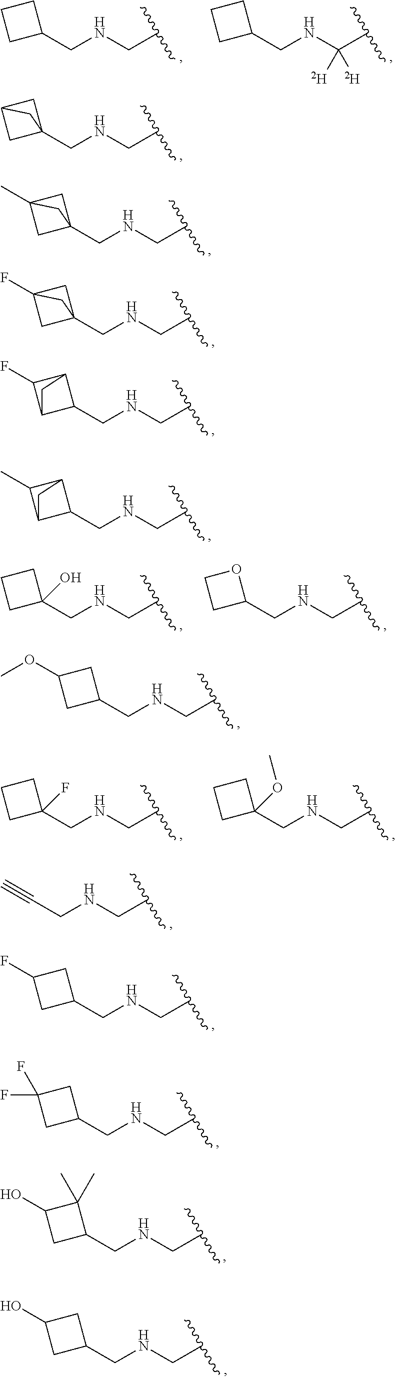

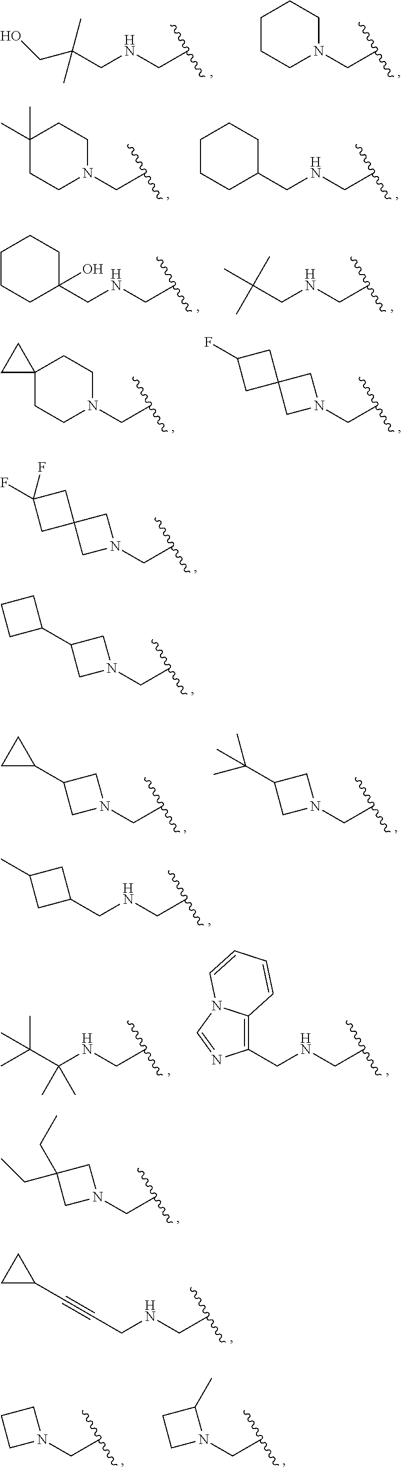

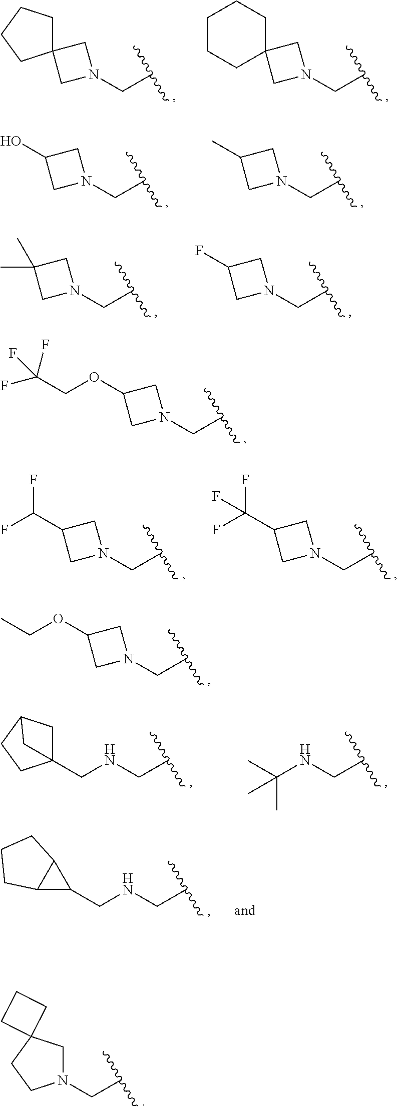

(23) R_{1b}, R_{1d} and R_{1f} are selected from:

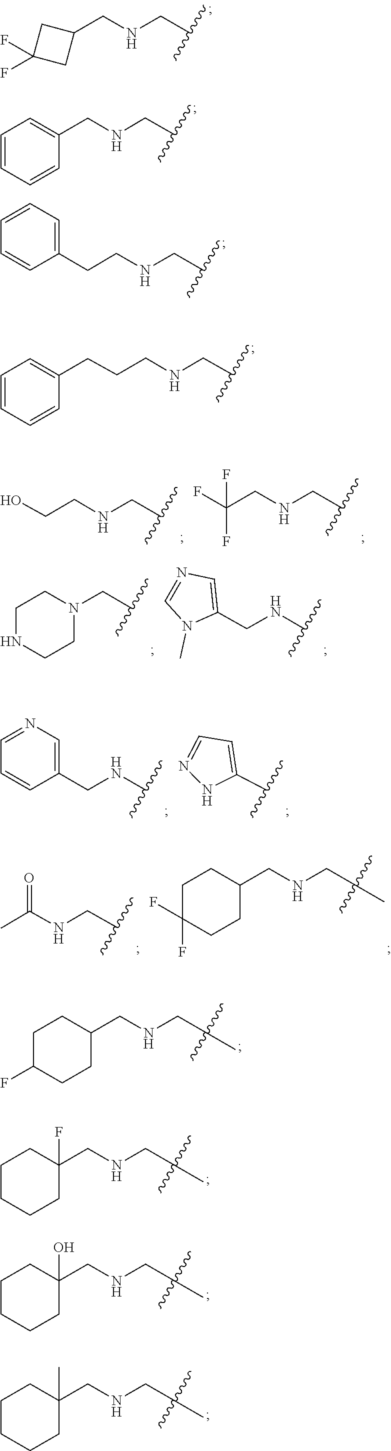

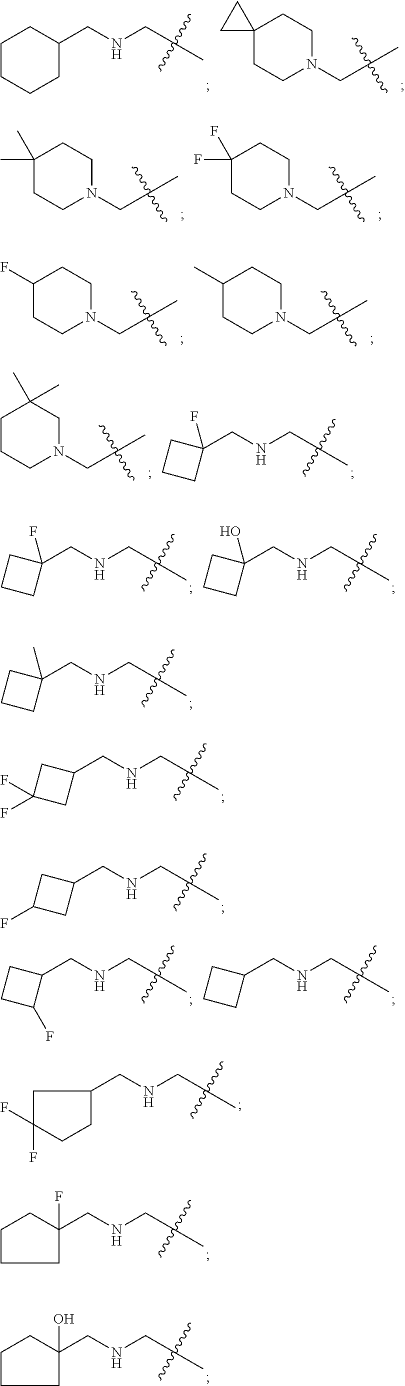

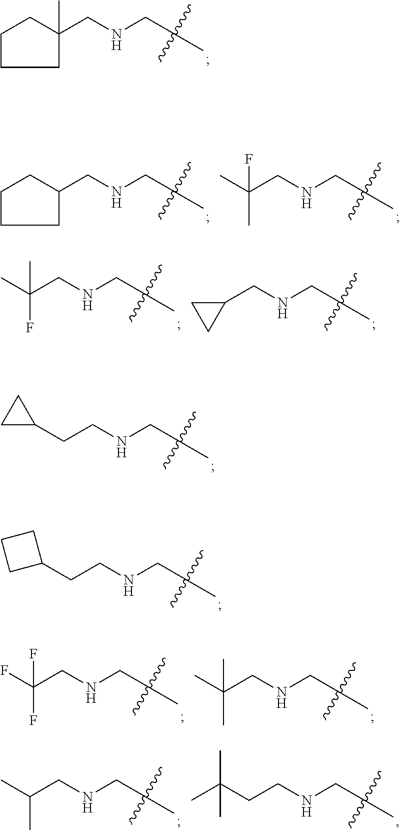

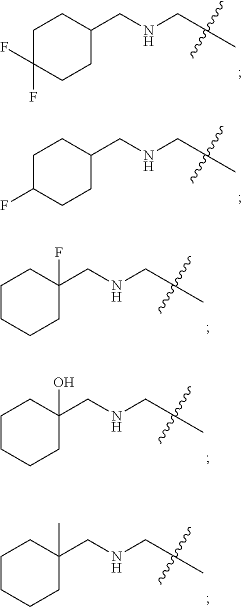 [SMILES: *CNCC1CCC(F)(F)CC1 ; *CNCC1CCC(F)CC1 ; *CNCC1(F)CCCCC1 ; *CNCC1(O)CCCCC1 ; *CNCC1(C)CCCCC1]

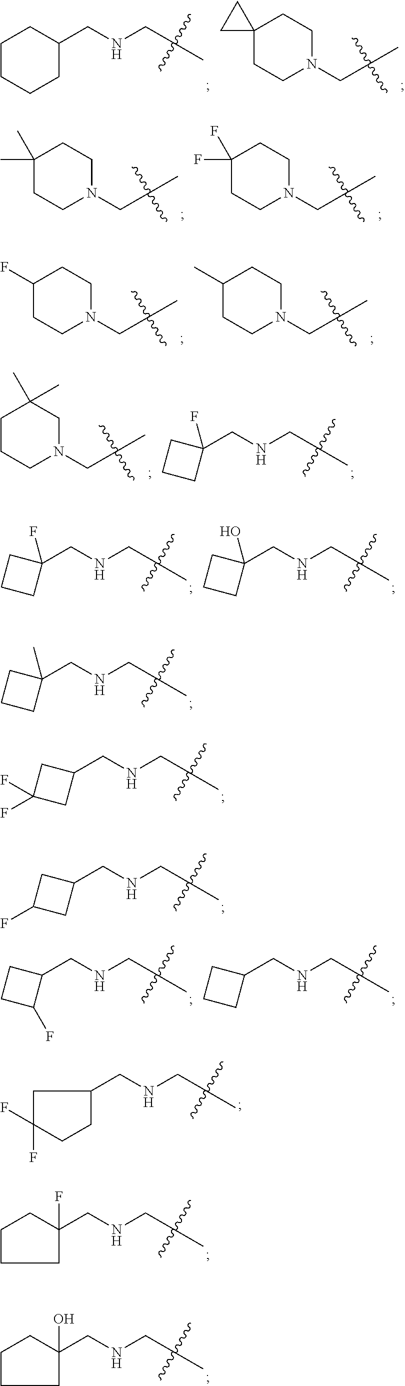

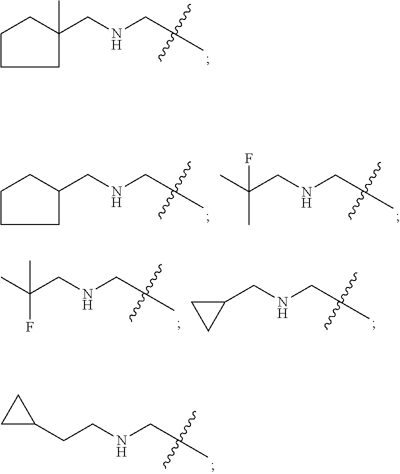

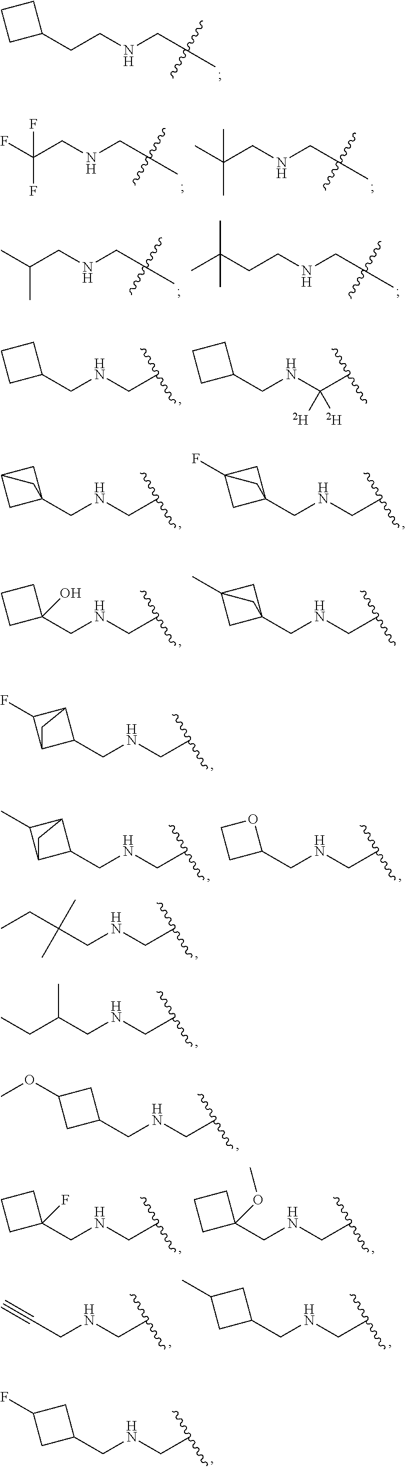

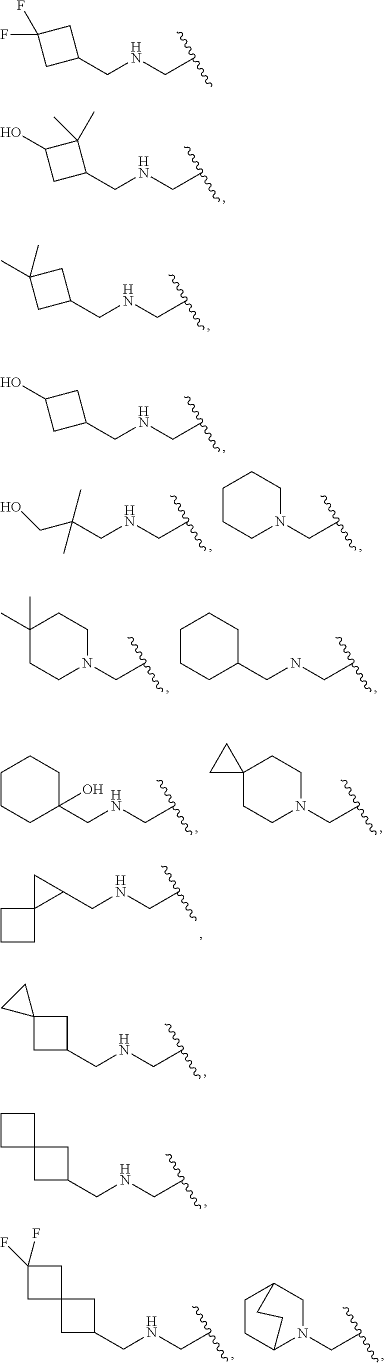

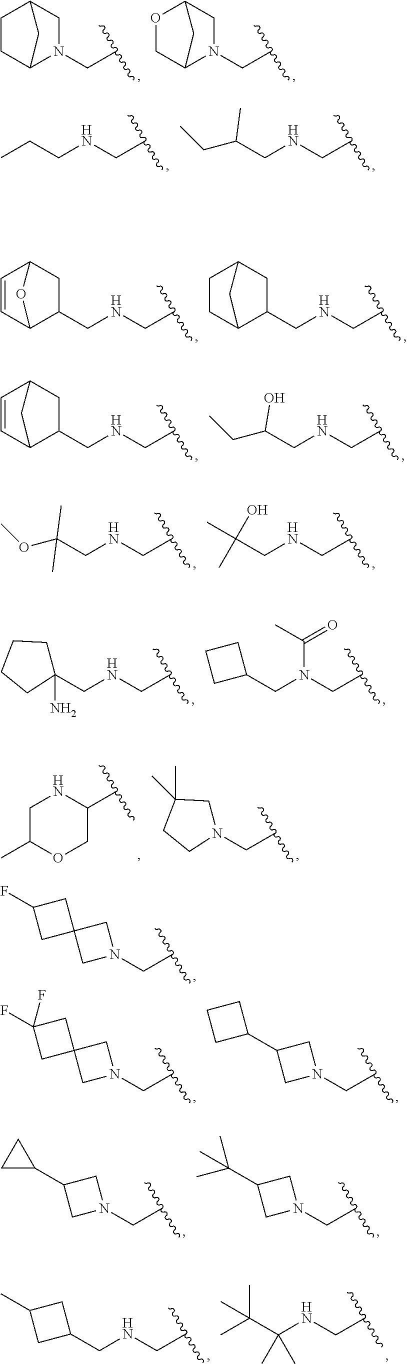

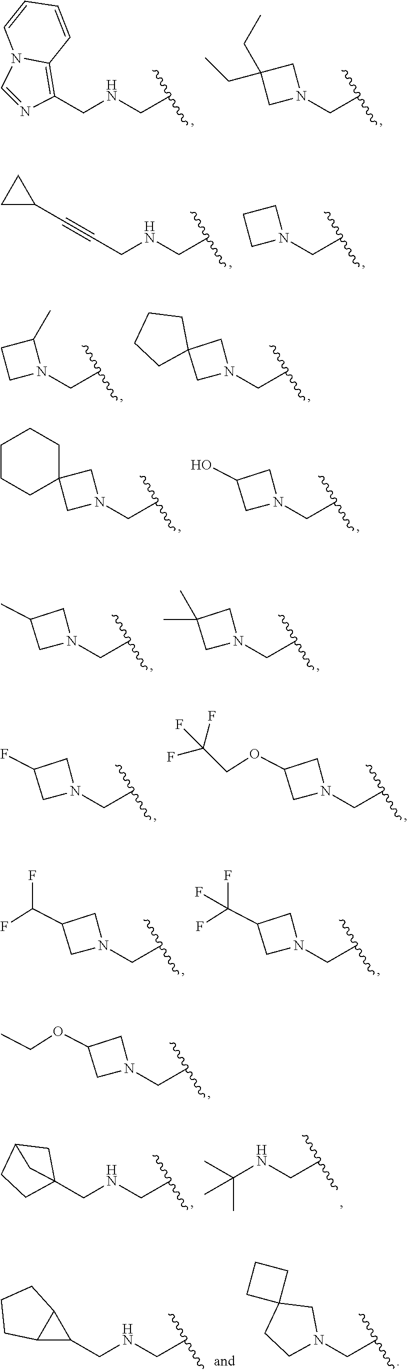

(24) R_{1b}, R_{1d} and R_{1f} are selected from:

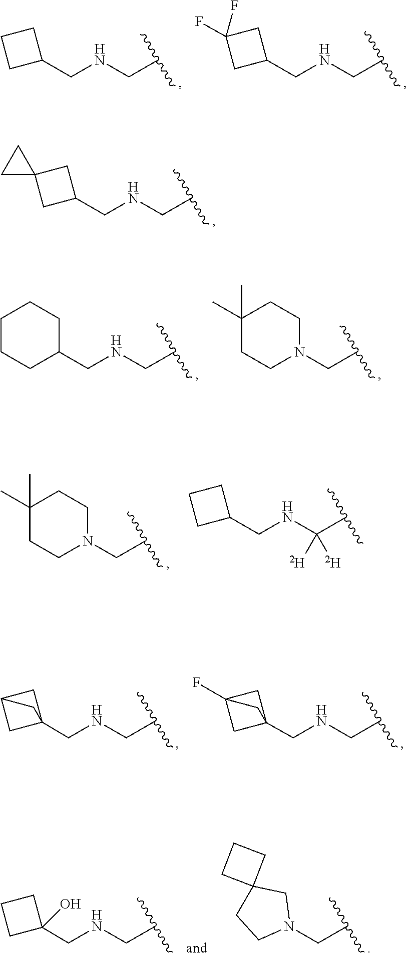

(26) R_{1b}, R_{1d} and R_{1f} are selected from:

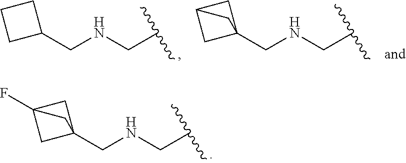

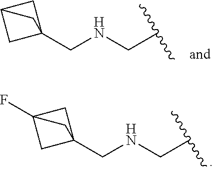 [SMILES: *CNCC12CC(C1)C2 ; *CNCC12CC(F)(C1)C2]

(27) R_{1b}, R_{1d} and R_{1f} are:

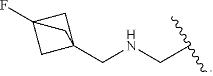 [SMILES: *CNCC12CC(F)(C1)C2]

(28) R_{1a′} is selected from hydrogen, halo and methyl;
(29) R_{1a′} is selected from hydrogen and methyl;
(30) R_{1a′} is hydrogen;
(31) R₂ₐ, R_{2b} and R_{2c} are selected from hydrogen, halo or a group of the formula:

-L₂ₐ-L_{2b}-Q₂
wherein
   L₂ₐ is absent or C₁₋₃ alkylene optionally substituted by C₁₋₂ alkyl or oxo;
   L_{2b} is absent or selected from O, S, SO, SO₂, N(Rₙ), C(O), C(O)O, OC(O), C(O)N(Rₙ), N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂ alkyl; and
   Q₂ is hydrogen, cyano, C₁₋₆ alkyl, C₃₋₆ cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄ alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)_{z}NRₚR_{q} (where z is 1, 2 or 3), wherein Rₚ and R_{q} are each independently selected from hydrogen or C₁₋₄ alkyl;
(32) R₂ₐ, R_{2b} and R_{2c} are hydrogen;
(33) X is

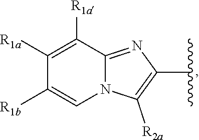

wherein R₁ₐ, R_{1b}, R_{1a′} and R₂ₐ are as herein defined;
(34) X is

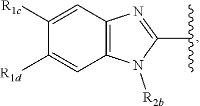

wherein R_{1c}, R_{1d} and R_{2b} are as herein defined;
(35) X is

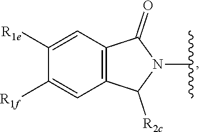

wherein R₁ₑ, R_{1f} and R_{2c} are as herein defined;
(36) X is selected from

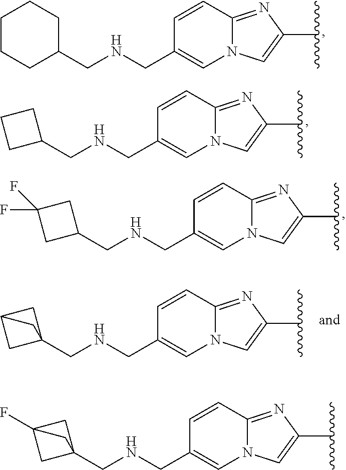

(37) X is

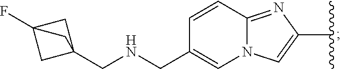 [SMILES: *c1cn2cc(CNCC34CC(F)(C3)C4)ccc2n1]

(38) R₃ₐ₁, R_{3b1}, R_{3d1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₐ₁ are independently selected from hydrogen, C₁₋₆ alkyl, C₃₋₄ cycloalkyl, hydroxy, and halo; and wherein C₁₋₆ alkyl, or C₃₋₄ cycloalkyl is optionally substituted with one or more substituents selected from halo, amino, cyano, and hydroxy; and R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂ R_{3q2}, R₃ᵣ₂ and R₃ₛ₂ are hydrogen;
(39) R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₐ₁ are independently selected from hydrogen and C₁₋₆ alkyl; and wherein C₁₋₆ alkyl is optionally substituted with one or more hydroxy substituents;
(40) R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₐ₁ are independently selected from hydrogen and methyl; and wherein methyl is optionally substituted with one or more hydroxy substituents;
(41) R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₐ₁ are independently selected from hydrogen and methyl; and wherein methyl is substituted with a hydroxy substituent;
(42) R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen;
(43) R₃ₐ₁ and R₃ₐ₂, R_{3b1} and R_{3b2}, R_{3c1} and R_{3c2}, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, R₃ᵣ₁ and R₃ᵣ₂, and R₃ₛ₁ and R₃ₛ₂ are hydrogen.
(44) R₃ₐ₁ and R₃ₐ₂, R_{3b1} and R_{3b2}, R_{3c1} and R_{3c2}, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, and R_{3q1} and R_{3q2}, R₃ᵣ₁ and R₃ᵣ₂, and R₃ₛ₁ and R₃ₛ₂ are linked to form a spiro-fused C₃₋₄ cycloalkyl which is optionally substituted with one or more substituents selected from halo, amino, cyano, and hydroxy;
(45) n is 0, 1 or 2;
(46) n is 1;
(47) Y is

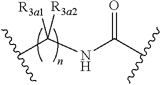

wherein R₃ₐ₁, R₃ₐ₂ and n are as herein defined;
(48) Y is

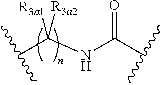

wherein n is 1, and R₃ₐ₁, R₃ₐ₂ are as herein defined;
(49) Y is

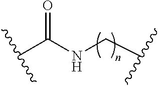

wherein n is herein defined;
(50) Y is

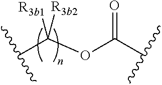

wherein R_{3b1}, R_{3b2} and n are as herein defined;
(51) Y is

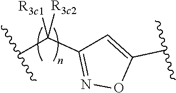

wherein R_{3c1}, R_{3d2} and n are as herein defined;
(52) Y is

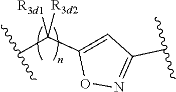

wherein R_{3d1}, R_{3d2} and n are as herein defined;
(53) Y is

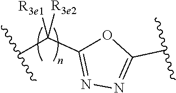

wherein R₃ₑ₁, R₃ₑ₂ and n are as herein defined;
(54) Y is

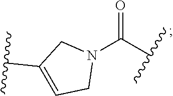 [SMILES: *C(=O)N1CC=C(*)C1]

(55) Y is

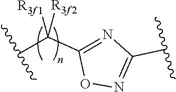

wherein R_{3f1}, R_{3f2} and n are as herein defined;
(56) Y is

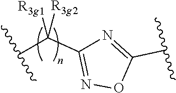

wherein R_{3g1}, R_{3g2} and n are as herein defined;
(57) Y is

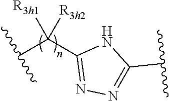

wherein R₃ₕ₁, R₃ₕ₂ and n are as herein defined
(58) Y is

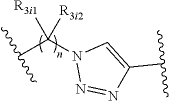

wherein R₃ᵢ₁, R₃ᵢ₂ and n are as herein defined;
(59) Y is

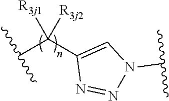

wherein R₃ⱼ₁, R₃ⱼ₂ and n are as herein defined;
(60) Y is

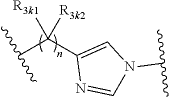

wherein R₃ₖ₁, R₃ₖ₂ and n are as herein defined;
(61) Y is

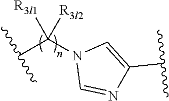

wherein R₃ₗ₁, R₃ₗ₂ and n are as herein defined;
(62) Y is

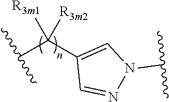

wherein R₃ₘ₁, R₃ₘ₂ and n are as herein defined;
(63) Y is

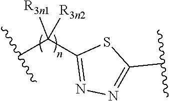

wherein R₃ₙ₁, R₃ₙ₂ and n are as herein defined;
(64) Y is

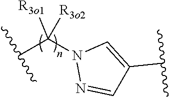

wherein R₃ₒ₁, R₃ₒ₂ and n are as herein defined;
(65) Y is

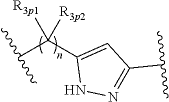

wherein R₃ₚ₁, R₃ₚ₂ and n are as herein defined;
(66) Y is

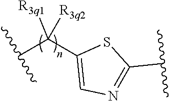

wherein R_{3q1}, R_{3q2} and n are as herein defined;
(67) Y is

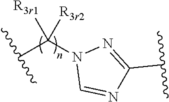

wherein R₃ᵣ₁, R₃ᵣ₂ and n are as herein defined;
(68) Y is

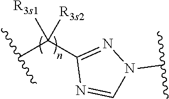

wherein R₃ₛ₁, R₃ₛ₂ and n are as herein defined;
(69) Y is selected from:

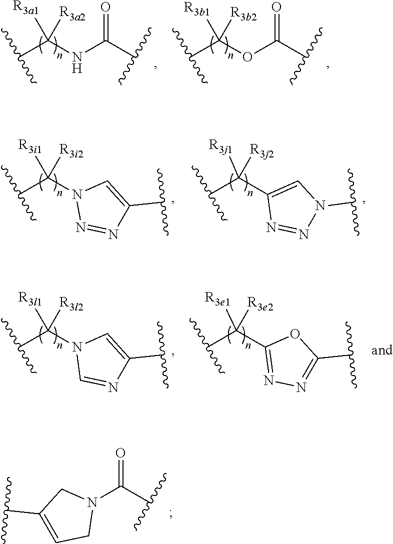

wherein R₃ₐ₁, R₃ₐ₂, R_{3b1}, R_{3b2}, R₃ₑ₁, R₃ₑ₂, R₃ᵢ₁, R₃ᵢ₂, R₃ⱼ₁, R₃ⱼ₂, R₃ᵢ₁ and R₃ᵢ₂, are as defined herein;
(70) Y is selected from:

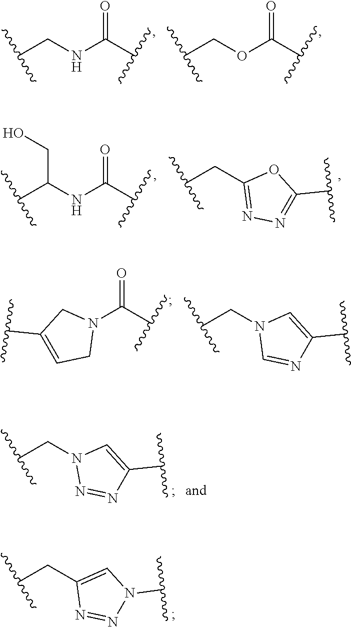

(71) Y is selected from

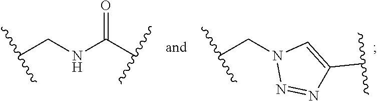 [SMILES: *CNC(*)=O ; *Cn1cc(*)nn1]

(72) Y is

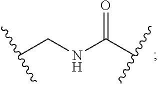 [SMILES: *CNC(*)=O]

(73) Z is selected from

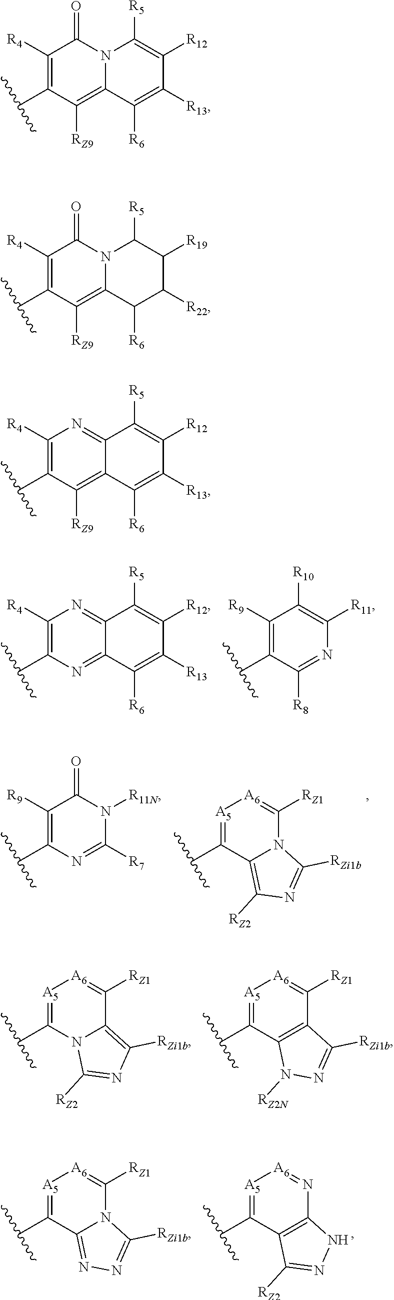

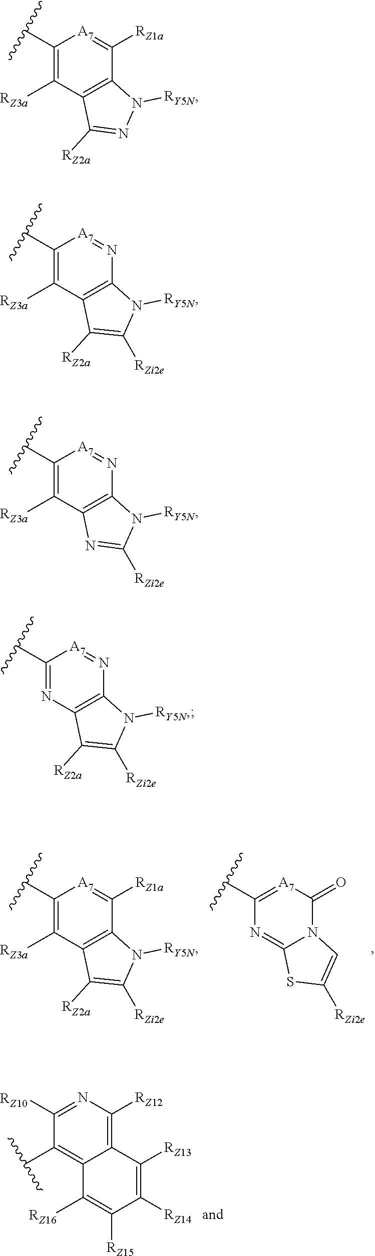

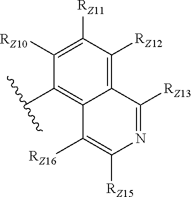

wherein:
R₄ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₅ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₆ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₁₂ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and C₁₋₄ alkyl);
R₁₃ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano, methoxy and methyl);
R₇, R₉ and R₁₁ are independently selected from hydrogen, NH₂, halo, cyano, C₁₋₄ alkoxy, C₁₋₄ haloalkoxy and C₁₋₆ alkyl;
R₈ and R₁₀ are independently selected from halo, cyano, C₁₋₄ alkoxy, C₁₋₄ haloalkoxy, C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl;
R_{11N} are independently selected from hydrogen and C₁₋₆ alkyl;
R_{Z1} and R_{Z1a} selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
R_{Z2} and R_{Z2a} are selected from hydrogen, C₁₋₄ alkyl, cyano, halo, NH₂ and C₁₋₄ alkoxy;
R_{Z3a} is selected from hydrogen, C₁₋₄ alkyl, cyano, halo, NH₂ and C₁₋₄ alkoxy;
R_{Zi1b} is selected from hydrogen, C₁₋₄ alkyl, cyano, halo, NH₂ and C₁₋₄ alkoxy;
R_{Zi2e} is selected from hydrogen, C₁₋₄ alkyl, cyano, halo, NH₂ and C₁₋₄ alkoxy;
R_{Z2N} is selected from hydrogen or C₁₋₄ alkyl
R_{Y5N} is selected from hydrogen or C₁₋₄ alkyl
R_{Z9} is selected from hydrogen, halo, cyano, C₁₋₄ alkyl, C₁₋₄ haloalkyl, C₁₋₄ alkoxy, C₁₋₄ haloalkoxy;
R_{Z10} is selected from hydrogen, halo, cyano, C₁₋₄ alkyl, C₁₋₄ haloalkyl, C₁₋₄ alkoxy, C₁₋₄ haloalkoxy;
R_{Z11} is selected from hydrogen, halo, cyano, C₁₋₄ alkyl, C₁₋₄ haloalkyl, C₁₋₄ alkoxy, C₁₋₄ haloalkoxy;
R_{Z12} is selected from hydrogen, halo, cyano, C₁₋₄ alkyl, C₁₋₄ haloalkyl, C₁₋₄ alkoxy, C₁₋₄ haloalkoxy;
R_{Z13} is selected from hydrogen, halo, cyano, C₁₋₄ alkyl, C₁₋₄ haloalkyl, C₁₋₄ alkoxy, C₁₋₄ haloalkoxy;
R_{Z14} is selected from hydrogen, halo, cyano, C₁₋₄ alkyl, C₁₋₄ haloalkyl, C₁₋₄ alkoxy, C₁₋₄ haloalkoxy;
R_{Z15} is selected from hydrogen, halo, cyano, C₁₋₄ alkyl, C₁₋₄ haloalkyl, C₁₋₄ alkoxy, C₁₋₄ haloalkoxy;
R_{Z16} is selected from hydrogen, halo, cyano, C₁₋₄ alkyl, C₁₋₄ haloalkyl, C₁₋₄ alkoxy, C₁₋₄ haloalkoxy;
A₅ is selected from CR₁₆ and N;
A₆ is selected from CR₁₇ and N;
A₇ is selected from CR₁₈ and N;
R₁₅ is selected from hydrogen, halo, cyano, methoxy and methyl;
R₁₆ and R₁₈, are selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄-cycloalkoxy;
R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₅ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring; wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O— heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
R₁₉ and R₂₀, R₂₅ and R₂₆ are selected from hydrogen, halo, cyano and C₁₋₄ alkyl;
R₂₂ and R₂₃ are selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl;
R₂₈ and R₂₉ are selected from hydrogen, halo, methoxy and methyl; and
R₂₁, R₂₄, and R₃₀ are independently selected from hydrogen or C₁₋₄ alkyl
(74) Z is selected from

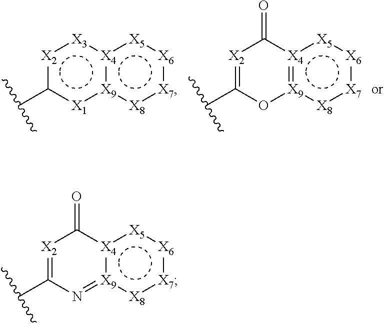

wherein X₂, X₃, X₄, X₅, X₆, X₇, X₈ and X₉ are as defined herein;
(75) Z is

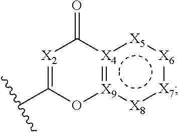

wherein X₂, X₄, X₅, X₆, X₇, X₈ and X₉ are as defined herein;
(76) Z is

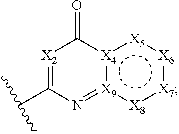

wherein X₂, X₄, X₅, X₆, X₇, X₈ and X₉ are as defined herein;
(77) Z is

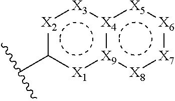

wherein X₂, X₃, X₄, X₅, X₆, X₇, X₈ and X₉ are as defined herein;
(78) Z is

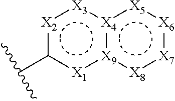

wherein
X₁ is N or CR_{Z9};
X₂ is CR₄:
X₃ is N;
X₄ is C;
X₅ is CR₅:
X₆ is A₁, wherein A₁ is CR₁₂
X₇ is A₂, wherein A₂ is CR₁₃
X₈ is N or CR₆,
X₄ is N or C;
wherein R_{Z9}, R₄, R₅, R₆, R₁₂ and R₁₃ are as defined herein.
(79) Z is:

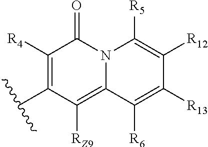

wherein R₄, R₅, R₆, R_{Z9}, R₁₂ and R₁₃ are as herein defined;
(80) Z is:

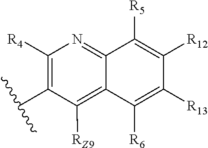

wherein R₄, R₅, R₆, R₁₂, R₁₃ and R_{Z9} are as herein defined;
(81) Z is:

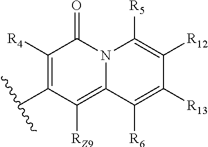

wherein R₄, R₅, R₈, R₁₉, R₂₂ and R_{Z9} are as herein defined;
(82) Z is:

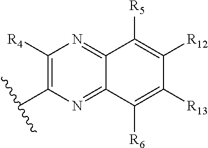

wherein R₄, R₅, R₆, R₁₂, R₁₃ and R_{Z9} are as herein defined;
(83) Z is

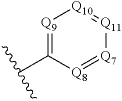

wherein Q₇, Q₈, Q₉, Q₁₀ and Q₁₁ are as defined herein;
(84) Z is

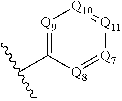

wherein Q₈, Q₉, Q₁₀ and Q₁₁ are as defined herein;
(84a) Z is selected from:

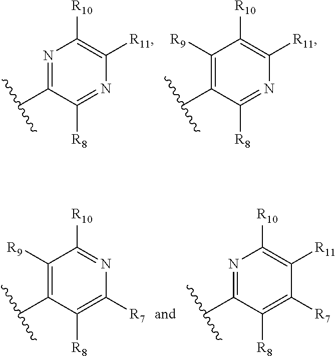

wherein R₈, R₉, R₁₀ and R₁₁ are as herein defined;
(85) Z is selected from:

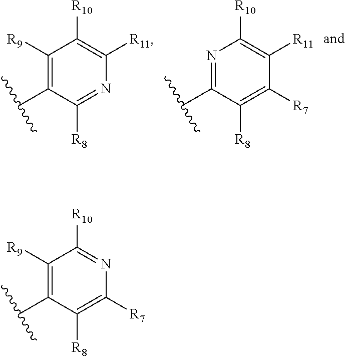

wherein R₈, R₉, R₁₀ and R₁₁ are as herein defined;
(86) Z is

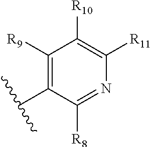

wherein R₈, R₉, R₁₀ and R₁₁ are as herein defined;
(87) Z is

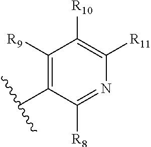

wherein:
R₈ is selected from cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl,
R₁₀ is C₁₋₄ alkoxy or C₁₋₄ haloalkoxy;
R₉ and R₁₁ are as herein defined;
(87a) Z is

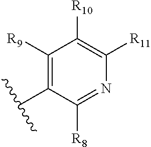

wherein:
R₈ is selected from hydrogen, cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl,
R₁₀ is C₁₋₄ alkoxy or C₁₋₄ haloalkoxy;
R₉ and R₁₁ are as herein defined;
(88) Z is

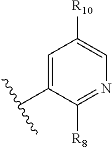

wherein R₈ is cyano and R₁₀ is C₁₋₄ alkoxy;
(88a) Z is

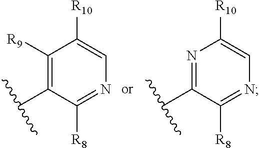

wherein
R₈ is hydrogen or cyano;
R₉ is hydrogen; and
R₁₀ is selected from C₁₋₄ alkoxy and heterocyclyl.
(88b) Z is

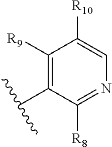

wherein
R₈ is hydrogen or cyano;
R₉ is hydrogen; and
R₁₀ is methoxy.
(89) Z is N

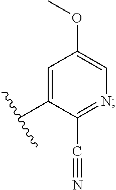 [SMILES: *c1cc(OC)cnc1C#N]

(89a) Z is

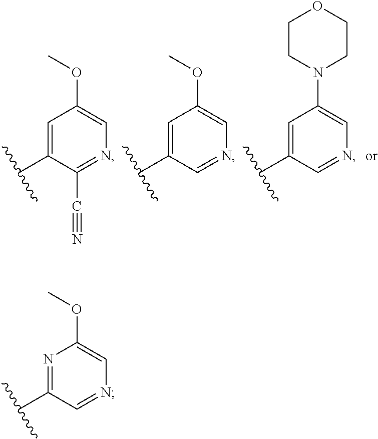

(89b) Z is

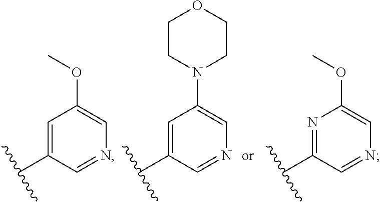

(89c) Z is

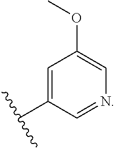 [SMILES: *c1cncc(OC)c1]

(90) Z is

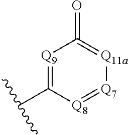

wherein Q₈, Q₉, Q₁₀ and Q₁₁ₐ are as defined herein;
(91) Z is

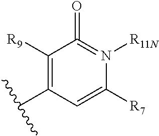

wherein R₇, R₉ and R_{11N} are as herein defined;
(92) Z is

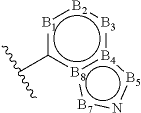

wherein B₁, B₂, B₃, B₄, B₅, B₇ and B₈ are as defined herein;
(93) Z is:

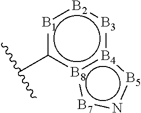

wherein
B₁ is A₅, wherein A₅ is N or CR₁₆;
B₂ is A₆, wherein A₆ is CR₁₇
B₃ is N or CR_{Z1};
B₄ is N or C;
B₅ is selected from CR_{Zi1b} or NR_{B5N}
B₇ is N, NR_{Z2N} or CR_{Z2};
B₈ is selected from C or N;
wherein R₁₆, R₁₇, R_{Z1}, R_{zi1b}, R_{B5N}, R_{Z2} are as defined herein
(94) Z is:

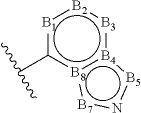

wherein
B₁ is A₅, wherein A₅ is CR₁₆;
B₂ is A₆, wherein A₆ is CR₁₇
B₃ is CR_{Z1};
B₄ is N or C;
B₅ is selected from CR_{Zi1b} or NR_{B5N}
B₇ is N, NH or CR_{Z2};
B₈ is C;
wherein R₁₆, R₁₇, R_{Z1}, R_{zi1b}, R_{B5N}, R_{Z2} are as defined herein;
(95) Z is:

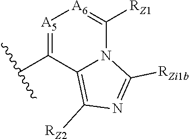

wherein R_{Z1}, R_{Z2}, R_{zi1b}, A₅ and A₆ are as herein defined;
(96)
(97) Z is:

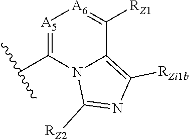

wherein R_{Z1}, R_{Z2}, R_{zi1b}, A₅ and A₆ are as herein defined;
(98) Z is

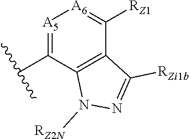

wherein A₅, A₆, R_{Z1}, R_{Z2N} are R_{zi1b} are as herein defined;
(99) Z is

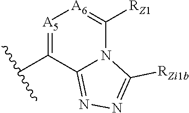

wherein A₅, A₆, R_{Z1}, R_{Z2} are R_{zi1b} are as herein defined;
(100) Z is:

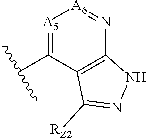

wherein R_{Z1}, R_{Z2} and A₆ are as herein defined;
(101) Z is

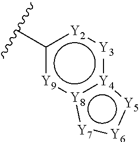

wherein
Y₂ is A₇, wherein A₇ is CR₁₈;
Y₃ is N or CR_{Z1}a;
Y₄ is C or N
Y₅ is NR_{Y5N};
Y₆ is C—R_{Zi2e} or N;
Y₇ is CR_{Z2a} or N;
Y₈ is C or N;
Y₉ is CR_{Z3a} or N;
wherein R₁₈, R_{z1a}, R_{Y5N}, R_{Zi2e}, R_{Z2a} and R_{Z3a} are as defined herein;
(102) Z is

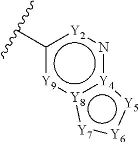

wherein
Y₂ is A₇, wherein A₇ is CR₁₃;
Y₄ is C or N
Y₅ is NR_{Y5N};
Y₆ is C—R_{Zi2e} or N;
Y₇ is CR_{Z2a} or N;
Y₈ is C or N;
Y₉ is CR_{Z3a} or N;
wherein R₁₈, R_{z1a}, R_{Y5N}, R_{Zi2e}, R_{Z2a} and R_{Z3a} are as defined herein;
(103) Z is

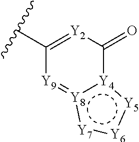

wherein
Y₂ is A₇, wherein A₇ is CR₁₈;
Y₄ is N
Y₅ is C—R_{Y5};
Y₆ is C—R_{Zi2e};
Y₇ is O or S;
Y₈ is C;
Y₉ is N;
wherein R₁₈, R_{Y5}, R_{Zi2e} are as defined herein;
(104) Z is

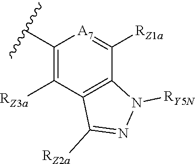

wherein A₇, R_{Z1a}, R_{Z2a} and R_{Z3a} are as herein defined, and R_{Y5N} is C₁₋₄ alkyl;
(105) Z is:

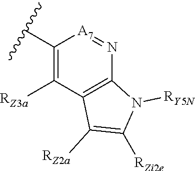

wherein R_{Y5N}, R_{Z2a}, R_{Z3a}, R_{Zi2e} and A₇ are as herein defined;
(106) Z is:

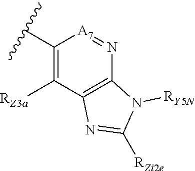

wherein R_{Y5N}, R_{Z2a}, R_{Z3a}, R_{Zi2e} and A₇ are as herein defined;
(107) Z is:

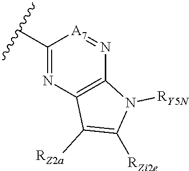

wherein R_{Y5N}, R_{Z2a}, R_{Zi2e} and A₇ are as herein defined;
(108) Z is:

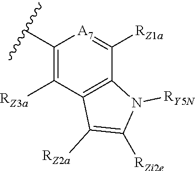

wherein R_{Y5N}, R_{Z1a}, R_{Z2a}, R_{Z3a}, R_{Zi2e} and A₇ are as herein defined;
(109) Z is:

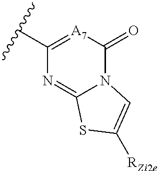

wherein A₇ and R_{Zi2e} are as defined herein;
(110) Z is

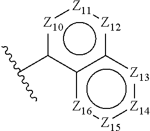

wherein Z₁₀, Z₁₁, Z₁₂, Z₁₃, Z₁₄, Z₁₅ and Z₁₆ are as defined herein;
(111) Z is

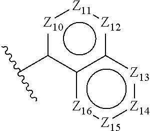

wherein
Z₁₀ is CR_{Z10},
Z₁₁ is N;
Z₁₂ is CR_{Z12};
Z₁₃ is CR_{Z13};
Z₁₄ is N or CR_{Z14};
Z₁₅ is N or CR_{Z15};
Z₁₆ is CR_{Z16};
wherein R_{Z10}, R_{Z12}, R_{Z13}, R_{Z14}, R_{Z15} and R_{Z16} are as defined herein;
(112) Z is

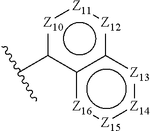

wherein
Z₁₀ is CR_{Z10},
Z₁₁ is CR_{Z10} or N;
Z₁₂ is CR_{Z12};
Z₁₃ is CR_{Z13};
Z₁₄ is N;
Z₁₅ is N or CR_{Z15};
Z₁₆ is CR_{Z16};
wherein R_{Z10}, R_{Z12}, R_{Z13}, R_{Z14}, R_{Z15} and R_{Z16} are as defined herein;
(113) Z is:

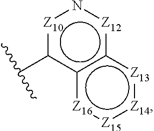

wherein Z₁₀, Z₁₂, Z₁₃, Z₁₄, Z₁₅ and Z₁₆ are as herein defined;
(114) Z is:

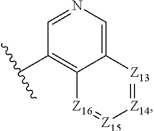

wherein Z₁₃, Z₁₄, Z₁₅ and Z₁₆ are as herein defined;
(115) Z is:

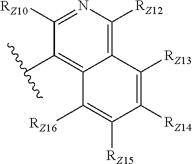

wherein R_{Z10}, R_{Z12}, R_{Z13}, R_{Z14}, R_{Z15} and R_{Z16} are as herein defined;
(116) Z is:

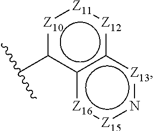

wherein Z₁₀, Z₁₁, Z₁₂, Z₁₃, Z₁₅ and Z₁₆ are as herein defined,
(117) Z is:

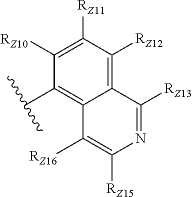

wherein R_{Z10}, R_{Z11}, R_{Z12}, R_{Z13}, R_{Z15} and R_{Z16} are as herein defined;
(118) Z is selected from:

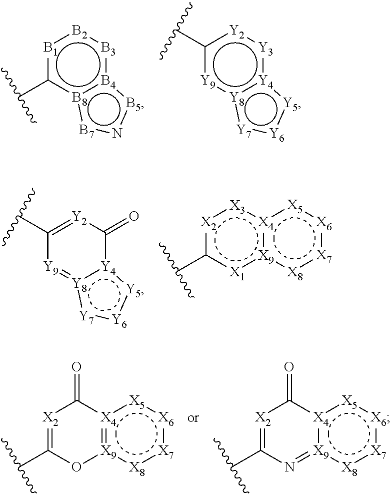

wherein B₁, B₂, B₃, B₄, B₅, B₇, B₈, Y₂, Y₃, Y₄, Y₅, Y₆, Y₇, Y₈, Y₉, X₁, X₂, X₃, X₄, X₅, X₆, X₇, X₈ and X₉ are as defined herein;
(119) Z is selected from:

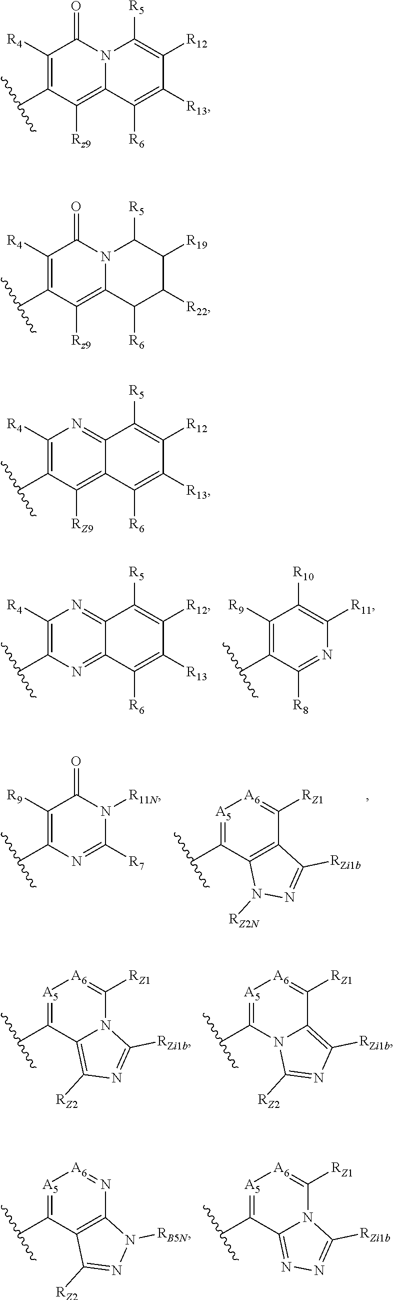

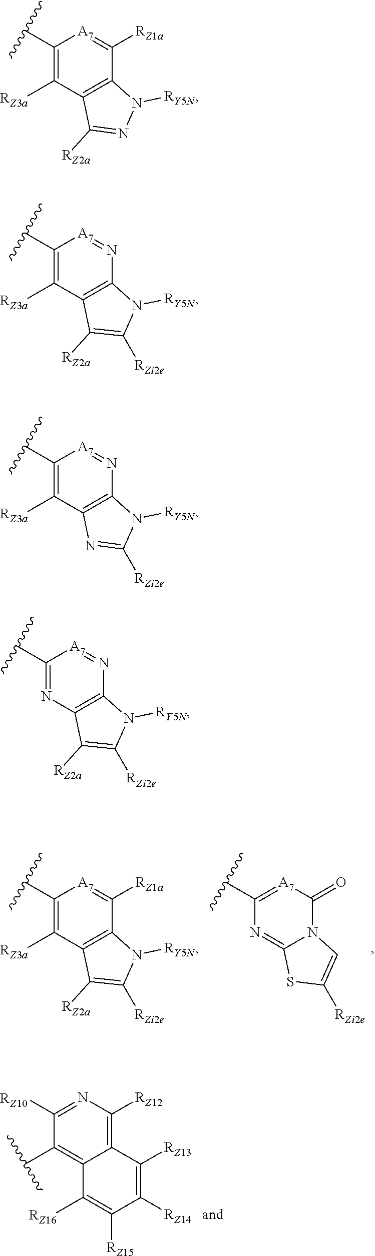

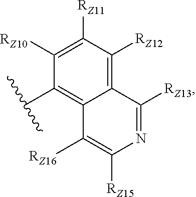

wherein
A₅, A₆, A₇, R₄, R₅, R₆, R₇, R₈, R₉, R₁₀, R₁₁, R_{11N}, R₁₂, R₁₃, R₁₉, R₂₂, R_{B5N}, R_{Y5N}, R_{Z1}, R_{Z2}, R_{Z9}, R_{Z10}, R_{Z11}, R_{Z12}, R_{Z13}, R_{Z14}, R_{Z15}, R_{Z16}, R_{Z2a}, R_{Z3a}, R_{Z1a}, R_{Zi1b} and R_{Zi2e} are as defined herein;
(120) Z is selected from:

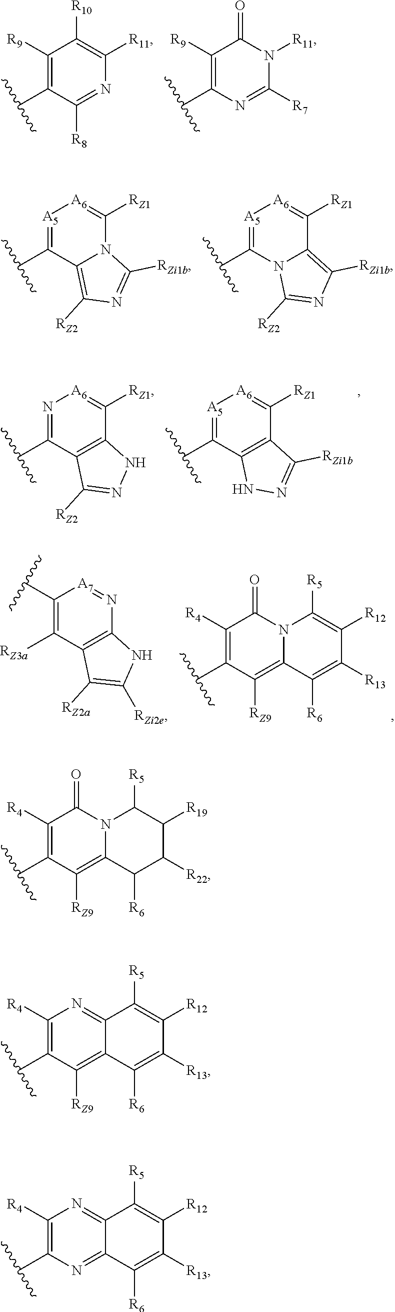

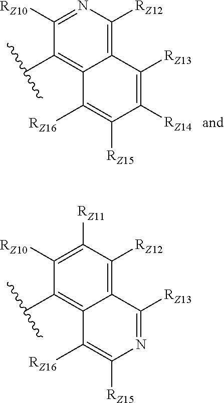

wherein A₅, A₆, A₇, R₄, R₅, R₆, R₇, R₈, R₉, R₁₀, R₁₁, R_{11N}, R₁₂, R₁₃, R₁₉, R₂₂, R_{Z1}, R_{Z2}, R_{Z9}, R_{Z10}, R_{Z11}, R_{Z12}, R_{Z13}, R_{Z14}, R_{Z15}, R_{Z16}, R_{Z2a}, R_{Z3a}, R_{Z1a}, R_{Zi1b} and R_{Zi2e} are as defined herein;
(121) Z is selected from:

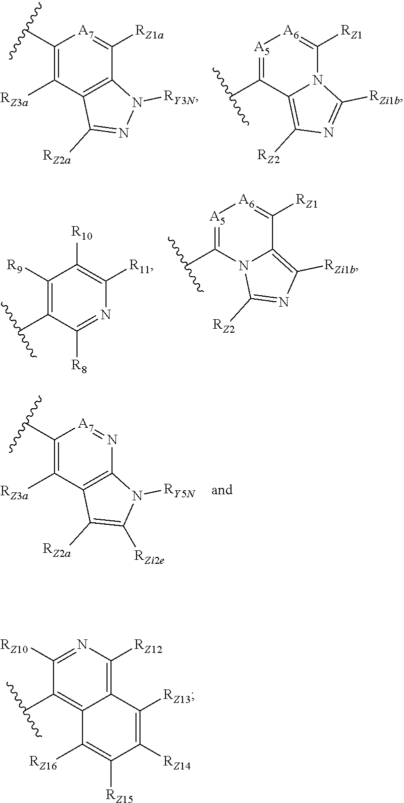

wherein A₅, A₆, A₇, R₈, R₉, R₁₀, R₁₁, R_{Y5N}, R_{Z1}, R_{Z2}, R_{Z10}, R_{Z12}, R_{Z13}, R_{Z14}, R_{Z15}, R_{Z16}, R_{Z1a}, R_{Z2a}, R_{Z3a}, R_{Zi1b} and R_{Zi2e} are as defined herein;
(122) Z is selected from:

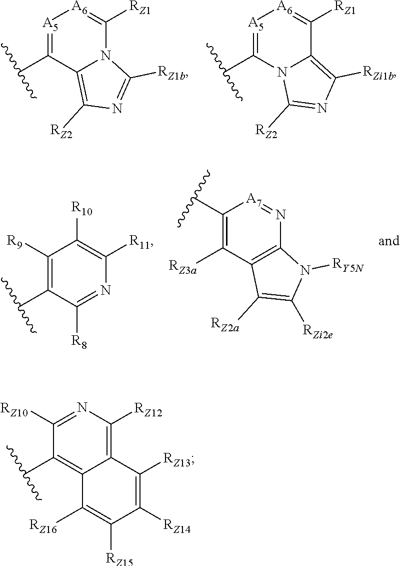

wherein A₅, A₆, A₇, R₈, R₉, R₁₀, R₁₁, R_{Y5N}, R_{Z1}, R_{Z2}, R_{Z10}, R_{Z12}, R_{Z13}, R_{Z14}, R_{Z15}, R_{Z16}, R_{Z2a}, R_{Z3a}, R_{Z1a}, R_{Zi1b} and R_{Zi2e} are as defined herein.
(123) Z is selected from:

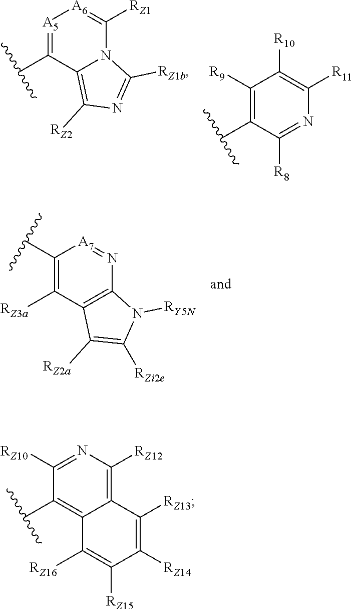

wherein A₅, A₆, A₇, R₈, R₉, R₁₀, R₁₁, R_{Y5N}, R_{Z1}, R_{Z2}, R_{Z10}, R_{Z12}, R_{Z13}, R_{Z14}, R_{Z15}, R_{Z16}, R_{Z2a}, R_{Z3a}, R_{Z1a}, R_{Zi1b} and R_{Zi2e} are as defined herein.
(124) Z is selected from:

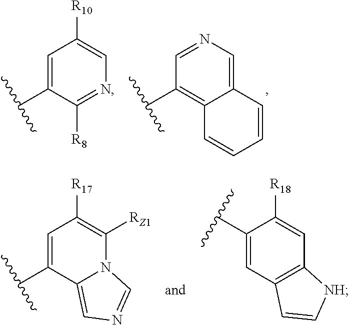

wherein R₈, R₁₀, R₁₇, R₁₈ and R_{Z1}, are as herein defined.
(125) Z is selected from:

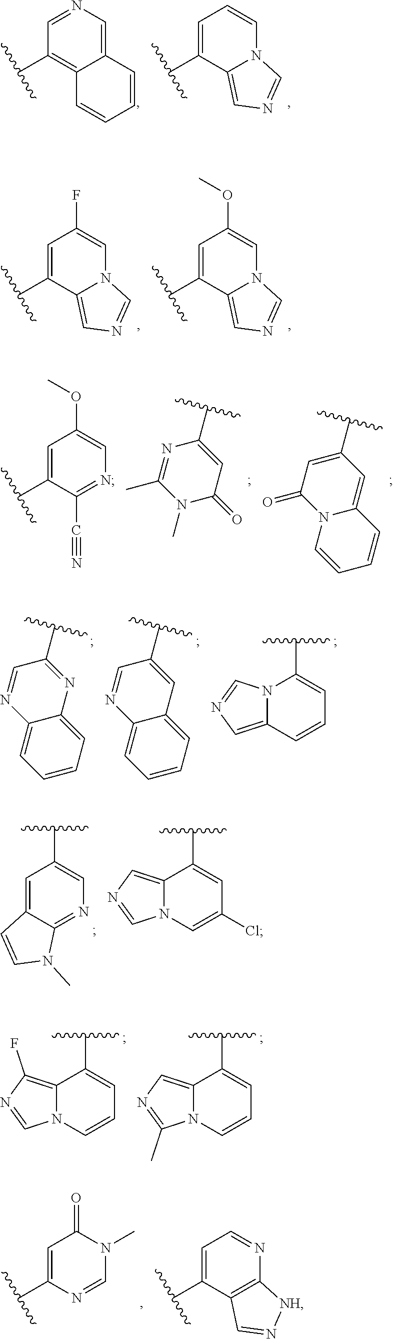

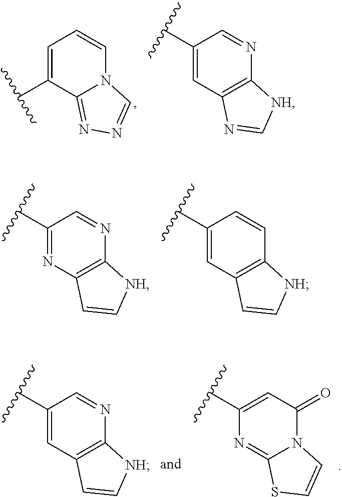

(126) Z is selected from:

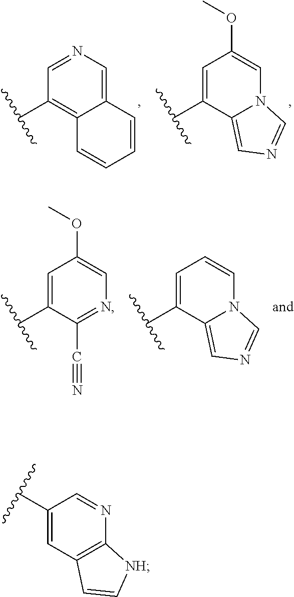

(127) R₄, R₅ and R₆ are independently selected from hydrogen and halo;
(128) R₄, R₅ and R₆ are hydrogen;
(129) when Z is

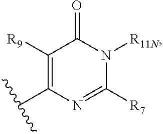

then R₇, R₉ are independently selected from hydrogen, methyl, halo and cyano; and R_{11N} is selected from hydrogen and methyl;
(129a) when Z is

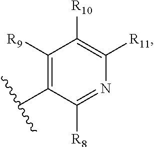

then R₈, R₉, R₁₀ and R₁₁ are independently selected from hydrogen, heterocyclyl, NH₂, halo, cyano, C₁₋₄ alkoxy, C₁₋₄ haloalkoxy and C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; with the proviso that at least one of R₈, R₉, R₁₀ and R₁₁ is hydroxy, NRᵥ₁Rᵥ₂, heterocyclyl, C₁₋₄ alkoxy or C₁₋₄ haloalkoxy;
(130) when Z is

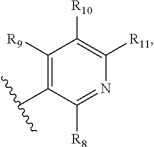

then R₈, R₉, R₁₀ and R₁₁ are independently selected from hydrogen, NH₂, halo, cyano, C₁₋₄ alkoxy, C₁₋₄ haloalkoxy and C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl with the proviso that at least one of R₈, R₉, R₁₀ and R₁₁ is C₁₋₄ alkoxy or C₁₋₄ haloalkoxy; (130a) when Z is

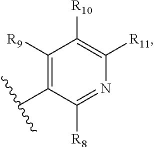

then R₈, R₉ and R₁₁ are independently selected from hydrogen, NH₂, halo, cyano, C₁₋₄ alkoxy, C₁₋₄ haloalkoxy, C₁₋₃ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; and
R₁₀ is selected from heterocyclyl, C₁₋₄ alkoxy or C₁₋₄ haloalkoxy.
(131) when Z is

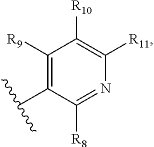

then R₈, R₉ and R₁₁ are independently selected from hydrogen, NH₂, halo, cyano, C₁₋₄ alkoxy, C₁₋₄ haloalkoxy, C₁₋₃ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; and R₁₀ is selected from C₁₋₄ alkoxy or C₁₋₄ haloalkoxy.
(132) when Z is

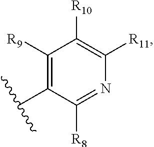

then R₈ is cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; R₉ is hydrogen; R₁₀ is C₁₋₄ alkoxy; and R₁₁ is hydrogen.
(133) when Z is

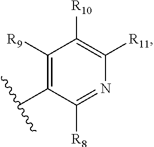

then R₈ is cyano; R₉ is hydrogen; R₁₀ is C₁₋₄ alkoxy or C₁₋₄ haloalkoxy; and R₁₁ is hydrogen.
(133a) when Z is

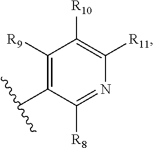

then R₈ is hydrogen or cyano; R₉ is hydrogen; R₁₀ is selected from C₁₋₄ alkoxy or heterocyclyl; and R₁₁ is hydrogen.
(133b) when Z is

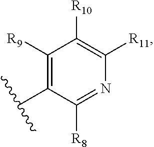

then R₈ is hydrogen or cyano; R₉ is hydrogen; R₁₀ is C₁₋₄ alkoxy; and R₁₁ is hydrogen.
(133c) when Z is

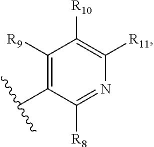

then R₈ is hydrogen or cyano; R₉ is hydrogen; R₁₀ is C₁₋₄ alkoxy; and R₁₁ is hydrogen.
(134) R_{Z1} and R_{Z1a} are selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
(135) R_{Z1} and R_{Z1a} are selected from hydrogen, C₁₋₂ alkyl, cyano, halo, C₁₋₂ haloalkyl, C₁₋₂ haloalkoxy, C₁₋₂ alkoxy;
(136) R_{Z1} and R_{Z1a} are selected from hydrogen, C₁₋₂ alkyl, cyano and halo;
(137) R_{Z1} and R_{Z1a} are hydrogen;
(138) R_{Z2}, R_{Z2a}, R_{Z3a}, R_{Zi1b} and R_{Zi2e} are independently selected from hydrogen, C₁₋₄ alkyl, cyano, halo or C₁₋₄ alkoxy;
(139) R_{Z2}, R_{Z2a}, R_{Z3a}, R_{Zi1b} and R_{Zi2e} are independently selected from hydrogen, cyano or halo;
(140) R_{Z2}, R_{Z2a}, R_{Z3a}, R_{Zi1b} and R_{Zi2e} are hydrogen;
(141) R_{Y5N}, R_{Z2N}, and R_{11N} are selected from hydrogen or methyl;
(142) R_{Y5N}, R_{Z2N}, and R_{11N} are hydrogen;
(143) R_{Y5N} is methyl;
(144) R_{Z4}, R_{Z5}, R_{Z6}, R_{Z7}, R_{Z8}, R_{Z9}, R_{Z10}, R_{Z11}, R_{Z12}, R_{Z13}, R_{Z14}, R_{Z15}, and R_{Z16} are independently selected from hydrogen, halo and cyano;
(145) R_{Z4}, R_{Z5}, R_{Z6}, R_{Z7}, R_{Z8}, R_{Z9}, R_{Z10}, R_{Z11}, R_{Z12}, R_{Z13}, R_{Z14}, R_{Z15} and R_{Z16} are independently selected from hydrogen and halo;
(146) R_{Z4}, R_{Z5}, R_{Z6}, R_{Z7} R_{Z8}, R_{Z9}, R_{Z10}, R_{Z11}, R_{Z12}, R_{Z13}, R_{Z14}, R_{Z15}, R_{Z16} are hydrogen;
(147) R₁₂, R₁₃, R₁₆, R₁₉, R₂₀, R₂₁, R₂₂ and R₂₃ are independently selected from hydrogen, halo, cyano and methyl;
(148) R₁₂, R₁₃, R₁₆, R₁₉, R₂₀, R₂₁, R₂₂ and R₂₃ are hydrogen;
(149) R₁₂ is selected from hydrogen, halo, cyano and C₁₋₄ alkyl;
(150) R₁₂ is selected from hydrogen and halo;
(151) R₁₂ is selected from hydrogen and chloro;
(152) R₁₂ is hydrogen;
(153) R₁₃ is selected from hydrogen, halo, methoxy, cyano and methyl;
(154) R₁₃ is selected from hydrogen, methoxy and methyl;
(155) R₁₃ is hydrogen;
(156) R₁₆ and R₁₈ are selected from hydrogen, halo, cyano, C₁₋₄ alkyl, C₁₋₄ alkoxy, C₁₋₄ haloalkyl and C₁₋₄ haloalkoxy;
(157) R₁₆ and R₁₈ are selected from hydrogen, halo, cyano and C₁₋₄ alkyl;
(158) R₁₆ and R₁₈ are selected from hydrogen and halo;
(159) R₁₆ and R₁₈ are hydrogen;
(160) R₁₇ is selected from hydrogen, halo, cyano, C₁₋₅ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₄ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring;
   wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.
(161) R₁₇ is selected from hydrogen, halo, cyano, C₁₋₂ alkyl, C₁₋₂ haloalkyl, C₁₋₂ alkoxy, C₁₋₂ haloalkoxy, C₂₋₃ alkenyl, C₂₋₃ alkynyl, phenyl a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₂ alkyl or R_{q} and Rᵣ are linked together such that, together with the nitrogen atom to which they are attached, they form a 3- to 6-membered heterocyclic ring;
   wherein any C₁-alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.
(162) R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is 1, 2 or 3, NR_{q}Rᵣ, wherein R_{q} and Rᵣ are each independently hydrogen, C₁₋₄ alkyl; or R_{q} and Rᵣ are linked together such that, together with the nitrogen atom to which they are attached, they form a 3- to 6-membered heterocyclic ring;
   wherein any C₁₋₄ alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, C₁₋₂ haloalkyl, cyano, hydroxy, C₁₋₂ alkoxy, halo and C₁₋₂ haloalkoxy.
(163) R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, C₃₋₆ cycloalkyl, —O—C₃₋₄ cycloalkyl, heterocyclyl, —(OCH₂CH₂)ₘ—OCH₃ wherein m is 1, 2 or 3, NR_{q}Rᵣ, wherein R_{q} and Rᵣ are each independently hydrogen or C₁₋₂ alkyl;
   wherein any C₁₋₄ alkyl, C₂₋₄ alkenyl, C₂₋₄ alkynyl, C₃₋₆ cycloalkyl, —O—C₃₋₄ cycloalkyl, heterocyclyl, system is optionally further substituted by one or more substituents selected from C₁₋₂ alkyl, C₁₋₂ haloalkyl, cyano, hydroxy, C₁₋₂ alkoxy, halo and C₁₋₂ haloalkoxy.
(164) R₁₇ is selected from hydrogen, halo, cyano, C₁₋₄ alkyl, C₁₋₄ alkoxy, C₂₋₄ alkenyl, C₂₋₄ alkynyl and a 5- or 6-membered aryl or heteroaryl; wherein any C₁₋₄ alkyl, C₂₋₄ alkenyl, C₂₋₄ alkynyl, 5- or 6-membered aryl or heteroaryl is optionally further substituted by one or more substituents selected from C₁₋₂ alkyl, C₁₋₂ haloalkyl, cyano, hydroxy, C₁₋₂ alkoxy, halo and C₁₋₂ haloalkoxy.
(165) R₁₇ is selected from hydrogen, halo, C₁₋₄ alkoxy, C₂₋₄ alkynyl and 5- or 6-membered aryl or heteroaryl.
(166) R₁₇ is selected from hydrogen, C₁₋₄ alkoxy, bromo, ethynyl, and pyrazoyl.
(167) R₁₉ is selected from hydrogen, halo or C₁₋₄ alkyl;
(168) R₁₉ is hydrogen or methyl;
(169) R₁₉ is hydrogen;
(170) R₂₁, R₂₄ and R₃₀, are independently selected from hydrogen or methyl;
(171) R₂₁, R₂₄ and R₃₀, are hydrogen;
(172) R₂₂ is selected from hydrogen, halo, cyano and methyl;
(173) R₂₂ is hydrogen or methyl;
(174) R₂₂ is hydrogen;
(175) R₂₈ and R₂₉ are selected from hydrogen or halo, methoxy and methyl;
(176) R₂₈ and R₂₉ are hydrogen;

In an embodiment:
when Xₐ is A₁, A₁ is CR₁₂;
when X₇ is A₂, A₂ is CR₁₃;
B₁ is A₅, wherein A₅ is CR₁₆;
B₂ is A₆, wherein A₆ is CR₁₇;
Y₂ is A₇, wherein A₇ is CR₁₈;
when X₆ is A₈, A₈ is CR₁ₑR₂₀;
when X₇ is A₉, A₉ is CR₂₂R₂₃;
when X₇ is A₁₁, A₁₁ is CR₂₈R₂₉;
   and wherein R₁₂, R₁₃, R₁₆, R₁₇, R₁₈, R₁₉, R₂₀, R₂₁, R₂₂, R₂₃, R₂₈ and R₂₉ are as defined herein.

In an embodiment: A₁ is CR₁₂; A₂ is CR₁₃; A₅ is CR₁₆; A₆ is CR₁₇; A₇ is CR₁₈; A₈ is CR₁₉R₂₀; A₉ is CR₂₂R₂₃; A₁₁ is CR₂₈R₂₉; wherein R₁₂, R₁₃, R₁₆, R₁₇, R₁₈, R₁₉, R₂₀, R₂₁, R₂₂, R₂₃, R₂₈ and R₂₉ are as defined herein

In an embodiment:
(i) R₄, R₅, R_{X5a}, R_{X5b}, R_{Y5}, R₆, R₇, R₉, R₁₁, R₁₁ₐ, R_{11b}, R_{Z2}, R_{Z2a}, R_{Z3a}, R_{Zi1b}, R_{Zi2e}, R_{Z9}, R_{Z10}, R_{Z11}, R_{Z12}, R_{Z13}, R_{Z14}, R_{Z15} and R_{Z16} are independently selected from hydrogen, methyl, cyano or halo; and R_{B5N}, R_{Y5N}, R_{Z2}N and R_{11N} are selected from methyl or hydrogen;
(ii) R₈ is selected from cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl;
(iii) R₁₀ is selected from C₁₋₄ alkoxy or C₁₋₄ haloalkoxy;
(iv) R_{Z1} and R_{Z1a} are selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl; (v) R₁₂, R₁₃, R₁₆, R₁₈, R₁₉, R₂₀, R₂₁, R₂₂, R₂₃, R₂₄ and R₃₀ are independently selected from hydrogen, halo, cyano and methyl;
(vi) R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl a 5- or 6-membered or heteroaryl, C₃₋₆ cycloalkyl, —O—C₃₋₆ cycloalkyl, heterocyclyl, —(OCH₂CH₂)ₘ—OCH₃ wherein m is an integer from 1 to 6, NR_{q}Rᵣ, wherein R_{q} and Rᵣ are each independently hydrogen, C₁₋₄ alkyl or R_{q} and Rᵣ are linked together such that, together with the nitrogen atom to which they are attached, they form a 3- to 6-membered heterocyclic ring; wherein any C₁₋₄ alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.
(vii) R₂₁, R₂₄ and R₃₀, are independently selected from hydrogen or methyl;
(viii) R₂₈ and R₂₉ are selected from hydrogen or halo, methoxy and methyl.

In an embodiment:
(i) R₄, R₅, R_{X5a}, R_{X5b}, R_{Y5}, R₆, R₇, R₉, R₁₁, R₁₁ₐ, R_{11b}, R_{Z2}, R_{Z2a}, R_{Z3a}, R_{Zi1b}, R_{Zi2e}, R_{Z9}, R_{Z10}, R_{Z11}, R_{Z12}, R_{Z13}, R_{Z14}, R_{Z15} and R_{Z16} are independently selected from hydrogen, methyl, cyano or halo; and R_{B5N}, R_{Y5N}, R_{Z2}N and R_{11N} are selected from methyl or hydrogen;
(ii) R₈, R₉ and R₁₁ are independently selected from hydrogen, NH₂, halo, cyano, C₁₋₄ alkoxy, C₁₋₄ haloalkoxy, C₁₋₃ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl;
(iii) R₁₀ is selected from C₁₋₄ alkoxy or C₁₋₄ haloalkoxy;
(iv) R_{Z1} and R_{Z1a} are selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl;
(v) R₁₂, R₁₃, R₁₆, R₁₈, R₁₉, R₂₀, R₂₁, R₂₂, R₂₃, R₂₄ and R₃₀ are independently selected from hydrogen, halo, cyano and methyl;
(vi) R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl a 5- or 6-membered or heteroaryl, C₃₋₆ cycloalkyl, —O—C₃₋₆ cycloalkyl, heterocyclyl, —(OCH₂CH₂)ₘ—OCH₃ wherein m is an integer from 1 to 6, NR_{q}Rᵣ, wherein R_{q} and Rᵣ are each independently hydrogen, C₁₋₄ alkyl or R_{q} and Rᵣ are linked together such that, together with the nitrogen atom to which they are attached, they form a 3- to 6-membered heterocyclic ring; wherein any C₁₋₄ alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.
(vii) R₂₁, R₂₄ and R₃₀, are independently selected from hydrogen or methyl;
(viii) R₂₈ and R₂₉ are selected from hydrogen or halo, methoxy and methyl.

In an embodiment:
(i) R₄, R₅, R_{X5a}, R_{X5b}, R_{Y5}, R_{B5N}, R_{Y5N}, R₆, R₇, R₈, R₉, R₁₀, R₁₁, R₁₁ₐ, R_{11b}, R_{Z2}, R_{Z2a}, R_{Z3a}, R_{Zi1b}, R_{Zi2e}, R_{Z9}, R_{Z10}, R_{Z11}, R_{Z12}, R_{Z13}, R_{Z14}, R_{Z15} and R_{Z16} are hydrogen; R_{Z2N} and R_{11N} are selected from methyl or hydrogen;
(ii) R₈ is cyano;
(iii) R₁₀ is methoxy;
(iv) R_{Z1} and R_{Z1a} are selected from hydrogen, cyano, halo, C₁₋₂ haloalkyl, C₁₋₂ haloalkoxy, C₁₋₂ alkoxy, C₃₋₆ cycloalkyl and —O—C₃₋₆ cycloalkyl;
(v) R₁₂, R₁₃, R₁₆, R₁₈, R₁₉, R₂₀, R₂₁, R₂₂, R₂₃, R₂₄ and R₃₀ are hydrogen;
(vi) R₁₇ is selected from hydrogen, halo, cyano, C₁₋₂ alkyl, C₁₋₂ haloalkyl, C₁₋₂ alkoxy, C₁₋₂ haloalkoxy, C₂₋₃ alkenyl, C₂₋₃ alkynyl, phenyl a 5- or 6-membered or heteroaryl, C₃₋₆ cycloalkyl, —O—C₃₋₆ cycloalkyl, heterocyclyl, —(OCH₂CH₂)ₘ—OCH₃ wherein m is an integer from 1 to 6, NR_{q}Rᵣ, wherein R_{q} and Rᵣ are each independently hydrogen, C₁₋₂ alkyl or R_{q} and Rᵣ are linked together such that, together with the nitrogen atom to which they are attached, they form a 3- to 6-membered heterocyclic ring; wherein any C₁₋₄ alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl
(vii) R₂₁, R₂₄ and R₃₀, are hydrogen;
(viii) R₂₈ and R₂₉ are hydrogen.

Suitably, a heteroaryl or heterocyclyl group as defined herein is a monocyclic heteroaryl or heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 4-, 5- or 6-membered heterocyclyl ring comprising one, two or three heteroatoms selected from N, O or S. Most suitably, a heterocyclyl group is a 5- or 6-membered ring comprising one, two or three heteroatoms selected from N, O or S [e.g. morpholinyl (e.g. 4-morpholinyl), oxetane, methyloxetane (e.g. 3-methyloxetane), pyrrolidinone (e.g. pyrrolidin-2-one)].

Suitably, an aryl group is phenyl.

Suitably, X is as defined in any one of paragraphs (33) to (37) above. More suitably, X is as defined in any one of paragraphs (33) or (36) to (37) above. Most suitably, X is as defined in paragraph (33) or (37).

Suitably, R₁ₐ, R_{1c}, and R₁ₑ are as defined in any one of paragraphs (1) to (5) above. Most suitably, R₁ₐ, R_{1c}, and R₁ₑ are as defined in paragraph (5).

Suitably, R_{1b}, R_{1d} and R_{1f} are as defined in any one of paragraphs (6) to (18) above. More suitably, R_{1b}, R_{1d} and R_{1f} are as defined in paragraph (16), (17) or (18). Most suitably, R_{1b}, R_{1d} and R_{1f} are as defined in paragraph (18).

Suitably, R_{1b}, R_{1d} and R_{1f} are as defined in any one of paragraphs (19) to (27) above. More suitably, R_{1b}, R_{1d} and R_{1f} are as defined in any one of paragraphs (25) to (27). Most suitably, R_{1b}, R_{1d} and R_{1f} are as defined in paragraph (27).

Suitably, R_{1a′} is as defined in any one of paragraphs (28) to (30) above. Most suitably, R_{1a′} is as defined in paragraph (30) above.

Suitably, R₂ₐ, R_{2b} and R_{2c} are as defined in any one of paragraphs (31) and (32) above. Most suitably, R₂ₐ, R_{2b} and R_{2c} are as defined in paragraph (32).

Suitably, Y is as defined in any one of paragraphs (47) to (72) above. Suitably, Y is as defined in any one of paragraphs (47), (48), (49), (50), (53), (58), (59) and (61). Suitably, Y is as defined in any one of paragraphs (69) to (72). Suitably, Y is as defined in paragraphs (71) to (72) above. Most suitably, Y is as defined in paragraph (72)

Suitably, n is as defined in any one of paragraphs (45) and (46) above. Most suitably, n is as defined in paragraph (46), i.e. n is 1.

Suitably, R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are as defined in any one of (38) to (41), and paragraphs (43) and (44) above. Most suitably, R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are as defined in paragraph (41) above).

Suitably, R₃ₐ₂, R_{3b2}, R₃ᵢ₂, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are as defined in any one of paragraphs (42), (43) and (44) above. Most suitably, R₃ₐ₂, R_{3b2}, R₃ᵢ₂, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are as defined in paragraph (42).

Suitably, Z is as defined in any one of paragraphs from (73) to (126) above. Suitably, Z is as defined in any one of paragraphs from (119) to (126) above. Suitably, Z is as defined in any one of paragraphs from (123) to (126) above. Most suitably, Z is as defined in paragraph (126) above.

Suitably, Z may also be as defined in any one of paragraphs (84a), (87a), (88a), (88b), (89a), (89b) or (89c) above.

Suitably, R₄ is as defined in any one of paragraphs (127) and (128) above. Most suitably, R₄ is as defined in paragraph (128).

Suitably, R₅ is as defined in any one of paragraphs (127) and (128) above. Most suitably, R₅ is as defined in paragraph (128).

Suitably, R₆ is as defined in any one of paragraphs (127) and (128) above. Most suitably, R₆ is as defined in paragraph (128).

Suitably, R₇, R₉ and R_{11N} are as defined in paragraph (129).

Suitably, R₈, R₉, R₁₀ and R₁₁ are as defined in any one of paragraphs (130) to (133). More suitably, R₈, R₉, R₁₀ and R₁₁ are as defined in any one of paragraphs (131) to (133) Suitably, R₈, R₉, R₁₀ and R₁₁ are as defined in paragraphs (132) or (133).

Suitably, R₈, R₉, R₁₀ and R₁₁ may also be as defined in any one of paragraphs (129a), (130a), (133a), (133b) or (133c) above. R₈, R₉, R₁₀ and R₁₁ may more suitably be defined in any one of paragraphs (133a), (133b) or (133c).

Suitably, R_{Z1} and R_{Z1a} are as defined in any one of paragraphs (134) to (137) above. Most suitably, R_{Z1} and R_{Z1a} are as defined in paragraph (137).

Suitably, R_{Z2} and R_{Z2a} are as defined in any one of paragraphs (138) to (140) above. Most suitably, R_{Z2} and R_{Z2a} are as defined in paragraph (140).

Suitably, R_{Z3a} is as defined in any one of paragraphs (138) to (140) above. Most suitably, R_{Z2} and R_{Z2a} are as defined in paragraph (140).

Suitably, R_{Zi1b} and R_{Zi2e} are as defined in any one of paragraphs (138) to (140) above. Most suitably, R_{Zi1b} and R_{Zi2e} are as defined in paragraph (140), i.e. R_{Zi1b} and R_{Zi2e} are hydrogen.

Suitably, R_{Z2N}, and R_{11N} are as defined in any one of paragraphs (141) and (142). Most suitably, R_{Z2N} and R_{11N} are as defined in paragraph (142).

Suitably, R_{Y5N} is as defined in any one of paragraphs (141), (142) and (143). Most suitably, R_{Y5N} is as defined in paragraph (142) or (143).

Suitably, R_{Z4}, R_{Z5}, R_{Z6}, R_{Z7}, R_{Z8}, R_{Z9}, R_{Z10}, R_{Z11}, R_{Z12}, R_{Z13}, R_{Z14}, R_{Z15}, and R_{Z16} are as defined in any one of paragraphs (144), (145) and (146). Most suitably, R_{Z4}, R_{Z5}, R_{Z6}, R_{Z7}, R_{Z8}, R_{Z9}, R_{Z10}, R_{Z11}, R_{Z12}, R_{Z13}, R_{Z14}, R_{Z15}, and R_{Z16} are as defined in paragraph (146).

Suitably, R₁₂ is as defined in any one of paragraphs (147), (148) and (149) to (152). Most suitably, R₁₂ is as defined in paragraph (152).

Suitably, R₁₃ is as defined in any one of paragraphs (147), (148) and (153) to (155). Most suitably, R₁₃ is as defined in paragraph (155).

Suitably, A₅ is CR₁₆, and R₁₆ is as defined in any one of paragraphs (147), (148) and (156) to (159). Most suitably, R₁₆ is as defined in paragraph (159).

Suitably, A₆ is CR₁₇ and R₁₇ is as defined in any one of paragraphs (160) to (166). More suitably, R₁₇ is as defined in any one of paragraphs (163) to (166). Most suitably, R₁₇ is as defined in paragraph (166).

Suitably, A₇ is CR₁₃, and R₁₈ is as defined in any one of paragraphs (147), (148) and (156) to (159). Most suitably, R₁₈ is as defined in paragraph (159).

Suitably, R_{1g} is as defined in any one of in any one of paragraphs (147), (148) and (167) to (169). Most suitably, R_{1g} is as defined in paragraph (169).

Suitably, R₂₁ is as defined in any one of in any one of paragraphs (147), (148) and (170) to (171). Most suitably, R_{1g} is as defined in paragraph (171).

Suitably, R₂₂ is as defined in any one of in any one of paragraphs (147), (148) and (172) to (174). Most suitably, R₂₂ is as defined in paragraph (174).

Suitably, R₂₄ and R₃₀ are as defined in any one of in any one of paragraphs (170) to (171). Most suitably, R₂₄ and R₃₀ are as defined in paragraph (171).

Suitably, R₂₈ and R₂₉ are as defined in any one of in any one of paragraphs (175) to (176). Most suitably, R₂₈ and R₂₉ are as defined in paragraph (176).

In an embodiment,
X is:

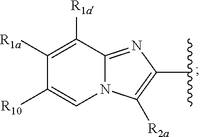

Y is

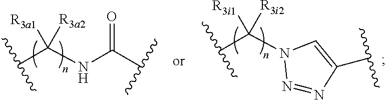

and
Z is selected from:

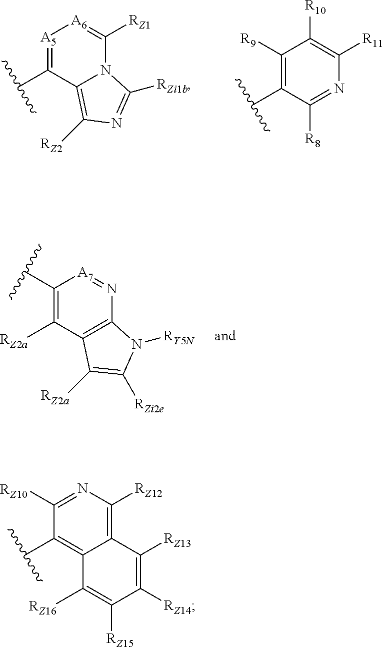

wherein
(i) R₁ₐ, R_{1a′}, R_{1b}, and R₂ₐ are as defined herein;
(ii) R₃ₐ₁, R₃ₐ₂, R₃ᵢ₁ and R₃ᵢ₂ are as defined herein;
(iii) A₅, A₆, A₇, R₈, R₉, R₁₀, R₁₁, R_{Z1}, R_{Z2}, R_{Zi1b}, R_{Z10}, R_{Z12}, R_{Z13}, R_{Z14}, R_{Z15}, R_{Z16}, R_{Y5N}, R_{Z2a}, R_{Z3a}, and R_{Zi2e} are as defined herein.

In an embodiment,
X is:

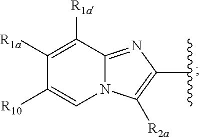

Y is:

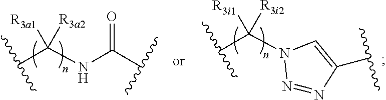

and
Z is selected from:

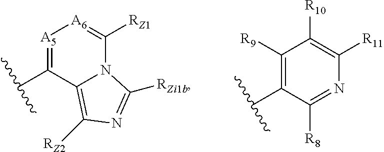

wherein
(i) R₁ₐ, R_{1a′}, R_{1b}, and R₂ₐ are as defined herein;
(ii) R₃ₐ₁, R₃ₐ₂, R₃ᵢ₁ and R₃ᵢ₂ are as defined herein;
(iii) R₈, R₉, R₁₀, R₁₁, A₅, A₆, R_{Z1}, R_{Z2} and R_{Zi1b} are as defined herein.

In an embodiment,
X is:

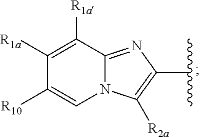

Y is:

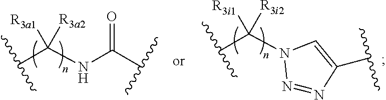

and
Z is selected from:

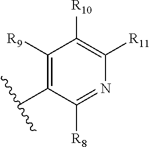

wherein
(i) R₁ₐ, R_{1a′}, R_{1b}, and R₂ₐ are as defined herein;
(ii) R₃ₐ₁, R₃ₐ₂, R₃ᵢ₁ and R₃ᵢ₂ are as defined herein;
(iii) R₈ is selected from cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; or
R₈ is hydrogen;
R₉ and R₁₁ are as herein defined;
R₁₀ is C₁₋₄ alkoxy or C₁₋₄ haloalkoxy.

In an embodiment,
X is:

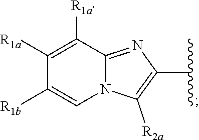

Y is:

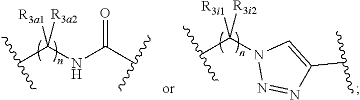

and
Z is selected from:

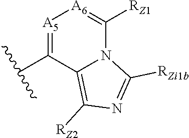

wherein
(i) R₁ₐ, R_{1a′}, R_{1b}, and R₂ₐ are as defined herein;
(ii) R₃ₐ₁, R₃ₐ₂, R₃ᵢ₁, R₃ᵢ₂, R₃ₗ₁, R₃ₗ₂, R₃ᵣ₁, R₃ᵣ₂, are as defined herein;
(iii) A₅, A₆, R_{Z1}, R_{Z2} and R_{Zi1b} are as defined herein.

In an embodiment,
X is:

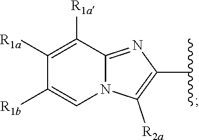

Y is:

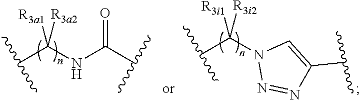

and
Z is selected from:

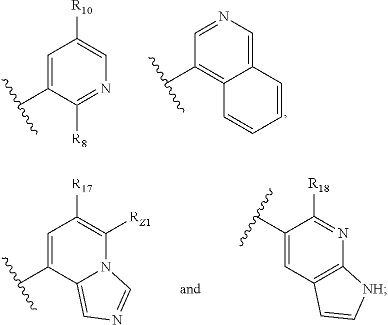

wherein
(i) R₁ₐ, R_{1a′}, R_{1b}, and R₂ₐ are as defined herein;
(ii) R₃ₐ₁, R₃ₐ₂, R₃ᵢ₁, R₃ᵢ₂, R₃ᵢ₁, R₃ₗ₂, R₃ᵣ₁, R₃ᵣ₂, R₈, R₁₀, R₁₇, R₁₈ and R_{Z1} are as defined herein;
(iii) R₈, R₁₀, R₁₇, R₁₈ and R_{Z1} are as defined herein.

In a particular group of compounds of the invention, Y is as defined in paragraph (47), i.e. the compounds have the structural formula (II) (a sub-definition of formula (I)) shown below:

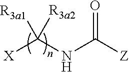

wherein X, R₃ₐ₁, R₃ₐ₂, n and Z each having any one of the meanings defined herein; or a pharmaceutically acceptable salt thereof.

In an embodiment of the compounds of formula (II):
X is as defined in any one of paragraphs (33) to (35) or (36) to (37) above, suitably X is as defined in paragraph (33) or (37); and R_{1b}, R_{1d} and R_{1f} are each as defined in any one of paragraphs (6) to (18) or (19) to (27) above
R₃ₐ₁ is as defined in any one of paragraphs from (38) to (41), and paragraphs (43) and (44) above;
R₃ₐ₂ is as defined in any one of paragraphs (42), (43) and (44) above;
n is as defined in any one of paragraphs (45) and (46) above; and
Suitably, Z is as defined in any one of paragraphs from (119) to (126) above. Suitably, Z is as defined in any one of paragraphs from (123) to (126) above.

In a particular group of the compounds of formula (II):
X is as defined in paragraph (33), (34) or (35) above and R_{1b}, R_{1d} and R_{1f} are each as defined in any one of paragraphs (6) to (18) or (25) to (27) above;
R₃ₐ₁ is as defined in paragraph (41) above;
R₃ₐ₂ is as defined in paragraph (42) above;
n is as defined in paragraph (46) above; and
Z is as defined in any one of paragraphs (123) to (126) above.

In an embodiment of the compounds of formula (II):
X is as defined in paragraph (33) above and R_{1b}, R_{1d} and R_{1f} are each as defined in any one of paragraphs (16) to (18) or (25) to (27) above;
R₃ₐ₁ is as defined in paragraph (41) above;
R₃ₐ₂ is as defined in paragraph (42) above;
n is as defined in paragraph (46) above; and
Z is as defined in paragraph (126) above.

In a particular group of compounds of the invention, Y is as defined in paragraph (58), i.e. the compounds have the structural formula (III) (a sub-definition of formula (I)) shown below:

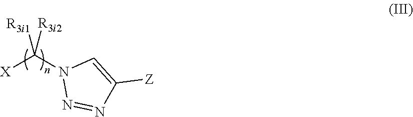

wherein X, R₃ᵢ₁, R₃ₗ₂, n and Z each having any one of the meanings defined herein; or a pharmaceutically acceptable salt thereof.

In an embodiment of the compounds of formula (III):
X is as defined in any one of paragraphs (33) to (35) or (36) to (37) above, suitably X is as defined in paragraph (33) or (37); and R_{1b}, R_{1d} and R_{1f} are each as defined in any one of paragraphs (6) to (18) or (19) to (27) above
R₃ᵢ₁ is as defined in any one of paragraphs from (38) to (41), and paragraphs (43) and (44) above;
R₃ᵢ₂ is as defined in any one of paragraphs (42), (43) and (44) above;
n is as defined in any one of paragraphs (45) and (46) above; and
Suitably, Z is as defined in any one of paragraphs from (119) to (126) above. Suitably, Z is as defined in any one of paragraphs from (123) to (126) above.

In a particular group of the compounds of formula (III):
X is as defined in paragraph (33), (34) or (35) above and R_{1b}, R_{1d} and R_{1f} are each as defined in any one of paragraphs (6) to (18) or (25) to (27) above;
R₃ᵢ₁ is as defined in paragraph (41) above;
R₃ᵢ₂ is as defined in paragraph (42) above;
n is as defined in paragraph (46) above; and
Z is as defined in any one of paragraphs (123) to (126) above.

In an embodiment of the compounds of formula (III):
X is as defined in paragraph (33) above and R_{1b}, R_{1d} and R_{1f} are each as defined in any one of paragraphs (16) to (18) or (25) to (27) above;
R₃ᵢ₁ is as defined in paragraph (41) above;
R₃ᵢ₂ is as defined in paragraph (42) above;
n is as defined in paragraph (46) above; and
Z is as defined in paragraph (126) above.

In a particular group of compounds of the invention, X is as defined in paragraph (33), i.e. the compounds have the structural formula (IV) (a sub-definition of formula (I)) shown below:

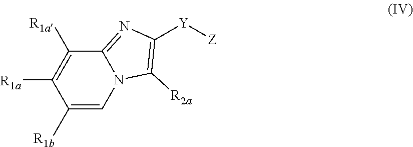

wherein R₁ₐ, R_{1b}, R_{1a′}, R₂ₐ, Y and Z each having any one of the meanings defined herein; or a pharmaceutically acceptable salt thereof.

In an embodiment of the compounds of formula (IV):
R₁ₐ is as defined in any one of paragraphs (1) to (5) above;
R_{1b} is as defined in any one of paragraphs (6) to (18) or (19) to (27) above;
R^{1a′} is as defined in any one of paragraphs (28) to (30) above;
R₂ₐ is as defined in any one of paragraphs (31) and (32) above;
Y is as defined in any one of paragraphs (47) to (72) above; and
Z is as defined in any one of paragraphs from (119) to (126) above.

In an embodiment of the compounds of formula (IV):
R₁ₐ is as defined in any one of paragraphs (3) to (5) above;
R_{1b} is as defined in any one of paragraphs (16) to (18) or (25) to (27) above;
R^{1a′} is as defined in any one of paragraphs (28) to (30) above;
R₂ₐ is as defined in any one of paragraphs (31) and (32) above;
Y is as defined in any one of paragraphs (69) to (72) above; and
Z is as defined in any one of paragraphs from (123) to (126) above.

In an embodiment of the compounds of formula (IV):
R₁ₐ is as defined in paragraph (5) above;
R_{1b} is as defined in paragraph ((18) above;
R_{1a′} is as defined in paragraph (30) above;
R₂ₐ is as defined in paragraph (32) above;
Y is as defined in any one of paragraphs (71) to (72) above; and
Z is as defined in any one of paragraphs from (123) to (126) above.

In an embodiment of the compounds of formula (IV):
R₁ₐ is as defined in any one of paragraphs (3) to (5) above;
R_{1b} is as defined in any one of paragraphs (16) to (18) or (25) to (27) above;
R_{1a′} is as defined in any one of paragraphs (28) to (30) above;
R₂ₐ is as defined in any one of paragraphs (31) and (32) above;
Y is as defined in any one of paragraphs (69) to (72) above; and
Z is as defined in any one of paragraphs from (84a), (87a), (88a), (88b), (89a), (89b) or (89c) above.

In an embodiment of the compounds of formula (IV):
R₁ₐ is as defined in paragraph (5) above;
R_{1b} is as defined in paragraph (18) above;
R_{1a′} is as defined in paragraph (30) above;
R₂ₐ is as defined in paragraph (32) above;
Y is as defined in any one of paragraphs (71) to (72) above; and
Z is as defined in any one of paragraphs (84a), (87a), (88a), (88b), (89a), (89b) or (89c) above.

In a particular group of compounds of the invention, X is as defined in paragraph (33) and Y is as defined in paragraph (47), i.e. the compounds have the structural formula (V) (a sub-definition of formula (I)) shown below:

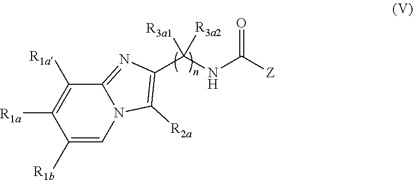

wherein R₁ₐ, R_{1b}, R_{1a′}, R₂ₐ, R₃ₐ₁, R₃ₐ₂, n and Z each having any one of the meanings defined herein; or a pharmaceutically acceptable salt thereof.

In an embodiment of the compounds of formula (V):
R₁ₐ is as defined in any one of paragraphs (1) to (5) above;
R_{1b} is as defined in any one of paragraphs (6) to (18) above or (19) to (27);
R_{1a′} is as defined in any one of paragraphs (28) to (30) above;
R₂ₐ is as defined in any one of paragraphs (31) and (32) above;
R₃ₐ₁ is as defined in any one of paragraphs from (38) to (41), and paragraphs (43) and (44) above;
R₃ₐ₂ is as defined in any one of paragraphs (42), (43) and (44) above;
n is as defined in any one of paragraphs (45) and (46) above; and
Z is as defined in any one of paragraphs from (119) to (126) above.

In a particular group of the compounds of formula (V):
R₁ₐ is as defined in any one of paragraphs (1) to (5) above;
R_{1b} is as defined in paragraph (10) to (18) above, or (25) to (27);
R_{1a′} is as defined in any one of paragraphs (28) to (30) above;
R₂ₐ is as defined in any one of paragraphs (31) and (32) above;
R₃ₐ₁ is as defined in any one of paragraphs from (38) to (41), and paragraphs (43) and (44) above;
R₃ₐ₂ is as defined in any one of paragraphs (42), (43) and (44) above;
n is as defined in any one of paragraphs (45) and (46) above; and
Z is as defined in any one of paragraphs (123) to (126) above.

In an embodiment of the compounds of formula (V):
R₁ₐ is as defined in paragraph (6) above;
R_{1a′} is as defined in paragraph (17) above;
R_{1b} is as defined in any one of paragraphs (16), (17) or (18) or above, or R_{1b} is as defined in any one of paragraphs (26) or (27);
R₂ₐ is as defined in paragraph (19) above;
R₃ₐ₁ is as defined in paragraph (41) above;
R₃ₐ₂ is as defined in paragraph (42) above;
n is as defined in paragraph (46) above; and
Z is as defined in paragraph (126) above.

In an embodiment of the compounds of formula (V):
R₁ₐ is as defined in any one of paragraphs (1) to (5) above;
R_{1b} is as defined in any one of paragraphs (6) to (18) above or (19) to (27);
R_{1a′} is as defined in any one of paragraphs (28) to (30) above;
R₂ₐ is as defined in any one of paragraphs (31) and (32) above;
R₃ₐ₁ is as defined in any one of paragraphs from (38) to (41), and paragraphs (43) and (44) above;
R₃ₐ₂ is as defined in any one of paragraphs (42), (43) and (44) above;
n is as defined in any one of paragraphs (45) and (46) above; and
Z is as defined in any one of paragraphs (84a), (87a), (88a), (88b), (89a), (89b) or (89c) above.

In a particular group of the compounds of formula (V):
R₁ₐ is as defined in any one of paragraphs (1) to (5) above;
R_{1b} is as defined in paragraph (10) to (18) above, or (25) to (27);
R_{1a′} is as defined in any one of paragraphs (28) to (30) above;
R₂ₐ is as defined in any one of paragraphs (31) and (32) above;
R₃ₐ₁ is as defined in any one of paragraphs from (38) to (41), and paragraphs (43) and (44) above;
R₃ₐ₂ is as defined in any one of paragraphs (42), (43) and (44) above;
n is as defined in any one of paragraphs (45) and (46) above; and
Z is as defined in any one of paragraphs (84a), (87a), (88a), (88b), (89a), (89b) or (89c) above.

In an embodiment of the compounds of formula (V):
R₁ₐ is as defined in paragraph (6) above;
R_{1a′} is as defined in paragraph (17) above;
R_{1b} is as defined in any one of paragraphs (16), (17) or (18) or above, or R_{1b} is as defined in any one of paragraphs (26) or (27);
R₂ₐ is as defined in paragraph (19) above;
R₃ₐ₁ is as defined in paragraph (41) above;
R₃ₐ₂ is as defined in paragraph (42) above;
n is as defined in paragraph (46) above; and
Z is as defined in any one of paragraphs (84a), (87a), (88a), (88b), (89a), (89b) or (89c) above.

In a particular group of compounds of the invention, X is as defined in paragraph (20), R_{1a′} is as defined in paragraph (17) and Y is as defined in paragraph (36), i.e. the compounds have the structural formula (VI) (a sub-definition of formula (I)) shown below:

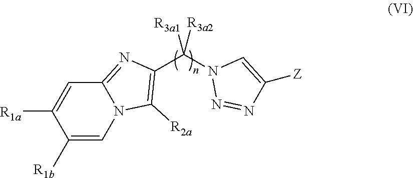

wherein R₁ₐ, R_{1b}, R₂ₐ, R₃ᵢ₁, R₃ᵢ₂, n and Z each having any one of the meanings defined herein; or a pharmaceutically acceptable salt thereof.

In an embodiment of the compounds of formula (VI):
R₁ₐ is as defined in any one of paragraphs (1) to (5) above;
R_{1b} is as defined in any one of paragraphs (6) to (18) above or (19) to (27) above;
R_{1a′} is as defined in any one of paragraphs (28) to (30) above;
R₂ₐ is as defined in any one of paragraphs (31) and (32) above;
R₃ᵢ₁ is as defined in any one of paragraphs from (38) to (41), and paragraphs (43) and (44) above;
R₃ᵢ₂ is as defined in any one of paragraphs (42), (43) and (44) above;
n is as defined in any one of paragraphs (45) and (46) above; and
Z is as defined in any one of paragraphs from (119) to (126) above.

In a particular group of the compounds of formula (VI):
R₁ₐ is as defined in any one of paragraphs (1) to (5) above;
R_{1b} is as defined in paragraph (10) to (18) above, or (25) to (27) above;
R_{1a′} is as defined in any one of paragraphs (28) to (30) above;
R₂ₐ is as defined in any one of paragraphs (31) and (32) above;
R₃ᵢ₁ is as defined in any one of paragraphs from (38) to (41), and paragraphs (43) and (44) above;
R₃ᵢ₂ is as defined in any one of paragraphs (42), (43) and (44) above;
n is as defined in any one of paragraphs (45) and (46) above; and
Z is as defined in any one of paragraphs (123) to (126) above.

In an embodiment of the compounds of formula (VI):
R₁ₐ is as defined in paragraph (6) above;
R_{1a′} is as defined in paragraph (17) above;
R_{1b} is as defined in any one of paragraphs (16), (17) or (18) or above, or R_{1b} is as defined in any one of paragraphs (26) or (27);
R₂ₐ is as defined in paragraph (19) above;
R₃ᵢ₁ is as defined in paragraph (41) above;
R₃ᵢ₂ is as defined in paragraph (42) above;
n is as defined in paragraph (46) above; and
Z is as defined in paragraph (126) above.

In an embodiment of the compounds of formula (VI):
R₁ₐ is as defined in any one of paragraphs (1) to (5) above;
R_{1b} is as defined in any one of paragraphs (6) to (18) above or (19) to (27) above;
R_{1a′} is as defined in any one of paragraphs (28) to (30) above;
R₂ₐ is as defined in any one of paragraphs (31) and (32) above;
R₃ᵢ₁ is as defined in any one of paragraphs from (38) to (41), and paragraphs (43) and (44) above;
R₃ᵢ₂ is as defined in any one of paragraphs (42), (43) and (44) above;
n is as defined in any one of paragraphs (45) and (46) above; and
Z is as defined in any one of paragraphs (84a), (87a), (88a), (88b), (89a), (89b) or (89c) above.

In a particular group of the compounds of formula (VI):
R₁ₐ is as defined in any one of paragraphs (1) to (5) above;
R_{1b} is as defined in paragraph (10) to (18) above, or (25) to (27) above;
R_{1a′} is as defined in any one of paragraphs (28) to (30) above;
R₂ₐ is as defined in any one of paragraphs (31) and (32) above;
R₃ᵢ₁ is as defined in any one of paragraphs from (38) to (41), and paragraphs (43) and (44) above;
R₃ᵢ₂ is as defined in any one of paragraphs (42), (43) and (44) above;
n is as defined in any one of paragraphs (45) and (46) above; and
Z is as defined in any one of paragraphs (84a), (87a), (88a), (88b), (89a), (89b) or (89c) above

In an embodiment of the compounds of formula (VI):
R₁ₐ is as defined in paragraph (6) above;
R_{1a′} is as defined in paragraph (17) above;
R_{1b} is as defined in any one of paragraphs (16), (17) or (18) or above, or R_{1b} is as defined in any one of paragraphs (26) or (27);
R₂ₐ is as defined in paragraph (19) above;
R₃ᵢ₁ is as defined in paragraph (41) above;
R₃ᵢ₂ is as defined in paragraph (42) above;
n is as defined in paragraph (46) above; and
Z is as defined in any one of paragraphs (84a), (87a), (88a), (88b), (89a), (89b) or (89c) above.

In a particular group of compounds of the invention, X is as defined in paragraph (20), R_{1a′} is as defined in paragraph (17) and Y is as defined in paragraph (36), i.e. the compounds have the structural formula (VII) (a sub-definition of formula (I)) shown below:

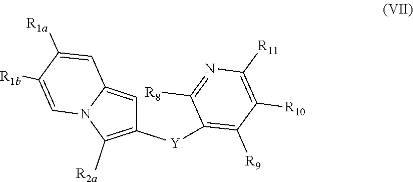

wherein R₁ₐ, R_{1b}, R₂ₐ, R₃ᵢ₁, R₃ᵢ₂, n and Z each having any one of the meanings defined herein; or a pharmaceutically acceptable salt thereof.

In an embodiment of the compounds of formula (VII):
R₁ₐ is as defined in any one of paragraphs (1) to (5) above;
R_{1b} is as defined in any one of paragraphs (6) to (18), or (19) to (27) above;
R₂ₐ is as defined in any one of paragraphs (31) to (32) above;
Y is as defined in any one of paragraphs (69) to (72) above;
R₈, R₉, R₁₀ and R₁₁ are as defined in any one of paragraphs (130) to (133) above;
In an embodiment of the compounds of formula (VII):
R₁ₐ is as defined in any one of paragraphs (1) to (5) above;
R_{1b} is as defined in paragraph (10) to (18) above, or (25) to (27) above;
R_{1a′} is as defined in any one of paragraphs (28) to (30) above;
R₂ₐ is as defined in any one of paragraphs (31) and (32) above;
Y is as defined in any one of paragraphs (69) to (72) above;
R₈, R₉, R₁₀ and R₁₁ are as defined in any one of paragraphs (132) to (133) above;
In particular group of the compounds of formula (VII):
R₁ₐ is as defined paragraph (5) above;
R_{1b} is as defined in any one of paragraphs (16), (17) or (18) or above, or R_{1b} is as defined in any one of paragraphs (26) or (27);
R₂ₐ is as defined in paragraph (32) above;
Y is as defined in paragraph (72) above;
R₈, R₉, R₁₀ and R₁₁ are as defined in paragraph (133) above;
In an embodiment of the compounds of formula (VII):
R₁ₐ is as defined in any one of paragraphs (1) to (5) above;
R_{1b} is as defined in any one of paragraphs (6) to (18), or (19) to (27) above;
R₂ₐ is as defined in any one of paragraphs (31) to (32) above;
Y is as defined in any one of paragraphs (69) to (72) above;
R₈, R₉, R₁₀ and R₁₁ are as defined in any one of paragraphs (129a), (130a), (133a), (133b) or (133c) above;
In an embodiment of the compounds of formula (VII):
R₁ₐ is as defined in any one of paragraphs (1) to (5) above;
R_{1b} is as defined in paragraph (10) to (18) above, or (25) to (27) above;
R_{1a′} is as defined in any one of paragraphs (28) to (30) above;
R₂ₐ is as defined in any one of paragraphs (31) and (32) above;
Y is as defined in any one of paragraphs (69) to (72) above;
R₈, R₉, R₁₀ and R₁₁ are as defined in any one of paragraphs (129a), (130a), (133a), (133b) or (133c) above;
In particular group of the compounds of formula (VII):
R₁ₐ is as defined paragraph (5) above;
R_{1b} is as defined in any one of paragraphs (16), (17) or (18) or above, or R_{1b} is as defined in any one of paragraphs (26) or (27);
R₂ₐ is as defined in paragraph (32) above;
Y is as defined in paragraph (72) above;
R₈, R₉, R₁₀ and R₁₁ are as defined in paragraph (129a), (130a), (133a), (133b) or (133c) above;
In a particular group of compounds of the invention, X is as defined in paragraph (20), R_{1a′} is as defined in paragraph (17) and Y is as defined in paragraph (36), i.e. the compounds have the structural formula (VII) (a sub-definition of formula (I)) shown below:

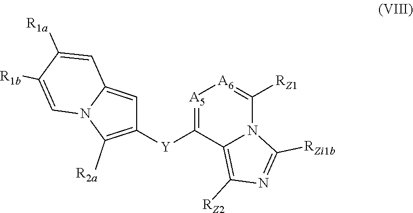

wherein R₁ₐ, R_{1b}, R₂ₐ, R₃ᵢ₁, R₃ᵢ₂, n and Z each having any one of the meanings defined herein; or a pharmaceutically acceptable salt thereof.

In an embodiment of the compounds of formula (VIII):
R₁ₐ is as defined in any one of paragraphs (1) to (5) above;
R_{1b} is as defined in any one of paragraphs (6) to (18), or (19) to (27) above;
R₂ₐ is as defined in any one of paragraphs (31) to (32) above;
Y is as defined in any one of paragraphs (69) to (72) above;
A₅ is CR₁₆, and R₁₆ is as defined in any one of paragraphs (147), (148) and (156) to (159). above;
A₆ is CR₁₇ and R₁₇ is as defined in any one of paragraphs (160) to (166) above;
R_{Z1} is as defined in any one of paragraphs (134) to (137) above;
R_{Z2} is as defined in any one of paragraphs (138) to (140) above; and
R_{zi1b} is as defined in any one of paragraphs (138) to (140) above.

In an particular group of the compounds of formula (VIII):
R₁ₐ is as defined in any one of paragraphs (1) to (5) above;
R_{1b} is as defined in paragraph (10) to (18) above, or (25) to (27) above;
R_{1a′} is as defined in any one of paragraphs (28) to (30) above;
R₂ₐ is as defined in any one of paragraphs (31) and (32) above;
Y is as defined in any one of paragraphs (69) to (72) above;
A₅ is CR₁₆, and R₁₆ is as defined in any one of paragraphs (156) to (159). above;
A₆ is CR₁₇ and R₁₇ is as defined in any one of paragraphs (163) to (166) above;
R_{Z1} is as defined in any one of paragraphs (135) to (137) above;
R_{Z2} is as defined in any one of paragraphs (139) to (140) above; and
R_{Zi1b} is as defined in any one of paragraphs (139) to (140) above.

In particular group of the compounds of formula (VIII):
R₁ₐ is as defined paragraph (5) above;
R_{1b} is as defined in any one of paragraphs (16), (17) or (18) or above, or R_{1b} is as defined in any one of paragraphs (26) or (27);
R₂ₐ is as defined in paragraph (32) above;
Y is as defined in paragraph (72) above;
A₅ is CR₁₆, and R₁₆ is as defined in paragraph (159) above;
A₆ is CR₁₇ and R₁₇ is as defined in paragraph (166) above;
R_{Z1} is as defined in paragraph (137) above;
R_{Z2} is as defined in paragraph (140) above, i.e. R_{Z2} is hydrogen; and
R_{Zi1b} is as defined in in paragraph (140) above, i.e. R_{zi1b} is hydrogen.

In certain embodiments of the invention, when
X is

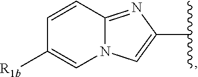

Y is

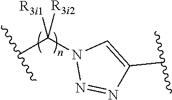

and
Z is

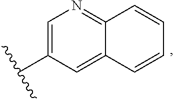 [SMILES: *c1cnc2ccccc2c1]

then R_{1b} is not methyl,

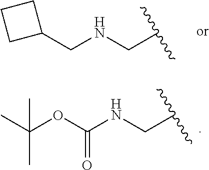

In certain embodiments of the invention, when
X is

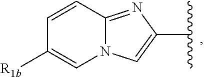

and
Z is

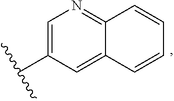 [SMILES: *c1cnc2ccccc2c1]

then Y is not

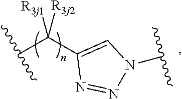

Particular compounds of the present invention include any of the compounds exemplified in the present application, or a pharmaceutically acceptable salt thereof, and, in particular, any of the following:
N-[(6-{[(cyclohexylmethyl)amino]methyl}imidazo[1,2-a]pyridin-2-yl)methyl]-1,2-dimethyl-6-oxo-1,6-dihydropyrimidine-4-carboxamide;
N-({6-methylimidazo[1,2-a]pyridin-2-yl}methyl)-4-oxo-4H-quinolizine-2-carboxamide;
N-[(6-{[(cyclohexylmethyl)amino]methyl}imidazo[1,2-a]pyridin-2-yl)methyl]-4-oxo-4H-quinolizine-2-carboxamide;
N-[(6-{[(cyclohexylmethyl)amino]methyl}imidazo[1,2-a]pyridin-2-yl)methyl]quinoxaline-2-carboxamide;
N-[(6-{[(cyclohexylmethyl)amino]methyl}imidazo[1,2-a]pyridin-2-yl)methyl]-1H-pyrazolo[3,4-b]pyridine-4-carboxamide;
N-[(6-{[(cyclohexylmethyl)amino]methyl}imidazo[1,2-a]pyridin-2-yl)methyl]quinoline-3-carboxamide;
N-[(6-{[(cyclobutylmethyl)amino]methyl}imidazo[1,2-a]pyridin-2-yl)methyl]imidazo[1,5-a]pyridine-8-carboxamide;
N-[(6-{[(cyclobutylmethyl)amino]methyl}imidazo[1,2-a]pyridin-2-yl)methyl]imidazo[1,5-a]pyridine-5-carboxamide;
N-[(6-{[(cyclobutylmethyl)amino]methyl}imidazo[1,2-a]pyridin-2-yl)methyl]-3H-imidazo[4,5-b]pyridine-6-carboxamide;
N-[(6-{[(cyclobutylmethyl)amino]methyl}imidazo[1,2-a]pyridin-2-yl)methyl]-1H-pyrrolo[2,3-b]pyridine-5-carboxamide;
N-{[6-({[(3,3-difluorocyclobutyl)methyl]amino}methyl)imidazo[1,2-a]pyridin-2-yl]methyl}-1H-pyrrolo[2,3-b]pyridine-5-carboxamide;
N-[(6-{[(cyclobutylmethyl)amino]methyl}imidazo[1,2-a]pyridin-2-yl)methyl]-1-methyl-1H-pyrrolo[2,3-b]pyridine-5-carboxamide
N-[(6-{[(cyclobutylmethyl)amino]methyl}imidazo[1,2-a]pyridin-2-yl)methyl]-1H-indole-5-carboxamide;
N-[(6-{[(cyclobutylmethyl)amino]methyl}imidazo[1,2-a]pyridin-2-yl)methyl]-5H-pyrrolo[2,3-b]pyrazine-2-carboxamide;
N-[(6-{[({3-fluorobicyclo[1.1.1]pentan-1-yl}methyl)amino]methyl}imidazo[1,2-a]pyridin-2-yl)methyl]-5-oxo-5H-[1,3]thiazolo[3,2-a]pyrimidine-7-carboxamide;
4-[1-[(6-methylimidazo[1,2-a]pyridin-2-yl)methyl]triazol-4-yl]isoquinoline;
1-({6-methylimidazo[1,2-a]pyridin-2-yl}methyl)-4-{1H-pyrrolo[2,3-b]pyridin-5-yl}-1H-1,2,3-triazole;
(cyclobutylmethyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{imidazo[1,5-a]pyridin-8-yl}-1H-1,2,3-triazol-1-yl)methyl]imidazo[1,2-a]pyridin-6-yl}methyl)amine;
({2-[(4-{6-chloroimidazo[1,5-a]pyridin-8-yl}-1H-1,2,3-triazol-1-yl)methyl]imidazo[1,2-a]pyridin-6-yl}methyl)(cyclobutylmethyl)amine;
(cyclobutylmethyl)({2-[(4-{1-fluoroimidazo[1,5-a]pyridin-8-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{[1,2,4]triazolo[4,3-a]pyridin-8-yl}-1H-1,2,3-triazol-1-yl)methyl]imidazo[1,2-a]pyridin-6-yl}methyl)amine;
({bicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
2-({2-[(4-{imidazo[1,5-a]pyridin-8-yl}-1H-1,2,3-triazol-1-yl)methyl]imidazo[1,2-a]pyridin-6-yl}methyl)-2-azaspiro[3.3]heptane;
2-({2-[(4-{6-methoxyimidazo[1,5-a]pyridin-8-yl}-1H-1,2,3-triazol-1-yl)methyl]imidazo[1,2-a]pyridin-6-yl}methyl)-2-azaspiro[3.3]heptane;
N-(cyclobutylmethyl)-1-[2-[[4-(4-isoquinolyl)triazol-1-yl]methyl]imidazo[1,2-a]pyridin-6-yl]methanamine;
bis(cyclobutylmethyl)[(2-{[4-(isoquinolin-4-yl)-1H-1,2,3-triazol-1-yl]methyl}imidazo[1,2-a]pyridin-6-yl)methyl]amine;
(cyclobutylmethyl)({2-[(4-{1H-pyrrolo[2,3-b]pyridin-5-yl}-1H-1,2,3-triazol-1-yl)methyl]imidazo[1,2-a]pyridin-6-yl}methyl)amine;
(cyclobutylmethyl)({2-[(4-{1H-pyrrolo[2,3-b]pyridin-5-yl}-1H-imidazol-1-yl)methyl]imidazo[1,2-a]pyridin-6-yl}methyl)amine;
cyclobutylmethyl)({2-[(4-{imidazo[1,5-a]pyridin-5-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{3-methylimidazo[1,5-a]pyridin-8-yl}-1H-1,2,3-triazol-1-yl)methyl]imidazo[1,2-a]pyridin-6-yl}methyl)amine;
3-{1-[(6-{[({bicyclo[1.1.1]pentan-1-yl}methyl)amino]methyl}imidazo[1,2-a]pyridin-2-yl)methyl]-1H-1,2,3-triazol-4-yl}-5-methoxypyridine-2-carbonitrile.
Particular compounds of the present invention include any of the compounds exemplified in the present application, or a pharmaceutically acceptable salt thereof, and, in particular, any of the following:
N-[(6-{[(cyclohexylmethyl)amino]methyl}imidazo[1,2-a]pyridin-2-yl)methyl]-1,2-dimethyl-6-oxo-1,6-dihydropyrimidine-4-carboxamide;
N-({6-methylimidazo[1,2-a]pyridin-2-yl}methyl)-4-oxo-4H-quinolizine-2-carboxamide;
N-[(6-{[(cyclohexylmethyl)amino]methyl}imidazo[1,2-a]pyridin-2-yl)methyl]-4-oxo-4H-quinolizine-2-carboxamide;
N-[(6-{[(cyclohexylmethyl)amino]methyl}imidazo[1,2-a]pyridin-2-yl)methyl]quinoxaline-2-carboxamide;
N-[(6-{[(cyclohexylmethyl)amino]methyl}imidazo[1,2-a]pyridin-2-yl)methyl]-1H-pyrazolo[3,4-b]pyridine-4-carboxamide;
N-[(6-{[(cyclohexylmethyl)amino]methyl}imidazo[1,2-a]pyridin-2-yl)methyl]quinoline-3-carboxamide;
N-[(6-{[(cyclobutylmethyl)amino]methyl}imidazo[1,2-a]pyridin-2-yl)methyl]imidazo[1,5-a]pyridine-8-carboxamide;
N-[(6-{[(cyclobutylmethyl)amino]methyl}imidazo[1,2-a]pyridin-2-yl)methyl]imidazo[1,5-a]pyridine-5-carboxamide;
N-[(6-{[(cyclobutylmethyl)amino]methyl}imidazo[1,2-a]pyridin-2-yl)methyl]-3H-imidazo[4,5-b]pyridine-6-carboxamide;
N-[(6-{[(cyclobutylmethyl)amino]methyl}imidazo[1,2-a]pyridin-2-yl)methyl]-1H-pyrrolo[2,3-b]pyridine-5-carboxamide;
N-{[6-({[(3,3-difluorocyclobutyl)methyl]amino}methyl)imidazo[1,2-a]pyridin-2-yl]methyl}-1H-pyrrolo[2,3-b]pyridine-5-carboxamide;
N-[(6-{[(cyclobutylmethyl)amino]methyl}imidazo[1,2-a]pyridin-2-yl)methyl]-1-methyl-1H-pyrrolo[2,3-b]pyridine-5-carboxamide
N-[(6-{[(cyclobutylmethyl)amino]methyl}imidazo[1,2-a]pyridin-2-yl)methyl]-1H-indole-5-carboxamide;
N-[(6-{[(cyclobutylmethyl)amino]methyl}imidazo[1,2-a]pyridin-2-yl)methyl]-5H-pyrrolo[2,3-b]pyrazine-2-carboxamide;
N-[(6-{[({3-fluorobicyclo[1.1.1]pentan-1-yl}methyl)amino]methyl}imidazo[1,2-a]pyridin-2-yl)methyl]-5-oxo-5H-[1,3]thiazolo[3,2-a]pyrimidine-7-carboxamide;
4-[1-[(6-methylimidazo[1,2-a]pyridin-2-yl)methyl]triazol-4-yl]isoquinoline;
1-({6-methylimidazo[1,2-a]pyridin-2-yl}methyl)-4-{1H-pyrrolo[2,3-b]pyridin-5-yl}-1H-1,2,3-triazole;
(cyclobutylmethyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{imidazo[1,5-a]pyridin-8-yl}-1H-1,2,3-triazol-1-yl)methyl]imidazo[1,2-a]pyridin-6-yl}methyl)amine;
({2-[(4-{6-chloroimidazo[1,5-a]pyridin-8-yl}-1H-1,2,3-triazol-1-yl)methyl]imidazo[1,2-a]pyridin-6-yl}methyl)(cyclobutylmethyl)amine;
(cyclobutylmethyl)({2-[(4-{1-fluoroimidazo[1,5-a]pyridin-8-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{[1,2,4]triazolo[4,3-a]pyridin-8-yl}-1H-1,2,3-triazol-1-yl)methyl]imidazo[1,2-a]pyridin-6-yl}methyl)amine;
({bicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
2-({2-[(4-{imidazo[1,5-a]pyridin-8-yl}-1H-1,2,3-triazol-1-yl)methyl]imidazo[1,2-a]pyridin-6-yl}methyl)-2-azaspiro[3.3]heptane;
2-({2-[(4-{6-methoxyimidazo[1,5-a]pyridin-8-yl}-1H-1,2,3-triazol-1-yl)methyl]imidazo[1,2-a]pyridin-6-yl}methyl)-2-azaspiro[3.3]heptane;
N-(cyclobutylmethyl)-1-[2-[[4-(4-isoquinolyl)triazol-1-yl]methyl]imidazo[1,2-a]pyridin-6-yl]methanamine;
bis(cyclobutylmethyl)[(2-{[4-(isoquinolin-4-yl)-1H-1,2,3-triazol-1-yl]methyl}imidazo[1,2-a]pyridin-6-yl)methyl]amine;
(cyclobutylmethyl)({2-[(4-{1H-pyrrolo[2,3-b]pyridin-5-yl}-1H-1,2,3-triazol-1-yl)methyl]imidazo[1,2-a]pyridin-6-yl}methyl)amine;
(cyclobutylmethyl)({2-[(4-{1H-pyrrolo[2,3-b]pyridin-5-yl}-1H-imidazol-1-yl)methyl]imidazo[1,2-a]pyridin-6-yl}methyl)amine;
cyclobutylmethyl)({2-[(4-{imidazo[1,5-a]pyridin-5-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{3-methylimidazo[1,5-a]pyridin-8-yl}-1H-1,2,3-triazol-1-yl)methyl]imidazo[1,2-a]pyridin-6-yl}methyl)amine;
3-{1-[(6-{[({bicyclo[1.1.1]pentan-1-yl}methyl)amino]methyl}imidazo[1,2-a]pyridin-2-yl)methyl]-1H-1,2,3-triazol-4-yl}-5-methoxypyridine-2-carbonitrile
3-{1-[(6-{[(cyclobutylmethyl)amino]methyl}imidazo[1,2-a]pyridin-2-yl)methyl]-1H-1,2,3-triazol-4-yl}-5-methoxypyridine-2-carbonitrile;
3-[1-({6-[(4,4-dimethylpiperidin-1-yl)methyl]imidazo[1,2-a]pyridin-2-yl}methyl)-1H-1,2,3-triazol-4-yl]-5-methoxypyridine-2-carbonitrile;
3-(1-{[6-({6-azaspiro[3.4]octan-6-yl}methyl)imidazo[1,2-a]pyridin-2-yl]methyl}-1H-1,2,3-triazol-4-yl)-5-methoxypyridine-2-carbonitrile;
3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}-5-methoxypyridine-2-carbonitrile;
3-(1-{[6-({6-azaspiro[3.4]octan-6-yl}methyl)imidazo[1,2-a]pyridin-2-yl]methyl}-1H-1,2,3-triazol-4-yl)-5-fluoropyridine-2-carbonitrile;
5-fluoro-3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}pyridine-2-carbonitrile;
({3-fluorobicyclo[1.1.1]pentan-1-yl}methyl)[(2-{[4-(6-methoxypyrazin-2-yl)-1H-1,2,3-triazol-1-yl]methyl}imidazo[1,2-a]pyridin-6-yl)methyl]amine;
({3-fluorobicyclo[1.1.1]pentan-1-yl}methyl)[(2-{[4-(5-methoxypyridin-3-yl)-1H-1,2,3-triazol-1-yl]methyl}imidazo[1,2-a]pyridin-6-yl)methyl]amine;
N-[(6-{[(cyclobutylmethyl)amino]methyl}imidazo[1,2-a]pyridin-2-yl)methyl]-5-methoxypyridine-3-carboxamide;
({3-fluorobicyclo[1.1.1]pentan-1-yl}methyl)[(2-{[4-(4-methoxypyridin-3-yl)-1H-1,2,3-triazol-1-yl]methyl}imidazo[1,2-a]pyridin-6-yl)methyl]amine;
({3-fluorobicyclo[1.1.1]pentan-1-yl}methyl)[(2-{[4-(6-methoxypyridin-3-yl)-1H-1,2,3-triazol-1-yl]methyl}imidazo[1,2-a]pyridin-6-yl)methyl]amine;
3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}-5-methoxypyridine-2-carboxamide;
(cyclobutylmethyl)[(2-{[4-(1-methyl-1H-indazol-7-yl)-1H-1,2,3-triazol-1-yl]methyl}imidazo[1,2-a]pyridin-6-yl)methyl]amine;
3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}-5-(morpholin-4-yl)pyridine-2-carbonitrile;
({3-fluorobicyclo[1.1.1]pentan-1-yl}methyl)({[2-({4-[5-(morpholin-4-yl)pyridin-3-yl]-1H-1,2,3-triazol-1-yl}methyl)imidazo[1,2-a]pyridin-6-yl]methyl})amine.

Further compounds of the invention include the following compound or a pharmaceutically acceptable salt thereof:
3-[1-({5-[({(3-methylbicyclo[1.1.1]pent-1-yl)methyl}amino)methyl]-1,3a-diaza-2-indenyl}methyl)-1H-1,2,3-triazol-4-yl]-5-methoxy-2-pyridinecarbonitrile.

In certain embodiments of the invention, the compound is not:
4-[1-[(6-methylimidazo[1,2-a]pyridin-2-yl)methyl]triazol-4-yl]isoquinoline;
tert-butyl N-[[2-[[4-(4-isoquinolyl)triazol-1-yl]methyl]imidazo[1,2-a]pyridin-6-yl]methyl]carbamate;
N-(cyclobutylmethyl)-1-[2-[[4-(4-isoquinolyl)triazol-1-yl]methyl]imidazo[1,2-a]pyridin-6-yl]methanamine.

In certain embodiments of the invention, Z is not one of the following structures:

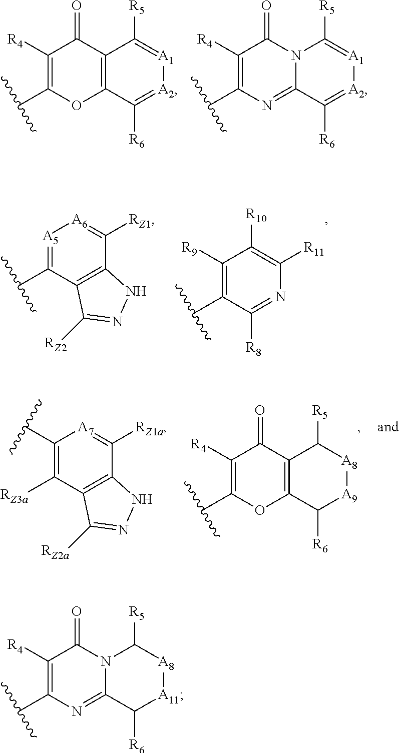

wherein:
R₄, is selected from hydrogen, halo, cyano and methyl;
R₅ is selected from hydrogen, halo, cyano and methyl;
R₆ is selected from hydrogen, halo, cyano and methyl;
R₈, R₉, R₁₀ and R₁₁ are independently selected from hydrogen, NH₂, halo, cyano, and C₁₋₆ alkyl; or
R₉ and R₁₀ may be linked together to form a fused 5- or 6-membered saturated or unsaturated ring system or R₁₀ and R₁₁ may be linked together to form a fused 5- or 6-membered saturated or unsaturated ring system; wherein either of the fused 5- or 6-membered saturated or unsaturated ring system may be optionally substituted by one or more substituents selected from C₁₋₂ alkyl, cyano, C₁₋₂ haloalkyl, hydroxy, C₁₋₂ alkoxy, halo, C₁₋₂ haloalkoxy, NR₁ᵢₐR₁ⱼₐ or —S(O)₀₋₂R₁ᵢₐR₁ⱼₐ, wherein R₁ᵢₐ and R₁ⱼₐ are H or C₁₋₂ alky;
R_{Z1} and R_{Z1a} are selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
R_{Z2} and R_{Z2a} are selected from hydrogen, C₁₋₄ alkyl, cyano, halo, NH₂ and C₁₋₄ alkoxy;
R_{Z3a} is selected from hydrogen, C₁₋₄ alkyl, cyano, halo, NH₂ and C₁₋₄ alkoxy
A₁ is selected from CR₁₂ and N;
A₂ is selected from CR₁₃ and N;
A₅ is selected from CR₁₆ and N;
A₆ is selected from CR₁₇ and N;
A₇ is selected from CR₁₈ and N;
A₈ is selected from CR₁₉R₂₀ and NR₂₁;
A₉ is selected from CR₂₂R₂₃ and NR₂₄;
A₁₁ is selected from CR₂₈R₂₉ and NR₃₀;
R₁₂ is selected from hydrogen, halo, cyano and C₁₋₄ alkyl;
R₁₃ is selected from hydrogen, halo, cyano, methoxy and methyl;
R₁₆ is selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄ cycloalkoxy;
R₁₇ is selected from hydrogen, hydroxy, halo, cyano, C₁₋₅ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—NR_{q}Rᵣ, —(OCH₂CH₂)ₘ—OCH₃ wherein m is an integer from 1 to 6, NR_{q}Rᵣ, —C(O)—NR_{q}Rᵣ, —C(O)OR_{q};
wherein R_{q} and Rᵣ are each independently hydrogen, C₁₋₅ alkyl, C₃₋₆ cycloalkyl, a 3- to 6-membered carbon-linked heterocyclyl, wherein C₁₋₅ alkyl, C₃₋₆ cycloalkyl, a 3- to 6-membered carbon-linked heterocyclyl may be optionally substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
or R_{q} and Rᵣ are linked together such that, together with the nitrogen atom to which they are attached, they form a 3- to 6-membered heterocyclic ring, which may be optionally substituted by one or more substituents selected from C₁₋₂ alkyl, cyano, C₁₋₂ haloalkyl, hydroxy, C₁₋₂ alkoxy, halo, C₁₋₂ haloalkoxy;
wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
R₁₈ is selected from hydrogen, halo, cyano, C₁₋₄ alkyl, C₂₋₄ alkenyl, C₂₋₄ alkynyl, a 5- or 6-membered heteroaryl, C₁₋₄ alkoxy, C₁₋₄ haloalkyl, C₁₋₄ haloalkoxy, C₃₋₄ cycloalkyl, a 3- to 4-membered heterocyclyl and C₃₋₄ cycloalkoxy;
R₁₉ and R₂₀ are selected from hydrogen, halo, cyano and C₁₋₄ alkyl;
R₂₂ and R₂₃ are selected from hydrogen, halo, cyano and methyl;
R₂₈ and R₂₉ are selected from hydrogen, methoxy and methyl;
R₂₁, R₂₄ and R₃₀ are hydrogen.

Particular compounds of the present invention include any of the compounds exemplified in the present application, or a pharmaceutically acceptable salt thereof, and, in particular, any of the following:

The various functional groups and substituents making up the compounds of the formula (I) are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶O and ¹⁸O; and the like.

It is also to be understood that certain compounds of the formula (I) (and compounds of formula (II), (III) and (IV)) may exist in solvated as well as unsolvated forms such as, for example, hydrated forms. It is to be understood that the invention encompasses all such solvated forms that possess antiproliferative activity.

It is also to be understood that certain compounds of the formula (I) (and compounds of formula (II), (III) and (IV)) may exhibit polymorphism, and that the invention encompasses all such forms that possess antiproliferative activity.

Compounds of the formula (I) (and compounds of formula (II), (III) and (IV)) may exist in a number of different tautomeric forms and references to compounds of the formula (I) include all such forms. For the avoidance of doubt, where a compound can exist in one of several tautomeric forms, and only one is specifically described or shown, all others are nevertheless embraced by formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

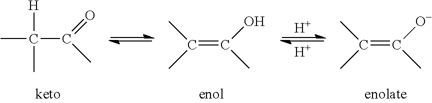

Compounds of the formula (I) containing an amine function may also form N-oxides. A reference herein to a compound of the formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula (I) may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula (I) and in-vivo cleavable amide derivatives that may be formed at a carboxy group or an amino group in a compound of the formula (I).

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically acceptable pro-drug of a compound of the formula (I) is one that is based on reasonable medical judgement as being suitable for administration to the human or animal body without undesirable pharmacological activities and without undue toxicity.

Various forms of pro-drug have been described, for example in the following documents: —
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula (I) that possesses a carboxy group is, for example, an in vivo cleavable ester thereof. An in vivo cleavable ester of a compound of the formula (I) containing a carboxy group is, for example, a pharmaceutically acceptable ester which is cleaved in the human or animal body to produce the parent acid. Suitable pharmaceutically acceptable esters for carboxy include C₁₋₆ alkyl esters such as methyl, ethyl and tert-butyl, C₁._alkoxymethyl esters such as methoxymethyl esters, C₁₋₆alkanoyloxymethyl esters such as pivaloyloxymethyl esters, 3-phthalidyl esters, C₃₋₈ cycloalkylcarbonyloxy-C₁₋₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters.

A suitable pharmaceutically acceptable pro-drug of a compound of the formula (I) that possesses a hydroxy group is, for example, an in vivo cleavable ester or ether thereof. An in vivo cleavable ester or ether of a compound of the formula (I) containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀ alkanoyl groups such as acetyl, benzoyl, phenylacetyl and substituted benzoyl and phenylacetyl groups, C₁₋₁₀ alkoxycarbonyl groups such as ethoxycarbonyl, N,N—(C₁₋₆)₂ carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula (I) that possesses a carboxy group is, for example, an in vivo cleavable amide thereof, for example an amide formed with an amine such as ammonia, a C₁₋₄ alkylamine such as methylamine, a (C₁₋₄ alkyl)₂ amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof.

A suitable pharmaceutically acceptable pro-drug of a compound of the formula (I) that possesses an amino group is, for example, an in vivo cleavable amide derivative thereof. Suitable pharmaceutically acceptable amides from an amino group include, for example an amide formed with C₁₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl.

The in vivo effects of a compound of the formula (I) may be exerted in part by one or more metabolites that are formed within the human or animal body after administration of a compound of the formula (I). As stated hereinbefore, the in vivo effects of a compound of the formula (I) may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### SYNTHESIS

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of formula I will vary depending on the nature of the variable groups. Suitable processes for their preparation are described further in the accompanying Examples.

Once a compound of formula I has been synthesised by any one of the processes defined herein, the processes may then further comprise the additional steps of:
(i) removing any protecting groups present;
(ii) converting the compound formula I into another compound of formula I;
(iii) forming a pharmaceutically acceptable salt, hydrate or solvate thereof; and/or
(iv) forming a prodrug thereof.

The resultant compounds of formula I can be isolated and purified using techniques well known in the art.

### Biological Activity

The METTL3 enzyme and cell assays described in accompanying Example section may be used to measure the pharmacological effects of the compounds of the present invention.

Although the pharmacological properties of the compounds of formula I vary with structural change, as expected, the compounds of the invention were found to be active in these METTL3 assays.

In general, the compounds of the invention demonstrate an IC₅₀ of 10 μM or less in the METTL3 enzyme assay described herein, with preferred compounds of the invention demonstrating an IC₅₀ of 5 μM or less and the most preferred compounds of the invention demonstrating an IC₅₀ of 2 μM or less.

In the METTL3 cell assay described in the Example section, the compounds of formula (I) suitably possess an activity of less than 10 μM, with preferred compounds of the invention demonstrating an IC₅₀ of 5 μM or less and the most preferred compounds demonstrating an activity of 2 μM or less.

### Pharmaceutical Compositions

According to a further aspect of the invention there is provided a pharmaceutical composition which comprises a compound of the invention as defined hereinbefore, or a pharmaceutically acceptable salt thereof, and one or more pharmaceutically acceptable excipients.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition and/or treat or prevent an autoimmune disease referred to herein, slow its progression and/or reduce the symptoms associated with the condition and/or disease.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula (I) will naturally vary according to the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of METTL3 activity.

The present invention therefore provides a method of inhibiting METTL3 activity in vitro or in vivo, said method comprising contacting a cell with an effective amount of a compound, or a pharmaceutically acceptable salt thereof, or a pharmaceutical composition as defined herein.

The present invention also provides a method of treating a disease or disorder in which METTL3 activity is implicated in a patient in need of such treatment, said method comprising administering to said patient a therapeutically effective amount of a compound, or a pharmaceutically acceptable salt thereof, or a pharmaceutical composition as defined herein. Suitably, the disease or disorder in which METTL3 activity is implicated is cancer, such as lung cancer, renal cancer, solid organ cancer, pancreatic cancer or leukaemia, type 2 diabetes, a neuropsychiatric behavioural disorder, infection (e.g. viral infection) or a depressive disorder.

The present invention provides a method of inhibiting cell proliferation, in vitro or in vivo, said method comprising contacting a cell with an effective amount of a compound, or a pharmaceutically acceptable salt thereof, as defined herein.

The present invention provides a method of treating a proliferative disorder, said method comprising administering to a subject in need thereof a therapeutically effective amount of a compound, or a pharmaceutically acceptable salt thereof, or a pharmaceutical composition as defined herein.

The present invention provides a method of treating cancer, said method comprising administering to a subject in need thereof a therapeutically effective amount of a compound, or a pharmaceutically acceptable salt thereof, or a pharmaceutical composition as defined herein. Suitably the cancer is lung cancer, renal cancer, solid organ cancer, pancreatic cancer or leukaemia suitably AML leukaemia or chronic myeloid leukaemia.

The present invention provides a method of treating leukaemia, said method comprising administering to a subject in need thereof a therapeutically effective amount of a compound, or a pharmaceutically acceptable salt thereof, or a pharmaceutical composition as defined herein.

The present invention provides a method of treating AML leukaemia, said method comprising administering to a subject in need thereof a therapeutically effective amount of a compound, or a pharmaceutically acceptable salt thereof, or a pharmaceutical composition as defined herein.

The present invention provides a method of treating an autoimmune disease, said method comprising administering to a subject in need thereof a therapeutically effective amount of a compound, or a pharmaceutically acceptable salt thereof, or a pharmaceutical composition as defined herein. Suitably the autoimmune disease is colitis, multiple sclerosis, rheumatoid arthritis, lupus, cirrhosis, or dermatitis.

The present invention provides a method of treating a neurological disease, said method comprising administering to a subject in need thereof a therapeutically effective amount of a compound, or a pharmaceutically acceptable salt thereof, or a pharmaceutical composition as defined herein.

The present invention provides a method of treating an infectious disease, said method comprising administering to a subject in need thereof a therapeutically effective amount of a compound, or a pharmaceutically acceptable salt thereof, or a pharmaceutical composition as defined herein.

The present invention provides a method of treating an inflammatory disease, said method comprising administering to a subject in need thereof a therapeutically effective amount of a compound, or a pharmaceutically acceptable salt thereof, or a pharmaceutical composition as defined herein.

The present invention provides a compound, or a pharmaceutically acceptable salt thereof, or a pharmaceutical composition as defined herein for use in therapy.

The present invention provides a compound, or a pharmaceutically acceptable salt thereof, or a pharmaceutical composition as defined herein for use in the treatment of a proliferative condition.

The present invention provides a compound, or a pharmaceutically acceptable salt thereof, or a pharmaceutical composition as defined herein for use in the treatment of cancer. In a particular embodiment, the cancer is human cancer. Suitably the cancer is lung cancer, renal cancer, solid organ cancer, pancreatic cancer or leukaemia suitably AML leukaemia or chronic myeloid leukaemia.

The present invention provides a compound, or a pharmaceutically acceptable salt thereof, or a pharmaceutical composition as defined herein for use in the treatment of leukaemia.

The present invention provides a compound, or a pharmaceutically acceptable salt thereof, or a pharmaceutical composition as defined herein for use in the treatment of AML leukaemia.

The present invention provides a compound, or a pharmaceutically acceptable salt thereof, or a pharmaceutical composition as defined herein for use in the inhibition of METTL3 activity.

The present invention provides a compound, or a pharmaceutically acceptable salt thereof, or a pharmaceutical composition as defined herein for use in the treatment of an autoimmune disease. Suitably the autoimmune disease is colitis, multiple sclerosis, rheumatoid arthritis, lupus, cirrhosis, or dermatitis.

The present invention provides a compound, or a pharmaceutically acceptable salt thereof, or a pharmaceutical composition as defined herein for use in the treatment of an neurological disease.

The present invention provides a compound, or a pharmaceutically acceptable salt thereof, or a pharmaceutical composition as defined herein for use in the treatment of an infectious disease.

The present invention provides a compound, or a pharmaceutically acceptable salt thereof, or a pharmaceutical composition as defined herein for use in the treatment of an inflammatory disease.

The present invention provides a compound, or a pharmaceutically acceptable salt thereof, as defined herein for use in the treatment of a disease or disorder in which METTL3 activity is implicated. Suitably, the disease or disorder in which METTL3 activity is implicated is cancer, such as lung cancer, renal cancer, solid organ cancer, pancreatic cancer or leukaemia, type 2 diabetes, a neuropsychiatric behavioural disorder or a depressive disorder.

The present invention provides a use of a compound, or a pharmaceutically acceptable salt thereof, as defined herein in the manufacture of a medicament for the treatment of a proliferative condition.

The present invention provides a use of a compound, or a pharmaceutically acceptable salt thereof, as defined herein in the manufacture of a medicament for the treatment of cancer. Suitably, the medicament is for use in the treatment of human cancers. Suitably the cancer is lung cancer, renal cancer, solid organ cancer, pancreatic cancer or leukaemia suitably AML leukaemia or chronic myeloid leukaemia.

The present invention provides a use of a compound, or a pharmaceutically acceptable salt thereof, as defined herein in the manufacture of a medicament for the treatment of leukaemia.

The present invention provides a use of a compound, or a pharmaceutically acceptable salt thereof, as defined herein in the manufacture of a medicament for the treatment of AML leukaemia.

The present invention provides a use of a compound, or a pharmaceutically acceptable salt thereof, as defined herein in the manufacture of a medicament for the treatment of an autoimmune disease. Suitably the autoimmune disease is colitis, multiple sclerosis, rheumatoid arthritis, lupus, cirrhosis, or dermatitis.

The present invention provides a use of a compound, or a pharmaceutically acceptable salt thereof, as defined herein in the manufacture of a medicament for the treatment of a neurological disease.

The present invention provides a use of a compound, or a pharmaceutically acceptable salt thereof, as defined herein in the manufacture of a medicament for the treatment of an inflammatory disease.

The present invention provides a use of a compound, or a pharmaceutically acceptable salt thereof, as defined herein in the manufacture of a medicament for the treatment of an infectious disease.

The present invention provides a use of a compound, or a pharmaceutically acceptable salt thereof, as defined herein in the manufacture of a medicament for the inhibition of METTL3 activity.

The present invention provides a use of a compound, or a pharmaceutically acceptable salt thereof, as defined herein in the manufacture of a medicament for the treatment of a disease or disorder in which METTL3 activity is implicated. Suitably, the disease or disorder in which METTL3 activity is implicated is cancer, such as lung cancer, renal cancer, solid organ cancer, pancreatic cancer or leukaemia, type 2 diabetes, a neuropsychiatric behavioural disorder or a depressive disorder.

The term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain and skin.

The anti-proliferative effects of the compounds of the present invention have particular application in the treatment of human cancers (by virtue of their inhibition of METTL3 activity).

The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

In a particular embodiment of the invention, the proliferative condition to be treated is cancer.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The antiproliferative treatment defined hereinbefore may be applied as a sole therapy or may involve, in addition to the compound of the invention, conventional surgery or radiotherapy or chemotherapy. Such chemotherapy may include one or more of the following categories of anti-tumour agents:—
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin ανβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies; and
(xi) Agents used to treat AML leukaemia, including for example, cytarabine, FLT3 inhibitors, BCL2 inhibitors or IDH1/2 inhibitors.

In a particular embodiment, the antiproliferative treatment defined hereinbefore may involve, in addition to the compound of the invention, conventional surgery or radiotherapy or chemotherapy.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt thereof, and another anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt thereof, and any one of the anti-tumour agents listed herein above.

In a further aspect of the invention there is provided a compound of the invention or a pharmaceutically acceptable salt thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt thereof, in combination with an anti-tumour agent (optionally selected from one listed herein above), in association with a pharmaceutically acceptable diluent or carrier.

In another embodiment, the invention relates to a therapeutic combination comprising a compound as defined herein and another agent used to treat AML leukaemia e.g., cytarabine, FLT3 inhibitors, BCL2 inhibitors or IDH1/2 inhibitors.

### EXAMPLES

The following abbreviations have been used in the Examples:
DCE—1,2-Dichloroethane
DCM—Dichloromethane
DIBAL—Diisobutylaluminium hydride
DIPEA—N-ethyl-N-isopropyl-propan-2-amine
DMAP—4-Dimethylaminopyridine
DMF—Dimethylformamide
HATU—[dimethylamino(triazolo[4,5-b]pyridin-3-yloxy)methylene]-dimethyl-ammonium; hexafluorophosphate
HPLC—High-performance liquid chromatography
IPA—Isopropanol
LCMS—Liquid chromatography mass spectrometry
RBF—Round bottomed flask
RM—Reaction mixture
RT—Retention Time
TFA—Trifluoroacetic acid
THF—Tetrahydrofuran

LCMS Method A refers to low pH analysis using a mobile phase consisting of 0.1% formic acid in a gradient of 5-100% MeCN in water over 1.2 min at a flow rate of 1.2 mL/min. The stationary phase consisted of a Kinetex Core-Shell C18, 2.1 mm×50 mm, 5 μm. The experiment was run at 40° C.

LCMS Method B refers to high pH analysis using a mobile phase consisting of 2 mM ammonium bicarbonate, buffered to pH10 in a gradient of 5-100% MeCN in water over 2.1 min at a flow rate of 1.0 mL/min. The stationary phase consisted of a Phenomenex Gemini-NX C18, 2.0×50 mm, 3 μm. The experiment was run at 40° C.

LCMS Method C refers to high pH analysis using a mobile phase consisting of 2 mM ammonium bicarbonate, buffered to pH10 in a gradient of 5-100% MeCN in water over 5.8 min at a flow rate of 0.6 mL/min. The stationary phase consisted of a Waters UPLC® BEH™ C18, 2.1×100 mm, 1.7 μm. The experiment was run at 40° C.

LCMS Method D refers to high pH analysis using a mobile phase consisting of 2 mM ammonium bicarbonate, buffered to pH10 in a gradient of 5-100% MeCN in water over 5.9 min at a flow rate of 0.6 mL/min. The stationary phase consisted of a Phenomenex Gemini-NX C18, 2.0×100 mm, 3 μm. The experiment was run at 40° C.

LCMS Method E refers to low pH analysis using a mobile phase consisting of 0.1% formic acid in a gradient of 5-100% MeCN in water over 5.3 min at a flow rate of 0.6 mL/min. The stationary phase consisted of a Phenomenex Kinetix-XB C18, 2.1 mm×100 mm, 1.7 μm. The experiment was run at 40° C.

LCMS Method F refers to high pH analysis using a mobile phase consisting of 2 mM ammonium bicarbonate, buffered to pH10 in a gradient of 5-100% MeCN in water over 0.75 min at a flow rate of 1.0 mL/min. The stationary phase consisted of a Waters UPLC® BEH™ C18, 2.1×100 mm, 1.7 μm. The experiment was run at 40° C.

LCMS Method G refers to low pH analysis using a mobile phase consisting of 0.1% Formic acid in water (pH=2.70) in a gradient of 3-100% of 0.1% formic acid in water:acetonitrile (10:90) over 3.00 min at a flow rate of 0.8 mL/min. The stationary phase consisted of C18, 50*2.1 mm, 1.6 μm column. The experiment was run at 35° C.

LCMS Method H refers to a high pH analysis using a mobile phase consisting of 5 mM ammonium bicarbonate, (pH 7.35) in a gradient of MeCN in water over 3.0 min at a flow rate of 0.5 mL/min. The stationary phase consisted of C18, 50*2.1 mm, 2.5 μm. The experiment was run at 35° C.

LCMS Method I refers to a high pH analysis using a mobile phase consisting of 5 mM ammonium bicarbonate, (pH 7.35) in a gradient of MeCN in water over 3.0 min at a flow rate of 0.5 mL/min. The stationary phase consisted of C18, 50*2.1 mm, 2.5 μm. The experiment was run at 35° C.

LCMS Method J refers to high pH analysis using a mobile phase consisting of 2 mM ammonium bicarbonate, buffered to pH10 in a gradient of 1-100% MeCN over 1.35 min at a flow rate of 1 mL/min. The stationary phase consisted of a Waters UPLC® BEH™ 018 2.1×30 mm, 1.7 μm. The experiment was run at 40° C.

LCMS Method K refers to a Low pH analysis using a mobile phase consisting of 0.1% Formic acid in water in a gradient of 5-100% of 0.1% formic acid in water: 0.1% formic acid in acetonitrile over 5.4 min at a flow rate of 0.6 mL/min. The stationary phase consisted of Phenomenex Kinetex Core-Shell C8 50×2.1 mm, 2.6 □m (protected by a “security Guard” column. The experiment was run at 40° C.

LCMS Method L refers to a Low pH analysis using a mobile phase consisting of 0.1% Formic acid in water in a gradient of 5-100% of 0.1% formic acid in water: 0.1% formic acid in acetonitrile over 1.1 min at a flow rate of 0.9 mL/min. The stationary phase consisted of Waters UPLC® BEH™ C18 2.1×50 mm, 1.7 μm. The experiment was run at 40° C.

LCMS Method M refers to a Low pH analysis using a mobile phase consisting of 0.1% Formic acid in water in a gradient of 5-100% of 0.1% formic acid in water: 0.1% formic acid in acetonitrile over 2.25 min at a flow rate of 1.2 mL/min. The stationary phase consisted of Phenomenex Kinetex Core-Shell C8 50×2.1 mm, 2.6 □m. The experiment was run at 40° C.

The following preparative HPLC Methodologies were used:
Preparative Method A refers to low pH purification using a mobile phase consisting of 0.1% Formic acid in a gradient of 10-95% MeCN in water over 14.4 min at a flow rate of 40 mL/min. The stationary phase consisted of a Waters Sunfire C18, 30×100 mm, 10 μm.
Preparative Method B refers to high pH purification using a mobile phase consisting of 0.2% ammonium hydroxide in a gradient of 30-95% MeCN in water over 10 min at a flow rate of 40 mL/repmin. The stationary phase consisted of a Waters XBridge™ C18 OBD™, 30×100 mm, 10 μm.

### Intermediate 1: (6-{[(cyclohexylmethyl)amino]methyl}imidazo[1,2-a]pyridin-2-yl)methanamine Trihydrochloride

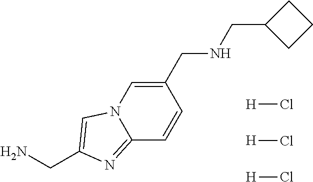

### Step 1: tert-butyl N-({6-cyanoimidazo[1,2-a]pyridin-2-yl}methyl)carbamate

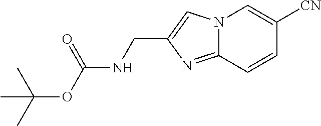 [SMILES: CC(C)(C)OC(=O)NCc1cn2cc(C#N)ccc2n1]

A suspension of 2-(aminomethyl)imidazo[1,2-a]pyridine-6-carbonitrile (2.60 g, 15.1 mmol) and N-ethyl-N-isopropyl-propan-2-amine (3.7 mL, 20.9 mmol) in THF (35 mL) and DCM (35 mL) was treated with N,N-dimethylpyridin-4-amine (0.085 g, 0.697 mmol) and tert-butoxycarbonyl tert-butyl carbonate (3.40 g, 15.6 mmol) at 0° C. and the mixture was allowed to warm to ambient temperature and stirred at ambient temperature for 65 hours. The reaction mixture was concentrated under vacuum, the residue was suspended in water-CH₃CN (1:1, 15 mL), and sonicated for 20 min. The solid was filtered, and then treated with water-MeCN again. The obtained solid was dried under vacuum to give the title compound (2.78 g, 63%) as an off white solid. The filtrate was purified by reverse phase chromatography (Biotage 400 g eluting with 5-100% (0.1% formic acid in MeCN) in 0.1% formic acid to provide a further crop of title compound (414 mg, 10%).

Method B: LC-MS (electrospray): m/z=273.3 (M+H)⁺, RT=1.38 min.

### Step 2: tert-butyl N-({6-formylimidazo[1,2-a]pyridin-2-yl}methyl)carbamate

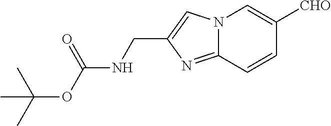 [SMILES: CC(C)(C)OC(=O)NCc1cn2cc(C=O)ccc2n1]

A suspension of tert-butyl N-[(6-cyanoimidazo[1,2-a]pyridin-2-yl)methyl]carbamate (5.00 g, 18.4 mmol) and sodium phosphinate hydrate (15.57 g, 0.147 mol) in a mixture of water (50 mL), Pyridine (100 mL) and Acetic acid (50 mL) was treated with Raney nickel (50%, 18.97 g, 0.162 mol) and the mixture was heated at 100° C. under a nitrogen atmosphere for an hour. The Raney nickel was removed by hot filtration through a bed of Celite (washing with water, followed by MeOH). The filtrate was concentrated under vacuum to remove the MeOH and the blue solution was extracted with DCM (50 mL×4). The extracts evaporated under vacuum to afford a beige gum which was triturated with water to furnish a white solid. The solid was collected by filtration, washed with water followed by ether and dried under vacuum overnight to provide the title compound (4.13 g, 82%) as an off white solid.

Method B: LC-MS (electrospray): m/z=276.2 (M+H)⁺, RT=1.33 min.

### Step 3: tert-butyl N-[(6-{[(cyclohexylmethyl)amino]methyl}imidazo[1,2-a]pyridin-2-yl)methyl]carbamate

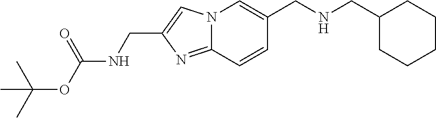 [SMILES: CC(C)(C)OC(=O)NCc1cn2cc(CNCC3CCCCC3)ccc2n1]

A solution of tert-butyl N-[(6-formylimidazo[1,2-a]pyridin-2-yl)methyl]carbamate (340 mg, 0.988 mmol) and cyclohexylmethanamine (224 mg, 1.98 mmol) in ethanol (6.8 mL) was stirred at 50° C. for an hour. The reaction mixture was cooled to 0° C. and NaBH₄ (75 mg, 1.98 mmol) was added in portions. The reaction mixture was allowed to warm to ambient temperature and stirring was continued for 2 hours. The reaction mixture was partitioned between DCM (80 mL) and aqueous sodium hydrogen carbonate (sat, 30 mL). The organic layer was separated, washed with brine (30 mL), dried (MgSO₄), concentrated under reduced pressure. The residue was purified by column chromatography on silica gel (Biotage KP-NH 28 g, eluting with 0-20% MeOH in EtOAc) to afford the title compound (360 mg, 97%) as a pale yellow oil.

Method B: LC-MS (electrospray): m/z=373.3 (M+H)⁺, RT=1.76 min.

### Step 4: (6-{[(cyclohexylmethyl)amino]methyl}imidazo[1,2-a]pyridin-2-yl)methanamine Trihydrochloride

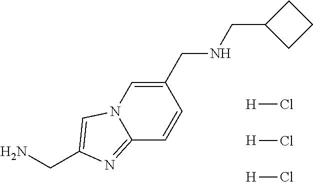

To a solution of tert-butyl N-[[6-[(cyclohexylmethylamino)methyl]imidazo[1,2-a]pyridin-2-yl]methyl]carbamate (360 mg, 0.908 mmol) in Methanol (4.1379 mL) was added HCl (4 M in dioxane, 2.4 mL, 9.6 mmol) at ambient temperature. The solution was stirred at 50° C. for 1 hour. The solution was cooled to ambient temperature, and concentrated to dryness under reduced pressure. The residue (pale yellow glass) was dissolved in MeOH (˜3 mL) and diethyl ether (˜20 mL) was added drop-wise to the stirred solution. The resulting precipitate was collected by filtration and dried in the vacuum oven at 40° C. for 4 hours to afford [the title compound (359 mg, 100%) as white solid.

Method B: LC-MS (electrospray): m/z=273.3 (M+H)⁺, RT=1.77 min.

The compounds in Table 1 were prepared in a similar manner to Intermediate 1.

TABLE 1

| | | | | LCMS | |
|---|---|---|---|---|---|
| | | | LCMS | Retention | mass |
| Intermediate | Name | Structure | Method | time | ion |
| 2 | 1-(6- {[(cyclobutylmethyl) amino]methyl}imidazo[1,2- a]pyridin-2- yl)methanamine trihydrochloride | 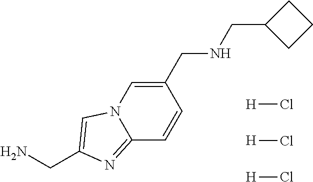 | C | 2.02 | 245.3 |
| 3 | 1-[6-({[(3,3- difluorocyclobutyl)methyl] amino}methyl)imidazo [1,2-a]pyridin-2- yl]methanamine trihydrochloride | 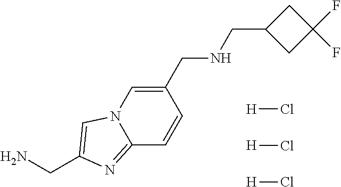 | D | 2.77 | 281.3 |
| 4 | 1-(6-{[({3- fluorobicyclo[1.1.1] pentan-1- yl}methyl)amino]methyl} imidazo[1,2-a]pyridin-2- yl)methanamine trihydrochloride | 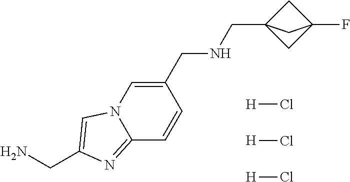 | B | 1.24 | 275.3 |

### Intermediate 5: 1,2-dimethyl-6-oxo-1,6-dihydropyrimidine-4-carboxylic Acid

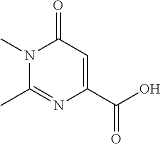

### Step 1: 2-methyl-6-oxo-3,6-dihydropyrimidine-4-carboxylic Acid

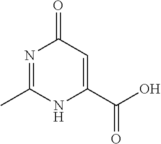

Sodium diethyl 2-oxobutanedioate (4.84 g, 23.0 mmol) was suspended in water (16 mL) and ethanol (4 mL) and sodium hydroxide (6.25 M, 3.6 mL, 22.5 mmol) was added and the mixture was stirred at ambient temperature until partial dissolution, pale brown. A solution of acetamidine hydrochloride (1.81 g, 19.1 mmol) in water (16 mL) was added and the mixture heated at 80° C. for 2 h, with addition of drops of NaOH (6.25 M aqueous) to maintain pH at 11-12. The reaction mixture was cooled in an ice and concentrated hydrochloric acid was added slowly until ˜pH 1, causing precipitation. The mixture was stirred briefly at ambient temperature, then the solids were collected by filtration, washed with water followed by Et₂O and dried under suction to afford the title compound (3.50 g, 97%) of the title compound as a pale grey solid.

Method B: LC-MS (electrospray): m/z=155.2 (M+H)⁺, RT=0.23 min.

### Step 2: Methyl 1,2-dimethyl-6-oxo-1,6-dihydropyrimidine-4-carboxylate

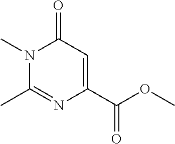

To a suspension of methyl 2-methyl-6-oxo-1H-pyrimidine-4-carboxylate (374 mg, 2.22 mmol) and K₂CO₃ (922 mg, 6.67 mmol) in acetonitrile (15 mL) was added iodomethane (0.28 mL, 4.45 mmol) at ambient temperature and the reaction mixture was stirred for 72 hours. The solids were collected by filtration and washed with Acetonitrile (15 mL). The filtrate was concentrated under reduced pressure to afford then title compound (410 mg, 96%) as a colorless oil that solidified upon standing.

Method B: LC-MS (electrospray): m/z=183.2 (M+H)⁺, RT=0.799 min.

### Step 3: 1,2-dimethyl-6-oxo-1,6-dihydropyrimidine-4-carboxylic Acid

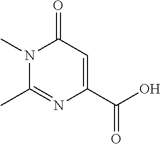

To a solution of methyl 1,2-dimethyl-6-oxo-pyrimidine-4-carboxylate (107 mg, 0.587 mmol) in methanol (3 mL) was added potassium hydroxide (2 M, 0.44 mL, 0.881 mmol) at ambient temperature. The reaction mixture was stirred at ambient temperature for 3 hours before hydrogen chloride (2 M, 0.59 mL, 1.17 mmol) was added and the reaction mixture was concentrated to dryness under reduced pressure. The residue was triturated with ethanol (˜10 mL). The solids were removed by filtration and washed with ethanol (˜5 mL). The filtrate was concentrated under vacuum and triturated with acetonitrile (˜3 mL) to afford the title compound (28 mg, 28%) as brown solid.

### Intermediate 6: 4-Oxopyrido[1,2-a]pyrimidine-2-carboxylic Acid

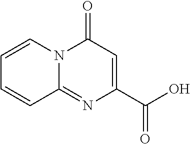 [SMILES: O=C(O)c1cc(=O)n2ccccc2n1]

Methyl 4-oxopyrido[1,2-a]pyrimidine-2-carboxylate[Tetrahedron (2014), 70(17), 2761-2765] (3.94 g, 19.3 mmol) was dissolved in hydrogen chloride solution (8 M, 7.5 mL) at ambient temperature (An exotherm was noted on addition) and the mixture was heated at reflux at for 2 hours.

The mixture was cooled to ambient temperature and the precipitate was collected by filtration and dried under vacuum to give the title compound (3.00 g, 81%) as a white solid.

Method A: LC-MS (electrospray): m/z=191.1 (M+H)⁺, RT=0.32 min

### Intermediate 7: 4-oxo-6,7,8,9-tetrahydropyrido[1,2-a]pyrimidine-2-carboxylic Acid Hydrochloride

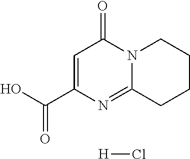 [SMILES: O=C(O)c1cc(=O)n2c(n1)CCCC2 ; Cl]

A suspension of rhodium (504 mg, 4.90 mmol) in ethanol (20 mL) was placed under a nitrogen atmosphere. 4-oxopyrido[1,2-a]pyrimidine-2-carboxylate (Intermediate 6, 1000 mg, 2.66 mmol) was added. The mixture was placed under an atmosphere of hydrogen and the mixture was stirred at ambient temperature for 4 hours. The catalyst was removed by filtration and the filtrate was evaporated under vacuum to afford a brown gum which crystallised on standing to give the title compound (600 mg, 97%) as beige solid.

Method B: LC-MS (electrospray): m/z=195.2 (M+H)⁺, RT=0.28 min. (M+H)⁺, RT=0.76 min.

### Intermediate 8: 4-oxo-4H-quinolizine-2-carboxylic Acid

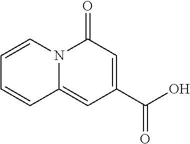 [SMILES: O=C(O)c1cc(=O)n2ccccc2c1]

### Step 1: dimethyl 2-(triphenyl-λ\hat{ }5-phosphanylidene)butanedioate

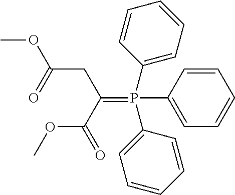 [SMILES: COC(=O)CC(C(=O)OC)=P(c1ccccc1)(c1ccccc1)c1ccccc1]

To a solution of dimethyl ({Z})-but-2-enedioate (2.5 mL, 20.0 mmol) in acetonitrile (25 mL) was added triphenylphosphonium-bromide (6.86 g, 20.0 mmol) and the mixture was heated at reflux for 2 hours (clear colourless solution). After cooling to ambient temperature, the reaction mixture was poured into water (200 mL). The cloudy mixture was washed with diethyl ether (2×50 mL). The clear aqueous layer was basified with NaOH (2 M) and extracted with dichloromethane (2×100 mL). The combined extracts were washed with brine (50 mL), dried (MgSO₄), filtered and concentrated under reduced pressure. The residue was triturated with ethyl acetate and dried in the vacuum oven at 40° C. for 2 hours to afford the title compound (5.10 g, 63%) as white solid.

Method A: LC-MS (electrospray): m/z=407.0 (M+H)⁺, RT=0.86 min.

### Step 2: 1,4-dimethyl (2E)-2-[(pyridin-2-yl)methylidene]butanedioate

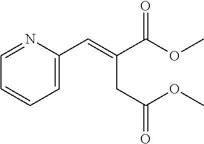 [SMILES: COC(=O)C/C(=C\c1ccccn1)C(=O)OC]

To a solution of pyridine-2-carbaldehyde (1.00 g, 9.34 mmol) in acetonitrile (20 mL) was added dimethyl 2-(triphenyl-λ\hat{ }5-phosphanylidene)butanedioate (3.79 g, 9.34 mmol) at ambient temperature. The mixture was heated at reflux for 6 hours. After cooling to ambient temperature, the volatiles were removed under reduced pressure. The residue was dissolved in a small amount of dichloromethane and purified by chromatography on silica gel (Biotage KP-Sil 50 g, eluting with 0-50% EtOAc in heptane) to afford the title compound (2.12 g, 96%) as pale yellow oil.

Method A: LC-MS (electrospray): m/z=236.0 (M+H)⁺, RT=0.96 min.

### Step 3: methyl 4-oxoquinolizine-2-carboxylate

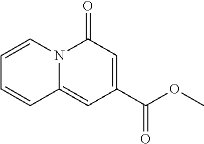

To a solution of dimethyl (2E)-2-(2-pyridylmethylene)butanedioate (2.01 g, 8.54 mmol) in toluene (25 mL) was added 4-methylbenzenesulfonic acid (15 mg, 0.0854 mmol) and the reaction mixture was heated at reflux for 6 hours. After cooling to ambient temperature, the volatiles were removed under reduced pressure. The residue was dissolved in a small amount of dichloromethane and purified by chromatography on silica gel (Biotage KP-Sil 25 g, eluting with 0-100% EtOAc in heptane). To afford the title compound (1.17 g, 67%) as bright yellow solid.

Method A: LC-MS (electrospray): m/z=204.0 (M+H)⁺, RT=0.86 min.

### Step 4: 4-oxo-4H-quinolizine-2-carboxylic Acid

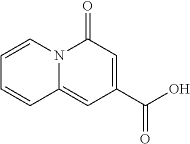 [SMILES: O=C(O)c1cc(=O)n2ccccc2c1]

To a solution of methyl 4-oxoquinolizine-2-carboxylate (750 mg, 3.69 mmol) in methanol (5 mL) was added potassium hydroxide (2 M, 2.8 mL, 5.54 mmol) at ambient temperature. The reaction mixture was stirred at ambient temperature for 1 hour. hydrogen chloride (2 M, 3.7 mL, 7.38 mmol) was added to the reaction mixture (a bright yellow. precipitate formed immediately). The precipitate was collected by vacuum filtration, washed with water (˜10 mL) and dried in the vacuum oven at 40° C. for 4 hours to afford the title compound (655 mg, 94%) as bright yellow solid.

Method A: LC-MS (electrospray): m/z=189.9

### Intermediate 9: 2-(azidomethyl)-6-methylimidazo[1,2-a]pyridine

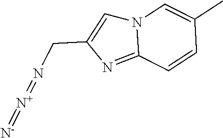

A suspension of 2-(chloromethyl)-6-methyl-imidazo[1,2-a]pyridine hydrochloride (532 mg, 2.45 mmol), triethylamine (1 mL, 7.35 mmol) and NaI (111 mg, 0.74 mmol) were suspended in DMF (5 mL) before sodium azide (175 mg, 2.70 mmol) was added and the mixture was stirred at ambient temperature. For 16 hours. The reaction mixture was quenched with aqueous sodium hydrogen carbonate (sat, 20 mL) and extracted with chloroform/isopropanol (3:1, 4×30 mL) and the combined organic extracts were dried over sodium sulfate and evaporated under vacuum. The residue was purified by chromatography on silica gel (Biotage 28 g kp-NH, eluting with EtOAc/heptane 0-100%) to afford the title compound (291 mg, 63%) as a colourless oil.

Method B: LC-MS (electrospray): m/z=188.2 (M+H)⁺, RT=1.33 min.

### Intermediate 10: tert-butyl N-[[2-(azidomethyl)imidazo[1,2-a]pyridin-6-yl]methyl]-N-(cyclobutylmethyl)carbamate

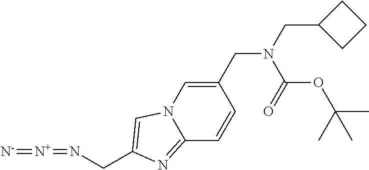 [SMILES: CC(C)(C)OC(=O)N(Cc1ccc2nc(CN=[N+]=[N-])cn2c1)CC1CCC1]

### Step 1: [(6-chloropyridin-3-yl)methyl](cyclobutylmethyl)amine

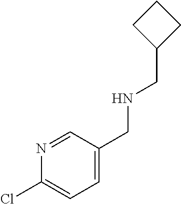 [SMILES: Clc1ccc(CNCC2CCC2)cn1]

A solution of 1-cyclobutylmethanamine (1.80 g, 21.2 mmol) in DCE (30 mL). was treated with 6-chloropyridine-3-carbaldehyde (2.50 g, 17.7 mmol) followed by magnesium sulfate (1.06 g, 8.83 mmol) and the reaction was stirred at ambient temperature for 1.5 hours. Sodium borohydride (0.67 g, 17.7 mmol) and MeOH (0.5 mL) were added and the mixture was stirred at ambient temperature for 17 hours. More Sodium borohydride (1.54 g, 40.7 mmol) and methanol (1.5 mL) were added and the mixture was stirred at ambient temperature for 5 hours and at 40° C., for 1.5 hours. The mixture was poured into NaHCO₃ (sat, 50 mL) and stirred for 20 minutes. The phases were separated and the aqueous phase extracted with DCM (2×25 mL). The combined organics were washed with brine (25 mL). The combined aqueous phases were extracted with DCM (25 mL). All the organic phases were combined, dried MgSO₄ and concentrated. to give a yellow oil which was purified by chromatography on SiO₂ (Biotage Kp-Sil 50 g) eluting with EtOAc. to give the title compound (1.89 g, 46%) as a pale yellow oil.

Method C: LC-MS (electrospray): m/z=210.9, (M+H)⁺, RT=2.95 min.

### Step 2: tert-butyl N-[(6-chloro-3-pyridyl)methyl]-N-(cyclobutylmethyl) carbamate

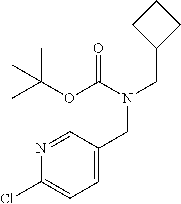 [SMILES: CC(C)(C)OC(=O)N(Cc1ccc(Cl)nc1)CC1CCC1]

A solution of Di-tert-butyl dicarbonate (3.76 g, 17.2 mmol) in MeCN (30 mL) was added to a flask containing 1-(6-chloro-3-pyridyl)-N-(cyclobutylmethyl)methanamine (3.44 g, 14.4 mmol) and the mixture was refluxed at 80° C. for 2 hours. The mixture was cooled to ambient temperature and concentrated to a yellow oil which was purified by chromatography on SiO₂ (Biotage Kp-Sil 100 g) eluting with EtOAc in heptane (0-15%) to give the title compound (4.47 g, 100%) as a colourless oil.

Method C: LC-MS (electrospray): m/z=311.2, (M+H)⁺, RT=4.32 min.

### Step 3: tert-butyl N-[(6-amino-3-pyridyl)methyl]-N-(cyclobutylmethyl)carbamate

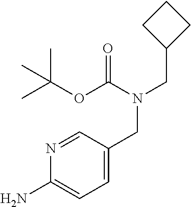 [SMILES: CC(C)(C)OC(=O)N(Cc1ccc(N)nc1)CC1CCC1]

An oven-dried 100 mL two-neck RBF was charged with tert-butyl N-[(6-chloro-3-pyridyl)methyl]-N-(cyclobutylmethyl)carbamate (4.40 g, 14.2 mmol), XPhos (1350 mg, 2.83 mmol) and Pd₂dba₃ (648 mg, 0.708 mmol). The flask was equipped with a reflux condenser and the assembly was flushed with nitrogen. THF (anhydrous, 47 mL) was added and the resulting solution was de-oxygenated by bubbling nitrogen for 30 minutes. [Bis(trimethylsilyl)amino]lithium (1 M in THF, 21 mL, 21.2 mmol) was added with vigorous stirring dropwise over 5 minutes and the mixture was placed in a pre-heated heating block at 70° C. and stirred for 2 hours.

The mixture was cooled to ambient temperature, poured into NaHCO₃ (sat, 50 mL) and stirred for 5 minutes before the mixture was extracted with DCM (2×50 mL). The aqueous phase was diluted with brine (30 mL) and extracted with DCM (2×50 mL). The combined organics were washed with brine (30 mL). The brine washings were extracted with DCM (50 mL). All the organics were combined, dried (MgSO₄) and concentrated to give the crude product as a viscous brown oil which was purified by chromatography on SiO₂ (Biotage, Amino Duo D 55 g) eluting with EtOAc in heptane (0-100%) to give the title compound (3.25 g, 75%) as a light orange solid.

Method D: LC-MS (electrospray): m/z=292.3, (M+H)⁺, RT=4.24 min.

### Step 4: tert-butyl N-[[2-(chloromethyl)imidazo[1,2-a]pyridin-6-yl]methyl]-N-(cyclobutylmethyl)carbamate

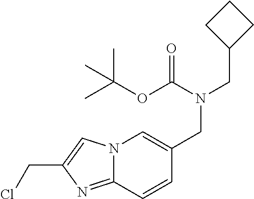 [SMILES: CC(C)(C)OC(=O)N(Cc1ccc2nc(CCl)cn2c1)CC1CCC1]

A mixture of tert-butyl N-[(6-amino-3-pyridyl)methyl]-N-(cyclobutylmethyl)carbamate (3.16 g, 10.3 mmol) in DME (anhydrous, 45 mL) was treated with 1,3-dichloropropan-2-one (1.44 g, 11.3 mmol) and the mixture was heated, with stirring, at 80° C. for 1.5 hours. More 1,3-dichloropropan-2-one (260 mg, 2 mmol) was added and the mixture was heated at 80° C. for 30 minutes. The mixture was cooled to ambient temperature and washed with NaHCO₃ (sat, 2×15 mL). The aqueous was extracted with EtOAc (2×20 mL) and the combined organics were dried (MgSO₄) and concentrated to give the crude product as an orange-brown semi-solid. The product was purified by chromatography on neutral alumina (˜250 g) eluting in a step gradient of EtOAc/heptane: 1:3 (500 mL), 1:2 (500 mL), 1:1 (1.5 L) to give the title compound (1.90 g, 49%) as a light yellow solid.

Method C: LC-MS (electrospray): m/z=364.2, (M+H)⁺, RT=3.89 min.

### Step 5: tert-butyl N-[[2-(azidomethyl)imidazo[1,2-a]pyridin-6-yl]methyl]-N-(cyclobutylmethyl)carbamate

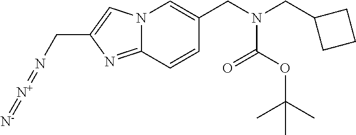 [SMILES: CC(C)(C)OC(=O)N(Cc1ccc2nc(CN=[N+]=[N-])cn2c1)CC1CCC1]

A vial was charged with sodium azide (300 mg, 4.62 mmol), sodium iodide (28 mg, 0.185 mmol), tert-butyl N-[[2-(chloromethyl)imidazo[1,2-a]pyridin-6-yl]methyl]-N-(cyclobutylmethyl)carbamate (700 mg, 1.85 mmol) and DMF (3.7 mL). The resulting suspension was stirred at ambient temperature for 2 hours. The mixture was diluted with water (3 mL) and extracted with EtOAc (3×10 mL). The combined organics were washed with brine (5 mL), dried (MgSO₄) and concentrated to give the crude product as a yellow oil which partially solidifies on standing. The material was purified by chromatography on SiO₂ (Botage 25 g) eluting with EtOAc/heptane (0-50%). The product to give the title compound (719 mg, 99%) as a yellow oil.

Method C: LC-MS (electrospray): m/z=371.4, (M+H)⁺, RT=3.91 min.

The compounds in Table 2 were prepared similarly to Intermediate 10 form the appropriate amine.

TABLE 2

| | | | | LCMS | |
|---|---|---|---|---|---|
| | | | LCMS | Retention | mass |
| Intermediate | Name | Structure | Method | time | ion |
| 11 | tert-butyl N-{[2- (azidomethyl)imidazo [1,2-a]pyridin-6- yl]methyl}-N-({3- fluorobicyclo[1.1.1] pentan-1- yl}methyl)carbamate | 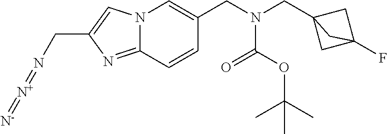 [SMILES: CC(C)(C)OC(=O)N(Cc1ccc2nc(CN=[N+]=[N-])cn2c1)CC12CC(F)(C1)C2] | J | 0.88 | 401.5 |
| 12 | tert-butyl N-{[2- (azidomethyl)imidazo [1,2-a]pyridin-6- yl]methyl}-N- ({bicyclo[1.1.1] pentan-1- yl}methyl)carbamate | 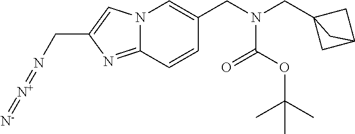 [SMILES: CC(C)(C)OC(=O)N(Cc1ccc2nc(CN=[N+]=[N-])cn2c1)CC12CC(C1)C2] | A | 1.15 | 383.2 |
| 13 | 2-{[2- (azidomethyl)imidazo [1,2-a]pyridin-6- yl]methyl}-2- azaspiro[3.3]heptane | 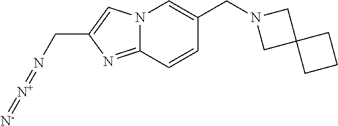 [SMILES: [N-]=[N+]=NCc1cn2cc(CN3CC4(CCC4)C3)ccc2n1] | C | 2.86 | 283.3 |
| 28 | 1-{[2- (azidomethyl)imidazo [1,2-a]pyridin-6- yl]methyl}-4,4- dimethylpiperidine | 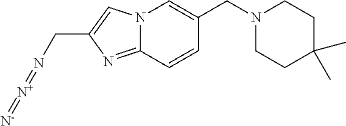 [SMILES: CC1(C)CCN(Cc2ccc3nc(CN=[N+]=[N-])cn3c2)CC1] | J | 0.79 | 299.3 |
| 29 | 6-{[2- (azidomethyl)imidazo [1,2-a]pyridin-6- yl]methyl}-6- azaspiro[3.4]octane | 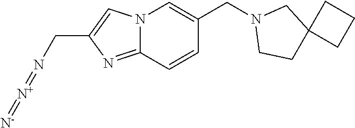 [SMILES: [N-]=[N+]=NCc1cn2cc(CN3CCC4(CCC4)C3)ccc2n1] | J | 0.75 | 297.3 |

### Intermediate 14: 5-ethynyl-1H-pyrrolo[2,3-b]pyridine

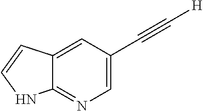 [SMILES: C#Cc1cnc2[nH]ccc2c1]

### Step 1: trimethyl-[2-(1H-pyrrolo[2,3-b]pyridin-5-yl)ethynyl]silane

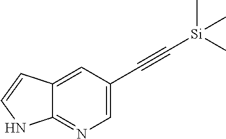

5-iodo-1H-pyrrolo[2,3-b]pyridine (500 mg, 2.05 mmol), CuI (39 mg, 0.20 mmol), PdCl₂ dppf (75 mg, 0.10 mmol) and triethylamine (1 mL, 7.17 mmol) were combined in DMF-Anhydrous (2 mL) and the mixture sparged with nitrogen for 5 minutes. Ethynyl(trimethyl)silane (568 μl, 4.10 mmol) was added, the mixture further sparged briefly and the vessel sealed and heated at 100° C. for 2 hours.

The mixture was cooled to ambient temperature, diluted with NaHCO₃ (sat., 50 mL) and extracted with chloroform/isopropanol (3:1, 3×50 mL). The combined organic extracts were dried over sodium sulfate and evaporated under vacuum.

The residue was purified by chromatography on SiO₂ (eluting with 0-100% EtOAc/heptane) to afford crude title compound (459 mg, 65%) as a brown solid.

Method B: LC-MS (electrospray): m/z=215.2, (M+H)⁺, RT=1.75 min.

### Step 2: 5-ethynyl-1H-pyrrolo[2,3-b]pyridine

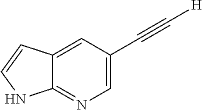 [SMILES: C#Cc1cnc2[nH]ccc2c1]

Trimethyl-[2-(1H-pyrrolo[2,3-b]pyridin-5-yl)ethynyl]silane (459 mg, 2.14 mmol) and K₂CO₃ (592 mg, 4.28 mmol) were combined in methanol (10 mL) and stirred at ambient temperature for 30 minutes.

The mixture was filtered and the residue rinsed with MeOH. The combined filtrate was evaporated under vacuum and purified by chromatography on SiO₂ (eluting with 0-100% EtOAc/heptane) to afford the title compound (113 mg, 33%) as a pale brown solid.

Method B: LC-MS (electrospray): m/z=143.1, (M+H)⁺, RT=1.33 min.

### Intermediate 15: 8-bromoimidazo[1,5-a]pyridine

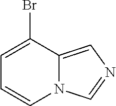

A mixture of formaldehyde (37%, 16 mL, 0.22 mol) and ammonium acetate (20.7 g, 0.27 mol) in acetic acid (100 mL) was stirred at ambient temperature for 10 minutes before 3-bromopyridine-2-carbaldehyde (5.0 g, 26.9 mmol) was added portionwise over 2 hours and the mixture was stirred at ambient temperature for 3 hours.

The mixture was partitioned between water (100 mL) and DCM (100 mL) and the phases separated. The aqueous phase was washed with DCM (3×100 mL), and the combined organic phases passed through an Isolute phase separator and concentrated under vacuum.

The residue was purified chromatography on SiO₂ (eluting with 0-100% EtOAc/heptane) to the title compound (2.51 g, 46%) as an off-white solid.

Method C: LC-MS (electrospray): m/z=196.9/198.9 (M+H)⁺, RT=2.28 min

### Intermediate 16: 8-bromo-6-chloro-imidazo[1,5-a]pyridine

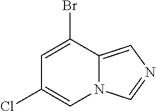 [SMILES: Clc1cc(Br)c2cncn2c1]

### Step 1: (3-bromo-5-chloro-2-pyridyl)methanamine

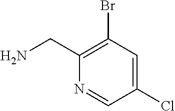

DIBAL in DCM (1 M, 14 mL, 13.8 mmol) was added to an ice-cooled solution of 3-bromo-5-chloropyridine-2-carbonitrile (1000 mg, 4.60 mmol) in DCE (10 mL). The mixture was warmed to ambient temperature and stirred for 2 hours.

The mixture was cooled to 0° C. and quenched with NH₄Cl (sat.), basified using 2NaOH (2 M) and extracted with DCM using a Telos phase separator. The organic phase was evaporated to dryness under reduced pressure to afford the title compound (690 mg, 99%) as a brown solid.

Method B: LC-MS (electrospray): m/z=221.3/223.3 (M+H)⁺, RT=1.31 mi

### Step 2: tert-butyl N-[(3-bromo-5-chloro-2-pyridyl)methyl]carbamate

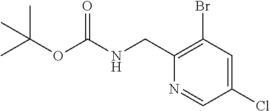 [SMILES: CC(C)(C)OC(=O)NCc1ncc(Cl)cc1Br]

A mixture of (3-bromo-5-chloro-2-pyridyl)methanamine (700 mg, 3.16 mmol), boc anhydride (828 mg, 3.79 mmol) and triethylamine (1.1 mL, 7.90 mmol) in acetonitrile (20 mL) was stirred at reflux for 2 hours.

The mixture was evaporated to dryness, diluted with water and extracted with DCM using a Telos phase separator. The organics were evaporated to dryness and purified by chromatography on SiO₂ eluting with 0-100% EtOAc in heptane) to afford the title compound (640 mg, 63%) as an orange oil.

### Step 3: 8-bromo-6-chloro-imidazo[1,5-a]pyridine

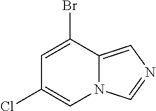 [SMILES: Clc1cc(Br)c2cncn2c1]

A mixture of tert-butyl N-[(3-bromo-5-chloro-2-pyridyl)methyl]carbamate (640 mg, 1.99 mmol), trimethoxymethane (11 mL, 0.1 mol) and TFA (0.59 mL, 7.96 mmol) was stirred at 100° C. for 2 hours.

The mixture was evaporated to dryness and purified by chromatography on SiO₂ (eluting with 0-100% EtOAc in heptane) to afford the title compound (340 mg, 74%) as an orange solid.

Method B: LC-MS (electrospray): m/z=231.3/233.3 (M+H)⁺, RT=1.49 min

### Intermediate 22: 8-bromo-6-methoxy-imidazo[1,5-a]pyridine

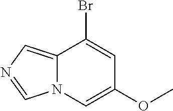

### Step 1: 3-bromo-5-methoxy-pyridine-2-carbonitrile

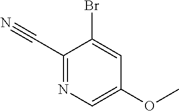

Sodium methoxide (296 mg, 5.47 mmol) was added to an ice-cooled solution of 3-bromo-5-fluoropyridine-2-carbonitrile (1000 mg, 4.98 mmol) in Methanol (15 mL). The Reaction mixture was warmed to ambient temperature and stirred for 1.5 hours Sodium methoxide (296 mg, 5.47 mmol) was added and the RM was stirred at ambient temperature for 1 h, heated to 40° C. for 4 h, then at over the weekend. Sodium methoxide (150 mg) was added and the mixture was stirred at 60° C. for 4 hours. The mixture was evaporated under reduced pressure, water was added and the mixture was extracted with ethyl acetate. The combined organic extracts were washed with saturated brine, and dried over anhydrous magnesium sulfate. The solvent was evaporated under reduced pressure. The residue was purified by chromatography on SiO₂ (25 g SNAP Duo; 0-100% EtOAc in heptane) to the title compound (1130 mg, 106%) as a white powder.

Method A: LC-MS (electrospray): m/z=213/215 (M+H)⁺, RT=1.05 min

### Step 2: (3-bromo-5-methoxy-2-pyridyl)methanamine

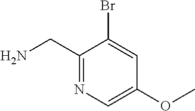

1 M DIBAL in DCM (16 mL, 15.9 mmol) was added to an ice-cooled solution of 3-bromo-5-methoxy-pyridine-2-carbonitrile (1130 mg, 5.30 mmol) in DCE (11 mL). The mixture was warmed to ambient temperature and stirred for 16 hours. The mixture was cooled to 0° C., quenched with saturated NH₄Cl (aq), basified using 2 M NaOH and diluted with DCM. The organic phase was collected using a Telos phase separator and evaporated to dryness under reduced pressure to afford the title compound (840 mg, 69%) as an orange oil.

Method B: LC-MS (electrospray): m/z=217/219 (M+H)⁺, RT=1.22 min

### Step 3: tert-butyl N-[(3-bromo-5-methoxy-2-pyridyl)methyl]carbamate

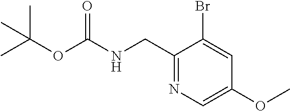 [SMILES: COc1cnc(CNC(=O)OC(C)(C)C)c(Br)c1]

A mixture of (3-bromo-5-methoxy-2-pyridyl)methanamine (830 mg, 3.82 mmol), boc anhydride (1001 mg, 4.59 mmol) and triethylamine (1.3 mL, 9.56 mmol) in Acetonitrile (26 mL) was stirred at reflux for 1 hours The mixture was evaporated to dryness, then water was added and the mixture was extracted with DCM using a Telos phase separator. The organics were evaporated to dryness to afford the title compound (1130 mg, 75%) as a brown oil.

Method A: LC-MS (electrospray): m/z=317/319 (M+H)⁺, RT=1.20 min

### Step 4: 8-bromo-6-methoxy-imidazo[1,5-a]pyridine

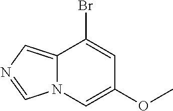

A mixture of tert-butyl N-[(3-bromo-5-methoxy-2-pyridyl)methyl]carbamate (500 mg, 1.58 mmol), trimethoxymethane (8.6 mL, 78.8 mmol) and TFA (0.47 mL, 6.31 mmol) was stirred at 100° C. for 0.5 hours The mixture was evaporated to dryness and purified by chromatography on SiO₂ (eluting with 0-100% EtOAc in heptane, then 10% MeOH in EtOAc) to afford the title compound (191 mg, 53%) as a light sticky solid.

Method A: LC-MS (electrospray): m/z=227/229 (M+H)⁺, RT=0.80 min

### Intermediate 24: 8-bromo-6-methoxy-imidazo[1,5-a]pyridine

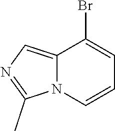

### Step 1: N-[(3-bromo-2-pyridyl)methyl]acetamide

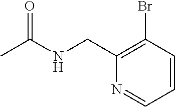

(3-bromo-2-pyridyl)methanamine dihydrochloride 6.11 g, 13.4 mmol) and triethylamine (11.2 mL, 80.4 mmol) were combined in DCM (50 mL) and cooled in an ice/water bath. Acetic anhydride (1.85 mL, 20.1 mmol) was added in a slow stream, and the mixture was stirred whilst warming to ambient temperature and stirred for 2 hours.

The mixture was quenched with NaHOC₃ (sat., 100 mL) and the phases were separated. The aqueous was extracted with DCM (2×80 mL) and the combined organic layers dried over sodium sulfate and evaporated under vacuum.

The residue was purified by chromatography on SiO₂ (eluting with 0-100% EtOAc/heptane followed by 0-20% MeOH/EtOAc 0-20%) to afford the title compound (452 mg, 14%) as a pale yellow solid

Method A: LC-MS (electrospray): m/z=228.9/230.9 (M+H)⁺, RT=0.75 min

### Step 2: 8-bromo-6-methoxy-imidazo[1,5-a]pyridine

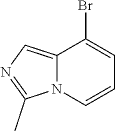

N-[(3-bromo-2-pyridyl)methyl]acetamide (640 mg, 2.79 mmol) was suspended in toluene (10 mL) and POCl₃ (912 μl, 9.78 mmol) was added dropwise whilst stirring. The mixture was then heated at reflux for 3 hours.

The mixture was cooled to ambient temperature, then in an ice/water bath and quenched by slow addition of. NaHCO₃ (sat., 50 mL) under vigorous stirring. The mixture was further stirred for 10 minutes, then extracted with EtOAc (3×50 mL). The combined extracts were washed with brine (50 mL), dried over sodium sulfate and evaporated under vacuum to afford the title compound (528 mg, 87%) as a very pale yellow solid.

Method A: LC-MS (electrospray): m/z=211.0/213.0 (M+H)⁺, RT=0.48 min

### Intermediate 26: 8-bromo-6-methoxy-imidazo[1,5-a]pyridine

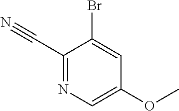

3-bromo-5-fluoropyridine-2-carbonitrile (18.3 g, 91.0 mmol) and sodium methoxide (12.3 g, 227.6 mmol) were combined in methanol (150 mL) and the mixture was heated at reflux for 2 hours. Further sodium methoxide (3 g, 55.5 mmol) was added and the heating was continued for 3 hours.

The mixture was cooled to ambient temperature and concentrated under vacuum. The residue was suspended in NaHCO₃ (sat., 150 mL) and extracted with DCM (3×150 mL). The combined organics were dried over sodium sulfate and evaporated under vacuum.

The residue was purified by Biotage chromatography on SiO₂ (eluting with 20-100% EtOAc/heptane) to afford the title compound (17.05 g, 88%) as a white solid.

Method A: LC-MS (electrospray): m/z=213.0/215.0 (M+H)⁺, RT=1.02 min

### Intermediate 36: 3-bromo-5-methoxypyridine-2-carboxamide

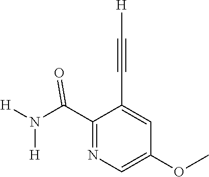

### Step 1: 3-bromo-5-methoxy-pyridine-2-carboxylic Acid

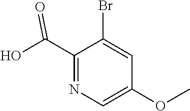

3-bromo-5-methoxy-pyridine-2-carbonitrile[Intermediate 22 Step 1] (2.0 g, 9.4 mmol) was suspended in NaOH (2 M aqueous, 23 ml, 46 mmol) and the mixture heated at reflux for 1.5 hours during which time the solids dissolved.

The mixture was cooled in ice and acidified to ˜pH1 by dropwise addition of c.HCl; precipitation occurs. The solids were collected by filtration, rinsed with a small volume of cold water, and Et₂O, and dried under vacuum at 40° C. to afford the title compound (2.0 g, 91%) as a white solid.

Method A: LC-MS (electrospray): m/z=231.9/233.0 (M+H)⁺, RT=0.80 min.

### Step 2: Methyl 3-bromo-5-methoxy-pyridine-2-carboxylate

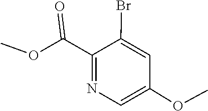

Acetyl chloride (1.49 ml, 21.01 mmol) was added dropwise to ice-cold Methanol (40 ml) and the mixture stirred under cooling for 10 minutes. 3-bromo-5-methoxy-pyridine-2-carboxylic acid (975 mg, 4.20 mmol) was added in a single portion and the mixture was stirred and allowed to warm to room temperature and stirred for a further 2 hours.

The mixture was heated at reflux for an hour and left standing for 65 hours.

The mixture was evaporated under vacuum. The residue was suspended in sat. NaHCO₃ (sat. 100 ml) and extracted with DCM (3×80 ml). The organic extracts were dried over sodium sulfate and evaporated under vacuum to afford the title compound (996 mg, 96%) as a white solid.

Method A: LC-MS (electrospray): m/z=245.9/247.9 (M+H)⁺, RT=0.98 min

### Step 3: Methyl 5-methoxy-3-(2-trimethylsilylethynyl)pyridine-2-carboxylate

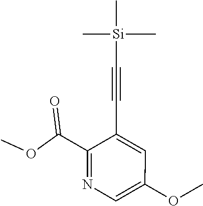

Methyl 3-bromo-5-methoxy-pyridine-2-carboxylate (990 mg, 4.0 mmol), CuI (77 mg, 0.40 mmol), PdCl₂ dppf (294 mg, 0.40 mmol) and triethylamine (2.5 ml, 17.9 mmol) were combined in DMF-Anhydrous (10 mL) and the mixture was sparged with nitrogen for 5 minutes. Before ethynyl(trimethyl)silane (862 μl, 6.04 mmol) was added, the mixture was further sparged briefly with nitrogen and heated at 100° C. under a nitrogen atmosphere for 1.5 hours.

The mixture was cooled to room temperature, diluted with EtOAc (80 mL) and brine (80 mL) and filtered through Celite. The phases were separated and the organic phase was washed with brine (5×80 ml), dried over sodium sulfate and evaporated under vacuum.

The residue was purified by chromatography on SiO₂ (eluting with EtOAc/heptane 0-100%) to afford the title compound (1.18 g, 92%) as a pale brown solid.

Method A: LC-MS (electrospray): m/z=264.0 (M+H)⁺, RT=1.21 min.

### Step 4: 3-Ethynyl-5-methoxypyridine-2-carboxylic Acid

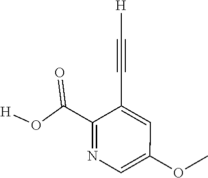

Methyl 5-methoxy-3-(2-trimethylsilylethynyl)pyridine-2-carboxylate (1 g, 3.15 mmol) and lithium hydroxide (151 mg, 6.30 mmol) were combined in THF (40 mL) and Water (10 mL) and stirred at room temperature for 18 hours.

The mixture was concentrated under vacuum and the residue was acidified HCl (3 M, ˜pH1). The mixture was extracted with chloroform/isopropanol (3:1, 3×50 ml) and the extracts were dried over sodium sulfate and evaporated under vacuum to afford the title compound (676 mg, 94%) as a yellow solid.

Method A: LC-MS (electrospray): m/z=178.0 (M+H)⁺, RT=0.76 min.

### Step 5: 3-Ethynyl-5-methoxypyridine-2-carboxamide

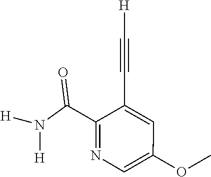

3-Ethynyl-5-methoxy-pyridine-2-carboxylic acid (150 mg, 0.85 mmol) and triethylamine (572 μL, 8.47 mmol) were combined in THF-Anhydrous (5 mL) and cooled in an ice/water bath. Isobutyl chloroformate (154 μL, 1.19 mmol) was added slowly and the mixture stirred under cooling for 15 minutes.

Ammonia (28% aqueous, 1 mL, 14.8 mmol) was added and the mixture stirred whilst warming to room temperature and for a further 15 minutes.

The mixture was quenched with NaHCO₃ (sat., 30 ml) and extracted with DCM (3×30 mL). The extracts were dried over sodium sulfate and evaporated under vacuum to afford the title compound (159 mg, 79%) as a pale brown residue.

Method A: LC-MS (electrospray): m/z=177.1 (M+H)⁺, RT=0.79 min.

### Intermediate 38: tert-butyl 7-bromo-2H,3H,4H-pyrido[3,2-b][1,4]oxazine-4-carboxylate

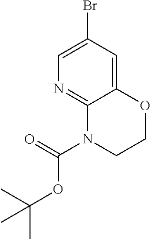 [SMILES: CC(C)(C)OC(=O)N1CCOc2cc(Br)cnc21]

7-Bromo-3,4-dihydro-2H-pyrido[3,2-b][1,4]oxazine (1.1 g, 5.12 mmol) and DMAP (50 mg, 0.41 mmol) were combined in DCM (40 ml) and Boc₂O (2.23 g, 10.2 mmol) was added, to give a yellow solution which was stirred at room temperature for 4 hours.

The mixture was evaporated under vacuum and the residue was purified by chromatography on SiO₂ (eluting with 0-100% EtOAc/heptane) to afford the title compound (1.54 g, 94%) as a white solid.

Method A: LC-MS (electrospray): m/z=314.9/316.9 (M+H)⁺, RT=1.18 min.

### Intermediate 40: 3-Bromo-5-(morpholin-4-yl)pyridine-2-carbonitrile

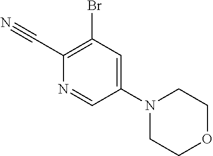 [SMILES: N#Cc1ncc(N2CCOCC2)cc1Br]

3-Bromo-5-fluoropyridine-2-carbonitrile (550 mg, 2.74 mmol), triethylamine (381 μL, 2.74 mmol) and morpholine (432 μL, 3.56 mmol) were combined in DMF (4 mL) and heated at 90° C. for 2 hours.

The reaction mixture was cooled to room temperature, diluted with EtOAc (80 mL) and washed with brine (5×50 mL). The organic layer dried over sodium sulfate and evaporated under vacuum.

The residue was triturated with DCM/heptane and the solids collected by filtration to afford the title compound (655 mg, 88%) as a very pale yellow solid.

Method A: LC-MS (electrospray): m/z=268.0/270.0 (M+H)⁺, RT=1.02 min.

### Intermediate 42: 5-Ethynylpyridin-3-ol

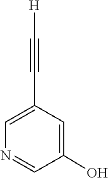

### Step 1: 3-bromo-5-[(tert-butyldimethylsilyl)oxy]pyridine

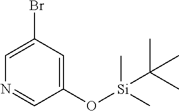

5-bromopyridin-3-ol (1 g, 5.75 mmol) and 1H-imidazole (783 mg, 11.5 mmol) were combined in DMF (10 mL), tert-butyl(chloro)dimethylsilane (1.13 g, 7.47 mmol) was added and the mixture was stirred at room temperature for 3 hours.

The mixture was diluted with EtOAc (80 mL) and washed with NaHCO₃ (sat., 50 mL) and brine (5×50 mL). The organic layer was dried over sodium sulfate and evaporated under vacuum.

The residue was purified by chromatography on SiO₂ (eluting with 0-100% EtOAc/heptane) to afford the title compound (1.45 g, 83%) as a colourless oil.

Method A: LC-MS (electrospray): m/z=278.9/289.9 (M+H)⁺, RT=1.44 min.

The title compound was prepared from intermediate 42 using the procedures described for Intermediate 14 to give (114 mg, 12%) as brown solid.

Method A: LC-MS (electrospray): m/z=119.9 (M+H)⁺, RT=0.47 min.

The compounds in Table 3 were prepared similarly to Intermediate 14 from the corresponding bromide or iodide, either commercial or described.

TABLE 3

| | | | | LCMS | |
|---|---|---|---|---|---|
| | | | LCMS | Retention | mass |
| Intermediate | Name | Structure | Method | time | ion |
| 17 | 8-ethynylimidazo[1,5- a]pyridine | 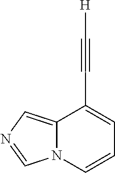 [SMILES: C#Cc1cccn2cncc12] | C | 2.01 | 285.2 |
| 18 | 6-chloro-8-ethynyl imidazo[1,5-a]pyridine | 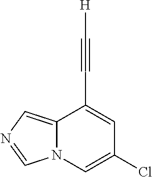 [SMILES: C#Cc1cc(Cl)cn2cncc12] | B | 1.45 | 177.2 |
| 19 | 8-ethynyl- [1,2,4]triazolo[4,3-a] pyridine | 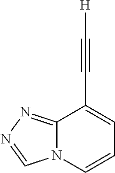 [SMILES: C#Cc1cccn2cnnc12] | A | 0.53 | 144.3 |
| 23 | 8-ethynyl-6-methoxy- imidazo[1,5-a]pyridine | 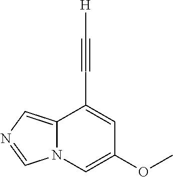 | A | 0.77 | 173.1 |
| 25 | 8-ethynyl-3-methyl- imidazo[1,5-a]pyridine | 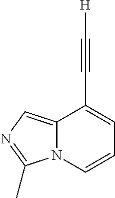 | A | 0.47 | 157.1 |
| 27 | 3-ethynyl-5-methoxy- pyridine-2-carbonitrile | 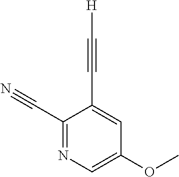 | A | 0.98 | 159.0 |
| 30 | 3-ethynyl-5- fluoropyridine-2- carbonitrile | 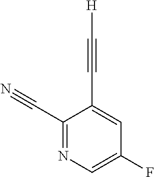 [SMILES: C#Cc1cc(F)cnc1C#N] | A | 0.99 | No mass ion |
| 31 | 2-ethynyl-6- methoxypyrazine | 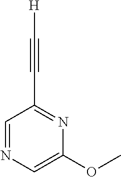 | A | 0.93 | 135.0 |
| 32 | 3-ethynyl-5- methoxypyridine | 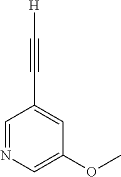 | A | 0.86 | 134.0 |
| 33 | 3-ethynyl-4- methoxypyridine | 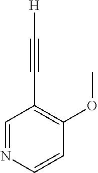 | A | 0.25 | 134.0 |
| 34 | 5-ethynyl-2- methoxypyridine | 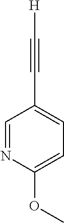 | A | 1.04 | 134.0 |
| 37 | 7-ethynyl-1-methyl- 1H-indazole | 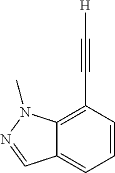 | A | 1.13 | 157.3 |
| 41 | 3-ethynyl-5- (morpholin-4- yl)pyridine-2- carbonitrile | 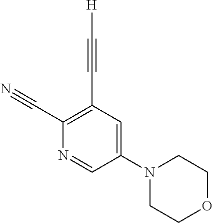 [SMILES: C#Cc1cc(N2CCOCC2)cnc1C#N] | A | 0.99 | 214.1 |

### Intermediate 20: tert-butyl N-{[2-(azidomethyl)imidazo[1,2-a]pyridin-6-yl]methyl}carbamate

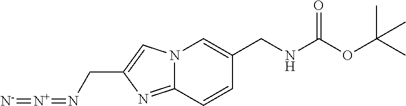 [SMILES: CC(C)(C)OC(=O)NCc1ccc2nc(CN=[N+]=[N-])cn2c1]

### Step 1: tert-butyl N-[(6-aminopyridin-3-yl)methyl]carbamate

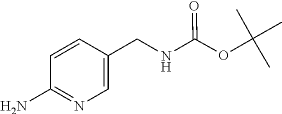 [SMILES: CC(C)(C)OC(=O)NCc1ccc(N)nc1]

A solution of 6-aminopyridine-3-carbonitrile (1.00 g, 8.39 mmol) and tert-butoxycarbonyl tert-butyl carbonate (3.66 g, 16.8 mmol) in MeOH (32 mL) was cooled to 0° C. and treated with nickel (II) chloride (1.10 g, 8.39 mmol) followed by the addition of sodium borohydride (2.86 g, 75.6 mmol) in small portions. The mixture was stirred and allowed to warm to ambient temperature overnight.

The stir was continued for 24 hours before the black suspension was evaporated under vacuum, quenched by the addition of NaHCO₃ (sat) and EtOAc was added. The solids were removed by filtration through Celite (washing with EtOAc). The phases were separated and the aqueous extracted with EtOAc. The organics were washed with brine, dried (MgSO₄) and evaporated under vacuum to a brown foam which was purified by chromatography on SiO₂ (Biotage KPNH 28 g) eluting with 0-100% EtOAc in heptane to give the title compound (734 mg, 28%) as a beige foam along with an earlier eluting product (178 mg).

Method B: LC-MS (electrospray): m/z=224.3 (M+H)⁺, RT=1.24 min.

### Step 2: tert-butyl N-[[2-(chloromethyl)imidazo[1,2-a]pyridin-6-yl]methyl]carbamate

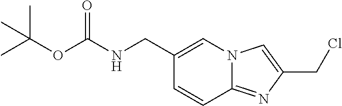 [SMILES: CC(C)(C)OC(=O)NCc1ccc2nc(CCl)cn2c1]

A solution of tert-butyl N-[(6-amino-3-pyridyl)methyl]carbamate (730 mg, 3.27 mmol) and 1,3-dichloropropan-2-one (450 uL, 4.90 mmol) in MeCN (20 mL) was stirred at 80° C. for 20 hours.

The mixture was cooled to ambient temperature, evaporated to dryness and purified by chromatography on SiO₂ (Biotage; 28 g KP-NH) eluting with EtOAc in heptane 50-100% to afford the title compound (581 mg, 52%) as beige solid.

Method B: LC-MS (electrospray): m/z=296.2 (M+H)⁺, RT=1.53 min.

### Step 3: tert-butyl N-{[2-(azidomethyl)imidazo[1,2-a]pyridin-6-yl]methyl}carbamate

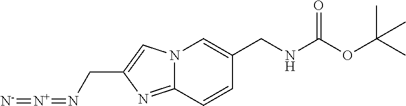 [SMILES: CC(C)(C)OC(=O)NCc1ccc2nc(CN=[N+]=[N-])cn2c1]

A solution of tert-butyl N-[[2-(chloromethyl)imidazo[1,2-a]pyridin-6-yl]methyl]carbamate (581 mg, 1.96 mmol) and NaI (30 mg, 0.196 mmol) in DMF (4 mL) was treated with NaN3 (255 mg, 3.93 mmol) and the mixture was stirred at ambient temperature 17 hours.

The brown suspension was quenched with NaHCO₃ (sat, 10 mL) and extracted with chloroform/isopropanol (3:1, 3×80 mL). The combined organic extracts were dried (Na₂SO₄) and evaporated under vacuum. The residue was triturated with EtOAc/heptane and the solids were collected by filtration to afford the title compound (400 mg, 67%) as a beige solid.

Method B: LC-MS (electrospray): m/z=303.2 (M+H)⁺, RT=1.42 min.

### Intermediate 21: [2-(azidomethyl)imidazo[1,2-a]pyridin-6-yl]methanol

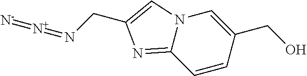 [SMILES: [N-]=[N+]=NCc1cn2cc(CO)ccc2n1]

### Step 1: methyl 2-(chloromethyl)imidazo[1,2-a]pyridine-6-carboxylate

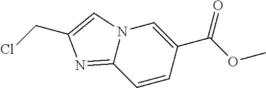

Methyl 6-aminopyridine-3-carboxylate (5.3 g, 34.8 mmol) and 1,3-dichloropropan-2-one (6.63 g, 52.3 mmol) were combined in acetonitrile (50 mL) and the mixture heated at reflux for 6 hours. The reaction mixture was cooled to ambient temperature and the residue suspended in aqueous sodium hydrogen carbonate (sat, 80 mL). The mixture was extracted with 3:1 chloroform/isopropanol (3×100 mL) and the combined organic extracts dried over sodium sulfate and evaporated under vacuum. The residue was purified by Biotage Isolera™ chromatography (100 g kp-Sil, eluting with EtOAc/heptane 0-100%) to afford a crude material which was triturated with EtOAc/heptane. The solids were collected by filtration and dried under suction to afford 3.35 g (43%) of the title compound as a white solid.

Method B: LC-MS (electrospray): m/z=225.1 (M+H)⁺, RT=1.32 min

### Step 2: [2-(chloromethyl)imidazo[1,2-a]pyridin-6-yl]methanol

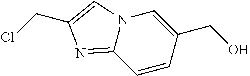 [SMILES: OCc1ccc2nc(CCl)cn2c1]

A solution of methyl 2-(chloromethyl)imidazo[1,2-a]pyridine-6-carboxylate (700 mg, 3.12 mmol) in anhydrous THF (20 mL) and cooled in an ice/water bath. DIBAL (1 M in PhMe, 8.6 mL, 8.6 mmol) was added slowly to give a yellow solution and the mixture stirred under cooling for 2 hours. The reaction mixture was quenched by dropwise addition of MeOH (5 mL), then diluted with DCM (70 mL) and aqueous sodium hydrogen carbonate (sat, 50 mL). The mixture was filtered through Celite and the residue rinsed well with DCM. The filtrates were phase-separated, the aqueous layer extracted with DCM (80 mL). The combined organic layers dried over sodium sulfate and evaporated under vacuum. The resultant residue was triturated with Et₂O and the solids collected by filtration to afford (384 mg 60%) of the title compound as a pale yellow solid

MS16 IPC: LC-MS (electrospray): m/z=196.8 (M+H)⁺, RT=0.31 min

### Step 3: [2-(azidomethyl)imidazo[1,2-a]pyridin-6-yl]methanol

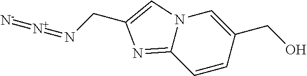 [SMILES: [N-]=[N+]=NCc1cn2cc(CO)ccc2n1]

[2-(chloromethyl)imidazo[1,2-a]pyridin-6-yl]methanol (380 mg, 1.93 mmol), NaI (29 mg, 0.19 mmol) and NaN₃ (300 mg, 4.61 mmol) were combined in DMF (2 mL) and the mixture stirred at ambient temperature for 3 hours. The reaction mixture was diluted with aqueous sodium hydrogen carbonate (sat, 30 mL) and extracted with DCM (3×50 mL). The combined organic extracts were dried over sodium sulfate and evaporated under vacuum. The solids were carefully triturated with EtOAc/heptane and the solids collected by filtration to afford 309 mg, 79%) of the title compound as a pale yellow solid

Method B: LC-MS (electrospray): m/z=204.2 (M+H)⁺, RT=1.10 min

### Reference Example 1: N-({6-methylimidazo[1,2-a]pyridin-2-yl}methyl)-1H-pyrazolo[4,3-c]pyridine-4-carboxamide

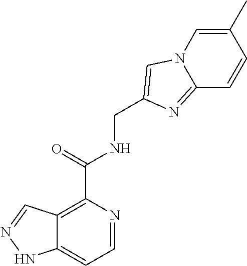 [SMILES: Cc1ccc2nc(CNC(=O)c3nccc4[nH]ncc34)cn2c1]

A solution of 1H-pyrazolo[4,3-c]pyridine-4-carboxylic acid (50 mg, 0.31 mmol), HATU (175 mg, 0.460 mmol) and DIPEA (0.054 mL, 0.307 mmol) in DMF (1 mL) was stirred at ambient temperature for 5 minutes before {6-methylimidazo[1,2-a]pyridin-2-yl}methanamine dihydrochloride (79 mg, 0.337 mmol) was added and the mixture was stirred at ambient temperature for 1 hour.

The mixture was evaporated to dryness under reduced pressure and purified by preparative HPLC (Method B) to afford the title compound (18 mg, 19%) as an off-white powder.

Method D: LC-MS (electrospray): m/z=307.2 (M+H)⁺, RT=2.90 min

Method C: LC-MS (electrospray): m/z=423.4 (M+H)⁺, RT=2.79 min

The compounds in Table 4 were prepared in a similar fashion to Example 1 using the appropriate carboxylic acid and aminomethyl imidazopyridine intermediate, or commercially available materials, including deprotection where necessary.

TABLE 4

| | | | | LCMS | |
|---|---|---|---|---|---|
| | | | | Retention | |
| Example | | | LCMS | time/mass | |
| No | Name | Structure | Method | ion | Intermediates |
| 2 | N-[(6- {[(cyclohexylmethyl) amino]methyl} imidazo [1,2-a]pyridin-2- yl)methyl]-1,2- dimethyl-6-oxo-1,6- dihydropyrimidine-4- carboxamide | 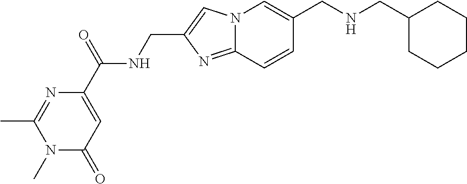 [SMILES: Cc1nc(C(=O)NCc2cn3cc(CNCC4CCCCC4)ccc3n2)cc(=O)n1C] | C | 2.79/423.4 | 1, 5 |
| 4 | N-({6- methylimidazo[1,2- a]pyridin-2- yl}methyl)-4-oxo- 4H-quinolizine-2- carboxamide | 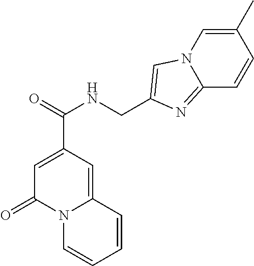 [SMILES: Cc1ccc2nc(CNC(=O)c3cc(=O)n4ccccc4c3)cn2c1] | C | 1.93/333.2 | 8 |
| 5 | N-[(6- {[(cyclohexylmethyl) amino]methyl} imidazo[1,2-a] pyridin-2- yl)methyl]-4-oxo- 4H-quinolizine-2- carboxamide | 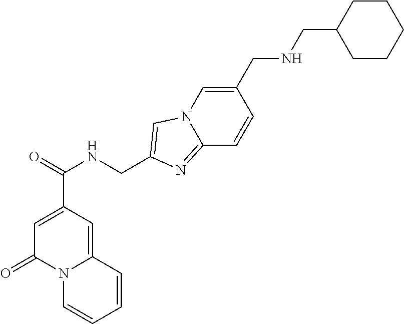 [SMILES: O=C(NCc1cn2cc(CNCC3CCCCC3)ccc2n1)c1cc(=O)n2ccccc2c1] | C | 2.80/444.4 | 1, 8 |
| 6 | N-[(6- {[(cyclohexylmethyl) amino]methyl} imidazo[1,2-a] pyridin-2-yl)methyl] quinoxaline-2- carboxamide | 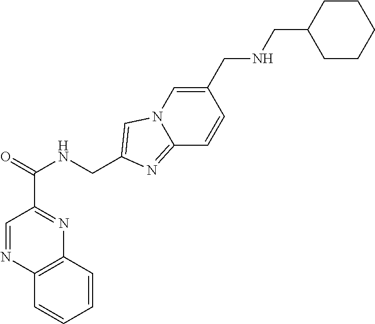 [SMILES: O=C(NCc1cn2cc(CNCC3CCCCC3)ccc2n1)c1cnc2ccccc2n1] | C | 3.43/429.4 | 1 |
| 7 | N-[(6- {[(cyclohexylmethyl) amino]methyl} imidazo[1,2-a] pyridin-2- yl)methyl]-1H- pyrazolo[3,4- b]pyridine-4- carboxamide | 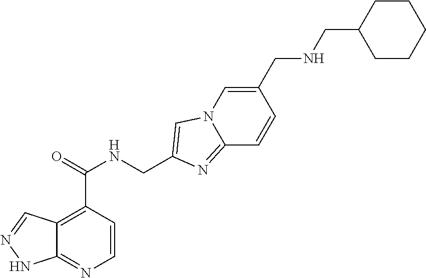 [SMILES: O=C(NCc1cn2cc(CNCC3CCCCC3)ccc2n1)c1ccnc2[nH]ncc12] | C | 2.72/418.4 | 1 |
| 8 | N-[(6- {[(cyclohexylmethyl) amino]methyl} imidazo[1,2-a] pyridin-2- yl)methyl] quinoline-3- carboxamide | 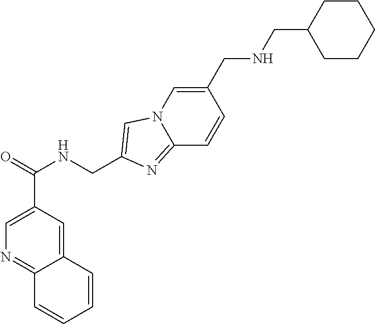 [SMILES: O=C(NCc1cn2cc(CNCC3CCCCC3)ccc2n1)c1cnc2ccccc2c1] | C | 3.14/428.4 | 1 |
| 10 | N-[(6- {[(cyclobutylmethyl) amino]methyl} imidazo[1,2-a] pyridin-2-yl) methyl]imidazo [1,5-a]pyridine-8- carboxamide | 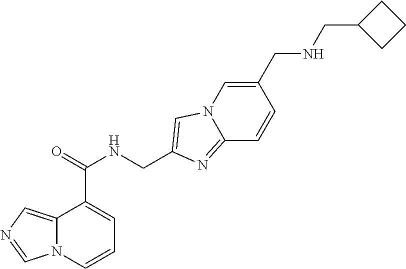 [SMILES: O=C(NCc1cn2cc(CNCC3CCC3)ccc2n1)c1cccn2cncc12] | C | 2.19/389.3 | 2 |
| 11 | N-[(6- {[(cyclobutylmethyl) amino]methyl} imidazo[1,2-a] pyridin-2- yl)methyl]imidazo [1,5-a]pyridine-5- carboxamide | 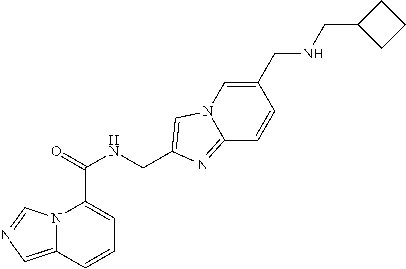 [SMILES: O=C(NCc1cn2cc(CNCC3CCC3)ccc2n1)c1cccc2cncn12] | C | 2.32/389.4 | 2 |
| 13 | N-[(6- {[(cyclobutylmethyl) amino]methyl} imidazo[1,2-a] pyridin-2-yl) methyl]-3H- imidazo[4,5- b]pyridine-6- carboxamide | 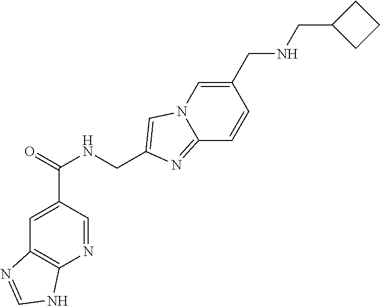 [SMILES: O=C(NCc1cn2cc(CNCC3CCC3)ccc2n1)c1cnc2[nH]cnc2c1] | D | 1.22/390.3 | 2 |
| 14 | N-[(6- {[(cyclobutylmethyl) amino]methyl} imidazo[1,2-a] pyridin-2-yl) methyl]-1H- pyrrolo[2,3-b] pyridine-5- carboxamide | 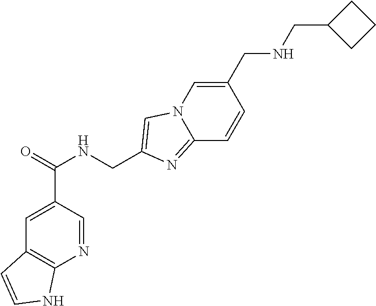 [SMILES: O=C(NCc1cn2cc(CNCC3CCC3)ccc2n1)c1cnc2[nH]ccc2c1] | D | 1.36/389.3 | 2 |
| 15 | N-{[6-({[(3,3- difluorocyclobutyl) methyl]amino} methyl)imidazo[1,2- a]pyridin-2-yl] methyl}-1H- pyrrolo[2,3- b]pyridine-5- carboxamide | 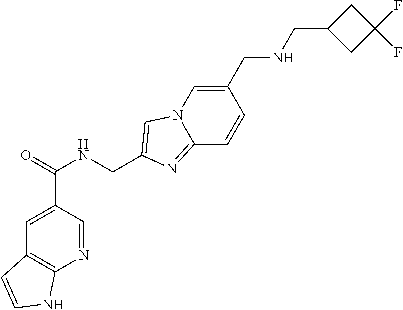 [SMILES: O=C(NCc1cn2cc(CNCC3CC(F)(F)C3)ccc2n1)c1cnc2[nH]ccc2c1] | C | 2.28/425.3 | 2 |
| 16 | N-[(6- {[(cyclobutylmethyl) amino]methyl} imidazo[1,2-a] pyridin-2-yl)methyl]- 1-methyl-1H- pyrrolo[2,3-b] pyridine-5- carboxamide | 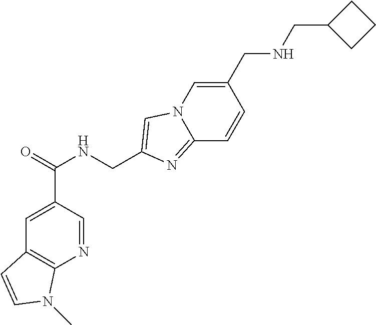 [SMILES: Cn1ccc2cc(C(=O)NCc3cn4cc(CNCC5CCC5)ccc4n3)cnc21] | C | 2.54/403.4 | 2 |
| 17 | N-[(6- {[(cyclobutylmethyl) amino]methyl} imidazo[1,2-a] pyridin-2- yl)methyl]-1H- indole-5- carboxamide | 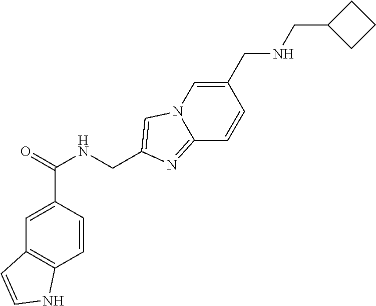 [SMILES: O=C(NCc1cn2cc(CNCC3CCC3)ccc2n1)c1ccc2[nH]ccc2c1] | C | 2.59/388.4 | 2 |
| 18 | N-[(6- {[(cyclobutylmethyl) amino]methyl} imidazo[1,2-a] pyridin-2-yl) methyl]-5H- pyrrolo[2,3-b] pyrazine-2- carboxamide | 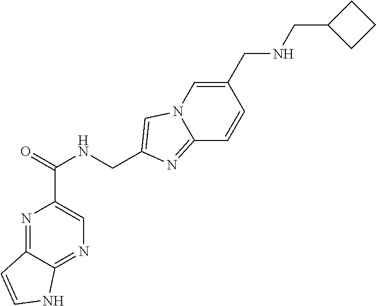 [SMILES: O=C(NCc1cn2cc(CNCC3CCC3)ccc2n1)c1cnc2[nH]ccc2n1] | C | 2.42/390.4 | 2 |
| 19 | N-[(6-{[({3- fluorobicyclo[1.1.1] pentan-1- yl}methyl)amino] methyl}imidazo[1,2- a]pyridin-2- yl)methyl]-5-oxo- 5H- [1,3]thiazolo[3,2- a]pyrimidine-7- carboxamide | 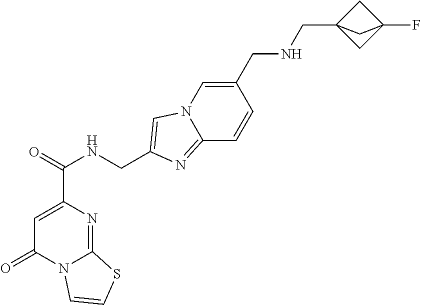 [SMILES: O=C(NCc1cn2cc(CNCC34CC(F)(C3)C4)ccc2n1)c1cc(=O)n2ccsc2n1] | C | 2.31/453.3 | 2 |

### Example 20: 4-[1-[(6-methylimidazo[1,2-a]pyridin-2-yl)methyl]triazol-4-yl]isoquinoline

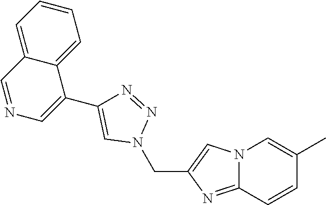 [SMILES: Cc1ccc2nc(Cn3cc(-c4cncc5ccccc45)nn3)cn2c1]

2-(azidomethyl)-6-methyl-imidazo[1,2-a]pyridine Intermediate 9 (50 mg, 0.27 mmol), 4-ethynylisoquinoline (49 mg, 0.32 mmol) and sodium ascorbate (80 mg, 0.40 mmol) were combined in DMF (2 mL) and Water (0.5 mL), CuSO₄ (4 mg, 0.03 mmol) was added and the mixture was stirred at ambient temperature for 30 minutes during which time a precipitate formed.

The mixture was diluted with water (15 mL) and the solids were collected by filtration. The residue was purified by reverse phase chromatography (Method B) to afford the title compound (24 mg (26%) as an off-white solid.

Method D: LC-MS (electrospray): m/z=341.3 (M+H)⁺, RT=3.33 min

The compounds in Table 5 were prepared in a similar manner to Example 20 using commercial building blocks or described intermediates

TABLE 5

| | | | | LCMS | |
|---|---|---|---|---|---|
| | | | | Retention | |
| Example | | | LCMS | time/mass | |
| No | Name | Structure | Method | ion | Intermediates |
| 21 | 1-({6- methylimidazo [1,2-a]pyridin-2- yl}methyl)-4-{1H- pyrrolo[2,3-b]pyridin- 5-yl}-1H-1,2,3- triazole | 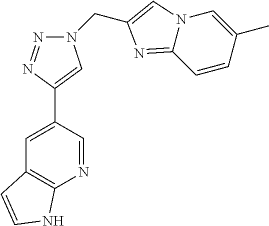 [SMILES: Cc1ccc2nc(Cn3cc(-c4cnc5[nH]ccc5c4)nn3)cn2c1] | D | 3.06/330.3 | |

### Example 22: (cyclobutylmethyl)({2-[(4-{6-methoxyimidazo[1,5-a]pyridin-8-yl}-1H-1,2,3-triazol-1-yl)methyl]imidazo[1,2-a]pyridin-6-yl}methyl)amine

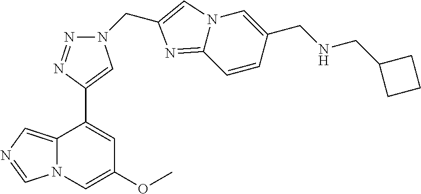 [SMILES: COc1cc(-c2cn(Cc3cn4cc(CNCC5CCC5)ccc4n3)nn2)c2cncn2c1]

### Step 1: tert-butyl N-(cyclobutylmethyl)-N-[[2-[[4-(6-methoxyimidazo[1,5-a]pyridin-8-yl)triazol-1-yl]methyl]imidazo[1,2-a]pyridin-6-yl]methyl]carbamate

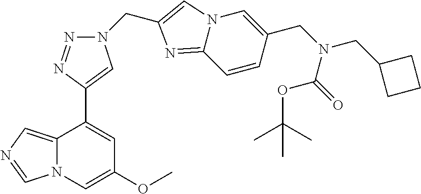 [SMILES: COc1cc(-c2cn(Cc3cn4cc(CN(CC5CCC5)C(=O)OC(C)(C)C)ccc4n3)nn2)c2cncn2c1]

A mixture of 8-ethynyl-6-methoxy-imidazo[1,5-a]pyridine Intermediate 23 (150 mg, 0.810 mmol), tert-butyl N-[[2-(azidomethyl)imidazo[1,2-a]pyridin-6-yl]methyl]-N-(cyclobutylmethyl)carbamate Intermediate 10 (300 mg, 0.810 mmol), copper sulfate (26 mg, 0.162 mmol) and sodium ascorbate (177 mg, 0.891 mmol) in DMF (9 mL) and water (2 mL) was stirred at ambient temperature overnight.

The mixture was diluted with water and 10% MeOH in DCM. The organic phase was collected using a Telos phase separator, evaporated to dryness under reduced pressure and purified by chromatography on SiO₂ (eluting with 0-100% EtOAc in heptane foolwed by 0-20% MeOH in EtOAc) to afford the title compound (240 mg, 51%) as an orange solid.

Method J: LC-MS (electrospray): m/z=543.4 (M+H)⁺, RT=0.63 min.

### Step 2: (cyclobutylmethyl)({2-[(4-{6-methoxyimidazo[1,5-a]pyridin-8-yl}-1H-1,2,3-triazol-1-yl)methyl]imidazo[1,2-a]pyridin-6-yl}methyl)amine

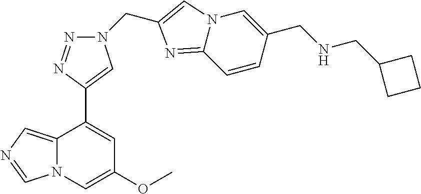 [SMILES: COc1cc(-c2cn(Cc3cn4cc(CNCC5CCC5)ccc4n3)nn2)c2cncn2c1]

HCl (4 M in dioxane 1.0 mL, 4.11 mmol) was added to a solution of tert-butyl N-(cyclobutylmethyl)-N-[[2-[[4-(6-methoxyimidazo[1,5-a]pyridin-8-yl)triazol-1-yl]methyl]imidazo[1,2-a]pyridin-6-yl]methyl]carbamate (240 mg, 0.411 mmol) in methanol (1.5 mL) and the mixture was stirred at ambient temperature for 16 hours.

The mixture was evaporated to dryness and purified by preparative HPLC (Method B). The organic solvent was removed from the product-containing fractions. The solid formed in the aqueous suspension thus obtained was collected by vacuum filtration and dried under vacuum to afford the title compound (60 mg, 33%) as a white powder.

Method C: LC-MS (electrospray): m/z=443.4 (M+H)⁺, RT=2.69 min.

The compounds in Table 6 were prepared in a similar manner to Example 22 using commercial building blocks or described intermediates.

TABLE 6

| | | | | LCMS | |
|---|---|---|---|---|---|
| | | | | Retention | |
| Example | | | LCMS | time/mass | |
| No | Name | Structure | Method | ion | Mass ion |
| 23 | (cyclobutylmethyl)({2- [(4-{imidazo[1,5- a]pyridin-8-yl}-1H- 1,2,3-triazol-1- yl)methyl]imidazo [1,2-a]pyridin-6- yl}methyl)amine | 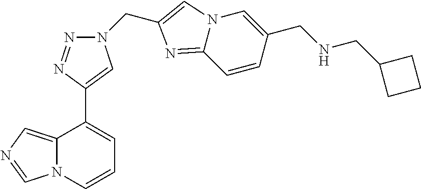 [SMILES: c1cc(-c2cn(Cc3cn4cc(CNCC5CCC5)ccc4n3)nn2)c2cncn2c1] | C | 2.54 | 413.4 |
| 24 | ({2-[(4-{6- chloroimidazo[1,5- a]pyridin-8-yl}-1H- 1,2,3-triazol-1- yl)methyl]imidazo [1,2-a]pyridin-6- yl}methyl) (cyclobutylmethyl) amine | 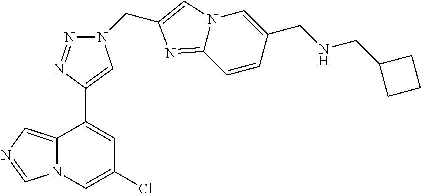 [SMILES: Clc1cc(-c2cn(Cc3cn4cc(CNCC5CCC5)ccc4n3)nn2)c2cncn2c1] | D | 3.83 | 447.2 |
| 25 | (cyclobutylmethyl)({2- [(4-{1- fluoroimidazo[1,5- a]pyridin-8-yl}-1H- 1,2,3-triazol-1- yl)methyl]imidazo [1,2-a]pyridin-6- yl}methyl)amine | 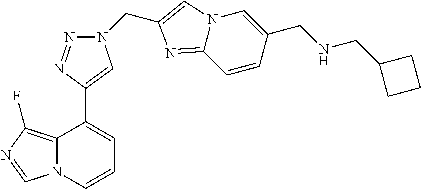 [SMILES: Fc1ncn2cccc(-c3cn(Cc4cn5cc(CNCC6CCC6)ccc5n4)nn3)c12] | C | 2.66 | 431.4 |
| 26 | ({3- fluorobicyclo[1.1.1] pentan-1- yl}methyl)({2-[(4-{6- methoxyimidazo[1,5- a]pyridin-8-yl}-1H- 1,2,3-triazol-1- yl)methyl]imidazo [1,2-a]pyridin-6- yl}methyl)amine | 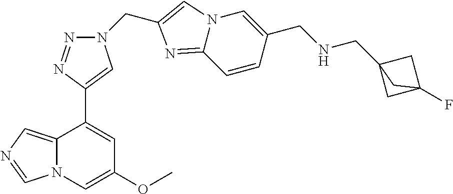 [SMILES: COc1cc(-c2cn(Cc3cn4cc(CNCC56CC(F)(C5)C6)ccc4n3)nn2)c2cncn2c1] | C | 2.57 | 473.4 |
| 27 | (cyclobutylmethyl)({2- [(4- {[1,2,4]triazolo[4,3- a]pyridin-8-yl}-1H- 1,2,3-triazol-1- yl)methyl]imidazo [1,2-a]pyridin-6- yl}methyl)amine | 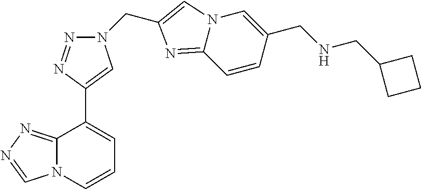 [SMILES: c1cc(-c2cn(Cc3cn4cc(CNCC5CCC5)ccc4n3)nn2)c2nncn2c1] | D | 3.21 | 414.2 |
| 28 | ({bicyclo[1.1.1]pentan- 1-yl}methyl)({2-[(4- {6- methoxyimidazo[1,5- a]pyridin-8-yl}-1H- 1,2,3-triazol-1- yl)methyl]imidazo [1,2-a]pyridin-6- yl}methyl)amine | 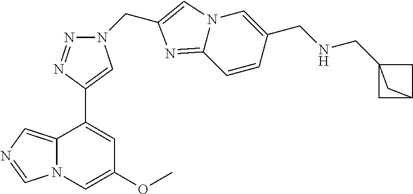 [SMILES: COc1cc(-c2cn(Cc3cn4cc(CNCC56CC(C5)C6)ccc4n3)nn2)c2cncn2c1] | C | 2.72 | 455.4 |
| 29 | 2-({2-[(4- {imidazo[1,5- a]pyridin-8-yl}-1H- 1,2,3-triazol-1- yl)methyl]imidazo[1,2- a]pyridin-6- yl}methyl)-2- azaspiro[3.3]heptane | 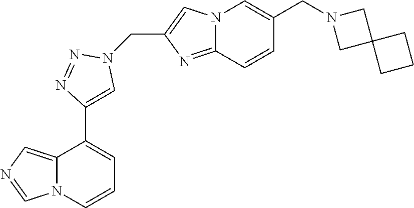 [SMILES: c1cc(-c2cn(Cc3cn4cc(CN5CC6(CCC6)C5)ccc4n3)nn2)c2cncn2c1] | C | 2.64 | 425.6 |
| 30 | 2-({2-[(4-{6- methoxyimidazo[1,5- a]pyridin-8-yl}-1H- 1,2,3-triazol-1- yl)methyl]imidazo [1,2-a]pyridin-6- yl}methyl)-2- azaspiro[3.3] heptane | 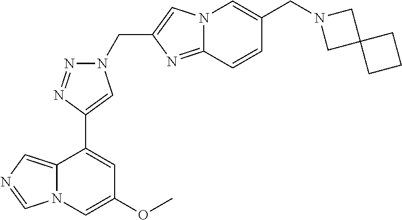 [SMILES: COc1cc(-c2cn(Cc3cn4cc(CN5CC6(CCC6)C5)ccc4n3)nn2)c2cncn2c1] | C | 2.90 | 455.5 |
| 36 | ({3- fluorobicyclo[1.1.1] pentan-1- yl}methyl)({2-[(4-{3- methylimidazo[1,5- a]pyridin-8-yl}-1H- 1,2,3-triazol-1- yl)methyl]imidazo [1,2-a]pyridin-6- yl}methyl)amine | 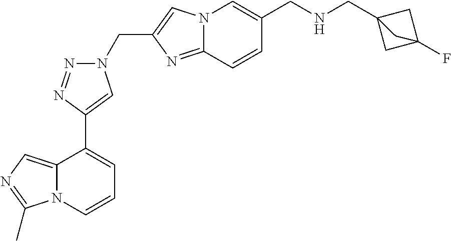 [SMILES: Cc1ncc2c(-c3cn(Cc4cn5cc(CNCC67CC(F)(C6)C7)ccc5n4)nn3)cccn12] | C | 2.53 | 457.4 |
| 37 | 3-{1-[(6- {[({bicyclo[1.1.1] pentan-1- yl}methyl)amino] methyl}imidazo [1,2-a]pyridin-2- yl)methyl]-1H-1,2,3- triazol-4-yl}-5- methoxypyridine-2- carbonitrile | 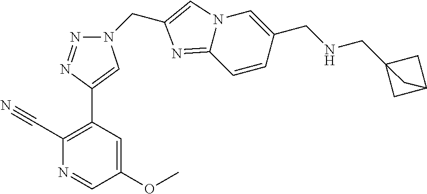 [SMILES: COc1cnc(C#N)c(-c2cn(Cc3cn4cc(CNCC56CC(C5)C6)ccc4n3)nn2)c1] | C | 3.13 | 441.5 |
| 38 | 3-{1-[(6- {[(cyclobutylmethyl) amino]methyl} imidazo[1,2-a]pyridin- 2-yl)methyl]-1H- 1,2,3-triazol-4-yl}-5- methoxypyridine-2- carbonitrile | 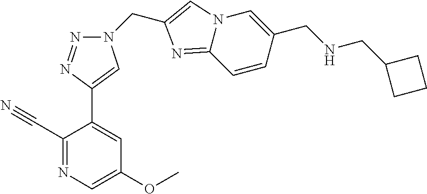 [SMILES: COc1cnc(C#N)c(-c2cn(Cc3cn4cc(CNCC5CCC5)ccc4n3)nn2)c1] | C | 2.95 | 429.5 |
| 39 | 3-[1-({6-[(4,4- dimethylpiperidin-1- yl)methyl]imidazo[1,2- a]pyridin-2- yl}methyl)-1H-1,2,3- triazol-4-yl]-5- methoxypyridine-2- carbonitrile | 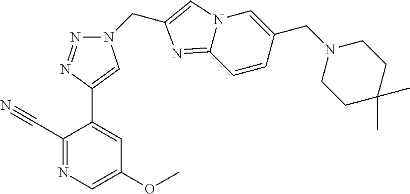 [SMILES: COc1cnc(C#N)c(-c2cn(Cc3cn4cc(CN5CCC(C)(C)CC5)ccc4n3)nn2)c1] | C | 3.45 | 457.6 |
| 40 | 3-(1-{[6-({6- azaspiro[3.4]octan- 6-yl}methyl)imidazo [1,2-a]pyridin-2- yl]methyl}-1H-1,2,3- triazol-4-yl)-5- methoxypyridine-2- carbonitrile | 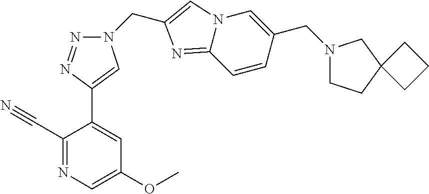 [SMILES: COc1cnc(C#N)c(-c2cn(Cc3cn4cc(CN5CCC6(CCC6)C5)ccc4n3)nn2)c1] | C | 3.45 | 455.4 |
| 41 | 3-{1-[(6-{[({3- fluorobicyclo[1.1.1] pentan-1-yl} methyl)amino] methyl}imidazo[1,2- a]pyridin-2- yl)methyl]-1H-1,2,3- triazol-4-yl}-5- methoxypyridine-2- carbonitrile | 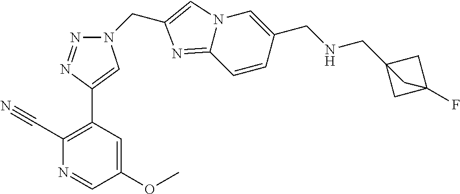 [SMILES: COc1cnc(C#N)c(-c2cn(Cc3cn4cc(CNCC56CC(F)(C5)C6)ccc4n3)nn2)c1] | C | 2.89 | 459.4 |
| 42 | 3-(1-{[6-({6- azaspiro[3.4]octan- 6- yl}methyl)imidazo [1,2-a]pyridin-2- yl]methyl}-1H-1,2,3- triazol-4-yl)-5- fluoropyridine-2- carbonitrile | 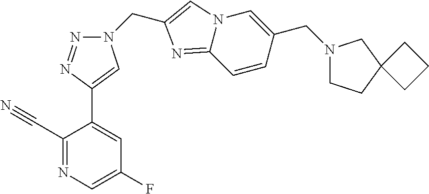 [SMILES: N#Cc1ncc(F)cc1-c1cn(Cc2cn3cc(CN4CCC5(CCC5)C4)ccc3n2)nn1] | C | 3.42 | 443.4 |
| 43 | 5-fluoro-3-{1-[(6- {[({3- fluorobicyclo[1.1.1] pentan-1- yl}methyl)amino] methyl}imidazo[1,2- a]pyridin-2- yl)methyl]-1H-1,2,3- triazol-4-yl}pyridine- 2-carbonitrile | 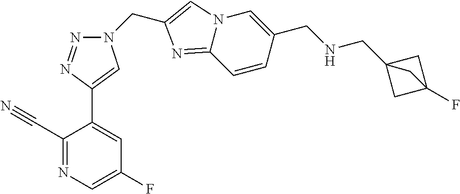 [SMILES: N#Cc1ncc(F)cc1-c1cn(Cc2cn3cc(CNCC45CC(F)(C4)C5)ccc3n2)nn1] | C | 2.90 | 447.4 |
| 44 | ({3- fluorobicyclo[1.1.1] pentan-1- yl}methyl)[(2-{[4-(6- methoxypyrazin-2- yl)-1H-1,2,3-triazol- 1- yl]methyl}imidazo [1,2-a]pyridin-6- yl)methyl]amine | 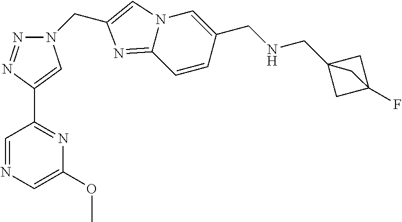 [SMILES: COc1cncc(-c2cn(Cc3cn4cc(CNCC56CC(F)(C5)C6)ccc4n3)nn2)n1] | C | 2.76 | 435.4 |
| 45 | ({3- fluorobicyclo[1.1.1] pentan-1- yl}methyl)[(2-{[4-(5- methoxypyridin-3-yl)- 1H-1,2,3-triazol-1- yl]methyl}imidazo [1,2-a]pyridin-6- yl)methyl]amine | 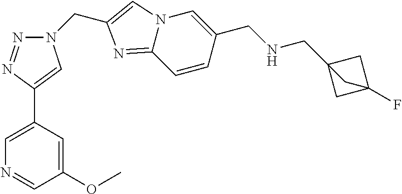 [SMILES: COc1cncc(-c2cn(Cc3cn4cc(CNCC56CC(F)(C5)C6)ccc4n3)nn2)c1] | C | 2.49 | 434.4 |
| 46 | N-[(6- {[(cyclobutylmethyl) amino]methyl}imidazo [1,2-a]pyridin-2- yl)methyl]-5- methoxypyridine-3- carboxamide | 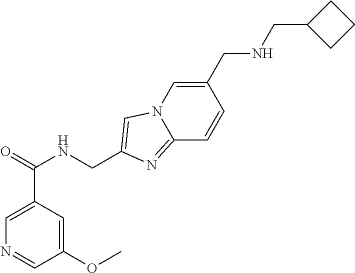 [SMILES: COc1cncc(C(=O)NCc2cn3cc(CNCC4CCC4)ccc3n2)c1] | C | 2.46 | 380.3 |
| 47 | ({3- fluorobicyclo[1.1.1] pentan-1- yl}methyl)[(2-{[4-(4- methoxypyridin-3-yl)- 1H-1,2,3-triazol-1- yl]methyl}imidazo [1,2-a]pyridin-6- yl)methyl]amine | 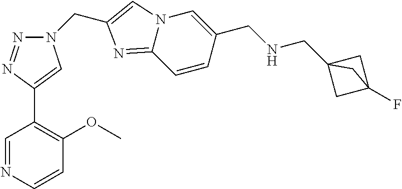 [SMILES: COc1ccncc1-c1cn(Cc2cn3cc(CNCC45CC(F)(C4)C5)ccc3n2)nn1] | C | 2.47 | 434.4 |
| 48 | ({3- fluorobicyclo[1.1.1] pentan-1- yl}methyl)[(2-{[4-(6- methoxypyridin-3-yl)- 1H-1,2,3-triazol-1- yl]methyl}imidazo [1,2-a]pyridin-6- yl)methyl]amine | 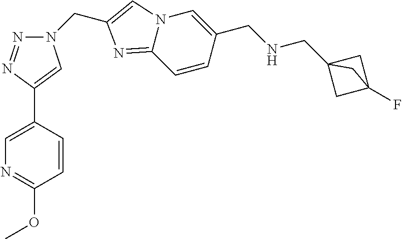 [SMILES: COc1ccc(-c2cn(Cc3cn4cc(CNCC56CC(F)(C5)C6)ccc4n3)nn2)cn1] | C | 2.80 | 434.4 |
| 49 | 3-{1-[(6-{[({3- fluorobicyclo[1.1.1] pentan-1- yl}methyl)amino] methyl}imidazo[1,2- a]pyridin-2- yl)methyl]-1H-1,2,3- triazol-4-yl}-5- methoxypyridine-2- carboxamide | 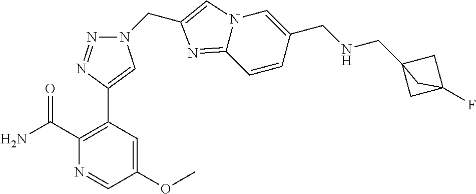 [SMILES: COc1cnc(C(N)=O)c(-c2cn(Cc3cn4cc(CNCC56CC(F)(C5)C6)ccc4n3)nn2)c1] | C | 2.30 | 477.5 |
| 50 | (cyclobutylmethyl)[(2- {[4-(1-methyl-1H- indazol-7-yl)-1H- 1,2,3-triazol-1- yl]methyl}imidazo [1,2-a]pyridin-6- yl)methyl]amine | 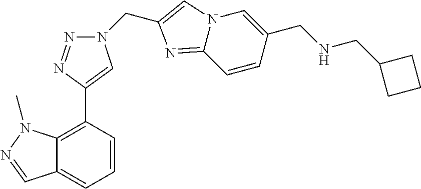 [SMILES: Cn1ncc2cccc(-c3cn(Cc4cn5cc(CNCC6CCC6)ccc5n4)nn3)c21] | D | 3.79 | 427.3 |
| 51 | 3-{1-[(6-{[({3- fluorobicyclo[1.1.1] pentan-1- yl}methyl)amino] methyl}imidazo[1,2- a]pyridin-2- yl)methyl]-1H-1,2,3- triazol-4-yl}-5- (morpholin-4- yl)pyridine-2- carbonitrile | 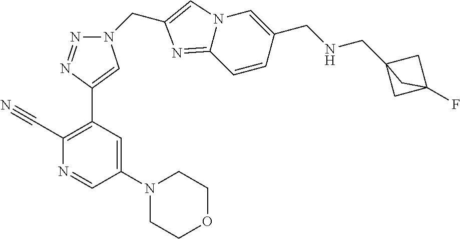 [SMILES: N#Cc1ncc(N2CCOCC2)cc1-c1cn(Cc2cn3cc(CNCC45CC(F)(C4)C5)ccc3n2)nn1] | E | 1.32 | 514.3 |
| 52 | ({3- fluorobicyclo[1.1.1] pentan-1-yl}methyl) ({[2-({4-[5- (morpholin-4- yl)pyridin-3-yl]-1H- 1,2,3-triazol-1- yl}methyl)imidazo [1,2-a]pyridin-6- yl]methyl})amine | 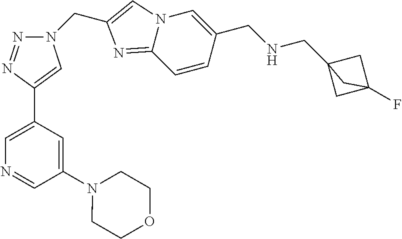 [SMILES: FC12CC(CNCc3ccc4nc(Cn5cc(-c6cncc(N7CCOCC7)c6)nn5)cn4c3)(C1)C2] | C | 2.54 | 489.5 |

### Example 31: N-(cyclobutylmethyl)-1-[2-[[4-(4-isoquinolyl)triazol-1-yl]methyl]imidazo[1,2-a]pyridin-6-yl]methanamine

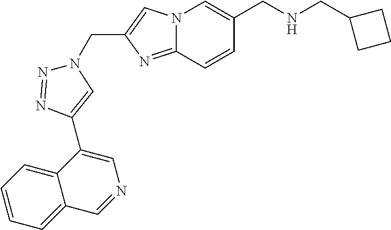 [SMILES: c1ccc2c(-c3cn(Cc4cn5cc(CNCC6CCC6)ccc5n4)nn3)cncc2c1]

### Step 1: tert-butyl N-[[2-[[4-(4-isoquinolyl)triazol-1-yl]methyl]imidazo[1,2-a]pyridin-6-yl]methyl]carbamate

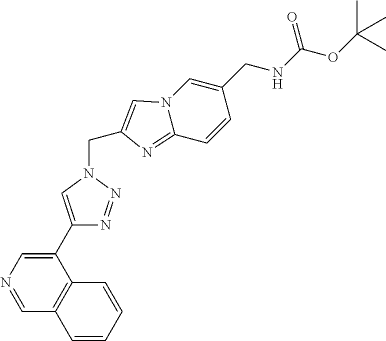 [SMILES: CC(C)(C)OC(=O)NCc1ccc2nc(Cn3cc(-c4cncc5ccccc45)nn3)cn2c1]

Under nitrogen atmosphere, isoquinoline-4-carbaldehyde (100 mg, 0.636 mmol) was suspended in MeOH (anhydrous, 1 mL) and THE (anhydrous, 1 mL), dimethyl (1-diazo-2-oxopropyl)phosphonate (245 mg, 1.28 mmol) and K₂CO₃ (264 mg, 1.91 mmol) were added to the reaction mixture and stirred for 5 hours.

Tert-butyl N-[[2-[[4-(4-isoquinolyl)triazol-1-yl]methyl]imidazo[1,2-a]pyridin-6-yl]methyl]carbamate

Intermediate 20 (193 mg, 0.636 mmol) and copper(1) iodide (25 mg, 0.131 mmol) were added and the mixture was stirred at ambient temperature for 2 hours.

Water was added and the mixture was extracted with CHCl₃/IPA×3. The organic extracts were combined, dried (MgSO₄) and evaporated under vacuum to give the crude material which was purified by chromatography on SiO₂ (KP-NH, eluting with 50 to 100% Heptane/EtOAc followed by 5% MeOH in EtOAc) to give the title compound (84 mg, 29%) as an off-white solid.

Method B: LC-MS (electrospray): m/z=456.4 (M+H)⁺, RT=1.42 min.

### Step 2: N-(cyclobutylmethyl)-1-[2-[[4-(4-isoquinolyl)triazol-1-yl]methyl]imidazo[1,2-a]pyridin-6-yl]methanamine

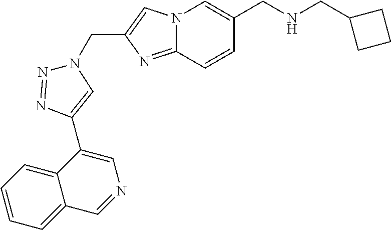 [SMILES: c1ccc2c(-c3cn(Cc4cn5cc(CNCC6CCC6)ccc5n4)nn3)cncc2c1]

To a solution of [2-[[4-(4-isoquinolyl)triazol-1-yl]methyl]imidazo[1,2-a]pyridin-6-yl]methanamine (13 mg, 0.0368 mmol) in DCE (1.0 mL) was added cyclobutanecarbaldehyde (3.1 mg, 0.0368 mmol) and the reaction was stirred at ambient temperature for 30 minutes, before sodium triacetoxyborohydride (16 mg, 0.0737 mmol) was added and the reaction was stirred at 40° C. for 2 hours.

The reaction was cooled to ambient temperature and the solvent was removed in vacuo. Water (5 mL) and NaOH (1 M, 2.0 mL) were added and the aqueous was extracted with IPA/CHCl₃ (1:4, 3×3 mL). The organics were dried (Na₂SO₄) and evaporated under reduced pressure to give a yellow gum, which was purified by preparative HPLC (Method B) to give the title compound (2.1 mg, 13%) as an off white solid along with the bis alkylated by-product.

Method C: LC-MS (electrospray): m/z=424.4 (M+H)⁺, RT=2.87 min.

### Example 32: bis(cyclobutylmethyl)[(2-{[4-(isoquinolin-4-yl)-1H-1,2,3-triazol-1-yl]methyl}imidazo[1,2-a]pyridin-6-yl)methyl]amine

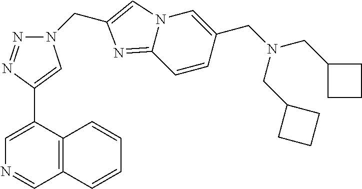 [SMILES: c1ccc2c(-c3cn(Cc4cn5cc(CN(CC6CCC6)CC6CCC6)ccc5n4)nn3)cncc2c1]

The title compound was isolated during the purification of Example 32 to give (6.4 mg, 35%) as an off white solid.

Method C: LC-MS (electrospray): m/z=492.5 (M+H)⁺, RT=4.60 min.

### Example 33: (cyclobutylmethyl)({2-[(4-{1H-pyrrolo[2,3-b]pyridin-5-yl}-1H-1,2,3-triazol-1-yl)methyl]imidazo[1,2-a]pyridin-6-yl}methyl)amine

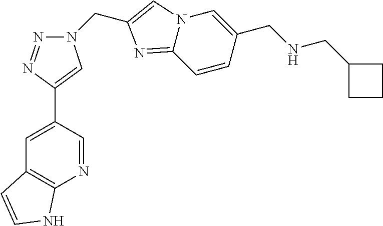 [SMILES: c1cc2cc(-c3cn(Cc4cn5cc(CNCC6CCC6)ccc5n4)nn3)cnc2[nH]1]

### Step 1: 2-[(5-iodopyrrolo[2,3-b]pyridin-1-yl)methoxy]ethyl-trimethyl-silane

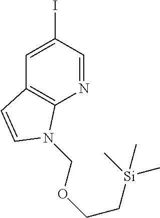 [SMILES: C[Si](C)(C)CCOCn1ccc2cc(I)cnc21]

5-iodo-1H-pyrrolo[2,3-b]pyridine (430 mg, 1.76 mmol) was dissolved in DMF-Anhydrous (6 mL) and NaH (60% wt in mineral oil, 211 mg, 8.81 mmol) was added—gas evolution. The mixture was stirred at ambient temperature for 10 minutes before [2-(chloromethoxy)ethyl](trimethyl)silane (588 mg, 3.52 mmol) was added and the mixture was stirred at ambient temperature for 30 minutes.

The mixture was quenched with NaHCO₃ (sat., 50 mL) and extracted with EtOAc (2×30 mL). The combined organic layers were washed with brine (5×20 mL), dried over sodium sulfate and evaporated under vacuum to a residue was purified by chromatography on SiO₂ (eluting with 0-50% EtOAc/heptane) to afford the title compound (539 mg, 73%) as a colourless oil.

Method B: LC-MS (electrospray): m/z=375.1 (M+H)⁺, RT=2.15 min.

### Step 2: Trimethyl-[2-[1-(2-trimethylsilylethoxymethyl)pyrrolo[2,3-b]pyridin-5-yl]ethynyl]silane

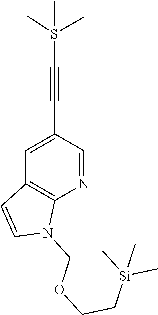 [SMILES: C[Si](C)(C)CCOCn1ccc2cc(C#CS(C)(C)C)cnc21]

2-[(5-iodopyrrolo[2,3-b]pyridin-1-yl)methoxy]ethyl-trimethyl-silane (539 mg, 1.28 mmol), CuI (24 mg, 0.13 mmol), PdCl₂ dppf (93 mg, 0.13 mmol) and triethylamine (2 mL, 14.3 mmol) were combined in DMF-Anhydrous (5 mL) and the mixture sparged with nitrogen for 5 minutes. Ethynyl(trimethyl)silane (355 μl, 2.56 mmol) was added and the mixture further sparged briefly, the vessel sealed and the mixture was stirred at ambient temperature for 2 hours.

The reaction mixture was diluted with EtOAc (100 mL) washed with NaHCO₃ (sat., 50 mL) and brine (5×50 mL). The organic layer was dried over sodium sulfate and evaporated under vacuum.

The residue was purified by chromatography on SiO₂ (eluting with 0-50% EtOAc/heptane) to afford the title compound (476 mg, 97%) as a black oil Method B: LC-MS (electrospray): m/z=345.4 (M+H)⁺, RT=2.49 min.

### Step 3: 2-[(5-Ethynylpyrrolo[2,3-b]pyridin-1-yl)methoxy]ethyl-trimethyl-silane

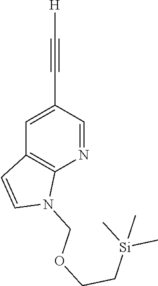 [SMILES: C#Cc1cnc2c(ccn2COCC[Si](C)(C)C)c1]

Trimethyl-[2-[1-(2-trimethylsilylethoxymethyl)pyrrolo[2,3-b]pyridin-5-yl]ethynyl]silane (470 mg, 1.36 mmol) was dissolved in methanol (20 mL), K₂CO₃ (377 mg, 2.72 mmol) was added and the mixture was stirred at ambient temperature for 10 minutes.

The mixture was filtered, washing with MeOH, and the combined filtrates evaporated under vacuum. The residue was purified by chromatography on SiO₂ (eluting with 0-100% EtOAc/heptane) to afford the title compound (355 mg, 84%) as a yellow oil Method B: LC-MS (electrospray): m/z=273.3 (M+H)⁺, RT=2.05 min.

### Step 4: [2-[[4-[1-(2-trimethylsilylethoxymethyl)pyrrolo[2,3-b]pyridin-5-yl]triazol-1-yl]methyl]imidazo[1,2-a]pyridin-6-yl]methanol

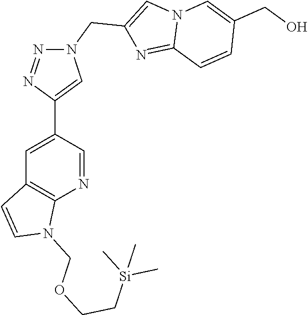 [SMILES: C[Si](C)(C)CCOCn1ccc2cc(-c3cn(Cc4cn5cc(CO)ccc5n4)nn3)cnc21]

2-[(5-Ethynylpyrrolo[2,3-b]pyridin-1-yl)methoxy]ethyl-trimethyl-silane (355 mg, 1.15 mmol), [2-(azidomethyl)imidazo[1,2-a]pyridin-6-yl]methanol Intermediate 21, Step 3 (233 mg, 1.15 mmol) and sodium ascorbate (273 mg, 1.38 mmol) were combined in DMF (5 mL) and Water (1 mL). CuSO₄ (4 mg, 0.03 mmol) was added and the mixture was stirred at ambient temperature for an hour.

The mixture was diluted with chloroform/isopropanol (3:1, 30 mL) and Water (10 mL) and the phases separated. The aqueous phase was extracted with chloroform/isopropanol (3:1, 3×30 mL) and the combined organic layers were dried over sodium sulfate and evaporated under vacuum.

The residue was purified by chromatography on SiO₂ (kp-NH, eluting with 0-100% EtOAc/heptane followed by 0-20% MeOH/EtOAc) to afford the title compound (372 mg, 62%) as a white solid.

Method B: LC-MS (electrospray): m/z=476.3 (M+H)⁺, RT=1.54 min.

### Step 5: 2-[[4-[1-(2-trimethylsilylethoxymethyl)pyrrolo[2,3-b]pyridin-5-yl]triazol-1-yl]methyl]imidazo[1,2-a]pyridine-6-carbaldehyde

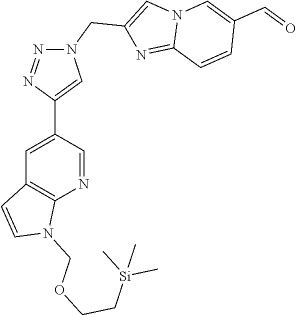 [SMILES: C[Si](C)(C)CCOCn1ccc2cc(-c3cn(Cc4cn5cc(C=O)ccc5n4)nn3)cnc21]

[2-[[4-[1-(2-trimethylsilylethoxymethyl)pyrrolo[2,3-b]pyridin-5-yl]triazol-1-yl]methyl]imidazo[1,2-a]pyridin-6-yl]methanol (372 mg, 0.78 mmol) and Dess-Martin periodinane (995 mg, 2.35 mmol) were combined in DCE (15 mL) and the mixture was heated at reflux for 2 hours during which time an orange colouration was noted.

The mixture was cooled to ambient temperature and quenched with NaHCO (sat., 50 mL). The mixture was extracted with chloroform/isopropanol (3:1, 3×80 mL). The combined organic extracts were dried over sodium sulfate and evaporated under vacuum to afford a residue which was triturated with EtOAc/heptane and the solids were collected by filtration to afford the title compound (472 mg, 96%) as a pale brown solid.

Method B: LC-MS (electrospray): m/z=474.3 (M+H)⁺, RT=1.66 min.

### Step 6: (cyclobutylmethyl)({2-[(4-{1H-pyrrolo[2,3-b]pyridin-5-yl}-1H-1,2,3-triazol-1-yl)methyl]imidazo[1,2-a]pyridin-6-yl}methyl)amine

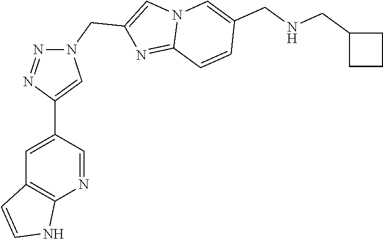 [SMILES: c1cc2cc(-c3cn(Cc4cn5cc(CNCC6CCC6)ccc5n4)nn3)cnc2[nH]1]

2-[[4-[1-(2-trimethylsilylethoxymethyl)pyrrolo[2,3-b]pyridin-5-yl]triazol-1-yl]methyl]imidazo[1,2-a]pyridine-6-carbaldehyde (200 mg, 0.42 mmol) and 1-cyclobutylmethanamine (72 mg, 0.84 mmol) were combined in 1,1,1,3,3,3-Hexafluoro-2-propanol (3 mL) and the mixture stirred at ambient temperature for 30 minutes

NaBH₄ (160 mg, 4.22 mmol) and a few drops of MeOH were added—gas evolution—and the mixture was stirred briefly at ambient temperature.

The mixture was quenched with MeOH (40 mL)—gas evolution—and evaporated under vacuum. The residue was suspended in MeOH (10 mL) and treated with HCl (4 M in Dioxane 5 mL) and stirred at ambient temperature for 30 minutes and heated at reflux for an hour.

The mixture was cooled to ambient temperature and evaporated under vacuum. The residue was suspended in water (2 mL) and treated with c.HCl (3 mL) and the mixture was incubated at ambient temperature for 16 hours. The mixture was heated at 90° C. for 6 hours.

The reaction mixture was cooled to ambient temperature and evaporated under vacuum. The residue was suspended in water (2 mL) and treated with NaOH (2 M) and incubated at ambient temperature for 2 days and then heated at 100° C. for an hour.

The reaction mixture was cooled to ambient temperature and extracted with chloroform/isopropanol (3:1, 3×30 mL). The combined organic extracts were dried over sodium sulfate and evaporated under vacuum.

The residue was purified by reverse phase chromatography (30 g C18-Ultra, eluting with Water+0.1% NH₃/Acetonitrile+0.1% NH₃ 10-100%) to afford the title compound (9 mg, 5%) as a pale yellow solid.

Method E: LC-MS (electrospray): m/z=413.3 (M+H)⁺, RT=1.11 min.

### Example 34: (cyclobutylmethyl)({2-[(4-{1H-pyrrolo[2,3-b]pyridin-5-yl}-1H-imidazol-1-yl)methyl]imidazo[1,2-a]pyridin-6-yl}methyl)amine

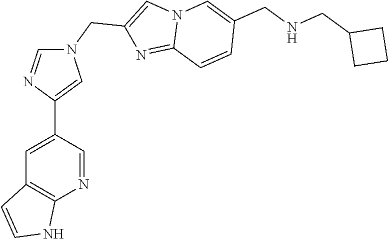 [SMILES: c1cc2cc(-c3cn(Cc4cn5cc(CNCC6CCC6)ccc5n4)cn3)cnc2[nH]1]

### Step 1: [2-[(4-bromoimidazol-1-yl)methyl]imidazo[1,2-a]pyridin-6-yl]methanol

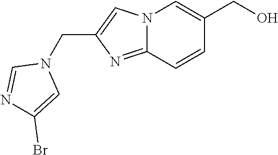 [SMILES: OCc1ccc2nc(Cn3cnc(Br)c3)cn2c1]

4-bromo-1H-imidazole (500 mg, 3.30 mmol), [2-(chloromethyl)imidazo[1,2-a]pyridin-6-yl]methanol Intermediate 21 Step 2 (714 mg, 3.63 mmol), Cs₂CO₃ (3.22 g, 9.90 mmol) and NaI (49 mg, 0.33 mmol) were combined in DMF (10 mL) and the mixture was heated at 80° C. for 2 hours.

The mixture was cooled to ambient temperature and diluted with water (50 mL). The mixture was extracted with chloroform/isopropanol (3:1, 3×50 mL) and the combined organic extracts were dried over sodium sulfate and evaporated under vacuum.

The residue was purified by chromatography on SiO₂ (kp-NH, eluting with 0-100% EtOAc/heptane followed by 0-20% MeOH/EtOAc) to afford the title compound (983 mg, 91%) as a white foam.

Method B: LC-MS (electrospray): m/z=307.1/309.1 (M+H)⁺, RT=1.14 min.

### Step 2: [2-[(4-bromoimidazol-1-yl)methyl]imidazo[1,2-a]pyridin-6-yl]methanol

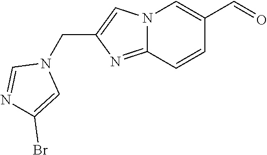 [SMILES: O=Cc1ccc2nc(Cn3cnc(Br)c3)cn2c1]

[2-[(4-bromoimidazol-1-yl)methyl]imidazo[1,2-a]pyridin-6-yl]methanol (1 g 3.26 mmol) and MnO₂ (2.83 g, 32.6 mmol) were combined in DCE (40 mL) and the mixture was heated at 80° C. for 2 hours.

Further MnO₂ (1 g, 11.5 mmol) added and heating was continued for 3 hours.

The mixture was cooled to ambient temperature and filtered through a plug of Celite and the residue washed with MeOH. The combined filtrates were evaporated under vacuum to afford the title compound (910 mg, 86%) as a grey solid.

Method B: LC-MS (electrospray): m/z=305.1/307.1 (M+H)⁺, RT=1.20 min.

### Step 3: [2-[(4-bromoimidazol-1-yl)methyl]imidazo[1,2-a]pyridin-6-yl]methanol

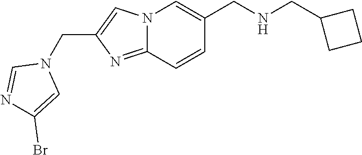 [SMILES: Brc1cn(Cc2cn3cc(CNCC4CCC4)ccc3n2)cn1]

2-[(4-bromoimidazol-1-yl)methyl]imidazo[1,2-a]pyridine-6-carbaldehyde (900 mg, 2.95 mmol) and 1-cyclobutylmethanamine (502 mg, 5.90 mmol) were combined in 1,1,1,3,3,3-Hexafluoro-2-propanol (10 mL) and the mixture was stood at ambient temperature for 1.5 hours. NaBH₄ (334 mg, 8.85 mmol) was added with a few drops MeOH—gas evolution—and the mixture was stirred at ambient temperature for an hour.

The mixture was quenched with MeOH (30 mL)—gas evolution—and evaporated under vacuum.

The residue was partitioned between NaHCO₃ (sat., 20 mL) and chloroform/isopropanol (3:1, 30 mL) and the phases separated. The aqueous was extracted with chloroform/isopropanol (3:1, 2×30 mL) and the combined organic extracts were dried over sodium sulfate and evaporated under vacuum to afford the title compound (1.10 g, 78%) as a pale yellow residue.

Method B: LC-MS (electrospray): m/z=374.2/376.2 (M+H)⁺, RT=1.40 min.

### Step 4: tert-butyl N-[[2-[(4-bromoimidazol-1-yl)methyl]imidazo[1,2-a]pyridin-6-yl]methyl]-N-(cyclobutylmethyl)carbamate

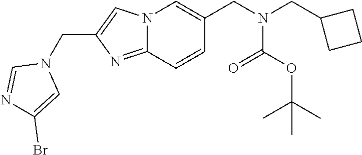 [SMILES: CC(C)(C)OC(=O)N(Cc1ccc2nc(Cn3cnc(Br)c3)cn2c1)CC1CCC1]

1-[2-[(4-bromoimidazol-1-yl)methyl]imidazo[1,2-a]pyridin-6-yl]-N-(cyclobutylmethyl)methanamine (1.1 g, 2.94 mmol) and Boc₂O (962 mg, 4.41 mmol) were combined in acetonitrile (50 mL) and heated at reflux for 2 hours.

The mixture was cooled to ambient temperature and evaporated under vacuum.

The residue was dissolved in EtOAc (50 mL), washed with NaHCO₃ (sat., 50 mL) and brine (2×50 mL), dried over sodium sulfate and evaporated under vacuum. The residue was purified by chromatography on SiO₂ (kpNH, eluting with 0-100% EtOAc/heptane) to give the title compound (1.1 g, 83%) as colourless oil.

Method A: LC-MS (electrospray): m/z=474.25/476.25 (M+H)⁺, RT=1.12 min.

### Step 5: tert-Butyl N-(cyclobutylmethyl)-N-[[2-[[4-(1H-pyrrolo[2,3-b]pyridin-5-yl)imidazol-1-yl]methyl]imidazo[1,2-a]pyridin-6-yl]methyl]carbamate

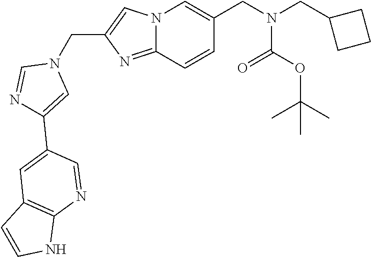 [SMILES: CC(C)(C)OC(=O)N(Cc1ccc2nc(Cn3cnc(-c4cnc5[nH]ccc5c4)c3)cn2c1)CC1CCC1]

5-(4,4,5,5-tetramethyl-1,3,2-dioxaborolan-2-yl)-1H-pyrrolo[2,3-b]pyridine (150 mg, 0.61 mmol), tert-butyl N-[[2-[(4-bromoimidazol-1-yl)methyl]imidazo[1,2-a]pyridin-6-yl]methyl]-N-(cyclobutylmethyl)carbamate (321 mg, 0.68 mmol), K₂CO₃ (1.2 M, 1.54 mL, 1.84 mmol) and PdCl₂ dppf (45 mg, 0.06 mmol) were combined in dioxane (6 mL) and the mixture was sparged with nitrogen for 5 minutes. The vessel was sealed and the mixture was heated at 100° C. for 4 hours.

The reaction mixture was cooled to ambient temperature and left standing overnight.

The mixture was diluted with NaHCO₃ (sat., 40 mL) and extracted with DCM (3×50 mL) and chloroform/isopropanol (3:1, 30 mL). The combined organic extracts were dried over sodium sulfate and evaporated under vacuum.

The residue was purified by chromatography on SiO₂ (kp-NH, eluting with 0-100% EtOAc/heptane followed by 0-20% MeOH/EtOAc) to afford the title compound (291 mg, 64%) as a brown solid Method B: LC-MS (electrospray): m/z=512.3 (M+H)⁺, RT=1.65 min.

### Step 6: tert-butyl 5-[1-[[6-[[tert-butoxycarbonyl(cyclobutylmethyl)amino]methyl]imidazo[1,2-a]pyridin-2-yl]methyl]imidazol-4-yl]pyrrolo[2,3-b]pyridine-1-carboxylate

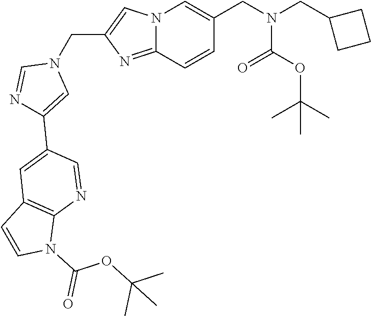 [SMILES: CC(C)(C)OC(=O)N(Cc1ccc2nc(Cn3cnc(-c4cnc5c(ccn5C(=O)OC(C)(C)C)c4)c3)cn2c1)CC1CCC1]

tert-butyl N-(cyclobutylmethyl)-N-[[2-[[4-(1H-pyrrolo[2,3-b]pyridin-5-yl)imidazol-1-yl]methyl]imidazo[1,2-a]pyridin-6-yl]methyl]carbamate (290 mg, 0.57 mmol), di-tert-butyl dicarbonate (161 mg, 0.74 mmol) and DMAP (20 mg, 0.16 mmol) were combined in cetonitrile (20 mL) and heated at 80° C. for an hour. Further di-tert-butyl dicarbonate (1 g, 4.59 mmol) was added—gas evolution—and the heating was continued for 3 hours. The mixture was allowed to stand for 2 days at ambient temperature. The mixture was mixture was heated to 80° C. for 2 hours. Further di-tert-butyl dicarbonate (3 g, 13.8 mmol) added—gas evolution—and the heating was continued for 3 hours.

The reaction mixture was cooled to ambient temperature and evaporated under vacuum.

The residue was dissolved in EtOAc (50 mL) and washed with. NaHCO₃ (sat., 2×30 mL) and brine (30 mL). The organic layer was dried over sodium sulfate and evaporated under vacuum.

The residue was purified by chromatography on SiO₂ (kp-NH, eluting with 0-100% EtOAc/heptane followed by 0-20% MeOH/EtOAc) to afford the title compound (183 mg, 53%) as an orange residue

Method B: LC-MS (electrospray): m/z=612.4 (M+H)⁺, RT=1.92 min.

### Step 7: (cyclobutylmethyl)({2-[(4-{1H-pyrrolo[2,3-b]pyridin-5-yl}-1H-imidazol-1-yl)methyl]imidazo[1,2-a]pyridin-6-yl}methyl)amine

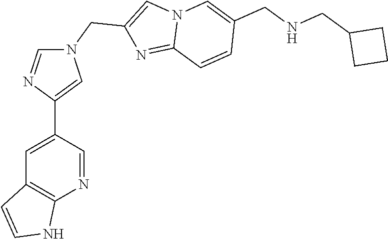 [SMILES: c1cc2cc(-c3cn(Cc4cn5cc(CNCC6CCC6)ccc5n4)cn3)cnc2[nH]1]

tert-Butyl 5-[1-[[6-[[tert-butoxycarbonyl(cyclobutylmethyl)amino]methyl]imidazo[1,2-a]pyridin-2-yl]methyl]imidazol-4-yl]pyrrolo[2,3-b]pyridine-1-carboxylate (180 mg, 0.29 mmol) was dissolved in methanol (3 mL) and treated with HCl (4 M in dioxane, 3 mL) and the mixture was heated at 70° C. for 2 hours.

The mixture was cooled to ambient temperature and evaporated under vacuum. The residue was dissolved in MeOh and purified by ion exchange (SCS-2, washing with MeOH and eluting with ammonia in MeOH). The basic eluent was evaporated under vacuum.

The residue was purified by basic preparative HPLC (Method B) to afford the title compound (18 mg, 14%) as a white solid.

Method D: LC-MS (electrospray): m/z=412.3 (M+H)⁺, RT=3.42 min.

### Example 35: (cyclobutylmethyl)({2-[(4-{imidazo[1,5-a]pyridin-5-yl}-1H-1,2,3-triazol-1-yl)methyl]imidazo[1,2-a]pyridin-6-yl}methyl)amine

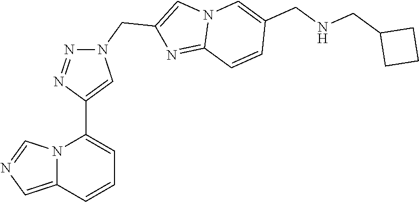 [SMILES: c1cc(-c2cn(Cc3cn4cc(CNCC5CCC5)ccc4n3)nn2)n2cncc2c1]

Under a nitrogen atmosphere, imidazo[1,5-a]pyridine-5-carbaldehyde (100 mg, 0.684 mmol) was suspended in methanol (dry, 2 mL) and THF (dry, 2 mL). Dimethyl (1-diazo-2-oxopropyl)phosphonate (0.21 mL, 1.37 mmol) and potassium carbonate (284 mg, 2.05 mmol) were added and the mixture was stirred at ambient temperature for 3.5 hours.

tert-butyl N-[[2-(azidomethyl)imidazo[1,2-a]pyridin-6-yl]methyl]-N-(cyclobutylmethyl)carbamate Intermediate 10 (253 mg, 0.684 mmol) and copper(1) iodide (26 mg, 0.137 mmol) were added, and the resulting mixture was stirred at ambient temperature for 24 hours.

The mixture was partitioned between water (30 mL) and DCM (30 mL), and the aqueous phase was extracted with DCM (4×30 mL) and chloroform/IPA (3:1, 3×30 mL). The combined organisms were dried over Na₂SO₄ and concentrated in vacuo.

The residue was dissolved in HCl (4 M in dioxane, 5 mL) and methanol (0.5 mL), and stirred at ambient temperature for an hour.

The solvent was removed in vacuo.

The residue was purified by preparative HPLC (Method B) to give (31 mg of crude product.

The material was further purified by preparative HPLC (Method A). The product containing fractions were combined and the pH adjusted to pH 10 by addition of NaHCO₃ and then NaOH(aq). The mixture was extracted with CHCl3/IPA (3:1, 5×20 mL), and the combined organic layers were concentrated in vacuo. The resulting solid was freeze dried to give the title compound (9.3 mg, 3.3%) as a yellow solid.

Method C: LC-MS (electrospray): m/z=413.4 (M+H)⁺, RT=2.72 min.

### Examples—Biochemical and Cell Activity

### METTL3/14 Methyltransferase Assay

### Biochemical Assay—METTL3_14 RFMS

The enzymatic assay was established to determine IC50 values for inhibition of RNA methyltransferase activity. The enzyme used was full-length his-tagged METTL3 co-expressed with full length FLAG-tagged METTL14 in a baculovirus expression system. Enzymatic reactions were performed at room temperature in 384-well plates using a final reaction volume of 20 μL containing 20 mM TrisCl pH 7.6, 1 mM DTT, 0.01% Tween-20.5 nM final concentration of METTL3/14 was pre-incubated with various compound concentrations for 10 minutes, followed by addition of 0.2 μM final concentration synthetic RNA substrate (5′P-uacacucgaucuggacuaaagcugcuc-3′) and 0.5 μM final concentration S-adenosyl-methionine (SAM). The reaction was incubated for further 60 minutes at room temperature, and then quenched by the addition of 40 μL 7.5% TCA with two internal product standards (D₄-SAH and ¹³C₁₀-SAH). After termination, plates were sealed, centrifuged and stored at 4° C. until analysis. Note: the lower limit of the assay is IC50=5 nM.

### Mass Spectrometry Analysis

RNA methyltransferase activity was measured label free using the RapidFire™ mass spectrometry (RF/MS) platform. Stopped and stable assay plates were analyzed on the Agilent RF300 integrated autosampler/solid-phase extraction (SPE) system coupled to an ABSciex 4000 mass spectrometer for the generation of the product S-adenosyl homocysteine (SAH) and normalized to the ratio of signal of the two internal product standards, respectively. Solvent A was water containing 0.1% (v/v) TCA. Solvent B was acetonitrile/0.1% ammonium acetate (8:2, v/v). More specifically, plates were centrifuged at 4350 rpm for 10 min, samples were aspirated under vacuum for 600 ms, then loaded onto a C18 solid-phase extraction cartridge and washed for 3 s with solvent A at a flow rate of 1.5 mL/min. Retained product and internal standards were eluted with solvent B at a flow rate of 1 mL/min for 3 s and finally the cartridge was reequilibrated with solvent A for 500 ms. The mass transition for the product (SAH) was 384.9/135.9 Da. Transitions of the two internal product standards (IS1: D₄-SAH and IS2: ¹³C₁₀-SAH) were 389.1/135.8 Da and 395.0/134.2 Da, respectively. Ratios of SAH/IS1 and SAH/IS2 were used for normalization of matrix effects. IC50 values were calculated based on dilution series of individual compounds. Potency of a compound was measured at varied inhibitor concentrations and normalized to control wells without RNA substrate and without inhibition (DMSO only).

### Results:

| | RapidFire |
|---|---|
| Example | METTL3_14 |
| No | IC50 nM |
| Reference | 5340 |
| Example 1 | |
| 2 | 719 |
| 4 | 3250 |
| 5 | 87.7 |
| 6 | 151 |
| 7 | 1400 |
| 8 | 1190 |
| 10 | 36.5 |
| 11 | 104 |
| 13 | 1070 |
| 14 | 14.53 |
| 15 | 27.3 |
| 16 | 1600 |
| 17 | 3820 |
| 18 | 14.1 |
| 19 | 6.1 |
| 20 | 5400 |
| 21 | 4580 |
| 22 | 6.1 |
| 23 | 6.1 |
| 24 | 6.1 |
| 25 | 15.8 |
| 26 | 6.1 |
| 27 | 236 |
| 28 | 6.1 |
| 29 | 6.5 |
| 30 | 6.1 |
| 31 | 74.2 |
| 32 | 1260 |
| 33 | 18.5 |
| 34 | 9.34 |
| 35 | 27.9 |
| 36 | 105 |
| 37 | 11.2 |
| 38 | 57 |
| 39 | 39 |
| 40 | 76 |
| 41 | 16 |
| 42 | 1180 |
| 43 | 235 |
| 44 | 8 |
| 45 | 6 |
| 46 | 74 |
| 47 | 13 |
| 48 | 300 |
| 49 | 85 |
| 50 | 2700 |
| 51 | 6 |
| 52 | 6 |

### Caov3 CTG Assay—Proliferation Assay

Cell culture: Caov-3 cells (HTB-75, Lot number: 70016791, ATCC) were grown in DMEM (11960-04431053-028, Gibco) supplemented with 10% fetal bovine serum (1600-44, Gibco), 1 mM sodium pyruvate (11360-039, Gibco) and 2 mM Glutamax (35050-038, Gibco) at 37° C. with 5% 002.

Cell treatment and cell growth assessment: 18 hours post-seeding in white 384-Viewplate (6007480, PerkinElmer) at 1500 cells/well, Caov3 cells were treated for 120 hours with compounds inhibiting the METTL3/14 activity (10 serial semi-log dilutions, 30 μM as top concentration). Upon treatment, Coav-3 cells were incubated for 10 min at RT with the CellTiter-Glo reagent (G7571, Promega). Measurement of the luminescence signal was performed on a microplate reader (Ensight, PerkinElmer).

| Example | Caov3 cells |
|---|---|
| No | IC50 nM |
| 18 | 2788 |
| 19 | 364 |
| 22 | 305 |
| 23 | 888 |
| 24 | 343 |
| 25 | 2721 |
| 26 | 109.8 |
| 28 | 102.5 |
| 29 | 472.9 |
| 30 | 598 |
| 34 | 1275 |
| 39 | 5529 |
| 41 | 4066 |
| 44 | 2444 |
| 45 | 918.1 |
| 47 | 1375 |
| 51 | 1334 |
| 52 | 540.3 |

### Kasumi Cell Assay

Cell culture: KASUMI-1 cells (ACC20, Leibniz-Institut DSMZ Deutsche Sammlung von Mikroorganismen und Zellkulturen GmbH) were grown in RPMI 1640 (31870-025, Gibco) supplemented with 20% fetal bovine serum (F1524, Gibco), 1 mM sodium pyruvate (11360-039, Gibco) and 2 mM Glutamax (35050-038, Gibco) in a 5% CO2 humidified incubator at 37° C.

Cell treatment and cell growth assessment: KASUMI-1 hours were seeded in ultra-low attachment 384-well culture plate (MS-9384WZ, SBio) at a final concentration of 250,000 cells/ml (35 μL/well) and treated for 120 hours with compounds inhibiting the METTL3/14 activity (10 serial semi-log dilutions, 30 μM as top concentration). Upon treatment, Kasumi1 cells were incubated for 10 min at RT with the CellTiter-Glo reagent (G7571, Promega). Measurement of the luminescence signal was performed on a microplate reader (Ensight, PerkinElmer).

| Example | KASUMI cells |
|---|---|
| No | IC50 nM |
| 19 | 1357 |
| 22 | 1153 |
| 23 | 2936 |
| 25 | 4822 |
| 26 | 449 |
| 27 | 30000 |
| 28 | 315.8 |
| 29 | 1567 |
| 30 | 2083 |
| 39 | 6210 |
| 41 | 2658 |
| 44 | 1993 |
| 45 | 2517 |
| 47 | 4450 |
| 51 | 1860 |
| 52 | 1612 |

### Numbered Paragraphs

The following numbered paragraphs serve to define particular aspects and embodiments of the invention.
Paragraph 1. A compound of the formula (I), or a pharmaceutically acceptable salt thereof,

X—Y—Z (I)

wherein:
X is selected from:

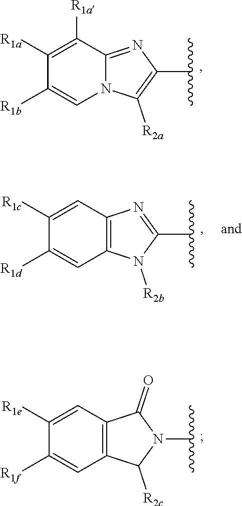

wherein:
R₁ₐ, R_{1c} and R₁ₑ are selected from hydrogen, halo, C₁₋₄ alkyl, C₂₋₃ alkenyl and —O—C₁₋₄ alkyl
R_{1b}, R_{1d} and R_{1f} are selected from:
   (i) C₁₋₄ alkyl or C₁₋₄ alkoxy, each of which being optionally substituted by halo, cyano, hydroxy, C₃₋₆ cycloalkyl, C₁₋₄ alkoxy, C₁₋₄ haloalkoxy, aryl or heteroaryl; or
   (ii) a group of the formula:

—(CR_{1c}R_{1d})ₚ—NR₁ₑR_{1f};

wherein
p is an integer selected from 0, 1, 2 or 3
R_{1c} and R_{1d} are independently selected from:
   (i) hydrogen (including deuterium),
   (ii) C₁₋₆ alkyl which is optionally substituted by one more substituents selected from cyano, oxo, hydroxy, C₁₋₄ alkoxy, halo, C₁₋₄ haloalkoxy, C₃₋₆ cycloalkyl, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkyl; and wherein C₃₋₆ cycloalkyl and —O—C₃₋₆ cycloalkyl are optionally further substituted with halo, cyano or hydroxy;
   (iii) C₃₋₄ cycloalkyl or 3 to 5 membered heterocyclyl, each of which is optionally substituted by C₁₋₄ alkyl, C₁₋₄ haloalkyl, cyano, hydroxy, C₁₋₂ alkoxy, halo, C₁₋₂ haloalkoxy, NR_{1ac}R_{1da} or —S(O)₀₋₂R_{1ca}R_{1da}, wherein R_{1ca} and R_{1da} are H or C₁₋₂ alkyl; and;
   (iv) or R_{1c} and R_{1d} are linked together such that, together with the carbon atom to which they are attached, they form a 3- to 6-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl;
R₁ₑ and R_{1f} are each independently selected from:
   (i) hydrogen (including deuterium);
   (ii) C₁₋₆ alkyl which is optionally substituted by one more substituents selected from cyano, oxo, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
   (iii) a group with the formula:

—(CR_{1g}R₁ₕ)_{q}-T₁

wherein:
q is 0, 1, 2, 3, 4, 5 or 6;
R_{1g} and R₁ₕ are independently selected from:
   a) hydrogen (including deuterium);
   b) C₁₋₆ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
   c) an aryl-C₁₋₆ alkyl, heteroarylC₁₋₆ alkyl, C₃₋₆ cycloalkyl or C₃₋₆ cycloalkylC₁₋₆ alkyl group, each of which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl; or
   d) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
   and T₁ is selected from hydrogen, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
R_{1a′} is selected from hydrogen, halo and methyl;
R₂ₐ, R_{2b} and R_{2c} are selected from hydrogen, halo or a group of the formula:

-L₂ₐ-L_{2b}-Q₂

wherein
L₂ₐ is absent or C₁₋₃ alkylene optionally substituted by C₁₋₂ alkyl or oxo;
L_{2b} is absent or selected from O, S, SO, SO₂, N(Rₙ), C(O), C(O)O, OC(O), C(O)N(Rₙ),
N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂ alkyl; and
Q₂ is hydrogen, cyano, C₁₋₆ alkyl, C₃₋₆ cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄ alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)_{z}NRₚR_{q} (where z is 1, 2 or 3), wherein Rₚ and R_{q} are each independently selected from hydrogen or C₁₋₄ alkyl;
Y is selected from:

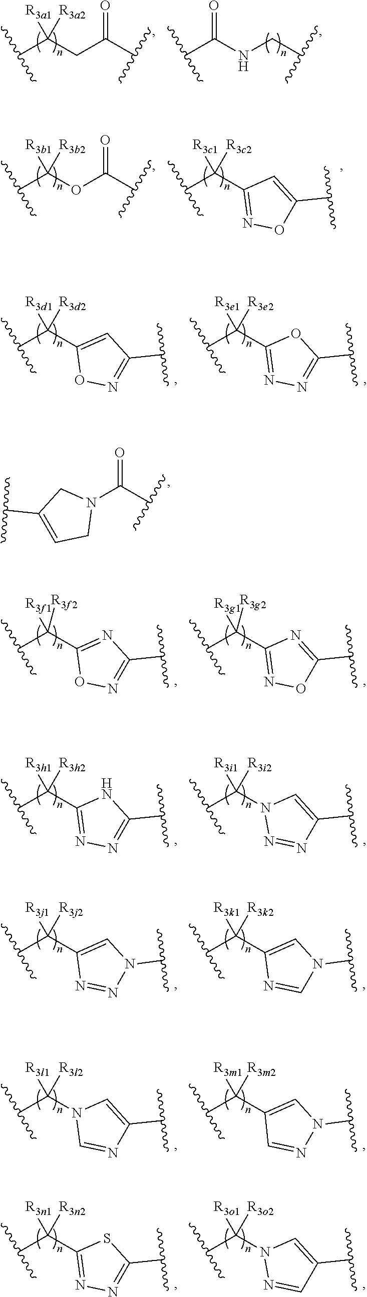

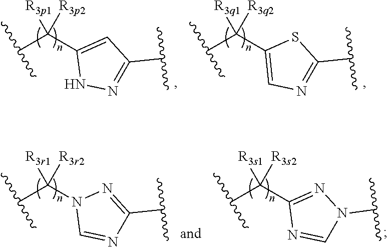

wherein:
R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen (including deuterium), C₁₋₆ alkyl, C₃₋₄ cycloalkyl, hydroxy, and halo; and wherein C₁₋₆ alkyl, or C₃₋₄ cycloalkyl is optionally substituted with one or more substituents selected from halo, amino, cyano, and hydroxy;
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen or halo; with the proviso that R₃ₐ₁, R_{3b1}, R₃ᵢ₁, R₃ₗ₁, R₃ₒ₁, R₃ᵣ₁, R₃ₐ₂, R_{3b2}, R₃ᵢ₂, R₃ₗ₂, R₃ₒ₂ and R₃ₛ₁ cannot be halo when n=1 or when n=2 and the carbon atom to which they are attached is linked to an oxygen or nitrogen atom;
or R₃ₐ₁ and R₃ₐ₂, R_{3b1} and R_{3b2}, R₃ₐ₁ and R_{3c2}, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, or R₃ᵣ₁ and R₃ᵣ₂ or R₃ₛ₁ and R₃ₛ₂ may be linked such that, together with the carbon atom to which they are attached, they form a spiro-fused C₃₋₄ cycloalkyl which is optionally substituted with one or more substituents selected from halo, methyl, amino, cyano, and hydroxy;
n is 0, 1 or 2; and
Z is selected from:

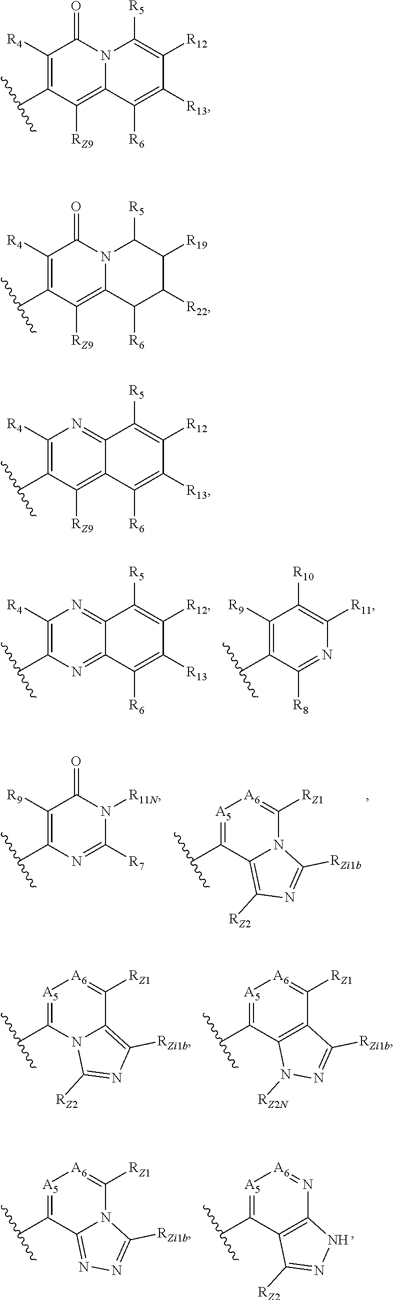

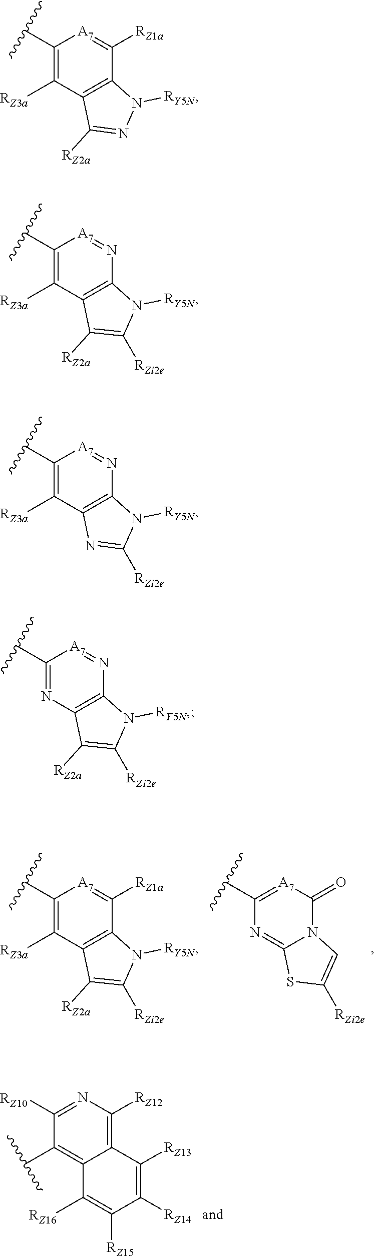

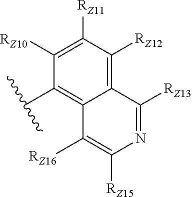

wherein:
R₄ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₅ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₆ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₁₂ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and C₁₋₄ alkyl);
R₁₃ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano, methoxy and methyl);
R₇, R₉ and R₁₁ are independently selected from hydrogen, NH₂, halo, cyano, C₁₋₄ alkoxy, C₁₋₄ haloalkoxy and C₁₋₆ alkyl; R₈ and R₁₀ are independently selected from halo, cyano, C₁₋₄ alkoxy, C₁₋₄ haloalkoxy, C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl;
R_{11N} are independently selected from hydrogen and C₁₋₆ alkyl;
R_{Z1} and R_{Z1a} selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
R_{Z2} and R_{Z2a} are selected from hydrogen, C₁₋₄ alkyl, cyano, halo, NH₂ and C₁₋₄ alkoxy;
R_{Z3a} is selected from hydrogen, C₁₋₄ alkyl, cyano, halo, NH₂ and C₁₋₄ alkoxy;
R_{Zi1b} is selected from hydrogen, C₁₋₄ alkyl, cyano, halo, NH₂ and C₁₋₄ alkoxy;
R_{Zi2e} is selected from hydrogen, C₁₋₄ alkyl, cyano, halo, NH₂ and C₁₋₄ alkoxy;
R_{Z2N} is selected from hydrogen or C₁₋₄ alkyl
R_{Y5N} is selected from hydrogen or C₁₋₄ alkyl
R_{Z9} is selected from hydrogen, halo, cyano, C₁₋₄ alkyl, C₁₋₄ haloalkyl, C₁₋₄ alkoxy, C₁₋₄ haloalkoxy;
R_{Z10} is selected from hydrogen, halo, cyano, C₁₋₄ alkyl, C₁₋₄ haloalkyl, C₁₋₄ alkoxy, C₁₋₄ haloalkoxy;
R_{Z11} is selected from hydrogen, halo, cyano, C₁₋₄ alkyl, C₁₋₄ haloalkyl, C₁₋₄ alkoxy, C₁₋₄ haloalkoxy;
R_{Z12} is selected from hydrogen, halo, cyano, C₁₋₄ alkyl, C₁₋₄ haloalkyl, C₁₋₄ alkoxy, C₁₋₄ haloalkoxy;
R_{Z13} is selected from hydrogen, halo, cyano, C₁₋₄ alkyl, C₁₋₄ haloalkyl, C₁₋₄ alkoxy, C₁₋₄ haloalkoxy;
R_{Z14} is selected from hydrogen, halo, cyano, C₁₋₄ alkyl, C₁₋₄ haloalkyl, C₁₋₄ alkoxy, C₁₋₄ haloalkoxy;
R_{Z15} is selected from hydrogen, halo, cyano, C₁₋₄ alkyl, C₁₋₄ haloalkyl, C₁₋₄ alkoxy, C₁₋₄ haloalkoxy;
R_{Z16} is selected from hydrogen, halo, cyano, C₁₋₄ alkyl, C₁₋₄ haloalkyl, C₁₋₄ alkoxy, C₁₋₄ haloalkoxy;
A₅ is selected from CR₁₆ and N;
A₆ is selected from CR₁₇ and N;
A₇ is selected from CR₁₈ and N;
R₁₅ is selected from hydrogen, halo, cyano, methoxy and methyl;
R₁₆ and R₁₈, are selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄-cycloalkoxy;
R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₅ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring; wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O— heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl; R₁₉ is selected from hydrogen, halo, cyano and C₁₋₄ alkyl; R₂₂ is selected from hydrogen, halo, cyano, C₁₋₄ alkyl, C₁₋₄ haloalkyl, C₁₋₄ alkoxy, C₁₋₄ haloalkoxy.
Paragraph 2. A compound according to paragraph 1, or a pharmaceutically acceptable salt thereof, wherein R_{1b}, R_{1d} and R_{1f} are a group of the formula:

—(CR_{1c}R_{1d})ₚ—NR₁ₑR_{1f};

wherein
p is an integer selected from 1 or 2;
R₁ₑ and R_{1d} are independently selected from:
   (i) hydrogen (including deuterium),
   (ii) C₁₋₃ alkyl which is optionally substituted by one more substituents selected from cyano, oxo, hydroxy, C₁₋₃ alkoxy, halo, C₁₋₄₃ haloalkoxy, —O—C₃₋₄ cycloalkyl, or NH₂; wherein —O—C₃₋₆ cycloalkyl is optionally substituted with halo, cyano or hydroxy,
   (iii) or R_{1c} and R_{1d} are linked together such that, together with the carbon atom to which they are attached, they form a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ca}R_{1da} or —S(O)₀₋₂R_{1ac}R_{1da}, wherein R_{1ca} and R_{1da} are H or C₁₋₂ alkyl;
R₁ₑ is selected from:
   (i) hydrogen (including deuterium);
   (ii) C₁₋₃ alkyl which is optionally substituted by one more substituents selected from cyano, oxo, hydroxy, C₁₋₂ alkoxy, halo, C₁₋₂ haloalkoxy and NH₂;
and R_{1f} is selected from:
   (i) C₁₋₆ alkyl which is optionally substituted by one more substituents selected from cyano, oxo, hydroxy, C₁₋₂ alkoxy, halo, C₁₋₂ haloalkoxy and NH₂;
   (ii) a group with the formula:

—(CR_{1g}R₁ₕ)_{q}-T₁

wherein:
q is 1, 2 or 3;
R_{1g} and R₁ₕ are independently selected from:
   a) hydrogen (including deuterium); or
   b) C₁₋₆ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkylNR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
   c) or R_{1g} and R₁ₕ are optionally linked together such that, together with the carbon atom to which they are attached, they form a 3- to 4-membered cycloalkyl or heterocyclic ring which is optionally substituted by one or more substituents selected from C₁₋₂ alkyl, C₁₋₂-haloalkyl, cyano, hydroxy, C₁₋₂ alkoxy, halo, C₁₋₂-haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
   and T₁ is selected from hydrogen, halo, C₁₋₄ alkyl, C₁₋₄ haloalkyl, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂-haloalkyl, cyano, hydroxy, C₁₋₂ alkoxy, halo, C₁₋₂ haloalkoxy, C₃₋₆ cycloalkyl, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring; which in turn is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ᵢ, wherein R₁ₖ and R₁ₗ are H or C₁₋₄ alkyl.
Paragraph 3. A compound according to paragraph 1 or 2, or a pharmaceutically acceptable salt thereof, wherein R_{1b}, R_{1d} and R_{1f} are a group of the formula:

—(CR_{1c}R_{1d})ₚ—NR₁ₑR_{1f};

wherein
p is 1;
R_{1ca}nd R_{1d} are independently selected from hydrogen (including deuterium) or C₁₋₂ alkyl;
R₁ₑ is selected from hydrogen (including deuterium) or C₁₋₂ alkyl; and
R_{1f} is a group with the formula:

—(CR_{1g}R₁ₕ)_{q}-T₁
wherein:
q is 1 or 2;
R_{1g} and R₁ₕ are independently selected from hydrogen (including deuterium) or C₁₋₂ alkyl;
and T₁ is selected from C₃₋₄ cycloalkyl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy or C₃₋₆ cycloalkyl;
or R₁ₑ and R_{1f} are linked such that, together with the nitrogen atom to which they are attached, they form a mono- or bicyclic-heterocyclic ring, which is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, and/or the mono- or bicyclic heterocyclic ring formed by R₁ₑ and R_{1f} is optionally spiro-fused to a C₃₋₆ cycloalkyl or a heterocyclic ring; which in turn is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₖ are H or C₁₋₄ alkyl.
Paragraph 4. A compound according to any one of the preceding paragraphs, or a pharmaceutically acceptable salt thereof, wherein R₁ₐ, R_{1c} and R₁ₑ are selected from hydrogen, fluoro, bromo, methyl, ethenyl and —O-methyl.
Paragraph 5. A compound according to any one of the preceding paragraphs, or a pharmaceutically acceptable salt thereof, wherein R₁ₐ, R_{1c} and R₁ₑ are hydrogen.
Paragraph 6. A compound according to any one of the preceding paragraphs, or a pharmaceutically acceptable salt thereof, wherein R₂ₐ, R_{2b} and R_{2c} are hydrogen.
Paragraph 7. A compound according to any one of the preceding paragraphs, or a pharmaceutically acceptable salt thereof, wherein R_{1b}, R_{1d} and R_{1f} are selected from

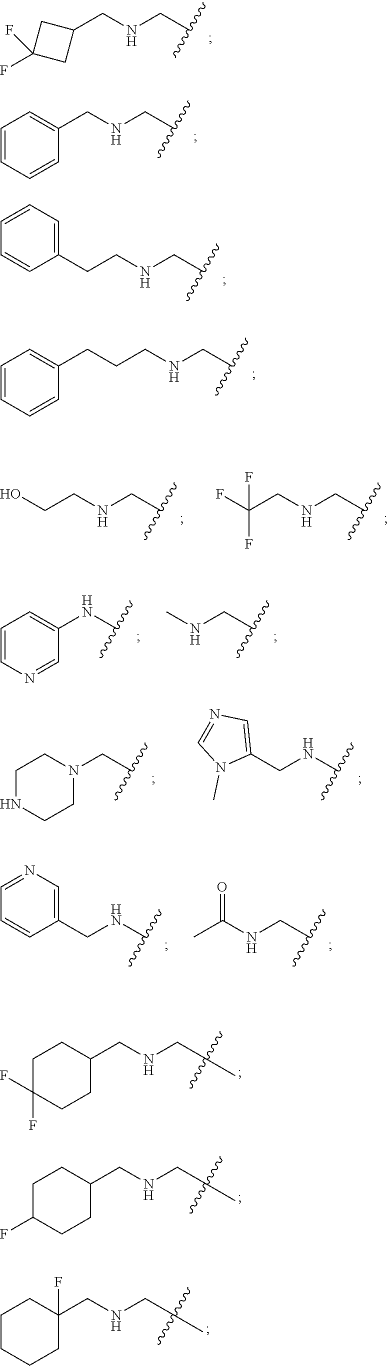

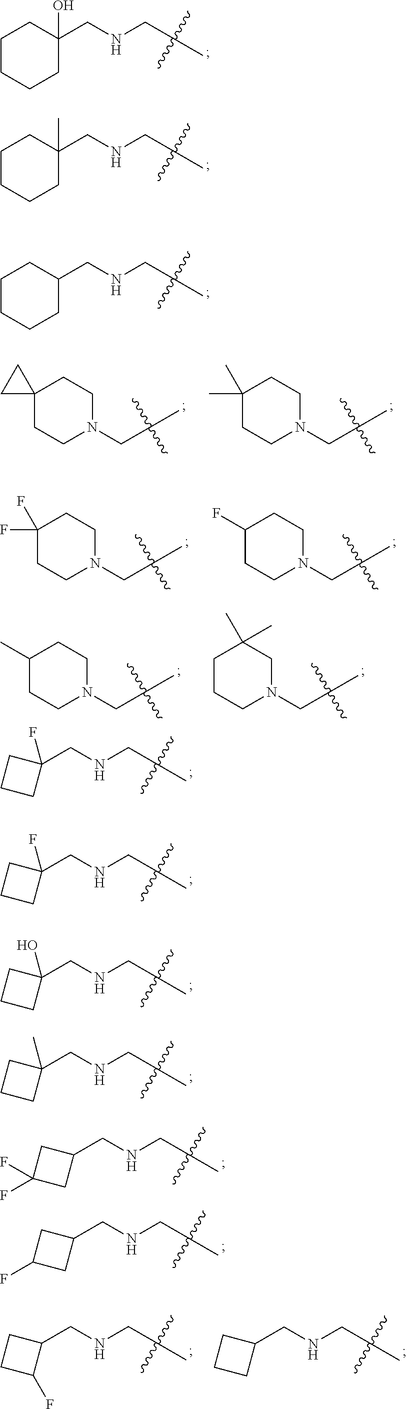

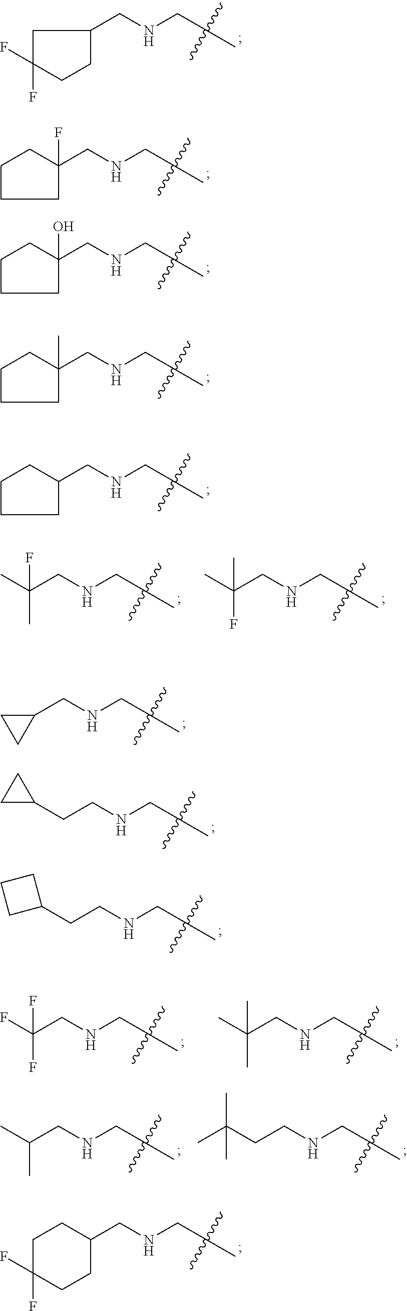

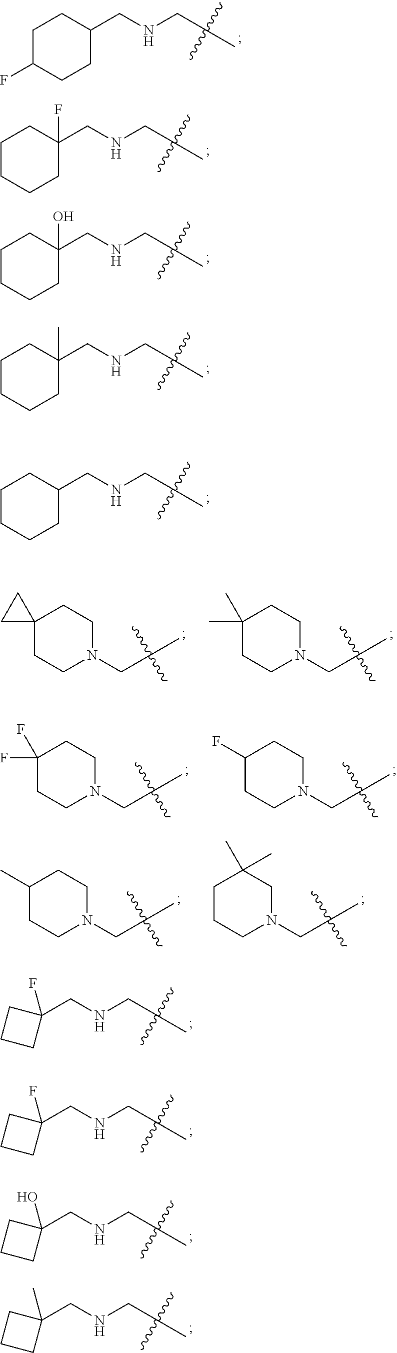

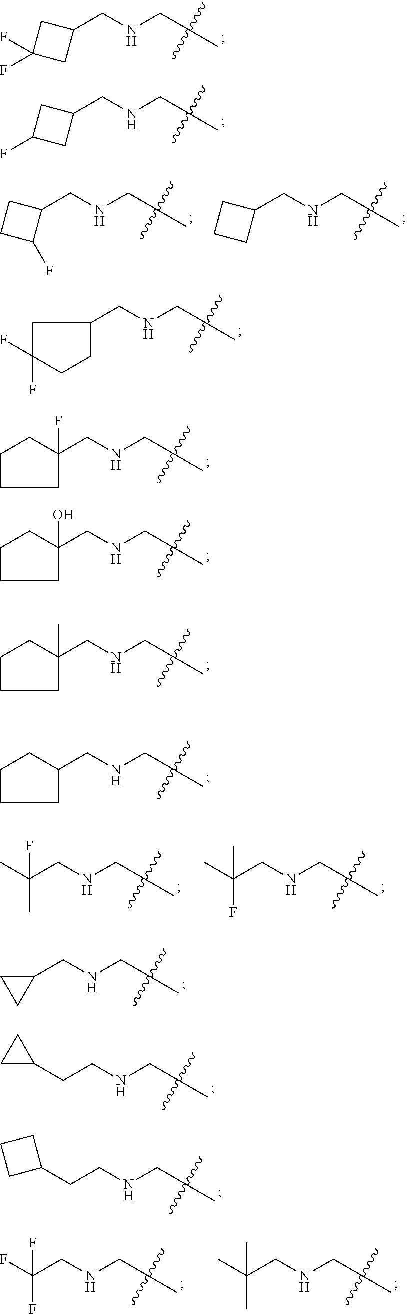

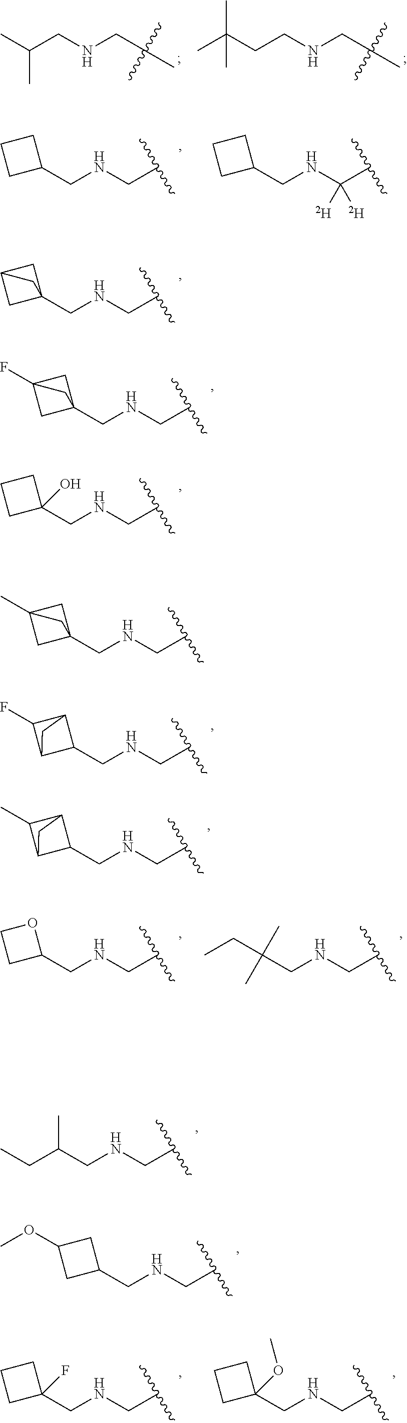

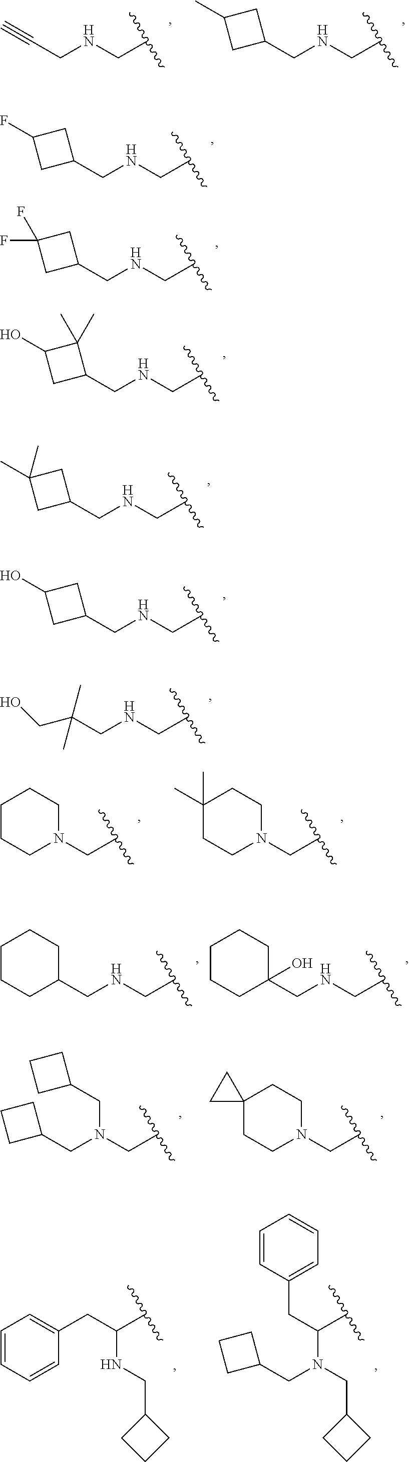

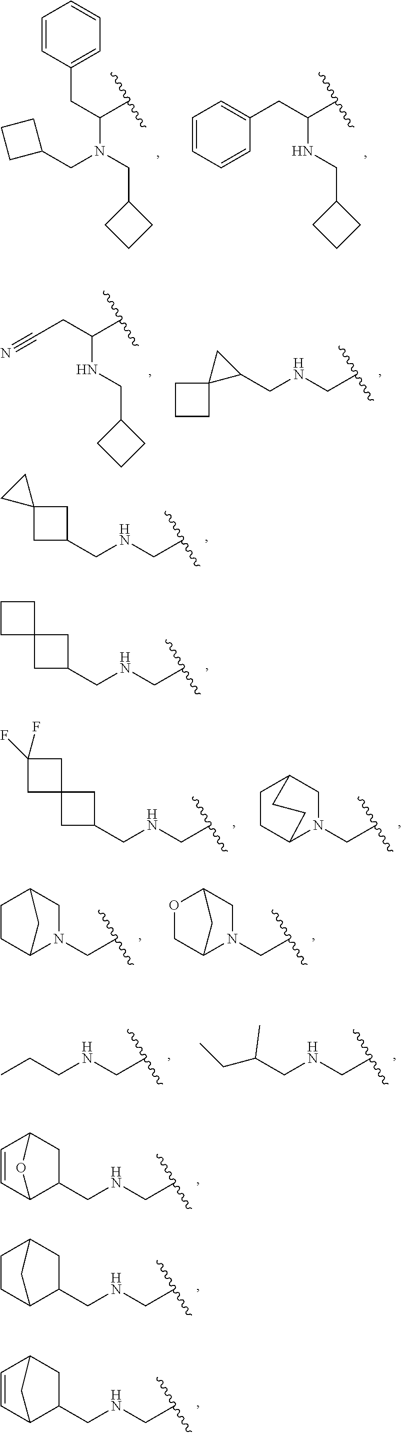

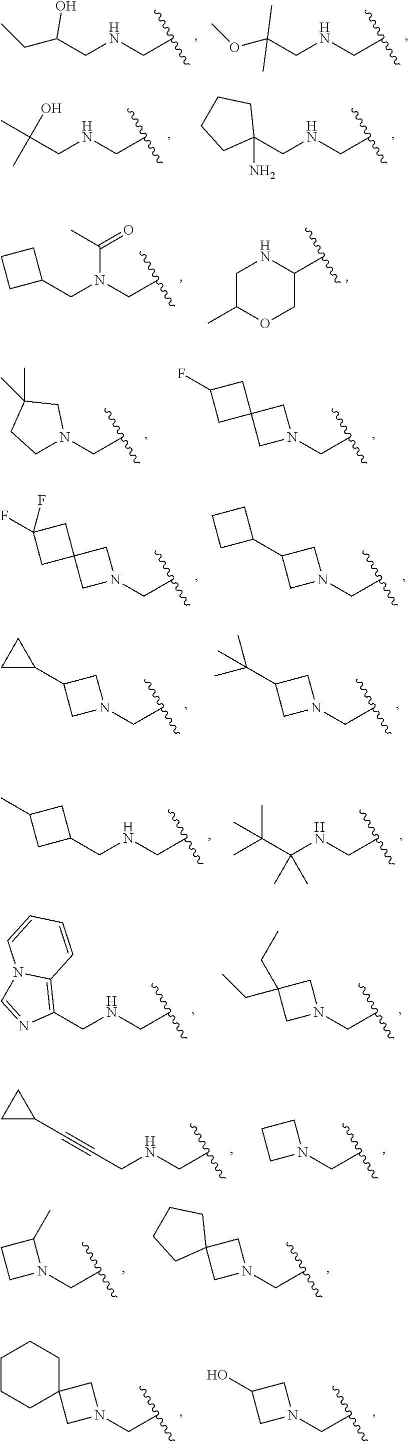

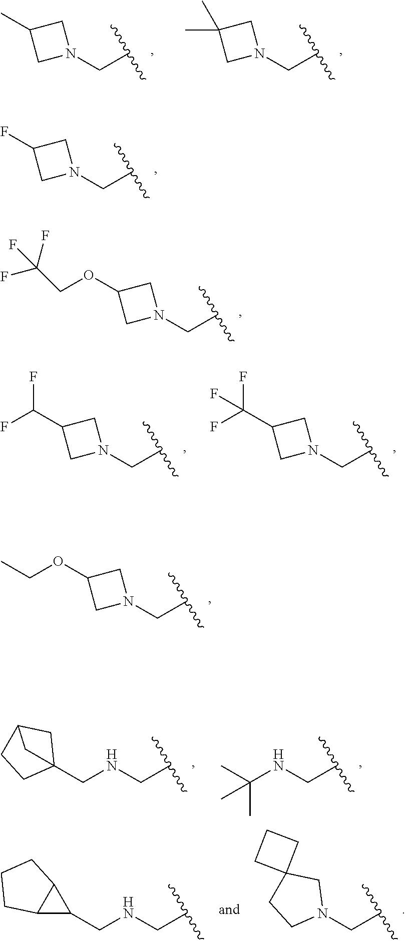

Paragraph 8. A compound according to any one of the preceding paragraphs, or a pharmaceutically acceptable salt thereof, wherein R_{1b}, R_{1d} and R_{1f} are selected from:

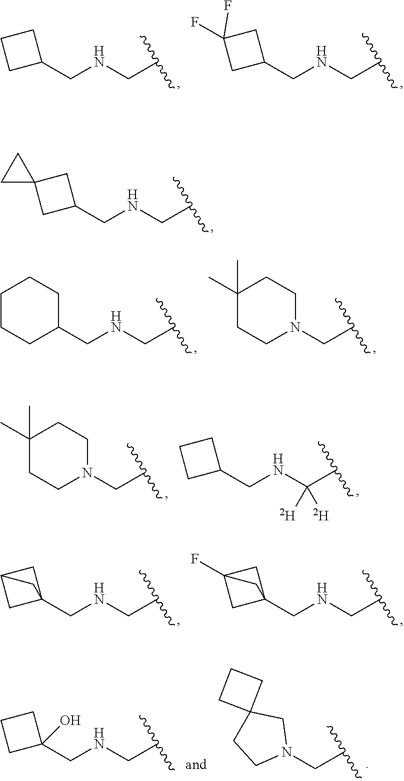

Paragraph 9. A compound according to any one of the preceding paragraphs, or a pharmaceutically acceptable salt thereof, wherein R_{1b}, R_{1d} and R_{1f} are selected from;

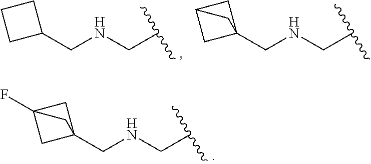

Paragraph 10. A compound according to any one of the preceding paragraphs, or a pharmaceutically acceptable salt thereof, wherein X is:

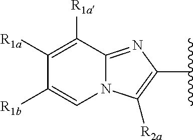

and R₁ₐ, R_{1b}, R_{1a′} and R₂ₐ are as defined in any of the preceding paragraphs.
Paragraph 11. A compound according to any one of the preceding paragraphs, or a pharmaceutically acceptable salt thereof, wherein Y is selected from:

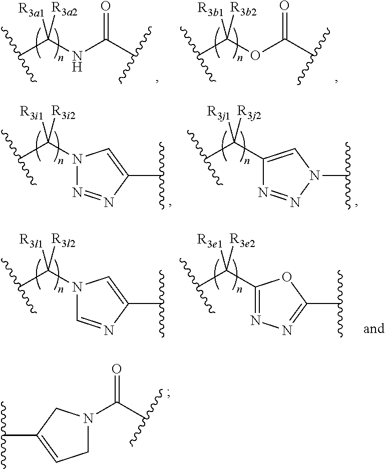

wherein R₃ₐ₁, R₃ₐ₂, R_{3b1}, R_{3b2}, R₃ₑ₁, R₃ₑ₂, R₃ᵢ₁, R₃ᵢ₂, R₃ⱼ₁, R₃ⱼ₂, R₃ᵢ₁ and R₃ᵢ₂, are as defined preceding paragraphs.
Paragraph 12. A compound according to any one of the preceding paragraphs, or a pharmaceutically acceptable salt thereof, wherein, R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen and C₁₋₆ alkyl; and wherein C₁₋₆ alkyl is optionally substituted with one or more hydroxy substituents.
Paragraph 13. A compound according to any one of the preceding paragraphs, or a pharmaceutically acceptable salt thereof, wherein R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen.
Paragraph 14. A compound according to any one of the preceding paragraphs, or a pharmaceutically acceptable salt thereof, wherein n is 1.
Paragraph 15. A compound according to any one of the preceding paragraphs, or a pharmaceutically acceptable salt thereof, wherein Y is selected from:

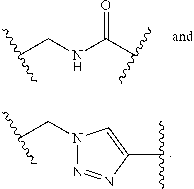 [SMILES: *CNC(*)=O ; *Cn1cc(*)nn1]

Paragraph 16. A compound according to any one of the preceding paragraphs, or a pharmaceutically acceptable salt thereof, wherein Y is

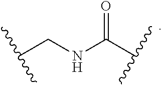 [SMILES: *CNC(*)=O]

Paragraph 17. A compound according to any one of the preceding paragraphs, or a pharmaceutically acceptable salt thereof, wherein
   A₅ is CR₁₆;
   A₆ is CR₁₇;
   A₇ is CR₁₃;
   and wherein R₁₂, R₁₃, R₁₆, R₁₇, R₁₈, R₁₉, and R₂₂ are as defined in any one of the preceding paragraphs.
Paragraph 18. A compound according to any one of the preceding paragraphs, or a pharmaceutically acceptable salt thereof, wherein:
   (i) R₄, R₅, R₆, R₇, R₉, R₁₁, R_{Z2}, R_{Z2a}, R_{Z3a}, R_{Zi1b}, R_{Zi2e}, R_{Z9}, R_{Z10}, R_{Z11}, R_{Z12}, R_{Z13}, R_{Z14}, R_{Z15} and R_{Z16} are independently selected from hydrogen, methyl, cyano or halo; and
   R_{B5N}, R_{Y5N}, and R_{11N} are selected from methyl or hydrogen;
   (ii) R₈ is selected from cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl;
   (iii) R₁₀ is selected from C₁₋₄ alkoxy or C₁₋₄ haloalkoxy;
   (iv) R_{Z1} and R_{Z1a} are selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl;
   (v) R₁₂, R₁₃, R₁₆, R₁₈, R₁₉ and R₂₂ are independently selected from hydrogen, halo, cyano and methyl;
   (vi) R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl a 5- or 6-membered or heteroaryl, C₃₋₆ cycloalkyl, —O—C₃₋₆ cycloalkyl, heterocyclyl, —(OCH₂CH₂)ₘ—OCH₃
   wherein m is an integer from 1 to 6, NR_{q}Rᵣ, wherein R_{q} and Rᵣ are each independently hydrogen, C₁₋₄ alkyl or R_{q} and Rᵣ are linked together such that, together with the nitrogen atom to which they are attached, they form a 3- to 6-membered heterocyclic ring; wherein any C₁₋₄ alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.
Paragraph 19. A compound according to any one of the preceding paragraphs, or a pharmaceutically acceptable salt thereof, wherein:
   (i) R₄, R₅, R₆, R₇, R₉, R₁₁, R_{Z2}, R_{Z2a}, R_{Z3a}, R_{Zi1b}, R_{Zi2e}, R_{Z9}, R_{Z10}, R_{Z11}, R_{Z12}, R_{Z13}, R_{Z14}, R_{Z15} and R_{Z16} are hydrogen;
   (ii) R₈ is cyano;
   (iii) R₁₀ is methoxy;
   (iv) R_{Z1} and R_{Z1a} are selected from hydrogen, cyano, halo, C₁₋₂ haloalkyl, C₁₋₂ haloalkoxy, C₁₋₂ alkoxy, C₃₋₆ cycloalkyl and —O—C₃₋₆ cycloalkyl;
   (v) R₁₂, R₁₃, R₁₆, R₁₈, R₁₉ and R₂₂ are hydrogen;
   (vi) R₁₇ is selected from hydrogen, halo, cyano, C₁₋₂ alkyl, C₁₋₂ haloalkyl, C₁₋₂ alkoxy, C₁₋₂ haloalkoxy, C₂₋₃ alkenyl, C₂₋₃ alkynyl, phenyl a 5- or 6-membered or heteroaryl, C₃₋₆ cycloalkyl, —O—C₃₋₆ cycloalkyl, heterocyclyl, —(OCH₂CH₂)ₘ—OCH₃ wherein m is an integer from 1 to 6, NR_{q}Rᵣ, wherein R_{q} and Rᵣ are each independently hydrogen, C₁₋₂ alkyl or R_{q} and Rᵣ are linked together such that, together with the nitrogen atom to which they are attached, they form a 3- to 6-membered heterocyclic ring; wherein any C₁₋₄ alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.
Paragraph 20. A compound according to any one of the preceding paragraphs, or a pharmaceutically acceptable salt thereof, wherein Z is selected from:

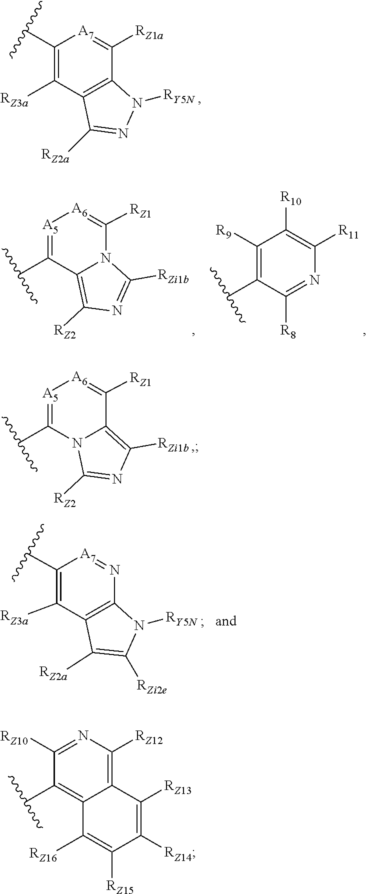

wherein A₅, A₆, A₇, R₄, R₅, R₆, R₇, R₈, R_{B5N}, R_{Y5N}, R_{Z1}, R_{Z2}, R_{Z10}, R_{Z12}, R_{Z13}, R_{Z14}, R_{Z15}, R_{Z16}, R_{Z2a}, R_{Z3a}, R_{Z1a}, R_{Zi1b} and R_{Zi2e} are as defined in any one of the preceding paragraphs.
Paragraph 21. A compound according to any one of the preceding paragraphs, or a pharmaceutically acceptable salt thereof, wherein Z is:

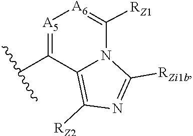

wherein A₅, A₆, R_{Z1}, R_{Z2} and R_{Zi1b} are as defined in any one of the preceding paragraphs.
Paragraph 22. A compound according to any one of the preceding paragraphs, or a pharmaceutically acceptable salt thereof, wherein A₆ is CR₁₇ and wherein R₁₇ is selected from selected from hydrogen, halo, cyano, C₁₋₅ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₄ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring; wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.
Paragraph 23. A compound according to paragraph 21, or a pharmaceutically acceptable salt thereof, wherein R₁₇ is selected from hydrogen, halo, C₁₋₄ alkoxy or C₁₋₄ haloalkoxy.
Paragraph 24. A compound according to paragraph 21, or a pharmaceutically acceptable salt thereof, wherein A₅ is R₁₆, and R₁₆ is selected from hydrogen, halo, cyano and C₁₋₄ alkyl.
Paragraph 25. A compound according to any one of the preceding paragraphs, or a pharmaceutically acceptable salt thereof, wherein Z is selected from:

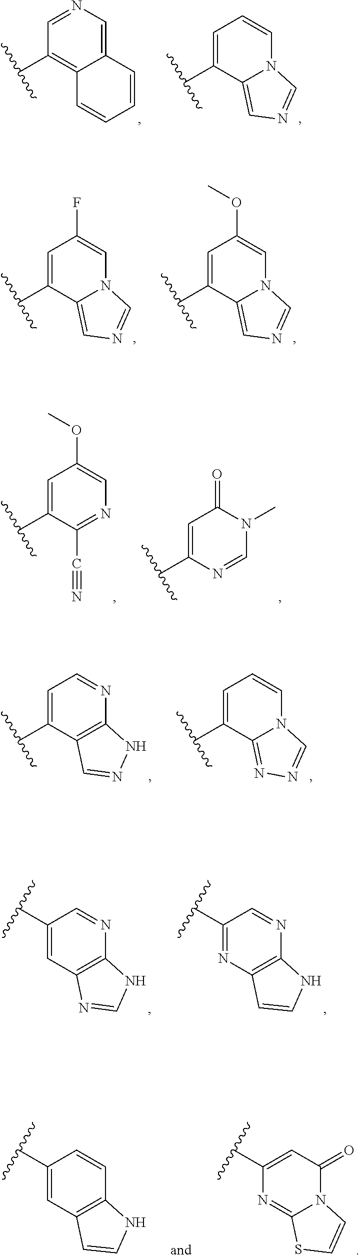

Paragraph 26. A compound according to paragraph 1, or a pharmaceutically acceptable salt thereof, wherein:

X is

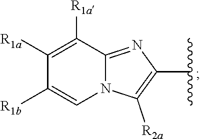

Y is selected from

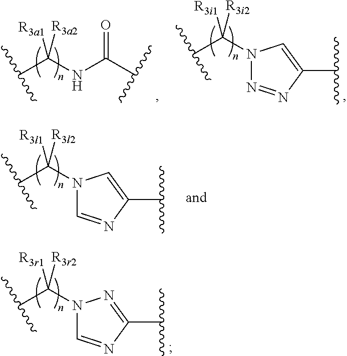

and
Z is selected from:

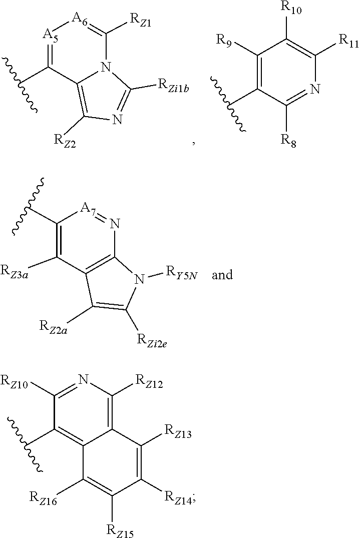

wherein
(i) R₁ₐ, R₁ₐ, R_{1b}, and R₂ₐ are as defined in any one of the preceding paragraphs;
(ii) R₃ₐ₁, R₃ₐ₂, R₃ᵢ₁, R₃ᵢ₂, R₃ₗ₁, R₃ₗ₂, R₃ᵣ₁, R₃ᵣ₂, are as defined in any one of the preceding paragraphs;
(iii) A₅, A₆, A₇, R₈, R₉, R₁₀, R₁₁, R_{Z1}, R_{Z2}, R_{Zi1b}, R_{Z10}, R_{Z12}, R_{Z13}, R_{Z14}, R_{Z15}, R_{Z16}, R_{Y5N}, R_{Z2a}, R_{Z3a}, and R_{Zi2e} are as defined in any one of the preceding paragraphs.
Paragraph 27. A compound according to paragraph 26, or a pharmaceutically acceptable salt thereof, wherein:
X is

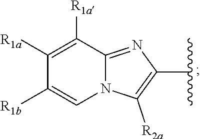

Y is

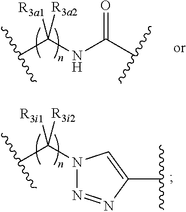

and
Z is selected from:

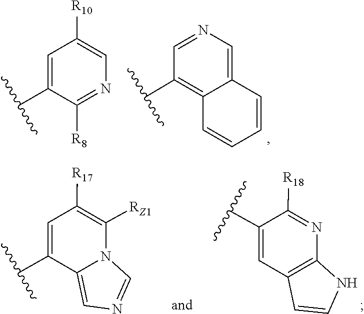

wherein
(i) R₁ₐ, R_{1a′}, R_{1b}, and R₂ₐ are as defined in any one of the preceding paragraphs;
(ii) R₃ₐ₁, R₃ₐ₂, R₃ᵢ₁, R₃ᵢ₂, R₃ₗ₁, R₃ₗ₂, R₃ᵣ₁, R₃ᵣ₂, are as defined in any one of the preceding paragraphs;
(iii) R₈, R₁₀, R₁₇, R₁₈ and R_{Z1}, are as defined in any one of the preceding paragraphs.
Paragraph 28. A compound according to paragraph 26, or a pharmaceutically acceptable salt thereof, wherein:
X is

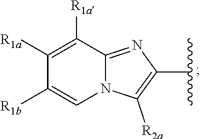

Y is

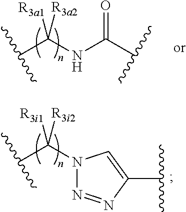

and
Z is
   wherein:
(i) R₁ₐ, R_{1a′}, R_{1b}, and R₂ₐ are as defined in any one of the preceding paragraphs;
(ii) R₃ₐ₁, R₃ₐ₂, R₃ᵢ₁ and R₃ᵢ₂ are as defined in any one of the preceding paragraphs;
(iii) A₅, A₆, R_{Z1}, R_{Z2} and R_{zi1b} are as defined in any one of the preceding paragraphs.
Paragraph 29. A compound according to paragraph 26, or a pharmaceutically acceptable salt thereof, wherein:
X is:

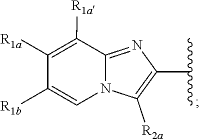

Y is:

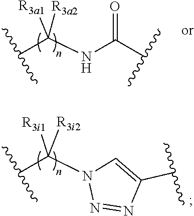

and
Z is selected from:

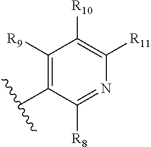

wherein
(i) R₁ₐ, R^{1a′}, R_{1b}, and R₂ₐ are as defined in any preceding paragraph;
(ii) R₃ₐ₁, R₃ₐ₂, R₃ᵢ₁, and R₃ᵢ₂ are as defined in any preceding paragraph;
(iii) R₃ is selected from cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl,
   R₉ and R₁₁ are as defined in any preceding paragraph;
   R₁₀ is C₁₋₄ alkoxy or C₁₋₄ haloalkoxy.
Paragraph 30. A compound, or a pharmaceutically acceptable salt thereof, and, in particular, selected from:
N-[(6-{[(cyclohexylmethyl)amino]methyl}imidazo[1,2-a]pyridin-2-yl)methyl]-1,2-dimethyl-6-oxo-1,6-dihydropyrimidine-4-carboxamide;
N-({6-methylimidazo[1,2-a]pyridin-2-yl}methyl)-4-oxo-4H-quinolizine-2-carboxamide;
N-[(6-{[(cyclohexylmethyl)amino]methyl}imidazo[1,2-a]pyridin-2-yl)methyl]-4-oxo-4H-quinolizine-2-carboxamide;
N-[(6-{[(cyclohexylmethyl)amino]methyl}imidazo[1,2-a]pyridin-2-yl)methyl]quinoxaline-2-carboxamide;
N-[(6-{[(cyclohexylmethyl)amino]methyl}imidazo[1,2-a]pyridin-2-yl)methyl]-1H-pyrazolo[3,4-b]pyridine-4-carboxamide;
N-[(6-{[(cyclohexylmethyl)amino]methyl}imidazo[1,2-a]pyridin-2-yl)methyl]quinoline-3-carboxamide;
N-[(6-{[(cyclobutylmethyl)amino]methyl}imidazo[1,2-a]pyridin-2-yl)methyl]imidazo[1,5-a]pyridine-8-carboxamide;
N-[(6-{[(cyclobutylmethyl)amino]methyl}imidazo[1,2-a]pyridin-2-yl)methyl]imidazo[1,5-a]pyridine-5-carboxamide;
N-[(6-{[(cyclobutylmethyl)amino]methyl}imidazo[1,2-a]pyridin-2-yl)methyl]-3H-imidazo[4,5-b]pyridine-6-carboxamide;
N-[(6-{[(cyclobutylmethyl)amino]methyl}imidazo[1,2-a]pyridin-2-yl)methyl]-1H-pyrrolo[2,3-b]pyridine-5-carboxamide;
N-{[6-({[(3,3-difluorocyclobutyl)methyl]amino}methyl)imidazo[1,2-a]pyridin-2-yl]methyl}-1H-pyrrolo[2,3-b]pyridine-5-carboxamide;
N-[(6-{[(cyclobutylmethyl)amino]methyl}imidazo[1,2-a]pyridin-2-yl)methyl]-1-methyl-1H-pyrrolo[2,3-b]pyridine-5-carboxamide
N-[(6-{[(cyclobutylmethyl)amino]methyl}imidazo[1,2-a]pyridin-2-yl)methyl]-1H-indole-5-carboxamide;
N-[(6-{[(cyclobutylmethyl)amino]methyl}imidazo[1,2-a]pyridin-2-yl)methyl]-5H-pyrrolo[2,3-b]pyrazine-2-carboxamide;
N-[(6-{[({3-fluorobicyclo[1.1.1]pentan-1-yl}methyl)amino]methyl}imidazo[1,2-a]pyridin-2-yl)methyl]-5-oxo-5H-[1,3]thiazolo[3,2-a]pyrimidine-7-carboxamide;
4-[1-[(6-methylimidazo[1,2-a]pyridin-2-yl)methyl]triazol-4-yl]isoquinoline;
1-({6-methylimidazo[1,2-a]pyridin-2-yl}methyl)-4-{1H-pyrrolo[2,3-b]pyridin-5-yl}-1H-1,2,3-triazole;
(cyclobutylmethyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{imidazo[1,5-a]pyridin-8-yl}-1H-1,2,3-triazol-1-yl)methyl]imidazo[1,2-a]pyridin-6-yl}methyl)amine;
({2-[(4-{6-chloroimidazo[1,5-a]pyridin-8-yl}-1H-1,2,3-triazol-1-yl)methyl]imidazo[1,2-a]pyridin-6-yl}methyl)(cyclobutylmethyl)amine;
(cyclobutylmethyl)({2-[(4-{1-fluoroimidazo[1,5-a]pyridin-8-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{[1,2,4]triazolo[4,3-a]pyridin-8-yl}-1H-1,2,3-triazol-1-yl)methyl]imidazo[1,2-a]pyridin-6-yl}methyl)amine;
({bicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
2-({2-[(4-{imidazo[1,5-a]pyridin-8-yl}-1H-1,2,3-triazol-1-yl)methyl]imidazo[1,2-a]pyridin-6-yl}methyl)-2-azaspiro[3.3]heptane;
2-({2-[(4-{6-methoxyimidazo[1,5-a]pyridin-8-yl}-1H-1,2,3-triazol-1-yl)methyl]imidazo[1,2-a]pyridin-6-yl}methyl)-2-azaspiro[3.3]heptane;
N-(cyclobutylmethyl)-1-[2-[[4-(4-isoquinolyl)triazol-1-yl]methyl]imidazo[1,2-a]pyridin-6-yl]methanamine;
bis(cyclobutylmethyl)[(2-{[4-(isoquinolin-4-yl)-1H-1,2,3-triazol-1-yl]methyl}imidazo[1,2-a]pyridin-6-yl)methyl]amine;
(cyclobutylmethyl)({2-[(4-{1H-pyrrolo[2,3-b]pyridin-5-yl}-1H-1,2,3-triazol-1-yl)methyl]imidazo[1,2-a]pyridin-6-yl}methyl)amine;
(cyclobutylmethyl)({2-[(4-{1H-pyrrolo[2,3-b]pyridin-5-yl}-1H-imidazol-1-yl)methyl]imidazo[1,2-a]pyridin-6-yl}methyl)amine;
cyclobutylmethyl)({2-[(4-{imidazo[1,5-a]pyridin-5-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{3-methylimidazo[1,5-a]pyridin-8-yl}-1H-1,2,3-triazol-1-yl)methyl]imidazo[1,2-a]pyridin-6-yl}methyl)amine;
3-{1-[(6-{[({bicyclo[1.1.1]pentan-1-yl}methyl)amino]methyl}imidazo[1,2-a]pyridin-2-yl)methyl]-1H-1,2,3-triazol-4-yl}-5-methoxypyridine-2-carbonitrile;
3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}-5-methoxypyridine-2-carbonitrile;
3-{1-[(6-{[(cyclobutylmethyl)amino]methyl}imidazo[1,2-a]pyridin-2-yl)methyl]-1H-1,2,3-triazol-4-yl}-5-methoxypyridine-2-carbonitrile;
3-(1-{[6-({6-azaspiro[3.4]octan-6-yl}methyl)imidazo[1,2-a]pyridin-2-yl]methyl}-1H-1,2,3-triazol-4-yl)-5-methoxypyridine-2-carbonitrile;
3-[1-({6-[(4,4-dimethylpiperidin-1-yl)methyl]imidazo[1,2-a]pyridin-2-yl}methyl)-1H-1,2,3-triazol-4-yl]-5-methoxypyridine-2-carbonitrile.
   Paragraph 31. A pharmaceutical composition comprising a compound according to any one of paragraphs 1 to 30, or a pharmaceutically acceptable salt thereof, and one or more pharmaceutically acceptable excipients.
   Paragraph 32. A compound according to any one of paragraphs 1 to 30, or a pharmaceutically acceptable salt thereof, or a pharmaceutical composition according to paragraph 31, for use in therapy.
   Paragraph 33. A compound according to any one of paragraphs 1 to 30, or a pharmaceutically acceptable salt thereof, or a pharmaceutical composition according to paragraph 31, for use in the treatment of a proliferative condition.
   Paragraph 34. A compound according to any one of paragraphs 1 to 30, or a pharmaceutically acceptable salt thereof, or a pharmaceutical composition according to paragraph 31, for use in the treatment of cancer.
   Paragraph 35. A compound according to any one of paragraphs 1 to 30, or a pharmaceutically acceptable salt thereof, or a pharmaceutical composition according to paragraph 31, for use in the treatment of leukaemia.
   Paragraph 36. A compound according to any one of paragraphs 1 to 30, or a pharmaceutically acceptable salt thereof, or a pharmaceutical composition according to paragraph 31, for use in the treatment of AML leukaemia or chronic myeloid leukaemia.
   Paragraph 37. A compound according to any one of paragraphs 1 to 30, or a pharmaceutically acceptable salt thereof, or a pharmaceutical composition according to paragraph 31, for use in the inhibition of METTL3 activity.
   Paragraph 38. A compound according to any one of paragraphs 1 to 30, or a pharmaceutically acceptable salt thereof, or a pharmaceutical composition according to paragraph 31, for use in the treatment of an autoimmune disease, a neurological disease, an inflammatory disease or an infectious disease.
   Paragraph 39. Use of a compound according to any one of paragraphs 1 to 30, or a pharmaceutically acceptable salt thereof, in the manufacture of a medicament for the treatment of a proliferative condition.
   Paragraph 40. Use of a compound according to any one of paragraphs 1 to 30, or a pharmaceutically acceptable salt thereof, in the manufacture of a medicament for the treatment of cancer.
   Paragraph 41. Use of a compound according to any one of paragraphs 1 to 30, or a pharmaceutically acceptable salt thereof, in the manufacture of a medicament for the treatment of leukaemia.
   Paragraph 42. Use of a compound according to any one of paragraphs 1 to 30, or a pharmaceutically acceptable salt thereof, in the manufacture of a medicament for the treatment of AML leukaemia or chronic myeloid leukaemia.
   Paragraph 43. Use of a compound according to any one of paragraphs 1 to 30, or a pharmaceutically acceptable salt thereof, in the manufacture of a medicament for the treatment of an autoimmune disease, a neurological disease, an inflammatory disease or an infectious disease.
   Paragraph 44. Use of a compound according to any one of paragraphs 1 to 30, or a pharmaceutically acceptable salt thereof, in the manufacture of a medicament for the inhibition of METTL3 activity.
   Paragraph 45. A method of treating a proliferative disorder, said method comprising administering to a subject in need thereof a therapeutically effective amount of a compound according to any one of paragraphs 1 to 30, or a pharmaceutically acceptable salt thereof, or a pharmaceutical composition according to paragraph 31.
   Paragraph 46. A method of treating cancer, said method comprising administering to a subject in need thereof a therapeutically effective amount of a compound according to any one of paragraphs 1 to 30, or a pharmaceutically acceptable salt thereof, or a pharmaceutical composition according to paragraph 31.
   Paragraph 47. A method of treating leukaemia, said method comprising administering to a subject in need thereof a therapeutically effective amount of a compound according to any one of paragraphs 1 to 30, or a pharmaceutically acceptable salt thereof, or a pharmaceutical composition according to paragraph 31.
   Paragraph 48. A method of treating AML leukaemia or chronic myeloid leukaemia, said method comprising administering to a subject in need thereof a therapeutically effective amount of a compound according to any one of 1 to 30, or a pharmaceutically acceptable salt thereof, or a pharmaceutical composition according to paragraph 31.
   Paragraph 49. A method of treating an autoimmune disease, a neurological disease, an inflammatory disease or an infectious disease, said method comprising administering to a subject in need thereof a therapeutically effective amount of a compound according to any one of paragraphs 1 to 30, or a pharmaceutically acceptable salt thereof, or a pharmaceutical composition according to paragraph 31.
   Paragraph 50. A method of inhibiting METTL3 activity in vitro or in vivo, said method comprising contacting a cell with an effective amount of a compound according to any one of paragraphs 1 to 30, or a pharmaceutically acceptable salt thereof, or a pharmaceutical composition according to paragraph 31.
   Paragraph 51. A method of inhibiting metastasis in vitro or in vivo, said method comprising contacting a cell with an effective amount of a compound according to any one paragraphs 1 to 30, or a pharmaceutically acceptable salt thereof, or a pharmaceutical composition according to paragraph 31.
   Paragraph 52. A combination comprising a compound according to any one of paragraphs 1 to 30, or a pharmaceutically acceptable salt there, with one or more additional therapeutic agents.

1. A compound of the formula (I), or a pharmaceutically acceptable salt thereof,
X—Y—Z (I)
wherein:
X is selected from: 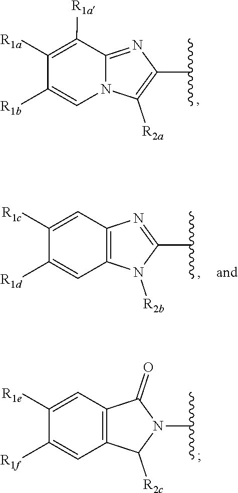
wherein:
R₁ₐ, R_{1c} and R₁ₑ are selected from hydrogen, halo, C₁₋₄ alkyl, C₂₋₃ alkenyl and —O—C₁₋₄ alkyl
R_{1b}, R_{1d} and R_{1f} are selected from:
(i) C₁₋₄ alkyl or C₁₋₄ alkoxy, each of which being optionally substituted by halo, cyano, hydroxy, C₃₋₆ cycloalkyl, C₁₋₄ alkoxy, C₁₋₄ haloalkoxy, aryl or heteroaryl; or
(ii) a group of the formula:
—(CR_{1c}R_{1d})ₚ—NR₁ₑR_{1f};
wherein
p is an integer selected from 0, 1, 2 or 3
R_{1c} and R_{1d} are independently selected from:
(i) hydrogen (including deuterium),
(ii) C₁₋₆ alkyl which is optionally substituted by one more substituents selected from cyano, oxo, hydroxy, C₁₋₄ alkoxy, halo, C₁₋₄ haloalkoxy, C₃₋₆ cycloalkyl, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkyl; and wherein C₃₋₆ cycloalkyl and —O—C₃₋₆ cycloalkyl are optionally further substituted with halo, cyano or hydroxy;
(iii) C₃₋₄ cycloalkyl or 3 to 5 membered heterocyclyl, each of which is optionally substituted by C₁₋₄ alkyl, C₁₋₄ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl; and;
or R_{1c} and R_{1d} are linked together such that, together with the carbon atom to which they are attached, they form a 3- to 6-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ca}R_{1da} or —S(O)₀₋₂R_{1ca}R_{1da}, wherein R_{1ca} and R_{1da} are H or C₁₋₂ alkyl;
R₁ₑ and R_{1f} are each independently selected from:
(i) hydrogen (including deuterium);
(ii) C₁₋₆ alkyl which is optionally substituted by one more substituents selected from cyano, oxo, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
(iii) a group with the formula:
—(CR_{1g}R₁ₕ)_{q}-T₁
wherein:
q is 0, 1, 2, 3, 4, 5 or 6;
R_{1g} and R₁ₕ are independently selected from:
a) hydrogen;
b) C₁₋₆ alkyl which is optionally substituted by one more substituents selected from cyano, oxo, hydroxy, C₁₋₄ alkoxy, halo, C₁₋₄-haloalkoxy, —O—C₃₋₆ cycloalkyl, NR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
c) an aryl-C₁₋₆ alkyl, heteroarylC₁₋₆ alkyl, C₃₋₆ cycloalkyl or C₃₋₆ cycloalkylC₁₋₆ alkyl group, each of which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl; or
d) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂ alkyl, cyano, C₁₋₂ haloalkyl, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
and T₁ is selected from hydrogen, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring, which in turn is optionally substituted by one or more substituents selected from C₁₋₄ alkyl, C₁₋₄ haloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢ and R₁ⱼ are H or C₁₋₄ alkyl;
R_{1a′} is selected from hydrogen, halo and methyl;
R₂ₐ, R_{2b} and R_{2c} are selected from hydrogen, halo or a group of the formula:
-L₂ₐ-L_{2b}-Q₂
wherein
L₂ₐ is absent or C₁₋₃ alkylene optionally substituted by C₁₋₂ alkyl or oxo;
L_{2b} is absent or selected from O, S, SO, SO₂, N(Rₙ), C(O), C(O)O, OC(O), C(O)N(Rₙ), N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂ alkyl; and
Q₂ is hydrogen, cyano, C₁₋₆ alkyl, C₃₋₆ cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄ alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)_{z}NRₚR_{q} (where z is 1, 2 or 3), wherein Rₚ and R_{q} are each independently selected from hydrogen or C₁₋₄ alkyl;
Y is selected from: 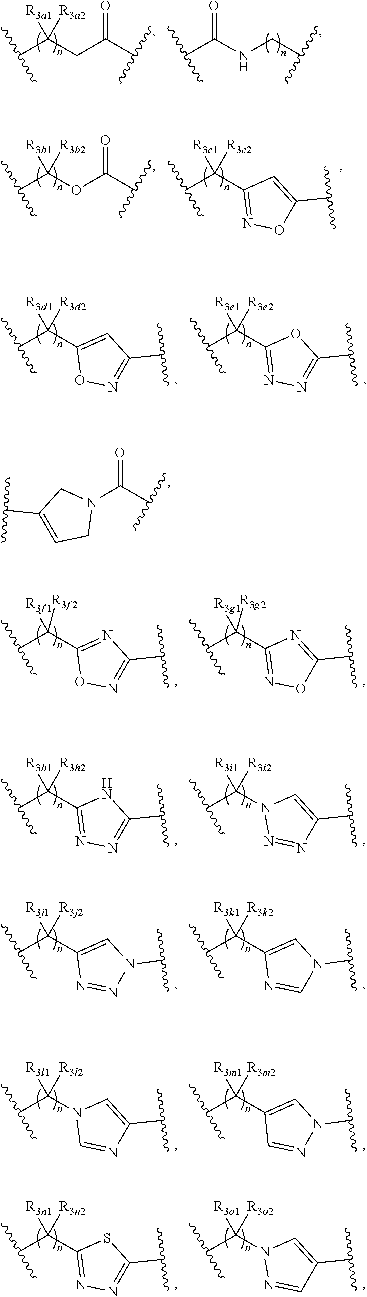 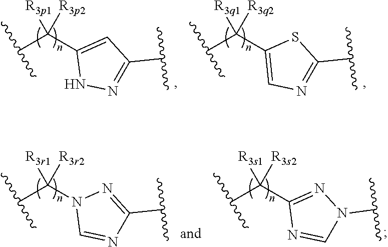
wherein:
R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen (including deuterium), C₁₋₆ alkyl, C₃₋₄ cycloalkyl, hydroxy, and halo; and wherein C₁₋₆ alkyl, or C₃₋₄ cycloalkyl is optionally substituted with one or more substituents selected from halo, amino, cyano, and hydroxy;
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen or halo;
with the proviso that R₃ₐ₁, R_{3b1}, R₃ᵢ₁, R₃ₗ₁, R₃ₒ₁, R₃ᵣ₁, R₃ₐ₂, R_{3b2}, R₃ᵢ₂, R₃ₗ₂, R₃ₒ₂ and R₃ₛ₁ cannot be halo when n=1 or when n=2 and the carbon atom to which they are attached is linked to an oxygen or nitrogen atom;
or R₃ₐ₁ and R₃ₐ₂, R_{3b1} and R_{3b2}, R₃ₐ₁ and R₃ₒ₂, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, or R₃ᵣ₁ and R₃ᵣ₂ or R₃₁ and R₃ₛ₂ may be linked such that, together with the carbon atom to which they are attached, they form a spiro-fused C₃₋₄ cycloalkyl which is optionally substituted with one or more substituents selected from halo, methyl, amino, cyano, and hydroxy;
n is 0, 1 or 2; and
Z is selected from: 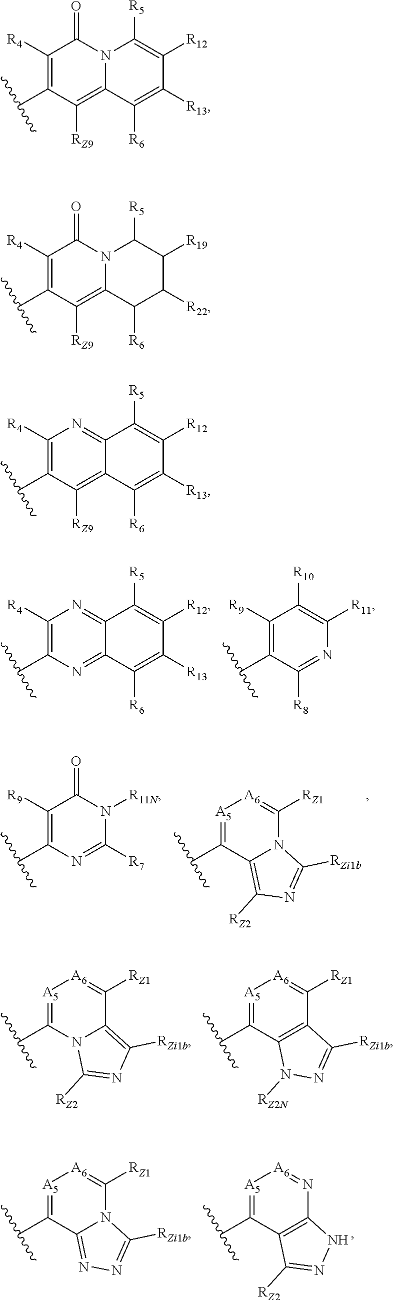 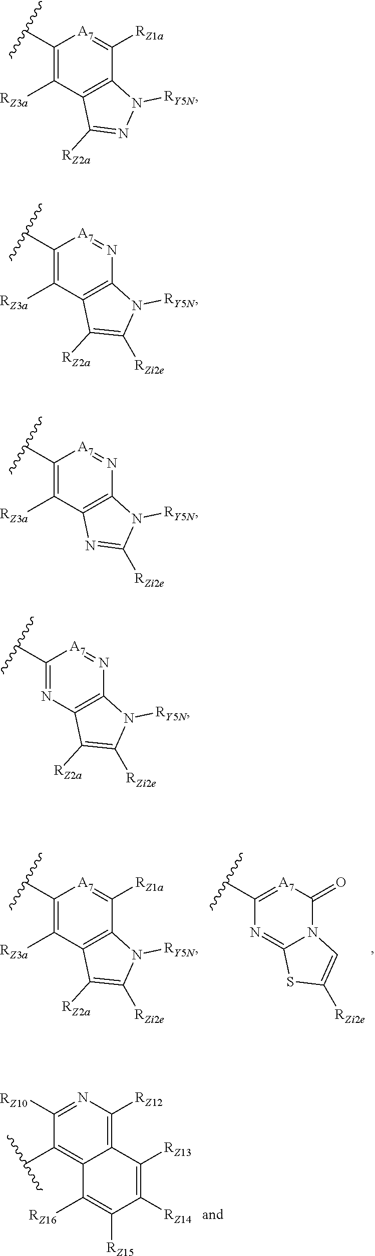 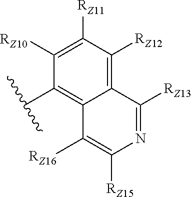
wherein:
R₄ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₅ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₆ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and methyl);
R₁₂ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano and C₁₋₄ alkyl);
R₁₃ is selected from hydrogen, halo, cyano, C₁₋₄ alkyl, C₁₋₄ haloalkyl, C₁₋₄ alkoxy, C₁₋₄ haloalkoxy (e.g. hydrogen, halo, cyano, methoxy and methyl);
either:
a) R₇, R₉ and R₁₁ are independently selected from hydrogen, NH₂, halo, cyano, C₁₋₄ alkoxy, C₁₋₄ haloalkoxy and C₁₋₆ alkyl; and
R₈ and R₁₀ are independently selected from halo, cyano, C₁₋₄ alkoxy, C₁₋₄ haloalkoxy, C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl; or
b) R₈, R₉, R₁₀ and R₁₁ are independently selected from hydrogen, NH₂, halo, cyano, C₁₋₄ alkoxy, C₁₋₄ haloalkoxy and C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl with the proviso that at least one of R₈, R₉, R₁₀ and R₁₁ is C₁₋₄ alkoxy or C₁₋₄ haloalkoxy;
R_{11N} is independently selected from hydrogen and C₁₋₆ alkyl;
R_{Z1} and R_{Z1a} selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl, wherein C₃₋₆ cycloalkyl and —O—C₃₋₆ cycloalkyl are optionally substituted by one or more of halo, methyl or methoxy;
R_{Z2} and R_{Z2a} are selected from hydrogen, C₁₋₄ alkyl, cyano, halo, NH₂ and C₁₋₄ alkoxy;
R_{Z3a} is selected from hydrogen, C₁₋₄ alkyl, cyano, halo, NH₂ and C₁₋₄ alkoxy;
R_{Zi1b} is selected from hydrogen, C₁₋₄ alkyl, cyano, halo, NH₂ and C₁₋₄ alkoxy;
R_{Zi2e} is selected from hydrogen, C₁₋₄ alkyl, cyano, halo, NH₂ and C₁₋₄ alkoxy;
R_{Z2N} is selected from hydrogen or C₁₋₄ alkyl
R_{Y5N} is selected from hydrogen or C₁₋₄ alkyl
R_{Z9} is selected from hydrogen, halo, cyano, C₁₋₄ alkyl, C₁₋₄ haloalkyl, C₁₋₄ alkoxy, C₁₋₄ haloalkoxy;
R_{Z10} is selected from hydrogen, halo, cyano, C₁₋₄ alkyl, C₁₋₄ haloalkyl, C₁₋₄ alkoxy, C₁₋₄ haloalkoxy;
R_{Z11} is selected from hydrogen, halo, cyano, C₁₋₄ alkyl, C₁₋₄ haloalkyl, C₁₋₄ alkoxy, C₁₋₄ haloalkoxy;
R_{Z12} is selected from hydrogen, halo, cyano, C₁₋₄ alkyl, C₁₋₄ haloalkyl, C₁₋₄ alkoxy, C₁₋₄ haloalkoxy;
R_{Z13} is selected from hydrogen, halo, cyano, C₁₋₄ alkyl, C₁₋₄ haloalkyl, C₁₋₄ alkoxy, C₁₋₄ haloalkoxy;
R_{Z14} is selected from hydrogen, halo, cyano, C₁₋₄ alkyl, C₁₋₄ haloalkyl, C₁₋₄ alkoxy, C₁₋₄ haloalkoxy;
R_{Z15} is selected from hydrogen, halo, cyano, C₁₋₄ alkyl, C₁₋₄ haloalkyl, C₁₋₄ alkoxy, C₁₋₄ haloalkoxy;
R_{Z16} is selected from hydrogen, halo, cyano, C₁₋₄ alkyl, C₁₋₄ haloalkyl, C₁₋₄ alkoxy, C₁₋₄ haloalkoxy;
A₅ is selected from CR₁₆ and N;
A₆ is selected from CR₁₇ and N;
A₇ is selected from CR₁₈ and N;
R₁₅ is selected from hydrogen, halo, cyano, methoxy and methyl;
R₁₆ and R₁₈, are selected from hydrogen, halo, cyano, C₁₋₄ alkyl, C₁₋₄ alkoxy, C₁₋₄ haloalkyl, C₁₋₄ haloalkoxy, C₃₋₄ cycloalkyl, a 3- to 4-membered heterocyclyl and C₃₋₄-cycloalkoxy;
R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O-heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₅ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring; wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O— heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl;
R₁₉ is selected from hydrogen, halo, cyano and C₁₋₄ alkyl;
R₂₂ is selected from hydrogen, halo, cyano, C₁₋₄ alkyl, C₁₋₄ haloalkyl, C₁₋₄ alkoxy, C₁₋₄ haloalkoxy.

2. A compound according to claim 1, or a pharmaceutically acceptable salt thereof, wherein R_{1b}, R_{1d} and R_{1f} are a group of the formula:
—(CR_{1c}R_{1d})ₚ—NR₁ₑR_{1f};
wherein
p is an integer selected from 1 or 2;
R_{1ca}nd R_{1d} are independently selected from:
(i) hydrogen (including deuterium),
(ii) C₁₋₃ alkyl which is optionally substituted by one more substituents selected from cyano, oxo, hydroxy, C₁₋₃ alkoxy, halo, C₁₋₄₃ haloalkoxy, —O—C₃₋₄ cycloalkyl, or NH₂; wherein —O—C₃₋₆ cycloalkyl is optionally substituted with halo, cyano or hydroxy,
(iii) or R_{1c} and R_{1d} are linked together such that, together with the carbon atom to which they are attached, they form a 3- to 5-membered cycloalkyl or heterocyclic ring, or a spirocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ca}R_{1da} or —S(O)₀₋₂R_{1ac}R_{1da}, wherein R_{1ca} and R_{1da} are H or C₁₋₂ alkyl;
R₁ₑ is selected from:
(i) hydrogen (including deuterium);
(ii) C₁₋₃ alkyl which is optionally substituted by one more substituents selected from cyano, oxo, hydroxy, C₁₋₂ alkoxy, halo, C₁₋₂ haloalkoxy and NH₂;
and R_{1f} is selected from:
(i) C₁₋₆ alkyl which is optionally substituted by one more substituents selected from cyano, oxo, hydroxy, C₁₋₂ alkoxy, halo, C₁₋₂ haloalkoxy and NH₂;
(ii) a group with the formula:
—(CR_{1g}R₁ₕ)_{q}-T₁
wherein:
q is 1, 2 or 3;
R_{1g} and R₁ₕ are independently selected from:
a) hydrogen (including deuterium); or
b) C₁₋₆ alkyl which is optionally substituted by one more substituents selected from cyano, oxo, hydroxy, C₁₋₄ alkoxy, halo, C₁₋₄ haloalkoxy, —O—C₃₋₆ cycloalkyl, NR_{1ca}R_{1da} or —S(O)₀₋₂R_{1ca}R_{1da}, wherein R_{1ca} and R_{1da} are H or C₁₋₂ alkylNR_{1ga}R₁ₕₐ or —S(O)₀₋₂R_{1ga}R₁ₕₐ, wherein R_{1ga} and R₁ₕₐ are H or C₁₋₂ alkyl; and wherein —O—C₃₋₆ cycloalkyl is optionally substituted with halo, cyano or hydroxy;
c) or R_{1g} and R₁ₕ are optionally linked together such that, together with the carbon atom to which they are attached, they form a 3- to 4-membered cycloalkyl or heterocyclic ring which is optionally substituted by one or more substituents selected from C₁₋₂ alkyl, C₁₋₂ haloalkyl, cyano, hydroxy, C₁₋₂ alkoxy, halo, C₁₋₂ haloalkoxy, NR_{1ga}R₁ₕₐ or —S(O)₀₋₂R_{1ga}R₁ₕₐ, wherein R_{1ga} and R₁ₕₐ are H or C₁₋₂ alkyl;
and T₁ is selected from hydrogen, halo, C₁₋₄ alkyl, C₁₋₄ haloalkyl, cyano, hydroxy, NR₁ₜR₂ₜ or —S(O)₀₋₂R₁ₜR₂ₜ (wherein R₁ₜ and R₂ₜ are H or C₁₋₄ alkyl), C₃₋₈ cycloalkyl, C₂₋₃ alkenyl, C₂₋₃ alkynyl, aryl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂-haloalkoxy, C₃₋₆ cycloalkyl, NR₃ₜR₄ₜ or —S(O)₀₋₂R₃ₜR₄ₜ, wherein R₃ₜ and R₄ₜ are H or C₁₋₂ alkyl;
or R₁ₑ and R_{1f} are linked such that, together with the nitrogen atom to which they are attached, they form a mono- or bicyclic-heterocyclic ring, which is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢR₁ⱼ are H or C₁₋₄ alkyl, and/or the mono- or bicyclic heterocyclic ring formed by R₁ₑ and R_{1f} is optionally spiro-fused to a C₃₋₆ cycloalkyl or a heterocyclic ring; which in turn is optionally substituted by one or more substituents selected from C₁₋₄ alkyl, C₁₋₄ haloalkyl, C₃₋₆ cycloalkyl, cyano, hydroxy, C₁₋₄ alkoxy, halo, C₁₋₄ haloalkoxy, NR₁ᵢR₁ⱼ or —S(O)₀₋₂R₁ᵢR₁ⱼ, wherein R₁ᵢR₁ⱼ are H or C₁₋₄ alkyl;
wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.

3. A compound according to any one of the preceding claims, or a pharmaceutically acceptable salt thereof, wherein R_{1b}, R_{1d} and R_{1f} are a group of the formula:
—(CR_{1c}R_{1d})ₚ—NR₁ₑR_{1f};
wherein
p is 1;
R_{1ca}nd R_{1d} are independently selected from hydrogen (including deuterium) or C₁₋₂ alkyl;
R₁ₑ is selected from hydrogen (including deuterium) or C₁₋₂ alkyl; and
R_{1f} is a group with the formula:
—(CR_{1g}R₁ₕ)_{q}-T₁
wherein:
q is 1 or 2;
R_{1g} and R₁ₕ are independently selected from hydrogen (including deuterium) or C₁₋₂ alkyl;
and T₁ is selected from C₃₋₄ cycloalkyl, heterocyclyl, a mono- or bicyclic heteroaryl, a spirocyclic carbocyclic or heterocyclic ring system, a bridged C₃₋₈ cycloalkyl, a bridged bicyclic C₅₋₁₂ cycloalkyl, or a bridged heterocyclic ring system, each of which is optionally substituted by one or more substituents selected from C₁₋₂ alkyl, C₁₋₂ haloalkyl, cyano, hydroxy, C₁₋₂ alkoxy, halo, C₁₋₂ haloalkoxy or C₃₋₆ cycloalkyl;
or R₁ₑ and R_{1f} are linked such that, together with the nitrogen atom to which they are attached, they form a mono- or bicyclic-heterocyclic ring, which is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, and/or the mono- or bicyclic heterocyclic ring formed by R₁ₑ and R_{1f} is optionally spiro-fused to a C₃₋₆ cycloalkyl or a heterocyclic ring; which in turn is optionally substituted by one or more substituents selected from C₁₋₂ alkyl, C₁₋₂ haloalkyl, C₃₋₆ cycloalkyl, cyano, hydroxy, C₁₋₂ alkoxy, halo or C₁₋₂ haloalkoxy, wherein any alkyl, alkoxy or C₃₋₆ cycloalkyl is further optionally substituted by one or more substituents selected from cyano, hydroxy, halo, NR₁ₖR₁ₗ or —S(O)₀₋₂R₁ₖR₁ₗ, wherein R₁ₖ and R₁ₗ are H or C₁₋₄ alkyl.

4. A compound according to any one of the preceding claims, or a pharmaceutically acceptable salt thereof, wherein R₁ₐ, R_{1c}, R₁ₑ R₂ₐ, R_{2b} and R_{2c} are hydrogen.

5. A compound according to any one of the preceding claims, or a pharmaceutically acceptable salt thereof, wherein R_{1b}, R_{1d} and R_{1f} are selected from:

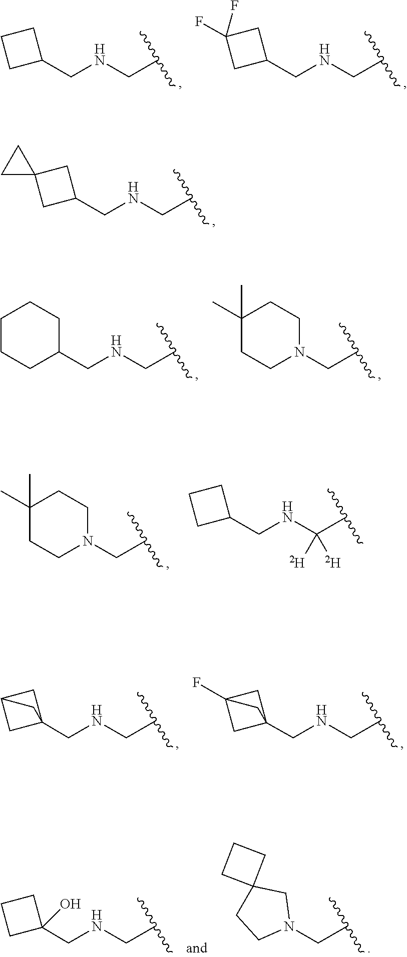

6. A compound according to any one of the preceding claims, or a pharmaceutically acceptable salt thereof, wherein X is: 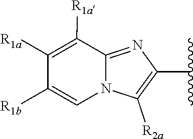
and R₁ₐ, R_{1b}, R_{1a′} and R₂ₐ are as defined in any of the preceding claims.

7. A compound according to any one of the preceding claims, or a pharmaceutically acceptable salt thereof, wherein Y is selected from: 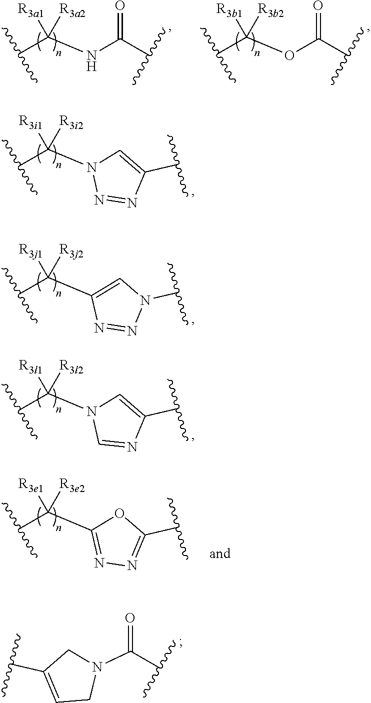
wherein R₃ₐ₁, R₃ₐ₂, R_{3b1}, R_{3b2}, R₃ₑ₁, R₃ₑ₂, R₃ᵢ₁, R₃ᵢ₂, R₃ⱼ₁, R₃ⱼ₂, R₃ᵢ₁ and R₃ᵢ₂, are as defined in any one of the preceding claims.

8. A compound according to any one of the preceding claims, or a pharmaceutically acceptable salt thereof, wherein, R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen and C₁₋₆ alkyl; and wherein C₁₋₆ alkyl is optionally substituted with one or more hydroxy substituents and/or
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen.

9. A compound according to any one of the preceding claims, or a pharmaceutically acceptable salt thereof, wherein n is 1.

10. A compound according to any one of the preceding claims, or a pharmaceutically acceptable salt thereof, wherein Y is selected from:

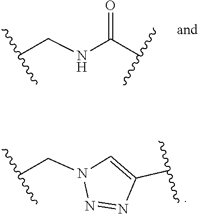 [SMILES: *CNC(*)=O ; *Cn1cc(*)nn1]

11. A compound according to any one of the preceding claims, or a pharmaceutically acceptable salt thereof, wherein
A₅ is CR₁₆;
A₆ is CR₁₇;
A₇ is CR₁₈; and
(i) R₄, R₅, R₆, R₇, R₉, R₁₁, R_{Z2}, R_{Z2a}, R_{Z3a}, R_{Zi1b}, R_{Zi2e}, R_{Z9}, R_{Z10}, R_{Z11}, R_{Z12}, R_{Z13}, R_{Z14}, R_{Z15} and R_{Z16} are independently selected from hydrogen, methyl, cyano or halo; and
R_{Y5N}, and R_{11N} are selected from methyl or hydrogen;
(ii) R₈ is selected from hydrogen, cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl;
(iii) R₁₀ is selected from C₁₋₄ alkoxy or C₁₋₄ haloalkoxy;
(iv) R_{Z1} and R_{Z1a} are selected from hydrogen, C₁₋₄ alkyl, cyano, halo, C₁₋₄ haloalkyl, C₁₋₄ haloalkoxy, C₁₋₄ alkoxy, C₃₋₆ cycloalkyl and —O—C₃₋₆ cycloalkyl;
(v) R₁₂, R₁₃, R₁₆, R₁₈, R₁₉, R₂₀, R₂₁, R₂₂, R₂₃, R₂₄ and R₃₀ are independently selected from hydrogen, halo, cyano and methyl;
(vi) R₁₇ is selected from hydrogen, halo, cyano, C₁₋₄ alkyl, C₁₋₄ haloalkyl, C₁₋₄ alkoxy, C₁₋₄ haloalkoxy, C₂₋₄ alkenyl, C₂₋₄ alkynyl, phenyl a 5- or 6-membered or heteroaryl, C₃₋₆ cycloalkyl, —O—C₃₋₆ cycloalkyl, heterocyclyl, —(OCH₂CH₂)ₘ—OCH₃ wherein m is an integer from 1 to 6, NR_{q}Rᵣ, wherein R_{q} and Rᵣ are each independently hydrogen, C₁₋₄ alkyl or R_{q} and Rᵣ are linked together such that, together with the nitrogen atom to which they are attached, they form a 3- to 6-membered heterocyclic ring; wherein any C₁₋₄ alkyl, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl.

12. A compound according to any one of the preceding claims, or a pharmaceutically acceptable salt thereof, wherein Z is selected from: 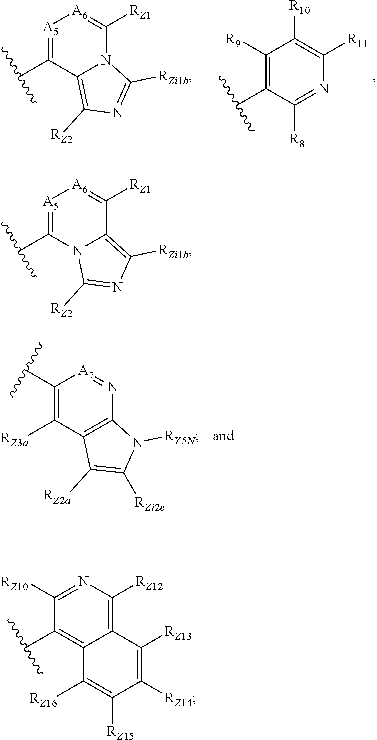
wherein A₅, A₆, A₇, R₄, R₅, R₆, R₇, R₈, R_{B5N}, R_{Y5N}, R_{Z1}, R_{Z2}, R_{Z10}, R_{Z12}, R_{Z13}, R_{Z14}, R_{Z15}, R_{Z16}, R_{Z2a}, R_{Z3a}, R_{Z1a}, R_{Zi1b} and R_{Zi2e} are as defined in any one of the preceding claims.

13. A compound according to any one of the preceding claims, or a pharmaceutically acceptable salt thereof, wherein Z is: 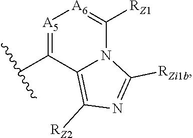
wherein A₅, A₆, R_{Z1}, R_{Z2} and R_{Zi1b} are as defined in any one of the preceding claims.

14. A compound according to any one of the preceding claims, or a pharmaceutically acceptable salt thereof, wherein A₆ is CR₁₇; and
wherein R₁₇ is selected from selected from hydrogen, halo, cyano, C₁₋₅ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆ cycloalkyl, —O—C₃₋₆ cycloalkyl, heterocyclyl, —O— heterocyclyl (carbon-linked), —(OCH₂CH₂)ₘ—OCH₃ wherein m is an integer from 1 to 6, NR_{q}Rᵣ, wherein R_{q} and Rᵣ are each independently hydrogen, C₁₋₄ alkyl, C₃₋₆ cycloalkyl, a 3- to 6-membered carbon-linked heterocyclyl, or R_{q} and Rᵣ are linked together such that, together with the nitrogen atom to which they are attached, they form a 3- to 6-membered heterocyclic ring;
wherein any C₁₋₅ alkyl, C₁₋₄ alkoxy, C₂₋₄ alkenyl, C₂₋₄ alkynyl, phenyl, 5- or 6-membered or heteroaryl, C₃₋₆ cycloalkyl, —O—C₃₋₆ cycloalkyl, heterocyclyl or —O-heterocyclyl (carbon-linked) is optionally further substituted by one or more substituents selected from C₁₋₂ alkyl, cyano, C₁₋₂ haloalkyl, hydroxy, C₁₋₂ alkoxy, halo, C₁₋₂ haloalkoxy, NR₁ₑₐR_{1fa} or —S(O)₀₋₂R₁ₑₐR_{1fa}, wherein R₁ₑₐ and R_{1fa} are H or C₁₋₂ alkyl; optionally wherein R₁₇ is selected from hydrogen, halo, C₁₋₄ alkoxy or C₁₋₄ haloalkoxy.

15. A compound according to claim 14, or a pharmaceutically acceptable salt thereof, wherein A₅ is R₁₆, and R₁₆ is selected from hydrogen, halo, cyano and C₁₋₄ alkyl.

16. A compound according to any one of the preceding claims, or a pharmaceutically acceptable salt thereof, wherein:
X is 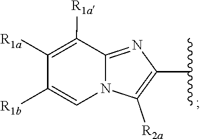
Y is selected from: 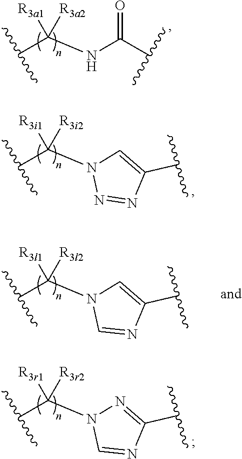
and
Z is selected from: 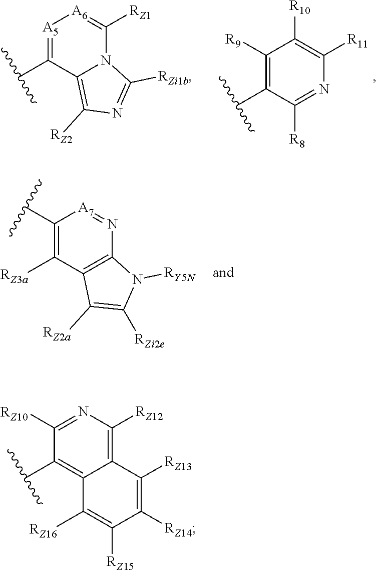
wherein
(i) R₁ₐ, R_{1a′}, R_{1b}, and R₂ₐ are as defined in any one of the preceding claims;
(ii) R₃ₐ₁, R₃ₐ₂, R₃ᵢ₁, R₃ᵢ₂, R₃ₗ₁, R₃ₗ₂, R₃ᵣ₁, R₃ᵣ₂, are as defined in any one of the preceding claims;
(iii) A₅, A₆, A₇, R₈, R₉, R₁₀, R₁₁, R_{Z1}, R_{Z2}, R_{Zi1b}, R_{Z10}, R_{Z12}, R_{Z13}, R_{Z14}, R_{Z15}, R_{Z16}, R_{Y5N}, R_{Z2a}, R_{Z3a}, and R_{Zi2e} are as defined in any one of the preceding claims.

17. A compound according to claim 16, or a pharmaceutically acceptable salt thereof, wherein:
X is 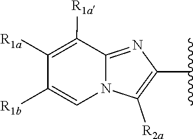
Y is 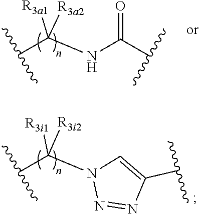
and
Z is 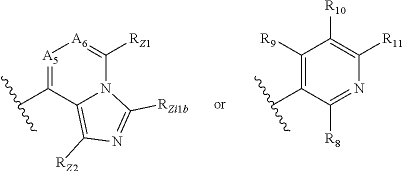
wherein:
(i) R₁ₐ, R_{1a′}, R_{1b}, and R₂ₐ are as defined in any one of the preceding claims;
(ii) R₃ₐ₁, R₃ₐ₂, R₃ᵢ₁ and R₃ᵢ₂ are as defined in any one of the preceding claims;
(iii) A₅, A₆, R_{Z1}, R_{Z2} and R_{Zi1b} are as defined in any one of the preceding claims;
R₈ is selected from hydrogen, cyano, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl,
R₉ and R₁₁ are as defined in any preceding claim;
R₁₀ is C₁₋₄ alkoxy or C₁₋₄ haloalkoxy.

18. A compound, or a pharmaceutically acceptable salt thereof, and, in particular, selected from:
N-[(6-{[(cyclohexylmethyl)amino]methyl}imidazo[1,2-a]pyridin-2-yl)methyl]-1,2-dimethyl-6-oxo-1,6-dihydropyrimidine-4-carboxamide;
N-({6-methylimidazo[1,2-a]pyridin-2-yl}methyl)-4-oxo-4H-quinolizine-2-carboxamide;
N-[(6-{[(cyclohexylmethyl)amino]methyl}imidazo[1,2-a]pyridin-2-yl)methyl]-4-oxo-4H-quinolizine-2-carboxamide;
N-[(6-{[(cyclohexylmethyl)amino]methyl}imidazo[1,2-a]pyridin-2-yl)methyl]quinoxaline-2-carboxamide;
N-[(6-{[(cyclohexylmethyl)amino]methyl}imidazo[1,2-a]pyridin-2-yl)methyl]-1H-pyrazolo[3,4-b]pyridine-4-carboxamide;
N-[(6-{[(cyclohexylmethyl)amino]methyl}imidazo[1,2-a]pyridin-2-yl)methyl]quinoline-3-carboxamide;
N-[(6-{[(cyclobutylmethyl)amino]methyl}imidazo[1,2-a]pyridin-2-yl)methyl]imidazo[1,5-a]pyridine-8-carboxamide;
N-[(6-{[(cyclobutylmethyl)amino]methyl}imidazo[1,2-a]pyridin-2-yl)methyl]imidazo[1,5-a]pyridine-5-carboxamide;
N-[(6-{[(cyclobutylmethyl)amino]methyl}imidazo[1,2-a]pyridin-2-yl)methyl]-3H-imidazo[4,5-b]pyridine-6-carboxamide;
N-[(6-{[(cyclobutylmethyl)amino]methyl}imidazo[1,2-a]pyridin-2-yl)methyl]-1H-pyrrolo[2,3-b]pyridine-5-carboxamide;
N-{[6-({[(3,3-difluorocyclobutyl)methyl]amino}methyl)imidazo[1,2-a]pyridin-2-yl]methyl}-1H-pyrrolo[2,3-b]pyridine-5-carboxamide;
N-[(6-{[(cyclobutylmethyl)amino]methyl}imidazo[1,2-a]pyridin-2-yl)methyl]-1-methyl-1H-pyrrolo[2,3-b]pyridine-5-carboxamide;
N-[(6-{[(cyclobutylmethyl)amino]methyl}imidazo[1,2-a]pyridin-2-yl)methyl]-1H-indole-5-carboxamide;
N-[(6-{[(cyclobutylmethyl)amino]methyl}imidazo[1,2-a]pyridin-2-yl)methyl]-5H-pyrrolo[2,3-b]pyrazine-2-carboxamide;
N-[(6-{[({3-fluorobicyclo[1.1.1]pentan-1-yl}methyl)amino]methyl}imidazo[1,2-a]pyridin-2-yl)methyl]-5-oxo-5H-[1,3]thiazolo[3,2-a]pyrimidine-7-carboxamide;
4-[1-[(6-methylimidazo[1,2-a]pyridin-2-yl)methyl]triazol-4-yl]isoquinoline;
1-({6-methylimidazo[1,2-a]pyridin-2-yl}methyl)-4-{1H-pyrrolo[2,3-b]pyridin-5-yl}-1H-1,2,3-triazole;
(cyclobutylmethyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{imidazo[1,5-a]pyridin-8-yl}-1H-1,2,3-triazol-1-yl)methyl]imidazo[1,2-a]pyridin-6-yl}methyl)amine;
({2-[(4-{6-chloroimidazo[1,5-a]pyridin-8-yl}-1H-1,2,3-triazol-1-yl)methyl]imidazo[1,2-a]pyridin-6-yl}methyl)(cyclobutylmethyl)amine;
(cyclobutylmethyl)({2-[(4-{1-fluoroimidazo[1,5-a]pyridin-8-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
(cyclobutylmethyl)({2-[(4-{[1,2,4]triazolo[4,3-a]pyridin-8-yl}-1H-1,2,3-triazol-1-yl)methyl]imidazo[1,2-a]pyridin-6-yl}methyl)amine;
({bicyclo[1.1.1]pentan-1-yl}methyl)({2-[(4-{6-methoxyimidazo[1,5-a]pyridin-8-yl}-1H-1,2,3-triazol-1-yl)methyl]imidazo[1,2-a]pyridin-6-yl}methyl)amine;
2-({2-[(4-{imidazo[1,5-a]pyridin-8-yl}-1H-1,2,3-triazol-1-yl)methyl]imidazo[1,2-a]pyridin-6-yl}methyl)-2-azaspiro[3.3]heptane;
2-({2-[(4-{6-methoxyimidazo[1,5-a]pyridin-8-yl}-1H-1,2,3-triazol-1-yl)methyl]imidazo[1,2-a]pyridin-6-yl}methyl)-2-azaspiro[3.3]heptane;
N-(cyclobutylmethyl)-1-[2-[[4-(4-isoquinolyl)triazol-1-yl]methyl]imidazo[1,2-a]pyridin-6-yl]methanamine;
bis(cyclobutylmethyl)[(2-{[4-(isoquinolin-4-yl)-1H-1,2,3-triazol-1-yl]methyl}imidazo[1,2-a]pyridin-6-yl)methyl]amine;
(cyclobutylmethyl)({2-[(4-{1H-pyrrolo[2,3-b]pyridin-5-yl}-1H-1,2,3-triazol-1-yl)methyl]imidazo[1,2-a]pyridin-6-yl}methyl)amine;
(cyclobutylmethyl)({2-[(4-{1H-pyrrolo[2,3-b]pyridin-5-yl}-1H-imidazol-1-yl)methyl]imidazo[1,2-a]pyridin-6-yl}methyl)amine;
cyclobutylmethyl)({2-[(4-{imidazo[1,5-a]pyridin-5-yl}-1H-1,2,3-triazol-1-yl)methyl]imidazo[1,2-a]pyridin-6-yl}methyl)amine;
({3-fluorobicyclo[1.1.1]pentan-1-yl}methyl)({2-[(4-{3-methylimidazo[1,5-a]pyridin-8-yl}-1H-1,2,3-triazol-1-yl)methyl]imidazo[1,2-a]pyridin-6-yl}methyl)amine;
3-{1-[(6-{[({bicyclo[1.1.1]pentan-1-yl}methyl)amino]methyl}imidazo[1,2-a]pyridin-2-yl)methyl]-1H-1,2,3-triazol-4-yl}-5-methoxypyridine-2-carbonitrile;
3-{1-[(6-{[(cyclobutylmethyl)amino]methyl}imidazo[1,2-a]pyridin-2-yl)methyl]-1H-1,2,3-triazol-4-yl}-5-methoxypyridine-2-carbonitrile;
3-[1-({6-[(4,4-dimethylpiperidin-1-yl)methyl]imidazo[1,2-a]pyridin-2-yl}methyl)-1H-1,2,3-triazol-4-yl]-5-methoxypyridine-2-carbonitrile;
3-(1-{[6-({6-azaspiro[3.4]octan-6-yl}methyl)imidazo[1,2-a]pyridin-2-yl]methyl}-1H-1,2,3-triazol-4-yl)-5-methoxypyridine-2-carbonitrile;
3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}-5-methoxypyridine-2-carbonitrile;
3-(1-{[6-({6-azaspiro[3.4]octan-6-yl}methyl)imidazo[1,2-a]pyridin-2-yl]methyl}-1H-1,2,3-triazol-4-yl)-5-fluoropyridine-2-carbonitrile;
5-fluoro-3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}pyridine-2-carbonitrile;
({3-fluorobicyclo[1.1.1]pentan-1-yl}methyl)[(2-{[4-(6-methoxypyrazin-2-yl)-1H-1,2,3-triazol-1-yl]methyl}imidazo[1,2-a]pyridin-6-yl)methyl]amine;
({3-fluorobicyclo[1.1.1]pentan-1-yl}methyl)[(2-{[4-(5-methoxypyridin-3-yl)-1H-1,2,3-triazol-1-yl]methyl}imidazo[1,2-a]pyridin-6-yl)methyl]amine;
N-[(6-{[(cyclobutylmethyl)amino]methyl}imidazo[1,2-a]pyridin-2-yl)methyl]-5-methoxypyridine-3-carboxamide;
({3-fluorobicyclo[1.1.1]pentan-1-yl}methyl)[(2-{[4-(4-methoxypyridin-3-yl)-1H-1,2,3-triazol-1-yl]methyl}imidazo[1,2-a]pyridin-6-yl)methyl]amine;
({3-fluorobicyclo[1.1.1]pentan-1-yl}methyl)[(2-{[4-(6-methoxypyridin-3-yl)-1H-1,2,3-triazol-1-yl]methyl}imidazo[1,2-a]pyridin-6-yl)methyl]amine;
3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}-5-methoxypyridine-2-carboxamide;
(cyclobutylmethyl)[(2-{[4-(1-methyl-1H-indazol-7-yl)-1H-1,2,3-triazol-1-yl]methyl}imidazo[1,2-a]pyridin-6-yl)methyl]amine;
3-{1-[(6-{[({3-fluorobicyclo[1.1.1]pentan-1-yl}methyl)amino]methyl}imidazo[1,2-a]pyridin-2-yl)methyl]-1H-1,2,3-triazol-4-yl}-5-(morpholin-4-yl)pyridine-2-carbonitrile;
({3-fluorobicyclo[1.1.1]pentan-1-yl}methyl)({[2-({4-[5-(morpholin-4-yl)pyridin-3-yl]-1H-1,2,3-triazol-1-yl}methyl)imidazo[1,2-a]pyridin-6-yl]methyl})amine.

19. A pharmaceutical composition comprising a compound according to any one of claims 1 to 18, or a pharmaceutically acceptable salt thereof, and one or more pharmaceutically acceptable excipients.

20. A compound according to any one of claims 1 to 18, or a pharmaceutically acceptable salt thereof, or a pharmaceutical composition according to claim 19, for use in therapy.

21. A compound according to any one of claims 1 to 18, or a pharmaceutically acceptable salt thereof, or a pharmaceutical composition according to claim 19, for use in the treatment of cancer.

22. A compound according to any one of claims 1 to 18, or a pharmaceutically acceptable salt thereof, or a pharmaceutical composition according to claim 19, for use in the treatment of leukaemia.

23. A compound according to any one of claims 1 to 18, or a pharmaceutically acceptable salt thereof, or a pharmaceutical composition according to claim 19, for use in the treatment of an autoimmune disease, a neurological disease, an inflammatory disease or an infectious disease.

24. A method of treating a proliferative disorder, said method comprising administering to a subject in need thereof a therapeutically effective amount of a compound according to any one of claims 1 to 18, or a pharmaceutically acceptable salt thereof, or a pharmaceutical composition according to claim 19.

25. A combination comprising a compound according to any one of claims 1 to 18, or a pharmaceutically acceptable salt there, with one or more additional therapeutic agents.
